SEQUENCE LISTING (199 sequences)

SEQ ID NO: 1 - protein, 500 aa, synthetic construct
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 2 - protein, 462 aa, synthetic construct
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 3 - protein, 1474 aa, synthetic construct
  CARBOHYD 1424: note = N is linked to the N-linked glycan structure Hex(6)HexNAc(5)Fuc(0)NeuAc(2)
  CARBOHYD 1424: note = N is linked to the N-linked glycan structure Hex(6)HexNAc(5)Fuc(0)NeuAc(3)
  CARBOHYD 1424: note = N is linked to the N-linked glycan structure Hex(6)HexNAc(5)Fuc(1)NeuAc(1)
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 4 - protein, 142 aa, synthetic construct
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 5 - protein, 119 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 6 - protein, 599 aa, synthetic construct
MKLLKLTGFIFFLFFLTESLTLPTQPRDIENFNSTQKFIEDNIEYITIIAFAQYVQEATFEEMEKLVKDMVEYKDRCMADKTLPECSKLPNNVLQEKICAMEGLPQKHNFSHCCSKVDAQRRLCFFYNKKSDVGFLPPFPTLDPEEKCQAYESNRESLLNHFLYEVARRNPFVFAPTLLTVAVHFEEVAKSCCEEQNKVNCLQTRAIPVTQYLKAFSSYQKHVCGALLKFGTKVVHFIYIAILSQKFPKIEFKELISLVEDVSSNYDGCCEGDVVQCIRDTSKVMNHICSKQDSISSKIKECCEKKIPERGQCIINSNKDDRPKDLSLREGKFTDSENVCQERDADPDTFFAKFTFEYSRRHPDLSIPELLRIVQIYKDLLRNCCNTENPPGCYRYAEDKFNETTEKSLKMVQQECKHFQNLGKDGLKYHYLIRLTKIAPQLSTEELVSLGEKMVTAFTTCCTLSEEFACVDNLADLVFGELCGVNENRTINPAVDHCCKTNFAFRRPCFESLKADKTYVPPPFSQDLFTFHADMCQSQNEELQRKTDRFLVNLVKLKHELTDEELQSLFTNFANVVDKCCKAESPEVCFNEESPKIGN

SEQ ID NO: 7 - protein, 19 aa, synthetic construct
VLSNNSDANLELINTWVAK

SEQ ID NO: 8 - protein, 16 aa, synthetic construct
ALPQPQNVTSLLGCTH

SEQ ID NO: 9 - protein, 33 aa, synthetic construct
IITILEEEMNVSVCGLYTYGKPVPGHVTVSICR

SEQ ID NO: 10 - protein, 15 aa, synthetic construct
VGAHAGEYGAEALER

SEQ ID NO: 11 - protein, 24 aa, synthetic construct
SVQEIQATFFYFTPNKTEDTIFLR

SEQ ID NO: 12 - protein, 9 aa, synthetic construct
HNFSHCCSK

SEQ ID NO: 13 - protein, 347 aa, synthetic construct
  CARBOHYD 70: note = T is linked to the N-linked glycan structure Hex(4)HexNAc(4)Fuc(0)NeuAc(1]
  CARBOHYD 107: note = L is linked to the N-linked glycan structure Hex(4)HexNAc(3)Fuc(1)NeuAc(1)
  CARBOHYD 271: note = S is linked to the N-linked glycan structure Hex(5)HexNAc(5)Fuc(0)NeuAc(2)
  CARBOHYD 107: note = L is linked to the N-linked glycan structure Hex(7)HexNAc(6)Fuc(2)NeuAc(4]
  CARBOHYD 107: note = L is linked to the N-linked glycan structure Hex(5)HexNAc(4)Fuc(0)NeuAc(3)
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 14 - protein, 201 aa, synthetic construct
MALSWVLTVLSLLPLLEAQIPLCANLVPVPITNATLDRITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 15 - protein, 201 aa, synthetic construct
MALSWVLTVLSLLPLLEAQIPLCANLVPVPITNATLDRITGKWFYIASAFRNEEYNKSVQEIQATFFYFTPNKTEDTIFLREYQTRQNQCFYNSSYLNVQRENGTVSRYEGGREHVAHLLFLRDTKTLMFGSYLDDEKNWGLSFYADKPETTKEQLGEFYEALDCLCIPRSDVMYTDWKKDKCEPLEKQHEKERKQEEGES

SEQ ID NO: 16 - protein, 485 aa, synthetic construct
MRKRAPQSEMAPAGVSLRATILCLLAWAGLAAGDRVYIHPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA

SEQ ID NO: 17 - protein, 367 aa, synthetic construct
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 18 - protein, 930 aa, synthetic construct
  CARBOHYD 81: note = N is linked to the N-linked glycan structure Hex(5)HexNAc(4)Fuc(1)NeuAc(2)
MKPPRPVRTCSKVLVLLSLLAIHQTTTAEKNGIDIYSLTVDSRVSSRFAHTVVTSRVVNRANTVQEATFQMELPKKAFITNFSMIIDGMTYPGIIKEKAEAQAQYSAAVAKGKSAGLVKATGRNMEQFQVSVSVAPNAKITFELVYEELLKRRLGVYELLLKVRPQQLVKHLQMDIHIFEPQGISFLETESTFMTNQLVDALTTWQNKTKAHIRFKPTLSQQQKSPEQQETVLDGNLIIRYDVDRAISGGSIQIENGYFVHYFAPEGLTTMPKNVVFVIDKSGSMSGRKIQQTREALIKILDDLSPRDQFNLIVFSTEATQWRPSLVPASAENVNKARSFAAGIQALGGTNINDAMLMAVQLLDSSNQEERLPEGSVSLIILLTDGDPTVGETNPRSIQNNVREAVSGRYSLFCLGFGFDVSYAFLEKLALDNGGLARRIHEDSDSALQLQDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSGSTFFKYYLQGAKIPKPEASFSPRRGWNRQAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 19 - protein, 406 aa, synthetic construct
  CARBOHYD 241: note = N is linked to the N-linked glycan structure Hex(4)HexNAc(3)Fuc(1)NeuAc(0)
  CARBOHYD 241: note = N is linked to the N-linked glycan structure Hex(4)HexNAc(5)Fuc(0)NeuAc(1)
  CARBOHYD 241: note = N is linked to the N-linked glycan structure Hex(5)HexNAc(3)Fuc(0)NeuAc(1)
  CARBOHYD 241: note = N is linked to the N-linked glycan structure Hex(5)HexNAc(4)Fuc(0)NeuAc(1)
  CARBOHYD 184: note = N is linked to the N-linked glycan structure Hex(5)HexNAc(5)Fuc(0)NeuAc(1)
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 20 - protein, 525 aa, synthetic construct
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 21 - protein, 644 aa, synthetic construct
MKLITILFLCSRLLLSLTQESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQIDPNGLSFNPISDFPDTTSPKCPGRPWKSVSEINPTTQMKESYYFDLTDGLS

SEQ ID NO: 22 - protein, 911 aa, synthetic construct
MDGAMGPRGLLLCMYLVSLLILQAMPALGSATGRSKSSEKRQAVDTAVDGVFIRSLKVNCKVTSRFAHYVVTSQVVNTANEAREVAFDLEIPKTAFISDFAVTADGNAFIGDIKDKVTAWKQYRKAAISGENAGLVRASGRTMEQFTIHLTVNPQSKVTFQLTYEEVLKRNHMQYEIVIKVKPKQLVHHFEIDVDIFEPQGISKLDAQASFLPKELAAQTIKKSFSGKKGHVLFRPTVSQQQSCPTCSTSLLNGHFKVTYDVSRDKICDLLVANNHFAHFFAPQNLTNMNKNVVFVIDISGSMRGQKVKQTKEALLKILGDMQPGDYFDLVLFGTRVQSWKGSLVQASEANLQAAQDFVRGFSLDEATNLNGGLLRGIEILNQVQESLPELSNHASILIMLTDGDPTEGVTDRSQILKNVRNAIRGRFPLYNLGFGHNVDFNFLEVMSMENNGRAQRIYEDHDATQQLQGFYSQVAKPLLVDVDLQYPQDAVLALTQNHHKQYYEGSEIVVAGRIADNKQSSFKADVQAHGEGQEFSITCLVDEEEMKKLLRERGHMLENHVERLWAYLTIQELLAKRMKVDREERANLSSQALQMSLDYGFVTPLTSMSIRGMADQDGLKPTIDKPSEDSPPLEMLGPRRTFVLSALQPSPTHSSSNTQRLPDRVTGVDTDPHFIIHVPQKEDTLCFNINEEPGVILSLVQDPNTGFSVNGQLIGNKARSPGQHDGTYFGRLGIANPATDFQLEVTPQNITLNPGFGGPVFSWRDQAVLRQDGVVVTINKKRNLVVSVDDGGTFEVVLHRVWKGSSVHQDFLGFYVLDSHRMSARTHGLLGQFFHPIGFEVSDIHPGSDPTKPDATMVVRNRRLTVTRGLQKDYSKDPWHGAEVSCWFIHNNGAGLIDGAYTDYIVPDIF

SEQ ID NO: 23 - protein, 2477 aa, synthetic construct
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 24 - protein, 381 aa, synthetic construct
MWRSLGLALALCLLPSGGTESQDQSSLCKQPPAWSIRDQDPMLNSNGSVTVVALLQASUYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSUCCHCRHLIFEKTGSAITUQCKENLPSLCSUQGLRAEENITESCQURLPPAAUQISQQLIPTEASASURUKNQAKKUEUPSN

SEQ ID NO: 25 - protein, 478 aa, synthetic construct
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 26 - protein, 622 aa, synthetic construct
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 27 - protein, 31 aa, synthetic construct
YLGNATAIFFLPDEGKLQHLENELTHDIITK

SEQ ID NO: 28 - protein, 7 aa, synthetic construct
ENGTISR

SEQ ID NO: 29 - protein, 6 aa, synthetic construct
NEEYNK

SEQ ID NO: 30 - protein, 21 aa, synthetic construct
VYIHPFHLVIHNESTCEQLAK

SEQ ID NO: 31 - protein, 22 aa, synthetic construct
AALAAFNAQNNGSNFQLEEISR

SEQ ID NO: 32 - protein, 16 aa, synthetic construct
ALPQPQNVTSLLGCTH

SEQ ID NO: 33 - protein, 24 aa, synthetic construct
MVSHHNLTTGATLINEQWLLTTAK

SEQ ID NO: 34 - protein, 14 aa, synthetic construct
HSHNNNSSDLHPHK

SEQ ID NO: 35 - protein, 43 aa, synthetic construct
FSVATQTCQITPAEGPVVTAQYDCLGCVHPISTQSPDLEPILR

SEQ ID NO: 36 - protein, 9 aa, synthetic construct
LGVYELLLK

SEQ ID NO: 37 - protein, 13 aa, synthetic construct
SLDFTELDVAAEK

SEQ ID NO: 38 - protein, 14 aa, synthetic construct
SYTITGLQPGTDYK

SEQ ID NO: 39 - protein, 8 aa, synthetic construct
VSLATVDK

SEQ ID NO: 40 - protein, 10 aa, synthetic construct
GQYCYELDEK

SEQ ID NO: 41 - protein, 26 aa, synthetic construct
WVLTAAHCLLYPPWDKNFTENDLLVR

SEQ ID NO: 42 - protein, 32 aa, synthetic construct
ADTHDEILEGLNFNLTEIPEAQIHEGFQELLR

SEQ ID NO: 43 - protein, 16 aa, synthetic construct
YLGNATAIFFLPDEGK

SEQ ID NO: 44 - protein, 16 aa, synthetic construct
YLGNATAIFFLPDEGK

SEQ ID NO: 45 - protein, 30 aa, synthetic construct
QLAHQSNSTNIFFSPVSIATAFAMLSLGTK

SEQ ID NO: 46 - protein, 13 aa, synthetic construct
LPPGLLANFTLLR

SEQ ID NO: 47 - protein, 19 aa, synthetic construct
VSNQTLSLFFTVLQDVPVR

SEQ ID NO: 48 - protein, 21 aa, synthetic construct
IITILEEEMNVSVCGLYTYGK

SEQ ID NO: 49 - protein, 21 aa, synthetic construct
IITILEEEMNVSVCGLYTYGK

SEQ ID NO: 50 - protein, 20 aa, synthetic construct
TLNQSSDELQLSMGNAMFVK

SEQ ID NO: 51 - protein, 20 aa, synthetic construct
TLNQSSDELQLSMGNAMFVK

SEQ ID NO: 52 - protein, 15 aa, synthetic construct
QDQCIYNTTYLNVQR

SEQ ID NO: 53 - protein, 6 aa, synthetic construct
NEEYNK

SEQ ID NO: 54 - protein, 24 aa, synthetic construct
SVQEIQATFFYFTPNKTEDTIFLR

SEQ ID NO: 55 - protein, 24 aa, synthetic construct
SVQEIQATFFYFTPNKTEDTIFLR

SEQ ID NO: 56 - protein, 21 aa, synthetic construct
VYIHPFHLVIHNESTCEQLAK

SEQ ID NO: 57 - protein, 19 aa, synthetic construct
FSEFWDLDPEVRPTSAVAA

SEQ ID NO: 58 - protein, 21 aa, synthetic construct
DKFSEFWDLDPEVRPTSAVAA

SEQ ID NO: 59 - protein, 15 aa, synthetic construct
AATVGSLAGQPLQER

SEQ ID NO: 60 - protein, 15 aa, synthetic construct
AATVGSLAGQPLQER

SEQ ID NO: 61 - protein, 17 aa, synthetic construct
VQAAVGTSAAPVPSDNH

SEQ ID NO: 62 - protein, 11 aa, synthetic construct
LGNWSAMPSCK

SEQ ID NO: 63 - protein, 11 aa, synthetic construct
LGNWSAMPSCK

SEQ ID NO: 64 - protein, 23 aa, synthetic construct
TELFSSSCPGGIMLNETGQGYQR

SEQ ID NO: 65 - protein, 30 aa, synthetic construct
FSLLGHASISCTVENETIGVWRPSPPTCEK

SEQ ID NO: 66 - protein, 20 aa, synthetic construct
ENLTAPGSDSAVFFEQGTTR

SEQ ID NO: 67 - protein, 20 aa, synthetic construct
ENLTAPGSDSAVFFEQGTTR

SEQ ID NO: 68 - protein, 17 aa, synthetic construct
ELHHLQEQNVSNAFLDK

SEQ ID NO: 69 - protein, 17 aa, synthetic construct
ELHHLQEQNVSNAFLDK

SEQ ID NO: 70 - protein, 13 aa, synthetic construct
MDGASNVTCINSR

SEQ ID NO: 71 - protein, 25 aa, synthetic construct
SPYEMFGDEEVMCLNGNWTEPPQCK

SEQ ID NO: 72 - protein, 21 aa, synthetic construct
TVLTPATNHMGNVTFTIPANR

SEQ ID NO: 73 - protein, 21 aa, synthetic construct
TVLTPATNHMGNVTFTIPANR

SEQ ID NO: 74 - protein, 7 aa, synthetic construct
LSSNSTK

SEQ ID NO: 75 - protein, 15 aa, synthetic construct
VCQDCPLLAPLNDTR

SEQ ID NO: 76 - protein, 22 aa, synthetic construct
AALAAFNAQNNGSNFQLEEISR

SEQ ID NO: 77 - protein, 16 aa, synthetic construct
HEEGHMLNCTCFGQGR

SEQ ID NO: 78 - protein, 17 aa, synthetic construct
NGTGHGNSTHHGPEYMR

SEQ ID NO: 79 - protein, 16 aa, synthetic construct
ALPQPQNVTSLLGCTH

SEQ ID NO: 80 - protein, 22 aa, synthetic construct
CSDGWSFDATTLDDNGTMLFFK

SEQ ID NO: 81 - protein, 13 aa, synthetic construct
NLFLNHSENATAK

SEQ ID NO: 82 - protein, 16 aa, synthetic construct
VVLHPNYSQVDIGLIK

SEQ ID NO: 83 - protein, 19 aa, synthetic construct
VIDFNCTTSSVSSALANTK

SEQ ID NO: 84 - protein, 8 aa, synthetic construct
DTFVNASR

SEQ ID NO: 85 - protein, 8 aa, synthetic construct
DTFVNASR

SEQ ID NO: 86 - protein, 21 aa, synthetic construct
VGQLQLSHNLSLVILVPQNLK

SEQ ID NO: 87 - protein, 22 aa, synthetic construct
PALEDLLLGSEANLTCTLTGLR

SEQ ID NO: 88 - protein, 22 aa, synthetic construct
PALEDLLLGSEANLTCTLTGLR

SEQ ID NO: 89 - protein, 9 aa, synthetic construct
TPLTANITK

SEQ ID NO: 90 - protein, 9 aa, synthetic construct
EEQYNSTYR

SEQ ID NO: 91 - protein, 9 aa, synthetic construct
EEQYNSTYR

SEQ ID NO: 92 - protein, 9 aa, synthetic construct
EEQYNSTYR

SEQ ID NO: 93 - protein, 9 aa, synthetic construct
EEQYNSTYR

SEQ ID NO: 94 - protein, 9 aa, synthetic construct
EEQYNSTYR

SEQ ID NO: 95 - protein, 9 aa, synthetic construct
EEQFNSTFR

SEQ ID NO: 96 - protein, 9 aa, synthetic construct
EEQYNSTFR

SEQ ID NO: 97 - protein, 9 aa, synthetic construct
EEQYNSTFR

SEQ ID NO: 98 - protein, 19 aa, synthetic construct
IIVPLNNRENISDPTSPLR

SEQ ID NO: 99 - protein, 21 aa, synthetic construct
GLTFQQNASSMCVPDQDTAIR

SEQ ID NO: 100 - protein, 22 aa, synthetic construct
STGKPTLYNVSLVMSDTAGTCY

SEQ ID NO: 101 - protein, 11 aa, synthetic construct
YKNNSDISSTR

SEQ ID NO: 102 - protein, 11 aa, synthetic construct
YKNNSDISSTR

SEQ ID NO: 103 - protein, 11 aa, synthetic construct
YKNNSDISSTR

SEQ ID NO: 104 - protein, 25 aa, synthetic construct
ICDLLVANNHFAHFFAPQNLTNMNK

SEQ ID NO: 105 - protein, 18 aa, synthetic construct
IYPGVDFGGEELNVTFVK

SEQ ID NO: 106 - protein, 21 aa, synthetic construct
IVGGTNSSWGEWPWQVSLQVK

SEQ ID NO: 107 - protein, 9 aa, synthetic construct
LDVDQALNR

SEQ ID NO: 108 - protein, 26 aa, synthetic construct
WVLTAAHCLLYPPWDKNFTENDLLVR

SEQ ID NO: 109 - protein, 13 aa, synthetic construct
CGLVPVLAENYNK

SEQ ID NO: 110 - protein, 21 aa, synthetic construct
QQQHLFGSNVTDCSGNFCLFR

SEQ ID NO: 111 - protein, 8 aa, synthetic construct
NGSLFAFR

SEQ ID NO: 112 - protein, 347 aa, synthetic construct; identical to SEQ ID NO: 13

SEQ ID NO: 113 - protein, 423 aa, synthetic construct
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 114 - protein, 99 aa, synthetic construct
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 115 - protein, 317 aa, synthetic construct [sequence omitted]

SEQ ID NO: 116 - protein, 345 aa, synthetic construct
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 117 - protein, 188 aa, synthetic construct
MFHQIWAALLYFYGIILNSIYQCPEHSQLTTLGVDGKEFPEVHLGQWYFIAGAAPTKEELATFDPVDNIVFNMAAGSAPMQLHLRATIRMKDGLCVPRKWIYHLTEGSTDLRTEGRPDMKTELFSSSCPGGIMLNETGQGYQRFLLYNRSPHPPEKCVEEFKSLTSCLDSKAFLLTPRNQEACELSNN

SEQ ID NO: 118 - protein, 597 aa, synthetic construct
MHPPKTPSGALHRKRKMAAWPFSRLWKVSDPILFQMTLIAALLPAVLGNCGPPPTLSFAAPMDITLTETRFKTGTTLKYTCLPGYVRSHSTQTLTCNSDGEWVYNTFCIYKRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCEKITCRKPDVSHGEMVSGFGPIYNYKDTIVFKCQKGFVLRGSSVIHCDADSKWNPSPPACEPNSCINLPDIPHASWETYPRPTKEDVYVVGTVLRYRCHPGYKPTTDEPTTVICQKNLRWTPYQGCEALCCPEPKLNNGEITQHRKSRPANHCVYFYGDEISFSCHETSRFSAICQGDGTWSPRTPSCGDICNFPPKIAHGHYKQSSSYSFFKEEIIYECDKGYILVGQAKLSCSYSHWSAPAPQCKALCRKPELVNGRLSVDKDQYVEPENVTIQCDSGYGVVGPQSITCSGNRTWYPEVPKCEWETPEGCEQVLTGKRLMQCLPNPEDVKMALEVYKLSLEIEQLELQRDSARQSTLDKEL

SEQ ID NO: 119 - protein, 1065 aa, synthetic construct
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 120 - protein, 1231 aa, synthetic construct
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 121 - protein, 1663 aa, synthetic construct
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 122 - protein, 934 aa, synthetic construct
  CARBOHYD 855: note = N is linked to the N-linked glycan structure Hex(5)HexNAc(4)Fuc(0)NeuAc(1)
MARRSVLYFILLNALINKGQACFCDHYAWTQWTSCSKTCNSGTQSRHRQIVVDKYYQENFCEQICSKQETRECNWQRCPINCLLGDFGPWSDCDPCIEKQSKVRSVLRPSQFGGQPCTAPLVAFQPCIPSKLCKIEEADCKNKFRCDSGRCIARKLECNGENDCGDNSDERDCGRTKAVCTRKYNPIPSVQLMGNGFHFLAGEPRGEVLDNSFTGGICKTVKSSRTSNPYRVPANLENVGFEVQTAEDDLKTDFYKDLTSLGHNENQQGSFSSQGGSSFSVPIFYSSKRSENINHNSAFKQAIQASHKKDSSFIRIHKVMKVLNFTTKAKDLHLSDVFLKALNHLPLEYNSALYSRIFDDFGTHYFTSGSLGGVYDLLYQFSSEELKNSGLTEEEAKHCVRIETKKRVLFAKKTKVEHRCTTNKLSEKHEGSFIQGAEKSISLIRGGRSEYGAALAWEKGSSGLEEKTFSEWLESVKENPAVIDFELAPIVDLVRNIPCAVTKRNNLRKALQEYAAKFDPCQCAPCPNNGRPTLSGTECLCVCQSGTYGENCEKQSPDYKSNAVDGQWGCWSSWSTCDATYKRSRTRECNNPAPQRGGKRCEGEKRQEEDCTFSIMENNGQPCINDDEEMKEVDLPEIEADSGCPQPVPPENGFIRNEKQLYLVGEDVEISCLTGFETVGYQYFRCLPDGTWRQGDVECQRTECIKPVVQEVLTITPFQRLYRIGESIELTCPKGFVVAGPSRYTCQGNSWTPPISNSLTCEKDTLTKLKGHCQLGQKQSGSECICMSPEEDCSHHSEDLCVFDTDSNDYFTSPACKFLAEKCLNNQQLHFLHIGSCQDGRQLEWGLERTRLSSNSTKKESCGYDTCYDWEKCSASTSKCVCLLPPQCFKGGNQLYCVKMGSSTSEKTLNICEVGTIRCANRKMEILHPGKCLA

SEQ ID NO: 123 - protein, 353 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 124 - protein, 340 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 125 - protein, 330 aa, synthetic construct [sequence omitted]

SEQ ID NO: 126 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 127 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 128 - protein, 159 aa, synthetic construct
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 129 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 130 - protein, 638 aa, synthetic construct
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 131 - protein, 402 aa, synthetic construct
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 132 - protein, 698 aa, synthetic construct
  CARBOHYD 432: note = N is linked to the N-linked glycan structure Hex(5)HexNAc(4)Fuc(0)NeuAc(2)
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 133 - protein, 752 aa, synthetic construct
MGPLMVLFCLLFLYPGLADSAPSCPQNVNISGGTFTLSHGWAPGSLLTYSCPQGLYPSPASRLCKSSGQWQTPGATRSLSKAVCKPVRCPAPVSFENGIYTPRLGSYPVGGNVSFECEDGFILRGSPVRQCRPNGMWDGETAVCDNGAGHCPNPGISLGAVRTGFRFGHGDKVRYRCSSNLVLTGSSERECQGNGVWSGTEPICRQPYSYDFPEDVAPALGTSFSHMLGATNPTQKTKESLGRKIQIQRSGHLNLYLLLDCSQSVSENDFLIFKESASLMVDRIFSFEINVSVAIITFASEPKVLMSVLNDNSRDMTEVISSLENANYKDHENGTGTNTYAALNSVYLMMNNQMRLLGMETMAWQEIRHAIILLTDGKSNMGGSPKTAVDHIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 134 - protein, 1676 aa, synthetic construct
  CARBOHYD 911: note = N is linked to the N-linked glycan structure Hex(5)HexNAc(4)Fuc(0)NeuAc(1)
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 135 - protein, 298 aa, synthetic construct
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

SEQ ID NO: 136 - protein, 7 aa, synthetic construct
ENGTISR

SEQ ID NO: 137 - protein, 7 aa, synthetic construct
ENGTISR

SEQ ID NO: 138 - protein, 16 aa, synthetic construct
SVQEIQATFFYFTPNK

SEQ ID NO: 139 - protein, 24 aa, synthetic construct
MVSHHNLTTGATLINEQWLLTTAK

SEQ ID NO: 140 - protein, 24 aa, synthetic construct
MVSHHNLTTGATLINEQWLLTTAK

SEQ ID NO: 141 - protein, 13 aa, synthetic construct
NLFLNHSENATAK

SEQ ID NO: 142 - protein, 13 aa, synthetic construct
NLFLNHSENATAK

SEQ ID NO: 143 - protein, 13 aa, synthetic construct
NLFLNHSENATAK

SEQ ID NO: 144 - protein, 13 aa, synthetic construct
NLFLNHSENATAK

SEQ ID NO: 145 - protein, 16 aa, synthetic construct
VVLHPNYSQVDIGLIK

SEQ ID NO: 146 - protein, 14 aa, synthetic construct
SSTTKPPFKPHGSR

SEQ ID NO: 147 - protein, 10 aa, synthetic construct
DLLLPQPDLR

SEQ ID NO: 148 - protein, 10 aa, synthetic construct
VNHVTLSQPK

SEQ ID NO: 149 - protein, 9 aa, synthetic construct
HAFILQDTK

SEQ ID NO: 150 - protein, 8 aa, synthetic construct
GIYGTISR

SEQ ID NO: 151 - protein, 6 aa, synthetic construct
AHYDLR

SEQ ID NO: 152 - protein, 17 aa, synthetic construct
GVTSVSQIFHSPDLAIR

SEQ ID NO: 153 - protein, 8 aa, synthetic construct
YFIDFVAR

SEQ ID NO: 154 - protein, 8 aa, synthetic construct
VSLATVDK

SEQ ID NO: 155 - protein, 8 aa, synthetic construct
YTACETAR

SEQ ID NO: 156 - protein, 10 aa, synthetic construct
CLAYDFYPGK

SEQ ID NO: 157 - protein, 764 aa, synthetic construct
  CARBOHYD 421: note = N is linked to the N-linked glycan structure Hex(5)HexNAc(4)Fuc(0)NeuAc(2)
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 158 - protein, 330 aa, synthetic construct [sequence omitted]

SEQ ID NO: 159 - protein, 326 aa, synthetic construct; identical to SEQ ID NO: 126
  CARBOHYD 176: note = N is linked to the N-linked glycan structure Hex(3)HexNAc(3)Fuc(1)NeuAc(0)
  CARBOHYD 176: note = N is linked to the N-linked glycan structure Hex(3)HexNAc(5)Fuc(1)NeuAc(0)

SEQ ID NO: 160 - protein, 327 aa, synthetic construct
  CARBOHYD 177: note = N is linked to the N-linked glycan structure Hex(3)HexNAc(3)Fuc(0)NeuAc(0)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - protein, 353 aa, synthetic construct; identical to SEQ ID NO: 123
  CARBOHYD 340: note = N is linked to the N-linked glycan structure Hex(3)HexNAc(2)Fuc(0)NeuAc(0)

SEQ ID NO: 162 - protein, 340 aa, synthetic construct; identical to SEQ ID NO: 124
  CARBOHYD 327: note = N is linked to the N-linked glycan structure Hex(6)HexNAc(2)Fuc(0)NeuAc(0)

SEQ ID NO: 163 - protein, 223 aa, synthetic construct
  CARBOHYD 51: note = N is linked to the N-linked glycan structure Hex(5)HexNAc(4)Fuc(0)NeuAc(2)
MNKPLLWISVLTSLLEAFAHTDLSGKVFVFPRESVTDHVNLITPLEKPLQNFTLCFRAYSDLSRAYSLFSYNTQGRDNELLVYKERVGEYSLYIGRHKVTSKVIEKFPAPVHICVSWESSSGIAEFWINGTPLVKKGLRQGYFVEAQPKIVLGQEQDSYGGKFDRSQSFVGEIGDLYMWDSVLPPENILSAYQGTPLPANILDWQALNYEIRGYVIIKPLVWV

SEQ ID NO: 164 - protein, 559 aa, synthetic construct
  CARBOHYD 415: note = N is linked to the N-linked glycan structure Hex(6)HexNAc(5)Fuc(1)NeuAc(3)
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

SEQ ID NO: 165 - protein, 4563 aa, synthetic construct
  CARBOHYD 3465: note = N is linked to the N-linked glycan structure Hex(5)HexNAc(4)Fuc(0)NeuAc(1)
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSYDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 166 - protein, 1744 aa, synthetic construct
  CARBOHYD 226: note = N is linked to the N-linked glycan structure Hex(9)HexNAc(2)Fuc(0)NeuAc(0)
  CARBOHYD 1328: note = N is linked to the N-linked glycan structure Hex(5)HexNAc(4)Fuc(0)NeuAc(2)
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAAAAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALTLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 167 - protein, 449 aa, synthetic construct
  CARBOHYD 354: note = N is linked to the N-linked glycan structure Hex(6)HexNAc(5)Fuc(1)NeuAc(2)
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 168 - protein, 16 aa, synthetic construct
  CARBOHYD 6: note = N is linked to the N-linked glycan structure Hex(4)HexNAc(3)Fuc(1)NeuAc(0)
VVLHPNYSQVDIGLIK

SEQ ID NO: 169 - protein, 16 aa, synthetic construct
  CARBOHYD 6: note = N is linked to the N-linked glycan structure Hex(4)HexNAc(5)Fuc(0)NeuAc(1)
VVLHPNYSQVDIGLIK

SEQ ID NO: 170 - protein, 16 aa, synthetic construct
  CARBOHYD 6: note = N is linked to the N-linked glycan structure Hex(5)HexNAc(3)Fuc(0)NeuAc(1)
VVLHPNYSQVDIGLIK

SEQ ID NO: 171 - protein, 26 aa, synthetic construct
  CARBOHYD 6: note = N is linked to the N-linked glycan structure Hex(5)HexNAc(4)Fuc(0)NeuAc(1)
VVLHPNYSQVDIGLIKLKQKVSVNER

SEQ ID NO: 172 - protein, 24 aa, synthetic construct
  CARBOHYD 6: note = N is linked to the N-linked glycan structure Hex(5)HexNAc(5)Fuc(0)NeuAc(1)
MVSHHNLTTGATLINEQWLLTTAK

SEQ ID NO: 173 - protein, 30 aa, synthetic construct; identical to SEQ ID NO: 45
  CARBOHYD 7: note = N is linked to the N-linked glycan structure Hex(4)HexNAc(4)Fuc(0)NeuAc(1)

SEQ ID NO: 174 - protein, 32 aa, synthetic construct; identical to SEQ ID NO: 42
  CARBOHYD 14: note = N is linked to the N-linked glycan structure Hex(4)HexNAc(3)Fuc(1)NeuAc(1)

SEQ ID NO: 175 - protein, 32 aa, synthetic construct; identical to SEQ ID NO: 42
  CARBOHYD 14: note = N is linked to the N-linked glycan structure Hex(5)HexNAc(4)Fuc(0)NeuAc(3)

SEQ ID NO: 176 - protein, 32 aa, synthetic construct; identical to SEQ ID NO: 42
  CARBOHYD 14: note = N is linked to the N-linked glycan structure Hex(7)HexNAc(6)Fuc(2)NeuAc(4)

SEQ ID NO: 177 - protein, 16 aa, synthetic construct
  CARBOHYD 4: note = N is linked to the N-linked glycan structure Hex(5)HexNAc(5)Fuc(0)NeuAc(2)
YLGNATAIFFLPDEGK

SEQ ID NO: 178 - protein, 33 aa, synthetic construct
  CARBOHYD 17: note = N is linked to the N-linked glycan structure Hex(6)HexNAc(5)Fuc(0)NeuAc(2)
TEVSSNHVLIYLDKVSNQTLSLFFTVLQDVPVR

SEQ ID NO: 179 - protein, 33 aa, synthetic construct; identical to SEQ ID NO: 178
  CARBOHYD 17: note = N is linked to the N-linked glycan structure Hex(6)HexNAc(5)Fuc(0)NeuAc(3)

SEQ ID NO: 180 - protein, 33 aa, synthetic construct; identical to SEQ ID NO: 178
  CARBOHYD 17: note = N is linked to the N-linked glycan structure Hex(6)HexNAc(5)Fuc(1)NeuAc(1)

SEQ ID NO: 181 - protein, 33 aa, synthetic construct
  CARBOHYD 23: note = N is linked to the N-linked glycan structure Hex(5)HexNAc(4)Fuc(0)NeuAc(1)
VEGSSSHLVTFTVLPLEIGLHNINFSLETWFGK

SEQ ID NO: 182 - protein, 22 aa, synthetic construct
  CARBOHYD 9: note = N is linked to the N-linked glycan structure Hex(5)HexNAc(4)Fuc(0)NeuAc(2)
LSLLEEPGNGTFTVILNQLTSR

SEQ ID NO: 183 - protein, 30 aa, synthetic construct
  CARBOHYD 5: note = N is linked to the N-linked glycan structure Hex(3)HexNAc(4)Fuc(1)NeuAc(0)
EEQYNSTYRVVSVLTVLHQDWLNGKEYKCK

SEQ ID NO: 184 - protein, 29 aa, synthetic construct
  CARBOHYD 5: note = N is linked to the N-linked glycan structure Hex(3)HexNAc(5)Fuc(1)NeuAc(0)
TKPREEQYNSTYRVVSVLTVLHQDWLNGK

SEQ ID NO: 185 - protein, 28 aa, synthetic construct
  CARBOHYD 5: note = N is linked to the N-linked glycan structure Hex(3)HexNAc(3)Fuc(1)NeuAc(0)
EEQFNSTFRVVSVLTVVHQDWLNGKEYK

SEQ ID NO: 186 - protein, 28 aa, synthetic construct
  CARBOHYD 5: note = N is linked to the N-linked glycan structure Hex(3)HexNAc(5)Fuc(1)NeuAc(0)
EEQFNSTFRVVSVLTVVHQDWLNGKEYK

SEQ ID NO: 187 - protein, 25 aa, synthetic construct
  CARBOHYD 5: note = N is linked to the N-linked glycan structure Hex(3)HexNAc(3)Fuc(0)NeuAc(0)
EEQFNSTYRVVSVLTVLHQDWLNGK

SEQ ID NO: 188 - protein, 22 aa, synthetic construct
  CARBOHYD 9: note = N is linked to the N-linked glycan structure Hex(3)HexNAc(2)Fuc(0)NeuAc(0)
LAGKPTHVNVSVVMAEVDGTCY

SEQ ID NO: 189 - protein, 22 aa, synthetic construct
  CARBOHYD 9: note = N is linked to the N-linked glycan structure Hex(6)HexNAc(2)Fuc(0)NeuAc(0)
MAGKPTHINVSVVMAEADGTCY

SEQ ID NO: 190 - protein, 32 aa, synthetic construct
  CARBOHYD 19: note = N is linked to the N-linked glycan structure Hex(5)HexNAc(4)Fuc(0)NeuAc(2)
ESVTDHVNLITPLEKPLQNFTLCFRAYSDLSR

SEQ ID NO: 191 - protein, 18 aa, synthetic construct
  CARBOHYD 3: note = N is linked to the N-linked glycan structure Hex(6)HexNAc(5)Fuc(1)NeuAc(3)
AVNITSENLIDDVVSLIR

SEQ ID NO: 192 - protein, 34 aa, synthetic construct
  CARBOHYD 33: note = N is linked to the N-linked glycan structure Hex(5)HexNAc(4)Fuc(0)NeuAc(2)
IMNGEADAMSLDGGFVYIAGKCGLVPVLAENYNK

SEQ ID NO: 193 - protein, 31 aa, synthetic construct
  CARBOHYD 16: note = N is linked to the N-linked glycan structure Hex(5)HexNAc(4)Fuc(0)NeuAc(1)
SKPTVSSSMEFKYDFNSSMLYSTAKGAVDHK

SEQ ID NO: 194 - protein, 26 aa, synthetic construct
  CARBOHYD 8: note = N is linked to the N-linked glycan structure Hex(9)HexNAc(2)Fuc(0)NeuAc(0)
FSDGLESNSSTQFEVKKYVLPNFEVK

SEQ ID NO: 195 - protein, 36 aa, synthetic construct
  CARBOHYD 28: note = N is linked to the N-linked glycan structure Hex(5)HexNAc(4)Fuc(0)NeuAc(2)
STQDTVIALDALSAYWIASHTTEERGLNVTLSSTGR

SEQ ID NO: 196 - protein, 20 aa, synthetic construct
  CARBOHYD 3: note = N is linked to the N-linked glycan structure Hex(6)HexNAc(5)Fuc(1)NeuAc(2)
MLNTSSLLEQLNEQFNWVSR

SEQ ID NO: 197 - protein, 7 aa, synthetic construct
  CARBOHYD 4: note = N is linked to the N-linked glycan structure Hex(5)HexNAc(4)Fuc(0)NeuAc(1)
LSSNSTK

SEQ ID NO: 198 - protein, 20 aa, synthetic construct
  CARBOHYD 5: note = N is linked to the N-linked glycan structure Hex(5)HexNAc(4)Fuc(1)NeuAc(2)
AFITNFSMIIDGMTYPGIIK

SEQ ID NO: 199 - protein, 7 aa, synthetic construct
ECDMHYK

(19) United States
(12) Patent Application Publication
(10) Pub. No.: US 2025/0149173 A1
(43) Pub. Date: May 8, 2025

(54) DIAGNOSIS OF COLORECTAL CANCER USING TARGETED QUANTIFICATION OF SITE-SPECIFIC PROTEIN GLYCOSYLATION

(71) Applicant: VENN BIOSCIENCES CORPORATION, South San Francisco, CA (US)

(72) Inventors: Daniel SERIE, South San Francisco, CA (US); Khushbu Yatin DESAI, South San Francisco, CA (US); Daniel Willem HOMMES, South San Francisco, CA (US); Alan Nicolas MITCHELL, South San Francisco, CA (US); Maurice Yu WONG, South San Francisco, CA (US); Mingqi LUI, San Carlos, CA (US); Bo ZHOU, Los Angeles, CA (US)

(21) Appl. No.: 18/837,706

(22) PCT Filed: Feb. 14, 2023
(86) PCT No.: PCT/US2023/062602

§ 371 Date: Aug. 12, 2024

Related U.S. Application Data
(60) Provisional application No. 63/267,995, filed on Feb. 14, 2022.
(60) Provisional application No. 63/364,257, filed on May 5, 2022.
(60) Provisional application No. 63/365,410, filed on May 26, 2022.
(60) Provisional application No. 63/368,153, filed on Jul. 11, 2022.
(60) Provisional application No. 63/393,703, filed on Jul. 29, 2022.
(60) Provisional application No. 63/375,355, filed on Sep. 12, 2022.
(60) Provisional application No. 63/377,330, filed on Sep. 27, 2022.
(60) Provisional application No. 63/384,566, filed on Nov. 21, 2022.
(60) Provisional application No. 63/478,869, filed on Jan. 6, 2023.
(60) Provisional application No. 63/478,905, filed on Jan. 6, 2023.

(51) Int. Cl. G16H 50/20; G01N 33/68; G16B 15/20; G16B 40/20; G16H 15/00; G16H 20/10; G16H 20/40; G16H 50/30; G16H 50/70

(52) U.S. Cl. CPC G16H 50/20; G01N 33/6848; G01N 33/6893; G16B 15/20; G16B 40/20; G16H 15/00; G16H 20/10; G16H 20/40; G16H 50/30; G16H 50/70

(57) ABSTRACT

The present disclosure encompasses systems, methods, and compositions for diagnosing a subject for a high-grade advanced pre-malignant lesions or colorectal cancer (CRC) disease state by ascertaining the presence of certain one or more glycosylated or aglycosylated peptides in liquid biopsy samples from the subject. Specific embodiments encompass methods of measuring certain one or more glycosylated or aglycosylated peptides in liquid biopsy samples from subjects known to have or suspected of having a high-grade advanced pre-malignant lesions or CRC disease state or subjects undergoing routine health care maintenance for possible presence of a high-grade advanced pre-malignant lesions or CRC disease state. The disclosure provides systems, methods, and compositions to identify subjects at-risk for CRC or high-grade advanced pre-malignant lesions and increases subject colonoscopy compliance, in specific embodiments.

### CROSS-REFERENCE TO RELATED APPLICATIONS

This application claims the priority benefit of U.S. Provisional Patent Application Ser. No. 63/267,995, filed Feb. 14, 2022 [Attorney Docket No.VENN.P0013US.P1/VENN-00029PR]; U.S. Provisional Patent Application Ser. No. 63/364,257, May 5, 2022; [Attorney Docket No. VENN.P0013US.P2/VENN-00029P1]; U.S. Provisional Patent Application Ser. No. 63/365,410, filed May 26, 2022; [Attorney Docket No. VENN.P0013US.P3/VENN-00029P2]; U.S. Provisional Patent Application Ser. No. 63/368,153, filed Jul. 11, 2022; [Attorney Docket No. VENN.P0024US.P1/VENN-00044PR]; U.S. Provisional Patent Application Ser. No. 63/375,355, filed Sep. 12, 2022; [Attorney Docket No. VENN.P0024US.P2/VENN-00044P1]; U.S. Provisional Patent Application Ser. No. 63/377,330, filed Sep. 27, 2022; [Attorney Docket No. VENN.P0024US.P3/VENN-00044P2]; U.S. Provisional Patent Application Ser. No. 63/384,566, filed Nov. 21, 2022; [Attorney Docket No. VENN.P0024US.P4/VENN-00044P3]; U.S. Provisional Patent Application Ser. No. 63/478,869, filed Jan. 6, 2023; [Attorney Docket No. VENN.P0024US.P5/VENN-00044P4]; U.S. Provisional Patent Application Ser. No. 63/478,905, filed Jan. 6, 2023; [Attorney Docket No. VENN.P0024US.P6/VENN-00044P5]; and U.S. Provisional Patent Application Ser. No. 63/393,703, filed Jul. 29, 2022; [Attorney Docket No. 16653-30024.00/VENN-00047PR], which are hereby all incorporated by reference herein in their entirety.

### FIELD

The present disclosure generally relates to methods and systems for analyzing peptide structures for diagnosing and/or treating adenomas, advanced precancerous lesions, high-grade advanced pre-malignant lesion, and/or colorectal cancer. More particularly, the present disclosure relates to analyzing quantification data for a set of peptide structures detected in a biological sample obtained from a subject for use in diagnosing and/or treating the subject, the set of peptide structures being associated with adenomas, advanced precancerous lesions, high-grade advanced pre-malignant lesion, and/or colorectal cancer.

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully identify disease processes.

Glycoprotein analysis is fraught with challenges on several levels. For example, a single glycan composition in a peptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a need for improved analytical methods that involve site-specific analysis of glycoproteins to obtain information about protein glycosylation patterns, which can in turn provide quantitative information that can be used to identify disease states. For example, there is a need to use such analysis to diagnose and/or treat colorectal cancer.

Colorectal cancers (CRCs) typically develop from colon adenomas, among which “advanced” colon adenomas are considered to be the clinically relevant precursors of CRCs. A colon adenoma is a type of polyp, or unusual growth of cells that form a small clump (i.e., colon mass or tumor) in the lining of the colon that is not cancer. While most of them are benign, or not dangerous, up to 10 percent of advanced colon adenomas can transform into cancer. Under certain circumstances, an advanced colon adenoma can be referred to as an advanced precancerous lesion (APL). Finding CRCs and/or advanced adenomas early can lead to better survival statistics for patients. Most CRCs and advanced adenomas are currently diagnosed using more invasive diagnostic techniques such as a colonoscopy and/or a tissue biopsy. Since many patients delay or are reluctant to undergo invasive-type diagnostic procedures, it is important to develop less invasive or non-invasive diagnostic methods that are able to identify patients who have colon masses of concern and classify those masses as CRCs (i.e., malignant) or advanced adenomas (i.e., non-malignant) so that they can be properly treated.

Thus, an approach that is non-invasive, accurate, and reliable and that enables early diagnosis is needed. An approach enabling early diagnosis may help reduce negative health outcomes in patients with colorectal cancer and/or increase the effectiveness of preventative treatment of precursors (i.e., advanced adenomas) to colorectal cancer. Such an approach can assist in guiding a patient to an urgency for further testing, for example, including for a colonoscopy procedure, for example. Thus, it may be desirable to have methods and systems capable of addressing one or more of the above-identified issues.

### SUMMARY

### Table 1

Embodiments of the disclosure encompass systems, methods, and compositions related to diagnosing a subject for an adenoma or colorectal cancer (CRC) disease state by ascertaining the presence of certain one or more glycosylated or aglycosylated peptides in liquid biopsy samples from the subject. Specific embodiments encompass methods of measuring certain one or more glycosylated or aglycosylated peptides in liquid biopsy samples from subjects known to have or suspected of having an adenoma or CRC disease state or subjects undergoing routine health care maintenance for possible presence of an adenoma or CRC disease state. Subjects suspected of having an adenoma or CRC disease state or those undergoing routine health care maintenance may or may not have one or more symptoms of an adenoma or CRC disease state, such as anemia. abdominal pain. dark or bloody stools. Rectal bleeding, constipation or diarrhea, unexplained weight loss, and/or feeling that the bowel does not empty all the way. Subject having the certain one or more glycosylated or aglycosylated peptides are directed for further testing, such as a colonoscopy.

In various embodiments, the present disclosure provides systems, methods, and compositions with the ability to identify subjects in need of further testing for an adenoma or CRC disease state, such as a colonoscopy, because their glycoproteomic profile indicates they are at risk for either advanced adenoma or CRC. Such embodiments allow for early detection and intervention (even at the advanced adenoma stage), leading to significantly better outcomes and survival rates for the subjects. These embodiments improve subject compliance, given the indication of a higher risk for advanced adenoma or CRC in subjects having the one or more certain glycosylated or aglycosylated peptide(s) and a need for a follow-up procedure, including a colonoscopy.

Various embodiments of the disclosure encompass methods for diagnosing a subject with respect to adenoma or colorectal cancer (CRC) disease state, the method comprising receiving peptide structure data corresponding to a biological sample obtained from the subject; analyzing the peptide structure data using at least one supervised machine learning model to generate a disease indicator that indicates whether the biological sample evidences an adenoma or CRC disease state based on at least one peptide structure selected from a group of peptide structures identified in Table 1; wherein the group of peptide structures in Table 1 is associated with the adenoma or CRC disease state; and wherein the group of peptide structures is listed in Table 1 with respect to relative significance to the disease indicator; and generating a diagnosis output based on the disease indicator. In specific embodiments, the disease indicator comprises a score. In specific embodiments, the generating of the diagnosis output comprises determining that the score falls above a selected threshold; and generating the diagnosis output based on the score falling above the selected threshold, wherein the diagnosis output includes a positive diagnosis for the adenoma or CRC disease state. In some embodiments, generating the diagnosis output comprises determining that the score falls below a selected threshold; and generating the diagnosis output based on the score falling below the selected threshold, wherein the diagnosis output includes a negative diagnosis for the adenoma or CRC disease state. In specific cases, the score comprises a probability score and the selected threshold is 0.3267. The selected threshold may fall within a range between 0 and 1, 0 and 0.9, 0 and 0.8, 0 and 0.7, 0 and 0.6, 0 and 0.5, 0 and 0.4, 0 and 0.3, 0 and 0.2, 0 and 0.1, 0.05 to 0.95, 0.05 and 0.85, 0.05 and 0.75, 0.05 and 0.65, 0.05 and 0.55, 0.05 and 0.45, 0.05 and 0.35, 0.05 and 0.25, 0.05 and 0.15, 0.1 and 1, 0.1 and 0.9, 0.1 and 0.8, 0.1 and 0.7, 0.1 and 0.6, 0.1 and 0.5, 0.1 and 0.4, 0.1 and 0.3, 0.1 and 0.2, 0.2 and 1.0, 0.2 and 0.9, 0.2 and 0.8, 0.2 and 0.7, 0.2 and 0.6, 0.2 and 0.5, 0.2 and 0.4, 0.2 and 0.3, 0.3 and 0.9, 0.3 and 0.8, 0.3 and 0.7, 0.3 and 0.6, 0.3 and 0.5, 0.3 and 0.4, 0.4 and 1, 0.4 and 0.9, 0.4 and 0.8, 0.4 and 0.7, 0.4 and 0.6, 0.4 and 0.5, 0.5 and 1.0, 0.6 and 1, 0.6 and 0.9, 0.6 and 0.8, 0.6 and 0.7, 0.7 and 1.0, 0.7 and 0.9, 0.7 and 0.8, 0.8 and 1.0, 0.8 and 0.9, or 0.9 and 1. In certain embodiments, analyzing the peptide structure data comprises analyzing the peptide structure data using a binary classification model. The at least one peptide structure may comprise a glycopeptide structure defined by a peptide sequence and a glycan structure linked to the peptide sequence at a linking site of the peptide sequence, as identified in Table 1, with the peptide sequence being one of SEQ ID NOS: 7-12 as defined in Table 1. In some embodiments, the method further comprises training the at least one supervised machine learning model using training data, wherein the training data comprises a plurality of peptide structure profiles for a plurality of subjects and a plurality of subject diagnoses for the plurality of subjects. In specific embodiments, the plurality of subject diagnoses may include a positive diagnosis for any subject of the plurality of subjects determined to have the adenoma or CRC disease state and a negative diagnosis for any subject of the plurality of subjects determined not to have the adenoma or CRC disease state, wherein the adenoma or CRC disease state comprises at least one of CRC generally, early stage CRC, late stage CRC, stage 1 CRC, stage 2 CRC, stage 3 CRC, stage 4 CRC, or adenoma. In some embodiments, the method may further comprise performing a differential expression analysis using initial training data to compare a first portion of the plurality of subjects diagnosed with the positive diagnosis for the CRC or adenoma disease state versus a second portion of the plurality of subjects having the negative diagnosis for the adenoma or CRC disease state; and identifying a training group of peptide structures based on the differential expression analysis for use as prognostic markers for the adenoma or CRC disease state; and forming the training data based on the training group of peptide structures identified. The peptide structure data may comprise at least one of a raw abundance, an adjusted raw abundance, a peptide concentration, a glycopeptide concentration, or a normalized concentration. The peptide structure data may comprise normalized concentration data, wherein the normalized concentration data is a function of at least one of peptide abundance data, corresponding internal standard abundance data, a spike-in concentration value, and a dilution factor. The at least one supervised machine learning model may comprise a logistic regression model, and wherein the at least one supervised learning model compares the negative diagnosis versus the positive diagnosis, wherein the comparison can be at least one non-adenoma or non-CRC state vs at least one adenoma or CRC state. In specific embodiments, the at least one supervised machine learning model comprises a logistic regression model, and wherein the at least one supervised learning model compares negative diagnoses versus positive diagnoses, wherein the comparison can be at least one healthy state versus adenoma or CRC generally, healthy state versus adenoma or early stage CRC, healthy state vs adenoma or stage 1 CRC, healthy state versus adenoma or stage 2 CRC, healthy state versus adenoma or stage 3 CRC, or healthy state versus adenoma or stage 4 CRC. The peptide structure data may be generated using multiple reaction monitoring mass spectrometry (MRM-MS). In some embodiments, the method further comprises creating a sample from the biological sample; and preparing the sample using reduction, alkylation, and enzymatic digestion to form a prepared sample that includes a set of peptide structures. The method may further comprise generating the peptide structure data from the prepared sample using multiple reaction monitoring mass spectrometry (MRM-MS). In some embodiments, generating the diagnosis output comprises generating a report identifying that the biological sample evidences the adenoma or CRC disease state. The method may further comprise generating a treatment output based on at least one of the diagnosis output or the disease indicator. In specific embodiments, the treatment output may comprise at least one of an identification of a treatment to treat the subject or a treatment plan, and the treatment may comprise at least one of radiation therapy, chemoradiotherapy, surgery, hormone therapy, or a targeted drug therapy and may also comprise further testing.

Embodiments of the disclosure include methods of training a model to diagnose a subject with respect to an adenoma or CRC disease state, the method comprising receiving peptide structure data for a panel of peptide structures for a plurality of subjects, wherein the plurality of subjects includes a first portion having a negative diagnosis of an adenoma or CRC disease state and a second portion having a positive diagnosis of the adenoma or CRC disease state; wherein the peptide structure data comprises a plurality of peptide structure profiles for the plurality of subjects; and training at least one machine learning model using the peptide structure data to diagnose a biological sample with respect to the adenoma or CRC disease state using a group of peptide structures associated with the adenoma or CRC disease state, wherein the group of peptide structures is identified in Table 1; and wherein the group of peptide structures is listed in Table 1 with respect to relative significance to diagnosing the biological sample. In specific embodiments, the at least one machine learning model may comprise a logistic regression model, and wherein the at least one machine learning model compares the negative diagnosis versus the positive diagnosis, wherein the comparison can be at least one non-adenoma or non-CRC state vs at least one adenoma or CRC state. In some embodiments, the at least one supervised machine learning model may comprise a logistic regression model, and wherein the at least one supervised learning model compares negative diagnoses versus positive diagnoses, wherein the comparison can be at least one healthy state versus adenoma or CRC generally, healthy state versus adenoma or early stage CRC, healthy state vs adenoma or stage 1 CRC, healthy state versus adenoma or stage 2 CRC, healthy state versus adenoma or stage 3 CRC, or healthy state versus adenoma or stage 4 CRC. Training the at least one machine learning model may comprise training the at least one machine learning model using a portion of the peptide structure data corresponding to a training group of peptide structures included in the plurality of peptide structures. The method may further comprise performing a differential expression analysis using the peptide structure data for the plurality of subjects. The method may further comprise identifying the training group of peptide structures based on the differential expression analysis, wherein the training group of peptide structures is a subset of the plurality of peptide structures that has been determined to be relevant to diagnosing the adenoma or CRC disease state. In specific embodiments, the peptide structure data may comprise at least one of a raw abundance, an adjusted raw abundance, a peptide concentration, a glycopeptide concentration, or a normalized concentration. The peptide structure data may comprise normalized concentration data, wherein the normalized concentration data is a function of at least one of peptide abundance data, corresponding internal standard abundance data, a spike-in concentration value, and a dilution factor.

Embodiments of the disclosure include methods of monitoring a subject for an adenoma or CRC disease state, the method may comprise receiving first peptide structure data for a first biological sample obtained from a subject at a first timepoint; analyzing the first peptide structure data using at least one supervised machine learning model to generate a first disease indicator based on at least one peptide structure selected from a group of peptide structures identified in Table 1, wherein the group of peptide structures in Table 1 comprises a group of peptide structures associated with an adenoma or CRC disease state; receiving second peptide structure data of a second biological sample obtained from the subject at a second timepoint; analyzing the second peptide structure data using the at least one supervised machine learning model to generate a second disease indicator based on the at least one peptide structure selected from the group of peptide structures identified in Table 1; and generating a diagnosis output based on the first disease indicator and the second disease indicator. In specific embodiments, generating the diagnosis output may comprise comparing the second disease indicator to the first disease indicator. In specific embodiments, the first disease indicator may indicate that the first biological sample evidences a negative diagnosis for the adenoma or CRC disease state and the second biological sample evidences a positive diagnosis for the adenoma or CRC disease state. In specific embodiments, the plurality of subject diagnoses may include a positive diagnosis for any subject of the plurality of subjects determined to have the adenoma or CRC disease state and a negative diagnosis for any subject of the plurality of subjects determined not to have the adenoma or CRC disease state, wherein the adenoma or CRC disease state comprises at least one of adenoma or CRC cancer generally, adenoma or early stage CRC, adenoma or late stage CRC, adenoma or stage 1 CRC, adenoma or stage 2 CRC, adenoma or stage 3 CRC, or adenoma or stage 4 CRC. The at least one supervised machine learning model may comprise a logistic regression model, and wherein the at least one supervised learning model compares the negative diagnosis versus the positive diagnosis, wherein the comparison can be at least one non-adenoma or non-CRC state vs at least one adenoma or CRC state. In specific embodiments, the at least one supervised machine learning model may comprise a logistic regression model, and wherein the at least one supervised learning model compares negative diagnoses versus positive diagnoses, wherein the comparison can be at least one healthy state versus adenoma or CRC generally, healthy state versus adenoma or early stage CRC, healthy state vs adenoma or stage 1 CRC cancer, healthy state versus adenoma or stage 2 CRC, healthy state versus adenoma or stage 3 CRC, or healthy state versus adenoma or stage 4 CRC.

Embodiments of the disclosure include compositions comprising at least one of peptide structures PS-1, PS-2, PS-3, PS-4, PS-5, or PS-6 identified in Table 1.

Embodiments of the disclosure include compositions comprising a peptide structure or a product ion, wherein the peptide structure or the product ion comprises an amino acid sequence having at least 90% sequence identity to any one of SEQ ID NOS: 7-12, corresponding to peptide structures PS-1, PS-2, PS-3, PS-4, PS-5, or PS-6 in Table 1; and the product ion is selected as one from a group consisting of product ions identified in Table 2 including product ions falling within an identified m/z range.

Embodiments of the disclosure include compositions comprising a glycopeptide structure selected as one peptide structure from a group consisting of PS-1, PS-2, PS-3, PS-4, PS-5, or PS-6 identified in Table 1, wherein the glycopeptide structure comprises an amino acid peptide sequence identified in Table 3A as corresponding to the glycopeptide structure; and a glycan structure identified in Table 5 as corresponding to the glycopeptide structure in which the glycan structure is linked to a residue of the amino acid peptide sequence at a corresponding position identified in Table 1; and wherein the glycan structure has a glycan composition. In specific cases, the glycan composition is identified in Table 5. In specific cases, the glycopeptide structure has a precursor ion having a charge identified in Table 3 as corresponding to the glycopeptide structure. The glycopeptide structure may have a precursor ion with an m/z ratio within ±1.5 of the m/z ratio listed for the precursor ion in Table 2 as corresponding to the glycopeptide structure. The glycopeptide structure may have a precursor ion with an m/z ratio within ±1.0 of the m/z ratio listed for the precursor ion in Table 2 as corresponding to the glycopeptide structure. In specific embodiments, the glycopeptide structure may have a precursor ion with an m/z ratio within ±0.5 of the m/z ratio listed for the precursor ion in Table 2 as corresponding to the glycopeptide structure. The glycopeptide structure may have a product ion with an m/z ratio within ±1.0 of the m/z ratio listed for the product ion in Table 2 as corresponding to the glycopeptide structure. In specific embodiments, the glycopeptide structure has a product ion with an m/z ratio within ±0.8 of the m/z ratio listed for the product ion in Table 3 as corresponding to the glycopeptide structure. The glycopeptide structure may have a product ion with an m/z ratio within ±0.5 of the m/z ratio listed for the product ion in Table 2 as corresponding to the glycopeptide structure. The glycopeptide structure may have a monoisotopic mass identified in Table 1 as corresponding to the glycopeptide structure.

Embodiments of the disclosure include compositions comprising a peptide structure selected as one from a plurality of peptide structures identified in Table 1, wherein the peptide structure has a monoisotopic mass identified as corresponding to the peptide structure in Table 1; and the peptide structure comprises the amino acid sequence of SEQ ID NOS: 7-12 identified in Table 1 as corresponding to the peptide structure. The peptide structure may have a precursor ion having a charge identified in Table 3 as corresponding to the peptide structure. The peptide structure may have a precursor ion with an m/z ratio within ±1.5 of the m/z ratio listed for the precursor ion in Table 2 as corresponding to the peptide structure. The peptide structure may have a precursor ion with an m/z ratio within ±1.0 of the m/z ratio listed for the precursor ion in Table 2 as corresponding to the peptide structure. The peptide structure may have a precursor ion with an m/z ratio within ±0.5 of the m/z ratio listed for the precursor ion in Table 2 as corresponding to the peptide structure. The peptide structure may have a product ion with an m/z ratio within ±1.0 of the m/z ratio listed for the product ion in Table 2 as corresponding to the peptide structure. The peptide structure may have a product ion with an m/z ratio within ±0.8 of the m/z ratio listed for the product ion in Table 2 as corresponding to the peptide structure. The peptide structure may have a product ion with an m/z ratio within ±0.5 of the m/z ratio listed for the product ion in Table 2 as corresponding to the peptide structure.

Embodiments of the disclosure include kits that may comprise at least one agent for quantifying at least one peptide structure identified in Table 1 to carry out part or all of any method encompassed herein.

Embodiments of the disclosure include kits that may comprise at least one of a glycopeptide standard, a buffer, or a set of peptide sequences to carry out part or all of the method of any one of claims 1-36, a peptide sequence of the set of peptide sequences identified by a corresponding one of SEQ ID NOS: 7-12, defined in Table 1.

Embodiments of the disclosure include systems comprising one or more data processors; and a non-transitory computer readable storage medium containing instructions which, when executed on the one or more data processors, cause the one or more data processors to perform part or all of any method encompassed herein.

Embodiments of the disclosure encompass a computer-program product tangibly embodied in a non-transitory machine-readable storage medium, including instructions configured to cause one or more data processors to perform part or all of any method encompassed herein.

Embodiments of the disclosure include methods of treating adenoma or CRC in a subject, the method comprising receiving a biological sample from the subject; determining a quantity of at least 1 peptide structure identified in Table 1 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has adenoma or CRC; and administering a therapeutically effective amount of the treatment for adenoma or CRC, respectively. In specific embodiments, the treatment comprises at least one of radiation therapy, chemotherapy, chemoradiotherapy, surgery, hormone therapy, or a targeted drug therapy. The method may further comprise preparing the biological sample to form a prepared sample comprising a set of peptide structures; and inputting the prepared sample into the MRM-MS system using a liquid chromatography system. The method may be further defined as determining a quantity of at least 1 peptide structure identified in Table 1 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has adenoma or CRC; and administering a therapeutically effective amount of the treatment for adenoma or CRC, respectively.

Embodiments of the disclosure include methods of identifying a need for one or more medical tests for a subject suspected of being at risk for or having an adenoma or CRC state, the method may comprise subjecting the subject to the one or more medical tests in response to measuring that a biological sample obtained from the subject evidences the state using part or all of any method encompassed herein. The one or more medical tests may comprise colonoscopy, physical exam, CT scan, MRI scan, PET scan, or a combination thereof.

Embodiments of the disclosure include methods of designing a treatment for a subject having an adenoma or CRC state, the method may comprise designing a therapeutic regimen for treating the subject in response to measuring that a biological sample obtained from the subject evidences the state using part or all of any method encompassed herein. The treatment may comprise at least one of radiation therapy, chemotherapy, chemoradiotherapy, surgery, hormone therapy, or a targeted drug therapy.

Embodiments of the disclosure include methods of treating a subject diagnosed with an adenoma or CRC state, and the method may comprise administering to the subject a therapeutic to treat the subject based on measuring that a biological sample obtained from the subject evidences the state using part or all of any method encompassed herein. The treatment may comprise at least one of radiation therapy, chemotherapy, chemoradiotherapy, surgery, hormone therapy, or a targeted drug therapy.

Embodiments of the disclosure include methods of treating a subject having an adenoma or CRC state, the method comprising: selecting a therapeutic to treat the subject based on determining that the subject is responsive to the therapeutic using any method encompassed herein. The treatment may comprise at least one of radiation therapy, chemotherapy, chemoradiotherapy, surgery, hormone therapy, or a targeted drug therapy.

Embodiments of the disclosure include methods of classifying a sample from an individual suspected of having, known to have, or at risk for an adenoma or CRC, comprising the step of measuring from the sample for one or more glycopeptides and/or non-glycosylated peptides in Table 1. The measuring may identify the individual as not having adenoma or CRC. In specific embodiments, the measuring identifies the individual as having adenoma or CRC. The measuring may identify the individual as having early stage CRC or late stage CRC. The measuring may comprise successive or concomitant steps of identifying that the individual has CRC and that the individual has early stage CRC. In specific cases, the sample may comprise stool, peripheral blood, plasma, or serum. The individual may be at risk for adenoma or CRC. In specific embodiments, the measuring may identify the individual as having adenoma or CRC, the individual is administered an effective amount of at least one of radiation therapy, chemotherapy, chemoradiotherapy, surgery, hormone therapy, or a targeted drug therapy. The sample may be measured for 1, 2, 3, 4, 5, or all of the glycopeptides and/or non-glycosylated peptides of Table 1.

Embodiments of the disclosure include methods of predicting a risk for adenoma or CRC in a subject, the method comprising receiving a biological sample from the subject; determining a quantity of at least 1 peptide structure identified in Table 1 in the biological sample using a multiple reaction monitoring mass spectrometry (MRM-MS) system; analyzing the quantity of each peptide structure using at least one machine learning model to generate a disease indicator; and generating a diagnosis output based on the disease indicator that classifies the biological sample as evidencing that the subject has a risk for adenoma or CRC.

Embodiments of the disclosure include methods of diagnosing adenoma or CRC or predicting a risk for adenoma or CRC in an individual, comprising the step of identifying one or more peptide structures identified in Table 1 from a sample from the individual.

Embodiments of the disclosure include methods of identifying and managing an at-risk subject for CRC, the method comprising measuring whether a biological sample obtained from the subject evidences a CRC state using part or all of any method encompassed herein and subjecting the subject to one or more medical tests in response to the identification of the CRC state.

In one aspect, a system is described according to various embodiments. In various embodiments, the system comprises one or more data processors and a non-transitory computer readable storage medium containing instructions which, when executed on the one or more data processors, cause the one or more data processors to perform part or all of any one or more of the methods described herein.

In one aspect, a computer-program product tangibly embodied in a non-transitory machine-readable storage medium, including instructions configured to cause one or more data processors to perform part or all of any one or more of the methods described herein.

In accordance with various embodiments, a method is provided for identifying and managing a subject at risk of an adenoma or CRC disease state. The method can comprise receiving a biological sample from the subject, determining a quantity of at least 1 peptide structure identified in Table 1 in the biological sample, analyzing the quantity of each peptide structure using at least one machine learning model to generate a disease indicator, generating a diagnosis output based on the disease indicator that classifies the biological sample as evidencing that the subject has a risk for adenoma or CRC, and identifying a need for a colonoscopy of the subject based on the classified risk of adenoma or CRC.

### Table 1B

The methods as described herein using the biomarkers of Table 1 may be applied similarly to using the biomarkers of Table 1B. The methods as described herein using the product ions or precursor ions of Table 2 may be applied similarly to using the product ions or precursor ions of Table 2B. The methods as described herein using the peptide sequence of Table 3A may be applied similarly to using the peptide sequence of Table 3C. The methods as described herein using the glycan structure and glycan composition of Table 5 may be applied similarly to using the glycan structure and glycan composition of Tables 5B and 5C.

In accordance with various embodiments, a method of screening a subject is described. The method includes analyzing a peptide structure data using at least one supervised machine learning model to generate a disease indicator that indicates whether the biological sample evidences an APL or CRC disease state based on at least one peptide structure selected from a group of peptide structures identified in Table 1B. The peptide structure data corresponds to a biological sample obtained from the subject. The method further includes outputting either a recommendation to perform a colonoscopy or to not perform the colonoscopy based on the disease indicator. In an aspect, the subject can be subjected to a colonoscopy when the recommendation to perform the colonoscopy is outputted. In another aspect, the subject does not have any symptoms of APL and/or CRC.

In accordance with the various screening embodiments, the group of peptide structures in Table 1B can be associated with the APL or CRC disease state. The group of peptide structures can be listed in Table 1B with respect to relative significance to the disease indicator. The method can further include receiving peptide structure data corresponding to the biological sample obtained from the subject.

In accordance with the various screening embodiments, the disease indicator can include a score, wherein generating the diagnosis output comprises determining that the score falls above a selected threshold; and generating the diagnosis output based on the score falling above the selected threshold, wherein the diagnosis output includes a positive diagnosis for the APL or CRC disease state.

In accordance with the various screening embodiments, analyzing the peptide structure data can include analyzing the peptide structure data using a binary classification model.

In accordance with the various screening embodiments, the at least one peptide structure can include a glycopeptide structure defined by a peptide sequence and a glycan structure linked to the peptide sequence at a linking site of the peptide sequence, as identified in Table 1B, with the peptide sequence being one of SEQ ID NOS: 27-41 as defined in Table 1B and Table 3C.

In accordance with the various screening embodiments, the peptide structure data can include at least one of a raw abundance, an adjusted raw abundance, a peptide concentration, a glycopeptide concentration, or a normalized concentration. The peptide structure data can include normalized concentration data, wherein the normalized concentration data is a function of at least one of peptide abundance data, corresponding internal standard abundance data, a spike-in concentration value, and a dilution factor. The peptide structure data can be generated using multiple reaction monitoring mass spectrometry (MRM-MS).

In accordance with the various screening embodiments, the method can include creating a sample from the biological sample; and preparing the sample using reduction, alkylation, and enzymatic digestion to form a prepared sample that includes a set of peptide structures. The method can further include generating the peptide structure data from the prepared sample using multiple reaction monitoring mass spectrometry (MRM-MS).

In accordance with the various screening embodiments, the recommendation can be a report identifying that the biological sample evidences the APL or CRC disease state.

In accordance with various embodiments, the binary classification model includes a first classification where the subject is healthy and a second classification where the subject has APL or CRC.

In regard to any of the embodiments, the biological sample can be in a tube that comprises an anticoagulant and a preserving agent. The method can further include isolating a plasma fraction from the tube to create a sample from the biological sample. The sample can be prepared using reduction, alkylation, and enzymatic digestion to form a prepared sample that includes a set of peptide structures.

In regard to the embodiments that use the biological sample and a tube including the anticoagulant and the preserving agent, the anticoagulant can include EDTA salt and the preserving agent can include imidazolidinyl urea.

In regard to the embodiments that use the biological sample and a tube including the anticoagulant and the preserving agent, the tube can further include glycine.

In regard to the embodiments that use the biological sample and a tube including the anticoagulant and the preserving agent, before the isolating the plasma fraction, the biological sample can contact the preserving agent for a period of time ranging from 24 hours to 7 days.

In regard to any of the embodiments, the biological sample can be in a tube that includes silica particles. The method further includes isolating a serum fraction from the tube to create a sample from the biological sample. The sample can be prepared using reduction, alkylation, and enzymatic digestion to form a prepared sample that includes a set of peptide structures.

In regard to the embodiments that use the biological sample and a tube including silica particles, the tube further includes a polyester gel configured to form a barrier between a serum fraction and blood cells during a centrifugation process.

In regard to the embodiments that use the biological sample and a tube including silica particles, the silica particles were spray-coated onto an inner surface of the tube.

In regard to the embodiments that use the biological sample and a tube including silica particles, the biological sample formed a clot in the tube before the isolating the serum fraction from the tube.

### Table 1C

The methods as described herein using the biomarkers of Table 1 may be applied similarly to using the biomarkers of Table 1C. The methods as described herein using the product ions or precursor ions of Table 2 may be applied similarly to using the product ions or precursor ions of Table 2C. The methods as described herein using the peptide sequence of Table 3A may be applied similarly to using the peptide sequence of Table 3E. The methods as described herein using the glycan structure and glycan composition of Table 5 may be applied similarly to using the glycan structure and glycan composition of Tables 5D and 5E.

In accordance with various embodiments, a method of screening a subject is described. The method includes analyzing a peptide structure data using at least one supervised machine learning model to generate a disease indicator that indicates whether the biological sample evidences a high-grade advanced pre-malignant lesion or CRC disease state based on at least one peptide structure selected from a group of peptide structures identified in Table 1C. The peptide structure data corresponds to a biological sample obtained from the subject. The method further includes outputting either a recommendation to perform a colonoscopy or to not perform the colonoscopy based on the disease indicator. In an aspect, the subject can be subjected to a colonoscopy when the recommendation to perform the colonoscopy is outputted. In another aspect, the subject does not have any symptoms of high-grade advanced pre-malignant lesion and/or CRC.

In accordance with the various screening embodiments, the group of peptide structures in Table 1C can be associated with the high-grade advanced pre-malignant lesion or CRC disease state. The group of peptide structures can be listed in Table 1C with respect to relative significance to the disease indicator. The method can further include receiving peptide structure data corresponding to the biological sample obtained from the subject.

In accordance with the various screening embodiments, the disease indicator can include a score, wherein generating the diagnosis output comprises determining that the score falls above a selected threshold; and generating the diagnosis output based on the score falling above the selected threshold, wherein the diagnosis output includes a positive diagnosis for the high-grade advanced pre-malignant lesion or CRC disease state.

In accordance with the various screening embodiments, analyzing the peptide structure data can include analyzing the peptide structure data using a binary classification model.

In accordance with the various screening embodiments, the at least one peptide structure can include a glycopeptide structure defined by a peptide sequence and a glycan structure linked to the peptide sequence at a linking site of the peptide sequence, as identified in Table 1C, with the peptide sequence being one of SEQ ID NOS: 42-111 as defined in Table 1C and/or Table 3E.

In accordance with the various screening embodiments, the peptide structure data can include at least one of a raw abundance, an adjusted raw abundance, a peptide concentration, a glycopeptide concentration, or a normalized concentration. The peptide structure data can include normalized concentration data, wherein the normalized concentration data is a function of at least one of peptide abundance data, corresponding internal standard abundance data, a spike-in concentration value, and a dilution factor. The peptide structure data can be generated using multiple reaction monitoring mass spectrometry (MRM-MS).

In accordance with the various screening embodiments, the method can include creating a sample from the biological sample; and preparing the sample using reduction, alkylation, and enzymatic digestion to form a prepared sample that includes a set of peptide structures. The method can further include generating the peptide structure data from the prepared sample using multiple reaction monitoring mass spectrometry (MRM-MS).

In accordance with the various screening embodiments, the recommendation can be a report identifying that the biological sample evidences the high-grade advanced pre-malignant lesion or CRC disease state.

In accordance with various embodiments, the binary classification model includes a first classification where the subject is healthy and a second classification where the subject has high-grade advanced pre-malignant lesion or CRC.

In regard to any of the embodiments, the biological sample can be in a tube that comprises an anticoagulant and a preserving agent. The method can further include isolating a plasma fraction from the tube to create a sample from the biological sample. The sample can be prepared using reduction, alkylation, and enzymatic digestion to form a prepared sample that includes a set of peptide structures.

In regard to the embodiments that use the biological sample and a tube including the anticoagulant and the preserving agent, the anticoagulant can include EDTA salt and the preserving agent can include imidazolidinyl urea.

In regard to the embodiments that use the biological sample and a tube including the anticoagulant and the preserving agent, the tube can further include glycine.

In regard to the embodiments that use the biological sample and a tube including the anticoagulant and the preserving agent, before the isolating the plasma fraction, the biological sample can contact the preserving agent for a period of time ranging from 24 hours to 7 days.

In regard to any of the embodiments, the biological sample can be in a tube that includes silica particles. The method further includes isolating a serum fraction from the tube to create a sample from the biological sample. The sample can be prepared using reduction, alkylation, and enzymatic digestion to form a prepared sample that includes a set of peptide structures.

In regard to the embodiments that use the biological sample and a tube including silica particles, the tube further includes a polyester gel configured to form a barrier between a serum fraction and blood cells during a centrifugation process.

In regard to the embodiments that use the biological sample and a tube including silica particles, the silica particles were spray-coated onto an inner surface of the tube.

In regard to the embodiments that use the biological sample and a tube including silica particles, the biological sample formed a clot in the tube before the isolating the serum fraction from the tube.

### Table 1D

The methods as described herein using the biomarkers of Table 1 may be applied similarly to using the biomarkers of Table 1D. The methods as described herein using the product ions or precursor ions of Table 2 may be applied similarly to using the product ions or precursor ions of Table 2D. The methods as described herein using the peptide sequence of Table 3A may be applied similarly to using the peptide sequence of Table 3G. The methods as described herein using the glycan structure and glycan composition of Table 5 may be applied similarly to using the glycan structure and glycan composition of Tables 5F and 5G.

In accordance with various embodiments, a method of screening a subject is described. The method includes analyzing a peptide structure data using at least one supervised machine learning model to generate a disease indicator that indicates whether the biological sample evidences a CRC disease state based on at least one peptide structure selected from a group of peptide structures identified in Table 1D. The peptide structure data corresponds to a biological sample obtained from the subject. The method further includes outputting either a recommendation to perform a colonoscopy or to not perform the colonoscopy based on the disease indicator. In an aspect, the subject can be subjected to a colonoscopy when the recommendation to perform the colonoscopy is outputted. In another aspect, the subject does not have any symptoms of CRC.

In accordance with the various screening embodiments, the group of peptide structures in Table 1D can be associated with the CRC disease state. The group of peptide structures can be listed in Table 1D with respect to relative significance to the disease indicator. The method can further include receiving peptide structure data corresponding to the biological sample obtained from the subject.

In accordance with the various screening embodiments, the disease indicator can include a score, wherein generating the diagnosis output comprises determining that the score falls above a selected threshold; and generating the diagnosis output based on the score falling above the selected threshold, wherein the diagnosis output includes a positive diagnosis for the CRC disease state.

In accordance with the various screening embodiments, analyzing the peptide structure data can include analyzing the peptide structure data using a binary classification model.

In accordance with the various screening embodiments, the at least one peptide structure can include a glycopeptide structure defined by a peptide sequence and a glycan structure linked to the peptide sequence at a linking site of the peptide sequence, as identified in Table 1D, with the peptide sequence being one of SEQ ID NOS: 136-156 as defined in Table 1D and/or Table 3G.

In accordance with the various screening embodiments, the peptide structure data can include at least one of a raw abundance, an adjusted raw abundance, a peptide concentration, a glycopeptide concentration, or a normalized concentration. The peptide structure data can include normalized concentration data, wherein the normalized concentration data is a function of at least one of peptide abundance data, corresponding internal standard abundance data, a spike-in concentration value, and a dilution factor. The peptide structure data can be generated using multiple reaction monitoring mass spectrometry (MRM-MS).

In accordance with the various screening embodiments, the method can include creating a sample from the biological sample; and preparing the sample using reduction, alkylation, and enzymatic digestion to form a prepared sample that includes a set of peptide structures. The method can further include generating the peptide structure data from the prepared sample using multiple reaction monitoring mass spectrometry (MRM-MS).

In accordance with the various screening embodiments, the recommendation can be a report identifying that the biological sample evidences the CRC disease state.

In accordance with various embodiments, the binary classification model includes a first classification where the subject is healthy and a second classification where the subject has CRC.

In regard to any of the embodiments, the biological sample can be in a tube that comprises an anticoagulant and a preserving agent. The method can further include isolating a plasma fraction from the tube to create a sample from the biological sample. The sample can be prepared using reduction, alkylation, and enzymatic digestion to form a prepared sample that includes a set of peptide structures.

In regard to the embodiments that use the biological sample and a tube including the anticoagulant and the preserving agent, the anticoagulant can include EDTA salt and the preserving agent can include imidazolidinyl urea.

In regard to the embodiments that use the biological sample and a tube including the anticoagulant and the preserving agent, the tube can further include glycine.

In regard to the embodiments that use the biological sample and a tube including the anticoagulant and the preserving agent, before the isolating the plasma fraction, the biological sample can contact the preserving agent for a period of time ranging from 24 hours to 7 days.

In regard to any of the embodiments, the biological sample can be in a tube that includes silica particles. The method further includes isolating a serum fraction from the tube to create a sample from the biological sample. The sample can be prepared using reduction, alkylation, and enzymatic digestion to form a prepared sample that includes a set of peptide structures.

In regard to the embodiments that use the biological sample and a tube including silica particles, the tube further includes a polyester gel configured to form a barrier between a serum fraction and blood cells during a centrifugation process.

In regard to the embodiments that use the biological sample and a tube including silica particles, the silica particles were spray-coated onto an inner surface of the tube.

In regard to the embodiments that use the biological sample and a tube including silica particles, the biological sample formed a clot in the tube before the isolating the serum fraction from the tube.

### Tables 13A and 13B

In some aspects, the present invention relates to diagnosis of colorectal cancer (CRC) based upon certain glycopeptide biomarkers provided herein, such as those in Tables 13A and 13B. In some embodiments, the methods provided herein are minimally invasive or non-invasive methods for diagnosing CRC that result in early detection of CRC and/or identification of a risk of CRC to enable early treatment for at risk individuals. In some embodiments, the method further comprises providing a recommendation to an individual determined to be at risk for CRC to undergo an endoscopy (e.g., colonoscopy) based upon the determined risk.

In some embodiments, the method further comprises performing an endoscopy on the individual to diagnose colorectal cancer. In some embodiments, the method further comprises administering an effective amount of a therapeutic agent (e.g., chemotherapy agent) to treat CRC based upon the disease indicator and/or determined risk.

Also provided herein is a method of treating colorectal cancer (CRC) in an individual comprising detecting the presence or amount of at least one peptide structure, wherein the at least one peptide structure comprises at least one peptide structure from Table 13A, and administering an effective amount of a therapeutic agent to treat CRC based upon the presence or amount of the peptide structure. In some embodiments, the method of treating CRC in an individual comprises detecting the presence or amount of at least one peptide structure, wherein the at least one peptide structure comprises at least one peptide structure from Table 13B, and administering an effective amount of a therapeutic agent to treat CRC based upon the presence or amount of the peptide structure.

In some embodiments, provided herein is a method of treating colorectal cancer (CRC) in an individual comprising detecting a presence or amount of at least one peptide structure to determine a risk of CRC, wherein the at least one peptide structure comprises at least one peptide structure from Table 13A, and administering a therapeutic agent to treat CRC based upon the determined risk of CRC. In some embodiments, the method of treating CRC in an individual comprising detecting a presence or amount of at least one peptide structure to determine a risk of CRC, wherein the at least one peptide structure comprises at least one peptide structure from Table 13B, and administering a therapeutic agent to treat CRC based upon the determined risk of CRC.

In some embodiments, provided herein is a method of diagnosing an individual with colorectal cancer (CRC) comprising detecting a presence or amount of at least one peptide structure, wherein the at least one peptide structure comprises at least one peptide structure from Table 13A or Table 13B, and diagnosing the individual with CRC based upon the presence or amount of the at least one peptide structure.

In some embodiments, provide herein is a method of determining a risk for developing colorectal cancer (CRC) comprising detecting a presence or amount of at least one peptide structure and determining the risk for developing CRC based upon the presence or amount of the at least one peptide structure, wherein the at least one peptide structure comprises at least one peptide structure from Table 13A or Table 13B.

In some embodiments, the presence or amount of the at least one peptide structure is detected using mass spectrometry or ELISA. In some embodiments, the amount of at least one peptide structure is none, or below a detection limit. In some embodiments, the colorectal cancer (CRC) is early-stage CRC, the CRC is late-stage CRC, or the CRC is severe CRC. In some embodiments, the biological sample is plasma sample, a serum sample, or a blood sample. In some embodiments, the biological sample is a stool sample.

In some embodiments, the at least one peptide structure comprises three or more peptide structures identified in Table 13A. In some embodiments, the at least one peptide structure comprises the sequence set forth in SEQ ID NOs: 168-198. In some embodiments, the at least one peptide structure comprises three or more peptide structures identified in Table 13B. In some embodiments, the at least one peptide structure comprises the sequence set forth in SEQ ID NOs: 168, 171, 172, 176, 181, 184, 187, 192, and 194.

In some embodiments, the method further comprises assessing one or more risk factors or clinical indicators of colorectal cancer (CRC). In some embodiments, the risk factor for CRC is selected from the group consisting of age, irritable bowel disease, type 2 diabetes, a family history of CRC, a genetic syndrome (e.g., Lynch syndrome), obesity, smoking, alcohol consumption, dietary choices, and limited physical activity. In some embodiments, the clinical indicator of CRC is selected from the group consisting of changes in bowel habits, bloody stool, diarrhea, constipation, persistent abdominal pain, persistent abdominal cramps, and unexplained weight loss.

In some embodiments, the individual is determined have a healthy state, wherein a healthy state comprises the absence of colorectal cancer (CRC) and/or a low risk for CRC.

In some embodiments, the method further comprises diagnosing a colon polyp, a colorectal adenoma, or an advanced colorectal adenoma.

In some embodiments, the method further comprises generating a report that includes a diagnosis based on the corresponding state detected for the subject.

In some embodiments, at least one of the peptide structures comprises a glycopeptide. In some embodiments, the at least one peptide comprising a glycopeptide is derived from a glycoprotein.

Also provided herein is a composition comprising one or more peptide structures from Table 13A or Table 13B.

Provided herein is a composition comprising one or more peptides comprising the sequence set forth in SEQ ID NOs: 168-198. Further provided herein is a composition comprising one or more peptides comprising the sequence set forth in SEQ ID NOs: 168, 171, 172, 176, 181, 184, 187, 192, and 194.

### BRIEF DESCRIPTION OF THE DRAWINGS

The present disclosure is described in conjunction with the appended figures:

FIG. 1 is a schematic diagram of an exemplary workflow 100 for the detection of peptide structures associated with a disease state for use in diagnosis and/or treatment in accordance with one or more embodiments.

FIG. 2A is a schematic diagram of a preparation workflow in accordance with one or more embodiments.

FIG. 2B is a schematic diagram of data acquisition in accordance with one or more embodiments.

FIG. 3 is a block diagram of an analysis system in accordance with one or more embodiments.

FIG. 4 is a block diagram of a computer system in accordance with various embodiments.

FIG. 5 is a flowchart of a process for diagnosing a subject with respect to an adenoma or colorectal cancer disease state and Table 1 in accordance with one or more embodiments.

FIG. 5B is a flowchart of a process for diagnosing a subject with respect to an APL colorectal cancer disease state and Table 1B in accordance with one or more embodiments.

FIG. 5C is a flowchart of a process for diagnosing a subject with respect to a high-grade advanced pre-malignant lesion or colorectal cancer disease state and Table 1C in accordance with one or more embodiments.

FIG. 5D is a flowchart of a process for diagnosing a subject with respect to a colorectal cancer disease state and Table 1D in accordance with one or more embodiments.

FIG. 6 is a flowchart of a process for training a model to diagnose a subject with respect to adenoma or CRC disease state and Table 1 in accordance with one or more embodiments.

FIG. 6B is a flowchart of a process for training a model to diagnose a subject with respect to APL or CRC disease state and Table 1B in accordance with one or more embodiments.

FIG. 6C is a flowchart of a process for training a model to diagnose a subject with respect to high-grade advanced pre-malignant lesion or CRC disease state and Table 1C in accordance with one or more embodiments.

FIG. 6D is a flowchart of a process for training a model to diagnose a subject with respect to the CRC disease state and Table 1D in accordance with one or more embodiments.

FIG. 7 is a flowchart of a process for monitoring a subject for an adenoma or CRC in accordance with one or more embodiments.

FIG. 7B is a flowchart of a process for monitoring a subject for an APL or CRC in accordance with one or more embodiments.

FIG. 7C is a flowchart of a process for monitoring a subject for a high-grade advanced pre-malignant lesion or CRC in accordance with one or more embodiments.

FIG. 7D is a flowchart of a process for monitoring a subject for a CRC in accordance with one or more embodiments.

FIG. 8 is a receiver operating characteristic (ROC) curve in accordance with various embodiments.

FIG. 9 demonstrates a probability of CRC or adenoma based on an examination of a Train & Test data set to determine the performance of the classifier model, utilizing samples of adenoma, ulcerative colitis control, healthy control, and colorectal cancer of a collection of stages.

FIG. 10 demonstrates a probability of advanced adenoma or CRC based on an examination of a Train & Test data set to determine the performance of the classifier model, utilizing samples of advanced adenoma (high-grade), advanced adenoma (low-grade), respective stages 1, 2, 3, and 4 of CRC, healthy control, and ulcerative colitis control. Equivalent probability distributions between training and test sets indicates a well-fit model, and application to advanced adenomas and stages 3 and 4 of CRC, exclusively considered in the test set, demonstrates a biologically-relevant score that tracks with the progression of the disease.

FIG. 11 shows a principal component analysis (PCA) plot to visualize various features that exhibit the intrinsic variation among different subgroups.

FIG. 12 shows a clustered heatmap of patients (color-coded along the x-axis by their disease indication) for all normalized abundance features that have an FDR<0.05. As indicated above, several potential biomarkers are differentially expressed between CRC/AA patients and healthy/UC controls.

FIG. 13 is a receiver operating characteristic (ROC) curve in accordance with various embodiments relating to the comparison of APL/CRC vs Non-APL/Ctrl.

FIG. 14 is a plot demonstrating a support vector machine (SVM) score for a training data set that classifies samples where the data set includes healthy controls, non-APL, APL, CRC stage 1/2, and CRC stage 3/4.

FIG. 15 is a plot demonstrating a support vector machine (SVM) score for a validation data set that classifies samples where the data set includes healthy controls, non-APL, APL, CRC stage 1/2, and CRC stage 3/4.

FIG. 16 is a plot demonstrating a support vector machine (SVM) score for a test data set that classifies samples where the data set includes healthy controls, non-APL, APL, CRC stage 1/2, and CRC stage 3/4.

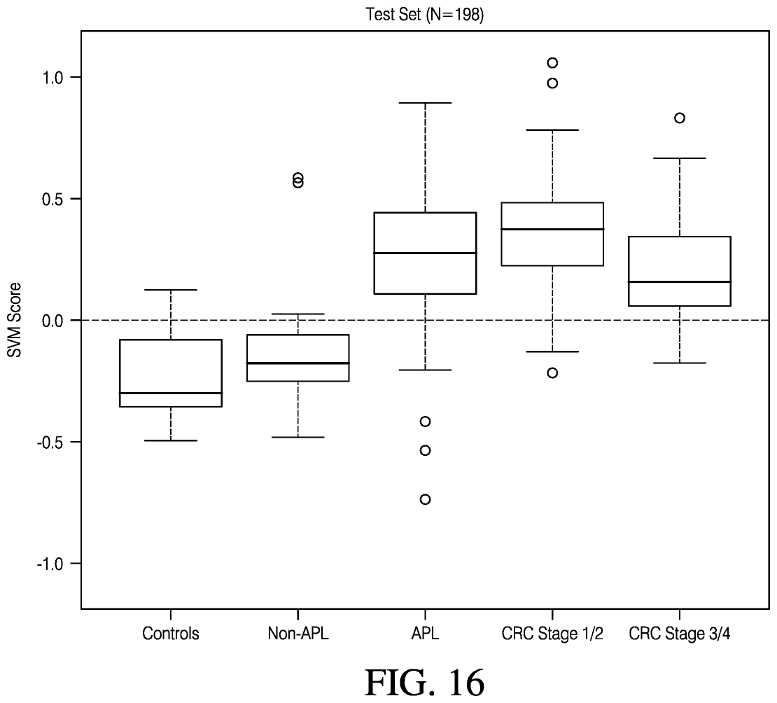

FIG. 17 is a plot showing low-grade adenoma sensitivity, high grade advanced pre-malignant lesions sensitivity, CRC 1 & 2 sensitivity, and specificity.

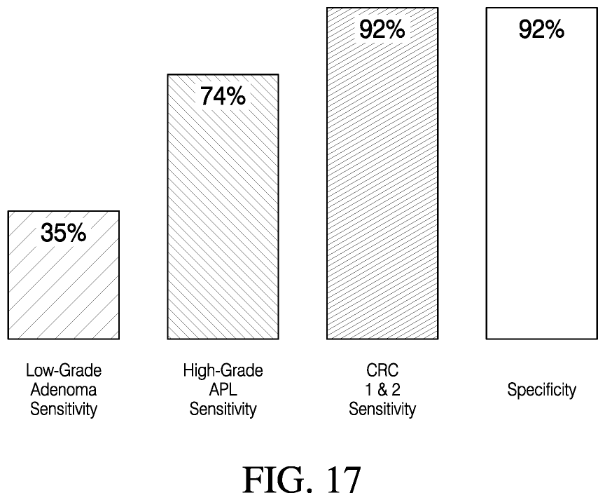

FIG. 18 is a ROC plot in accordance with various embodiments relating to the comparison of adenoma/CRC vs healthy control samples.

FIG. 19 is a probability plot showing train and test performance of the model for adenoma, healthy control, and CRC samples.

FIG. 20 is a probability plot showing train and test performance of the model for adenoma, healthy control, Stage 1, Stage 2, Stage 3, and Stage 4 CRC samples.

FIG. 21 shows an experimental workflow for sample preparation and analysis.

FIG. 22 shows the number of spectral matching for unique N-glycopeptides (N-glycopeptide abundance) for all colorectal cancer (CRC)N-glycopeptides (dotted trace) and select CRC biomarkers (triangles).

### DETAILED DESCRIPTION

### I. Overview

Colorectal cancer (CRC) is a leading cause of cancer-related deaths in the United States with over 150,000 diagnosed cases and over 53,000 deaths in 2020. According to a 2021 study, there are an estimated 1.85 million diagnoses per year and 850,000 deaths worldwide.

CRC results from uncontrolled cell growth in the lower gastrointestinal tract, such as the colon, rectum or appendix. CRC can develop from a colon polyp, which are typically benign cell growths on the lining of the large intestine or rectum. However, a polyp can progress to colorectal adenoma, advanced colorectal adenomas, and CRC if it is not diagnosed and treated.

Patient survival rates are highly dependent on when CRC is diagnosed. For example, the five-year survival rate is over 90% for those patients diagnosed with Stage I CRC, compared to just 13% for Stage IV diagnosis. Once identified, the cancerous tissue can be surgically removed, followed by chemotherapy if the CRC has metastasized beyond the initial tumor.

CRC is one of the most preventable cancers given its slow progression and available diagnostic tools (e.g., colonoscopy). Regular screenings are critical for effective treatment of CRC, but poor compliance with available screening approaches makes CRC one of the least prevented cancers.

Current screening approaches involve either stool sample analysis or direct observation via a colonoscopy or sigmoidoscopy. However, the highly invasive nature and the expense of these exams contribute to low compliance rates. As a result, CRC is often detected only after progressing past the point at which treatment success rates have declined substantially. Furthermore, these invasive procedures expose patients to risk of complications such as infection. Non-invasive options are available (e.g., the fecal occult blood test, FOBT), but these have proven unreliable with high false-positive rates and low sensitivity.

Given the life threatening consequences of CRC, and the high-likelihood of successful therapeutic intervention if detected early, there is clear need for a reliable, non-invasive screening approach that provides early and unambiguous diagnosis of CRC.

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, serum sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

However, to understand various disease conditions and to diagnose certain diseases, such as colorectal cancer, more accurately, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns that may be able to provide information about a disease state (e.g., a colorectal cancer disease state). This information can be used to distinguish the disease state from other states, diagnose a subject as having or not having the disease state, determine a likelihood that a subject has the disease state, or a combination thereof. For example, such analysis may be useful in diagnosing an adenoma or colorectal cancer disease state for a subject (e.g., a negative diagnosis for the adenoma or colorectal cancer (and/or advanced adenoma) disease state, a positive diagnosis for the adenoma or colorectal cancer disease state). Sample collection and analysis can be collected at different time points for comparing adenoma or colorectal cancer disease states over time for a subject. For example, the negative diagnosis may include a healthy state. An example of the positive diagnosis includes the subject suffering from colorectal cancer or adenoma disease state. A diagnosis can also assess a malignancy status of a previously identified colorectal tumor (or mass).

Accordingly, the embodiments described herein provide various methods and systems for analyzing proteins in subjects and, in particular, glycoproteins. In one or more embodiments, one or more machine learning models are trained to analyze peptide structure data and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

The embodiments described herein recognize that the abundance of selected peptide structures in a biological sample obtained from a subject may be used to determine the likelihood of that subject evidencing an adenoma or colorectal cancer disease state. An adenoma or colorectal cancer disease state may include any condition that can be diagnosed as an adenoma or cancer that occurs in the colon or rectum. Certain peptide structures that are associated with an adenoma or colorectal cancer disease state may be more relevant to that disease state than other peptide structures that are also associated with that disease state.

Analyzing the abundance of peptide sequences and glycosylated peptide sequences in a biological sample may provide a more accurate way in which to distinguish a positive colorectal cancer disease state (e.g., a state including the presence of colorectal cancer) from a negative colorectal cancer disease state (e.g., healthy state, an absence of colorectal cancer, etc.). This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

Further, the methods, systems, and compositions provided by the embodiments described herein may enable an earlier, more accurate and/or less invasive diagnosis of colorectal cancer in a subject as compared to currently available diagnostic modalities (e.g., colonoscopy, biopsies, imaging, biochemical tests) used for determining whether surgical intervention is indicated.

The description below provides exemplary implementations of the methods and systems described herein for the research, diagnosis, and/or treatment of a colorectal cancer disease state. Various examples implement the methods and systems described herein as a screening tool. Descriptions and examples of various terms, as used herein, are provided in Section II below.

### II. Exemplary Descriptions of Terms

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

The term “ones” means more than one.

As used herein, the term “plurality” is more than 1 and may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list is required to be included. The item may be a particular object, thing, step, operation, process, or category. In other words, “at least one of” means any combination of items or number of items may be used from the list, but not all of the items in the list may be required. For example, without limitation, “at least one of item A, item B, and item C” intends and includes any of item A; item A and item B; item B; item A, item B, and item C; item B and item C; item C; and item A and C. It is understood that “at least one of” includes instance where more than one of any listed item is present. For example, and without limitation, at least one of item A, item B, and item C include an embodiment in which two of item A is present, one of item B is present, and ten of item C is present.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

“Treating” or treatment of a disease or condition refers to executing a protocol, which may include administering one or more drugs to an individual, such as a patient (or subject), in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutically effective” as used throughout this application refers to anything that promotes or enhances the well-being of the subject with respect to the medical treatment of this condition. This includes, but is not limited to, a reduction in the frequency or severity of one or more signs or symptoms of a disease, including adenomas or colorectal cancer.

The term “colorectal cancer” as used herein refers to cancer that starts in the colon or the rectum.

The term “colorectal cancer (CRC) disease state” as used herein refers to the presence in an individual of colorectal cancer of any type and of any stage.

The term “early stage” as used herein refers to stage 0, stage 1, or stage 2 colorectal cancer, such as defined by the American Joint Committee on Cancer (AJCC) TNM system and based on the size of the tumor, whether or not it has spread to nearby lymph nodes, and whether or not it has spread to distant sites.

The term “late stage” as used herein refers to stage 3 or stage 4 colorectal cancer, such as defined by the American Joint Committee on Cancer (AJCC) TNM system and based on the size of the tumor, whether or not it has spread to nearby lymph nodes, and whether or not it has spread to distant sites.

The term “amino acid,” as used herein, generally refers to any organic compound that includes an amino group (e.g., —NH₂), a carboxyl group (—COOH), and a side chain group (R) which varies based on a specific amino acid. Thus, “amino acid” includes organic compounds of the formula NH₂—CH(R)—COOH where R represents an amino acid side chain group. In some instance R represents the side chain of a natural amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. Biological samples may include, but are not limited to stool, synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to stool, biopsy, blood and/or plasma. In some examples, biological samples include, but are not limited, to urine or stool. Biological samples include, but are not limited, to biopsy. Biological samples include, but are not limited, to tissue dissections and tissue biopsies. Biological samples include, but are not limited, any derivative or fraction of the aforementioned biological samples. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “biomarker,” as used herein, generally refers to any measurable substance taken as a sample from a subject whose presence, absence and/or amount is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a disease state, a health state, an asymptomatic state, a symptomatic state, etc). The term “biomarker” can be used interchangeably with the term “marker.”

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in its native state.

The terms “digestion” or “enzymatic digestion,” as used herein, generally refers to a biological process that employs enzymes to break specific amino acid peptide bonds. For example, digesting a peptide includes contacting the peptide with an digesting enzyme, e.g., trypsin to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing. Enzymatic digestion may be used in preparation for mass spectrometry using trypsin digestion protocols. Proteins may be digested using other proteases in preparation for mass spectrometry if access is limited to cleavage sites.

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

The term “fragment,” as used herein, generally refers to an ion fragmentation process which occurs in a MRM-MS instrument. Fragmenting may produce various fragments having the same mass but varying with respect to their charge, e.g., some biomarkers described herein produce more than one product m/z.

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refers to a peptide or polypeptide comprising at least one glycan residue. In various embodiments, glycopeptides comprise carbohydrate moieties (e.g., one or more glycans) covalently attached to a side chain (i.e. R group) of an amino acid residue.

The term “glycopeptide fragment” or “glycosylated peptide fragment” or “glycopeptide” as used herein, generally refers to a glycosylated peptide (or glycopeptide) having an amino acid sequence that is the same as part (but not all) of the amino acid sequence of the glycosylated protein from which the glycosylated peptide is obtained, e.g., ion fragmentation within a MRM-MS instrument. MRM refers to multiple-reaction-monitoring. Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins include but are not limited to the peptide structures including glycan molecules shown in the various Tables presented herein. A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “m/z” or “mass-to-charge ratio,” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. As used herein, the phrase “peptide,” is meant to include glycopeptides unless stated otherwise.

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ (H₂O)ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that is in need of therapy or suspected of needing therapy. A subject can be a patient. A subject can include a microorganism or microbe (e.g., bacteria, fungi, archaea, viruses). A subject may be one who has been previously identified as having a disease or a condition, and optionally has already undergone, or is undergoing, a therapeutic intervention for the disease or condition. Alternatively, a subject can also be one who has not been previously diagnosed as having a disease or a condition. For example, a subject can be one who exhibits one or more risk factors for a disease or a condition, or a subject who does not exhibit disease risk factors, or a subject who is asymptomatic for a disease or a condition. A subject can also be one who is suffering from or at risk of developing a disease or a condition. A subject may also be referred to as an individual or patient.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry.

As used herein, a “peptide data set,” may be used interchangeably with “peptide structure data” and can refer to any data of or relating to a peptide from a resulting mass spectrometry run. A peptide data set can comprise data obtained from a sample or biological sample using mass spectrometry. A peptide dataset can comprise data relating to an external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “a transition,” may refer to or identify a peptide structure. In some embodiments, a transition can refer to the specific pair of m/z values associated with a precursor ion and a product or fragment ion.

As used herein, a “non-glycosylated endogenous peptide” (“NGEP”) may refer to a peptide structure that does not comprise a glycan molecule. In various embodiments, an NGEP and a target glycopeptide analyte can originate from the same subject. In various embodiments, an NGEP and a target glycopeptide analyte may be derived from the same protein sequence. In some embodiments, the NGEP and the target glycopeptide analyte may be derived from or include the same peptide sequence. In various embodiments, an NGEP can be labeled with an isotope in preparation for mass spectrometry analysis.

As used herein, “abundance,” may refer to a quantitative value generated using mass spectrometry. In various embodiments, the quantitative value may relate to the amount of a particular peptide structure. In some embodiments, the quantitative value may comprise an amount of an ion produced using mass spectrometry. In some embodiments, the quantitative value may be expressed as an m/z value. In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

### III. Overview of Exemplary Workflow

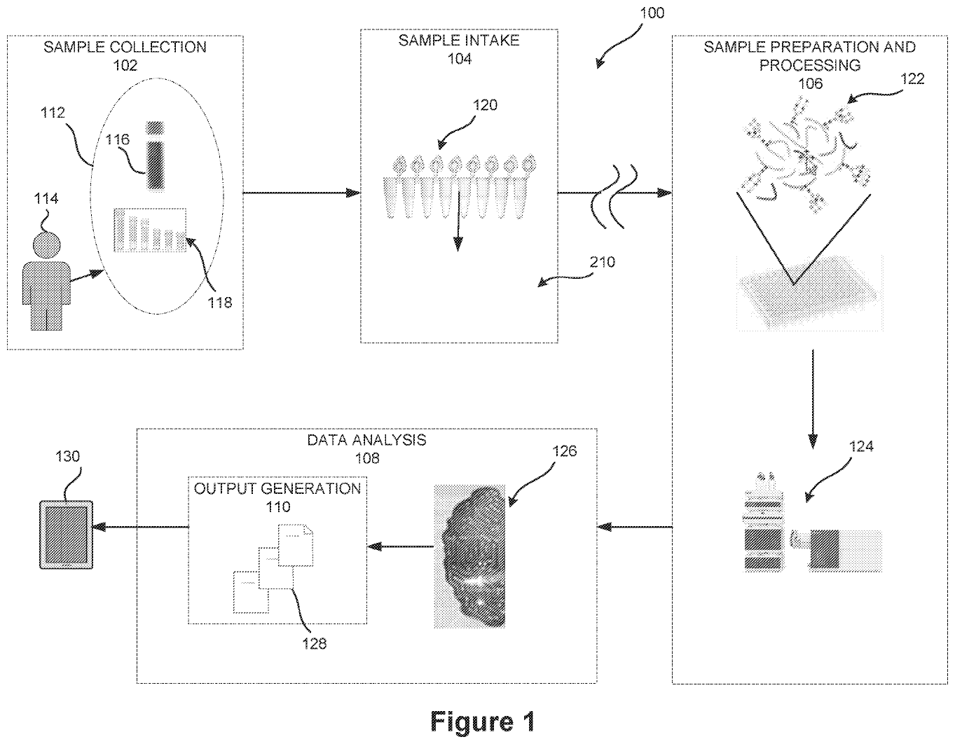

FIG. 1 is a schematic diagram of an exemplary workflow 100 for the detection of peptide structures associated with a disease state for use in diagnosis and/or treatment in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw into a tube. In some situations, a phlebotomist inserts a hollow needle into an arm of a subject such that the needle pierces a vein. The hollow needle is attached to one end of a flexible conduit and the other end of the flexible conduit can subsequently be coupled to the tube. The tube may be at a lower pressure than the ambient pressure outside of the tube causing a blood sample to flow into the tube. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

In various embodiments, the tube can be a Streck tube (La Vista, Nebraska, USA) or a Becton Dickinson (BD) Vacutainer SST tube (serum sample tubes, Franklin Lakes, New Jersey, USA). The Streck tube can be a RNA Complete BCT, Cell-Free DNA BCT, Cyto-Chex BCT, or ESR-Vacuum tube. In various embodiments of a method for collecting blood, the tubes described herein can be used for collecting a blood sample that is used for determining whether a subject has CRC/APL or is likely to develop CRC.

In various embodiments, the tube for collecting blood can include an anticoagulant and a preserving agent. The anticoagulant can prevent the formation of a clot with the biological sample. The anticoagulant may be one of citrate salt, EDTA salt, and a combination thereof. The salt of the anticoagulant can be one of lithium, potassium, and sodium, and combinations thereof. The preserving agent can be one that is configured to release a formaldehyde or other chemical species that includes an aldehyde moiety. The formaldehyde or aldehyde moiety can form a Schiff base with reactive amine groups on proteins or glycoproteins that in turn reduces metabolic activity in the blood sample and/or stabilizes the structural integrity of the cell membrane of the various cells in the blood sample. Under certain circumstances, the formaldehyde or aldehyde moiety may crosslink or partially crosslink a cell membrane and proteins and glycoproteins in the blood sample. An example of a preserving agent configured to release a formaldehyde or other chemical species that includes an aldehyde moiety is imidazolidinyl urea (IDU). For situations where the released amounts of formaldehyde or aldehyde moiety groups need to be limited, the preserving agent can also include a quenching agent such as, for example, glycine. Quenching agents such as glycine have amine groups that can react with any generated formaldehyde or other aldehyde moieties. In an embodiment, a combination that includes IDU and glycine may be referred to as an aldehyde-free preserving agent.

An embodiment of a DNA Complete BCT tube (or other non-Streck tube) can include about 50 μl to about 400 μl of a protective agent in a tube and be used as a container for collecting blood. The protective agent can include imidazolidinyl urea (IDU), ethylenediamine tetraacetic acid (EDTA), and glycine. A blood sample having a first concentration of a protein, a glycoprotein, a peptide, or a glycopeptide can be drawn into a tube, whereby it contacts the protective agent. A plasma fraction can be isolated from the contacted blood sample after the blood draw. The isolating of the plasma sample can be performed after the contacting of the blood with the protective agent for at least about 3 minutes, 5 minutes, 10 minutes, 1 hour, 24 hours, 5 days, 7 days, and 14 days. In another embodiment, a time in between the isolating of the plasma sample and the contacting of the blood with the protective agent ranges from about 3 minutes to 14 days, 30 minutes to 7 days, 12 hours to 7 days, 24 hours to 7 days, and 24 hours to 3 days. The concentration of the imidazolidinyl urea after the contacting step can be about or greater than 5 mg/ml. The concentration of the glycine after the contacting step can be about or below about 0.03 g/ml. The protective agent can be present in an amount that can be about or less than about 5% of an overall mixture volume of the protective agent and the drawn blood sample. In various embodiments, this method of collecting blood can be free of any step of cooling or refrigerating the contacted blood sample to a temperature below room temperature after it has been contacted with the protective agent composition. In various embodiments, this method of collecting blood can be performed at ambient room temperature (e.g., 20 to 25° C.). Optionally, after the isolating of the plasma fraction, the plasma fraction can then be stored at a reduced temperature than ambient (e.g., 15 to 3.3° C.) or frozen (e.g., <0° C.). The isolating of the plasma fraction can be performed by centrifuging the tube to cause the cells to aggregate at the bottom of the tube and leaving the plasma fraction at the top portion of the tube. In an embodiment, as a result of metabolic inhibition of the blood cells in the treated blood sample by one or all of the components of the protective agent, apoptotic and necrotic pathways are inhibited and the blood cells (e.g., red or white blood cells), proteins, glycoproteins, peptides, and/or glycopeptides are protected from degradation. In various embodiments, after at least 24 hours, the contacted blood sample has a second concentration of the protein, the glycoprotein, the peptide, or the glycopeptide where the second concentration is not lower or higher than the first concentration by any statistically significant value. For example, the p value can be >0.05 indicating that there is no statistical difference between the first and second concentrations. In another example, the first and second concentration can have a % difference change of less than a 10%, 20%, 30%, 40%, or 50% (absolute value).

In various embodiments, the tube can contain a concentration of the IDU prior to the contacting step that can be between about 0.1 g/mL and about 3 g/mL. A concentration of the protective agent after the contacting step can be less than about 0.8 g/mL. A concentration of the glycine after the contacting step can be below about 0.03 g/mL

The protective agent stabilizes blood cells in the blood sample to reduce or eliminate the rupture and/or degradation of the blood cells (e.g., white or red) so as to reduce or prevent the release of cellular components. In various embodiments, IDU releases an amount of a formaldehyde releaser preservative agent (e.g., formaldehyde) and the glycine is configured to quench any formaldehyde releaser preservative agent. In combination, IDU and glycine can form an aldehyde-free preservative agent. Under certain circumstances when an assay is designed to only measure circulating glycoproteins, proteins, peptides, and/or glycopeptides outside of the cells for classifying whether a subject has CRC/APL, it can be desirable to substantially reduce or eliminate the rupture and/or degradation of the blood cells. In addition, the rupture of red blood cells can release a relatively large concentration of the hemoglobin, which is a glycoprotein, and can compete or interfere with the measurement of circulating proteins, glycoproteins, peptides and/or glycopeptides. For example, a relatively high hemoglobin concentration can interfere with the efficiency of the proteolytic digestion process especially for the situation where the hemoglobin concentration is much greater than or similar to a concentration of a targeted glycoprotein, glycopeptide, protein, and/or peptide for measurement.

In various embodiments, EDTA will bind divalent ions such as Mg²⁺ and Ca²⁺ that can slow, stop, or prevent a coagulation process inside of a tube used for blood collection. The EDTA can be in the form of an ETDA salt having 1, 2, or 3 sodium or potassium ions such as for example K₃EDTA or K₂EDTA.

In another embodiment of a DNA Complete BCT tube (or other non-Streck tube) can include at least, or about, 200 grams per liter of a composition formulated for stabilizing proteins, glycoproteins, peptides, and/or glycopeptides within a blood sample. The composition can include a) about 50 to about 500 grams per liter of at least one formaldehyde releaser preservative agent; b) ethylenediaminetetraacetic acid (EDTA); and c) one or more solvent. The presence of the at least one formaldehyde releaser preservative agent results in release of at least some formaldehyde and up to, or about, 1% formaldehyde into the composition. The blood collection tube and composition located therein can be sent to a remote location for collection of a blood sample that contains proteins, glycoproteins, peptides, and/or glycopeptides that are stabilized by the composition. In an embodiment, stabilized can refer to a situation where the concentration does not change statistically significantly for a period of time from the contact of the blood with the composition to the time of the test measurement for the proteins, glycoproteins, peptides, and/or glycopeptides.

In various embodiments, the at least one formaldehyde releaser preservative agent may crosslink proteins or glycoproteins in the tube and then cause an interference with a subsequent measurement of targeted proteins or glycoproteins. For this reason, the at least one formaldehyde releaser preservative agent can be configured to release a targeted amount of formaldehyde such as at least 0.001%, 0.01%, 0.01%, 0.2%, 0.5%, 0.75%, or 1% formaldehyde into the composition.

In various embodiments, a method can include providing an evacuated blood collection tube including at least, or about, 200 grams per liter of a composition formulated for stabilizing proteins or glycoproteins within a blood sample. The composition can include about 50 to about 500 grams per liter of at least one formaldehyde releaser preservative agent, wherein the at least one formaldehyde releaser preservative agent includes imidazolidinyl urea (IDU); ethylenediaminetetraacetic acid (EDTA); one or more solvents; and at least some formaldehyde and up to about 1% formaldehyde as a result of the at least one formaldehyde releaser preservative agent. The blood can be drawn into the evacuated blood collection tube including the composition. The inside portion of an evacuated collection tube has a reduced pressure compared to a pressure outside the tube that facilitates a withdrawal of blood from a subject. After filling a portion of the blood collection tube with blood, the blood collection tube can be sent to a remote location for the isolation of the proteins and glycoproteins in a plasma portion from the stabilized blood sample. Once the blood collection tube with blood is received at the remote location, the plasma portion containing proteins and glycoproteins can be isolated from the stabilized blood sample. The isolated proteins and glycoproteins from the plasma portion of the stabilized blood sample can be tested to identify the presence, absence or severity of a CRC/APL disease state by performing one or more of the following: gel electrophoresis, capillary electrophoresis, western blot, mass spectrometry, liquid chromatography, fluorescence detection, ultraviolet spectrometry, immunoassay, or any combination thereof. The collected blood sample is storable for at least, or about 7 days without cell lysis and without glycoprotein or protein degradation of the blood sample due to metabolism after blood collection.

In various embodiments, solvents suitable for use in the tubes described herein include water, saline, dimethylsulfoxide, alcohol, and any mixture thereof.

In various embodiments, a method for identifying a characteristic of a glycoprotein or protein in a whole blood sample from a subject is described that uses a centrifuge. This method can include positioning a composition including whole blood and a protective agent. The protective agent including at least one preservative agent within a centrifuge. In various embodiments the preservative agent includes one of diazolidinyl urea, imidazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxymethoxymethyl-1-aza-3,7-dioxabicyclo[3.3.0]octane, 5-hydroxymethyl-1-aza-3,7-dioxabicyclo[3.3.0]octane, 5-hydroxypoly[methyleneoxy]methyl-1-aza-3,7dioxabicyclo[3.3.0]octane, quaternary adamantine, and any combination thereof. The composition can be centrifuged at a speed of at least about 1000 g and below about 4500 g for at least about 5 minutes and less than about 20 minutes to isolate a plasma fraction that includes the proteins and glycoproteins for further analysis. The isolated proteins and glycoproteins obtained from the plasma fraction can be tested to identify whether the subject has a CRC/APL disease state. In another embodiment, the composition can be centrifuged at a speed of about 1600 g for about 15 minutes to isolate a plasma fraction that includes the proteins and glycoproteins for further analysis.

An embodiment of a Cyto-Chex BCT tube (or other non-Streck tube) can include preloaded compounds consisting of or including ethylene diamine tetra acetic acid (EDTA) and diazolidinyl urea. The tube has an open end and a closed end that receives cells collected directly from a blood draw and wherein a majority of an interior portion of the tube is substantially free of contact with the preloaded components. A blood sample containing a plurality of blood cells can be drawn into the tube whereby it contacts the preloaded compounds to yield a final composition. A ratio of a volume of the preloaded compounds to a combined volume of the blood sample and the preloaded compounds can be from about 1:100 to about 2:100. The plurality of blood cells of the blood sample can be stabilized directly and immediately upon the blood draw. The blood sample can be transported, wherein the blood sample is drawn and transported in the same tube with no processing steps between the blood draw and transporting.

In another embodiment of a Cyto-Chex BCT tube (or other non-Streck tube), it can include a closed collection container having an internal pressure less than atmospheric pressure outside the container. The collection container contains preloaded compounds consisting of or including (i) ethylene diamine tetra acetic acid (EDTA); and (ii) diazolidinyl urea. A majority of an interior portion of the collection container is substantially free of contact with the preloaded component. A blood sample containing the blood cells can be drawn into the collection container whereby the blood sample contacts the preloaded compounds to yield a final composition. After collection of the blood cells in the container, a ratio of a volume of the preloaded compounds to a volume of the final composition can be from about 1:100 to about 2:100.

In yet another embodiment of a Cyto-Chex BCT tube (or other non-Streck tube), it can include a collection container for receiving a whole blood sample. Preloaded compounds can be introduced into the collection container. The preloaded compounds consist of or include (i) ethylene diamine tetra acetic acid (EDTA); and (ii) diazolidinyl urea. The collection container can be evacuated to an internal pressure that is less than atmospheric pressure outside the collection container. A volume of the whole blood sample can be drawn into the collection container, wherein a majority of an interior portion of the collection container is substantially free of contact with the preloaded compounds. The whole blood sample can contact the preloaded compounds to yield a final composition. A ratio of a volume of the preloaded compounds to a volume of the final composition can be from about 1:100 to about 2:100.

In one of the embodiments of the Cyto-Chex BCT tube (or other non-Streck tube), the ratio of the volume of the preloaded compounds to a combined volume of the blood sample and the preloaded compounds can be from about 1:1000 to about 1:10, about 5:1000 to about 5:100, about 1:100 to about 5:100, about 1:100 to about 5:100, and about 1:100 to about 2:100.

An embodiment of a BD Vacutainer® SST tube (or other non-BD tube) can include spray-coated silica and a polymer gel (e.g., polyester based) for serum separation. This type of tube can be used for isolating a serum sample. The spray-coated silica includes silica particles coating an inner surface of the tube. The silica particles are configured to initiate a clot activation in a blood samples. A blood sample itself typically has various components that can create a clot, but requires an activation trigger to start the clotting cascade. However, under certain circumstances, a triggering event can be caused by the contact of the blood with the silica particles coated on an inner wall of the tube. The tube may be inverted at least 5 times and the clotting process can occur, which can take about 30 minutes. After the clotting process has occurred, the tube can be centrifuged to create a serum fraction at a top portion of the tube separate from the blood cells at the bottom of the tube. The centrifugation process may be performed for about 10 minutes at about 1000-1300 RCG (g). The polymer gel forms a physical barrier between the serum fraction and the blood cells during centrifugation that can facilitate the aspiration of the serum fraction.

It is worthwhile to note that although the above description describes the use of a Streck tube, a tube, other than one from Streck, can be used containing one or more of the reagents as described above. Similarly, although the above description describes the use of a BD SST tube, a tube, other than one from BD, can be used containing one or more of the reagents as described above.

In various embodiments, a single run can analyze a sample (e.g., the sample including a peptide analyte), an external standard (e.g., an NGEP of a serum sample), and an internal standard. As such, abundance or raw abundance for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for determining research, diagnosis, and/or treatment.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, report can comprise a target glycopeptide analyte concentration as a function of the NGEP concentration value and the normalized abundance. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of a disease state.

### IV. Detection and Quantification of Peptide Structures

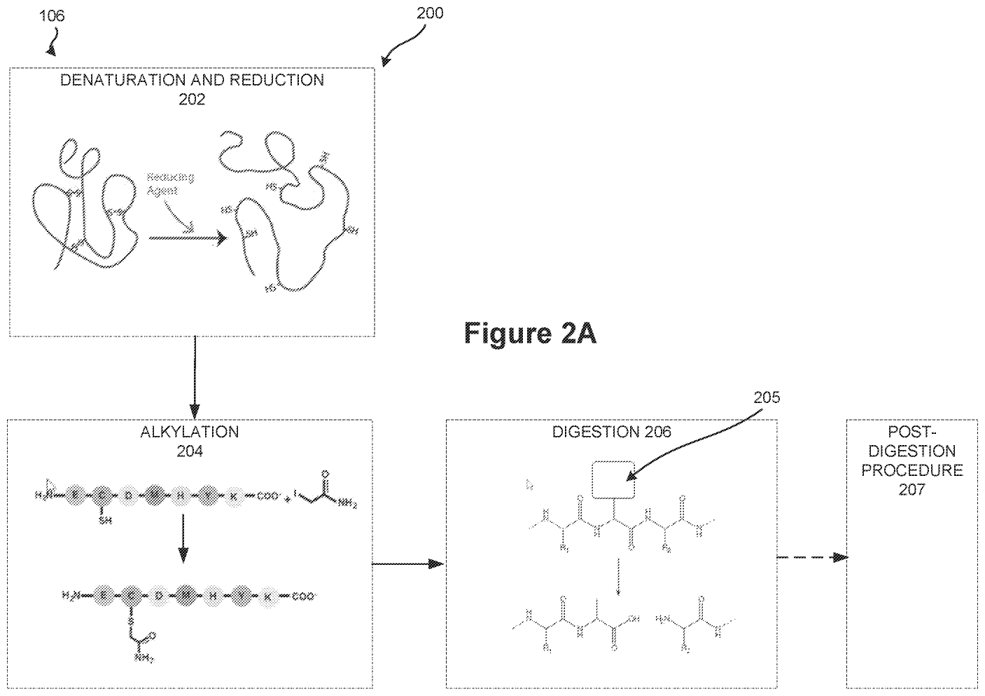

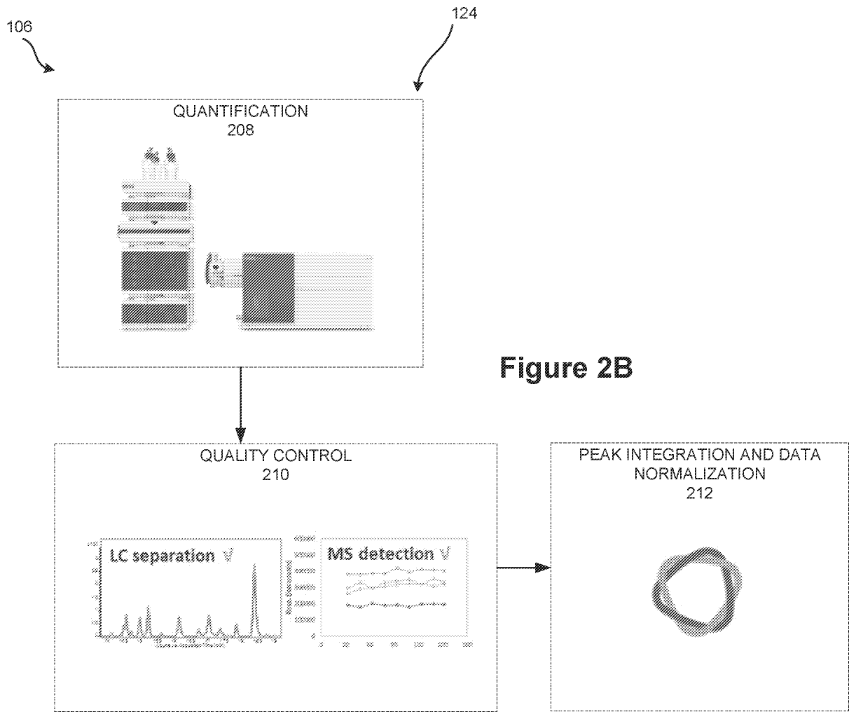

FIGS. 2A and 2B are schematic diagrams of a workflow for sample preparation and processing 106 in accordance with one or more embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

### IV.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206. All areas of the preparation workflow can cause inconsistency between different samples and different experiments, necessitating, the improved normalization systems and methods described herein and throughout.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In various embodiments, the denaturation procedure may include using one or more denaturing agents. In one or more embodiments, the denaturation procedure may include using temperature. In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### IV.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, an instrument or instrument system suited for identification and targeted quantification 208 may include, for example, a Triple Quadrupole LC/MS. In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and the abundances measured.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### V. Peptide Structure Data Analysis

### V.A. Exemplary System for Peptide Structure Data Analysis

### V.A.1. Analysis System for Peptide Structure Data Analysis

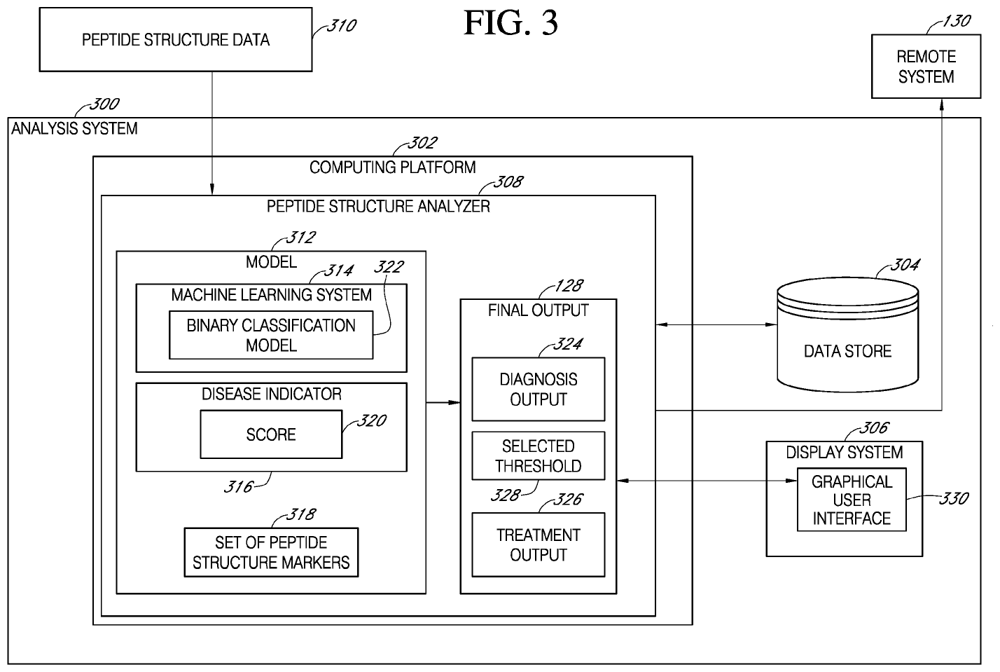

FIG. 3 is a block diagram of an analysis system 300 in accordance with one or more embodiments. Analysis system 300 can be used to both detect and analyze various peptide structures that have been associated to various disease states. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, peptide structure analyzer 308, which may be implemented using hardware, software, firmware, or a combination thereof. In one or more embodiments, peptide structure analyzer 308 is implemented using computing platform 302.

Peptide structure analyzer 308 receives peptide structure data 310 for processing. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112.

Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Peptide structure analyzer 308 includes model 312 that is configured to receive peptide structure data 310 for processing. Model 312 may be implemented in any of a number of different ways. Model 312 may be implemented using any number of models, functions, equations, algorithms, and/or other mathematical techniques.

In one or more embodiments, model 312 includes machine learning system 314, which may itself be comprised of any number of machine learning models and/or algorithms. For example, machine learning system 314 may include, but is not limited to, at least one of a deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a multivariable regression model, a penalized multivariable regression model, or another type of model. In various embodiments, model 312 includes a machine learning system 314 that comprises any number of or combination of the models or algorithms described above.

In various embodiments, model 312 analyzes peptide structure data 310 to generate disease indicator 316 that indicates whether the biological sample is positive for a colorectal cancer disease state based on set of peptide structures 318 identified as being associated with the colorectal cancer disease state. Peptide structure data 310 may include quantification data for the plurality of peptide structures. Quantification data for a peptide structures can include at least one of an abundance, a relative abundance, a normalized abundance, a relative quantity, an adjusted quantity, a normalized quantity, a relative concentration, an adjusted concentration, or a normalized concentration. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In one or more embodiments, the quantification metrics used are normalized abundances. In this manner, peptide structure data 310 may provide abundance information about the plurality of peptide structures with respect to biological sample 112.

Disease indicator 316 may take various forms. In some examples, disease indicator 316 includes a classification that indicates whether or not the subject is positive for the colorectal cancer disease state. In various embodiments, disease indicator 316 can include a score 320. Score 320 indicates whether the colorectal cancer disease state is present or not. For example, score 320 may be, a probability score that indicates how likely it is that the biological sample 112 evidences the presence of the colorectal cancer disease state.

In one or more embodiments, a peptide structure of set of peptide structures 318 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 318 comprises an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the colorectal cancer disease state based on training of model 312. In one or more embodiments, set of peptide structures 318 includes at least one, at least two, or at least three peptide structures from a group of peptide structures (peptide structures PS-1 through PS-6) identified in Table 1. For example, in one or more embodiments, set of peptide structures 318 includes at least 1, at least 2, at least 3, at least 4, at least 5, or all 6 of the peptide structures identified in Table 1. In some cases, the number of peptide structures selected from Table 1 for inclusion in set of peptide structures 318 may be based on, for example, a desired level of accuracy.

In various embodiments, machine learning system 314 takes the form of binary classification model 322. Binary classification model 322 may include, for example, but is not limited to, a regression model. Binary classification model 322 may include, for example, a penalized multivariable regression model that is trained to identify set of peptide structures 318 from a plurality of (or panel of) peptide structures identified in various subjects. Binary classification model 322 may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 318.

Peptide structure analyzer 308 may generate final output 128 based on disease indicator 316 output by model 312. In other embodiments, final output 128 may be an output generated by model 312.

In some embodiments, final output 128 includes disease indicator 316. In one or more embodiments, final output 128 includes diagnosis output 324, treatment output 326, or both. Diagnosis output 324 may include, for example, a diagnosis for the colorectal cancer disease state. The diagnosis can include a positive diagnosis or a negative diagnosis for the adenoma or colorectal cancer disease state.

In one or more embodiments, when disease indicator 316 and/or diagnosis output 324 indicate a positive diagnosis for the adenoma colorectal cancer disease state, a colonoscopy and/or biopsy may be recommended. For example, a colonoscopy and/or biopsy of the subject may be performed in response to disease indicator 316 and/or diagnosis output 324 indicating a positive diagnosis for the adenoma or colorectal cancer disease state. In some embodiments, peptide structure analyzer 308 (or another system implemented on computing platform 302) may generate a report recommending that a colonoscopy and/or biopsy is to be performed for the subject in response to disease indicator 316 and/or diagnosis output 324 indicating a positive diagnosis for the adenoma or colorectal cancer disease state. In other embodiments, peptide structure analyzer 308 may send diagnosis final output 128 to remote system 130 over one or more wireless, wired, and/or optical communications links and remote system 130 may generate a report recommending that a colonoscopy and/or biopsy is to be performed for the subject in response to disease indicator 316 and/or diagnosis output 324 indicating a positive diagnosis for the adenoma or colorectal cancer disease state. The biopsy may be used to confirm the diagnosis to determine whether or not to administer treatment and/or how quickly to administer treatment. When disease indicator 316 and/or diagnosis output 324 indicate a negative diagnosis for the colorectal cancer disease state (e.g., advanced colon adenoma), the report that is generated by peptide structure analyzer 308, remote system 130, or some other system implemented on computing platform 142 may recommend a period of monitoring for the subject. For example, a negative diagnosis indication by disease indicator 316 and/or diagnosis output 324 may thus help prevent unnecessary treatment or overtreatment of the subject.

Treatment output 326 may include, for example, at least one of an identification of a treatment for the subject, a treatment plan for administering the treatment, or both. Treatment for colorectal cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Final output 128 may be sent to remote system 130 for processing in some examples. In other embodiments, final output 128 may be displayed on graphical user interface 330 in display system 306 for viewing by a human operator.

### V.A.2. Computer Implemented System

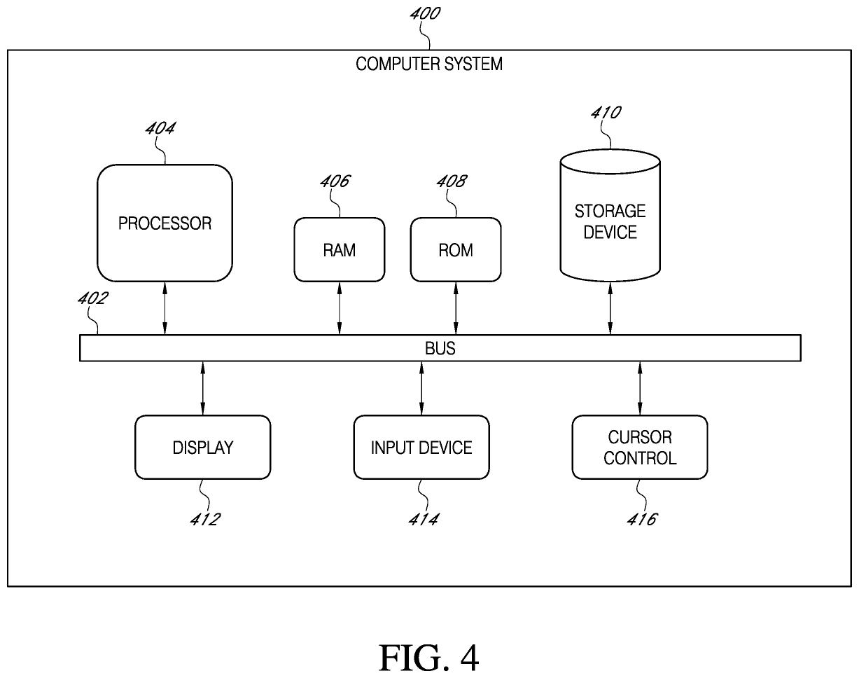

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### VI. Exemplary Methodologies Relating to Diagnosis Based on Peptide Structure Data Analysis

### VI.A.1 Exemplary Methodology—Based on Table 1

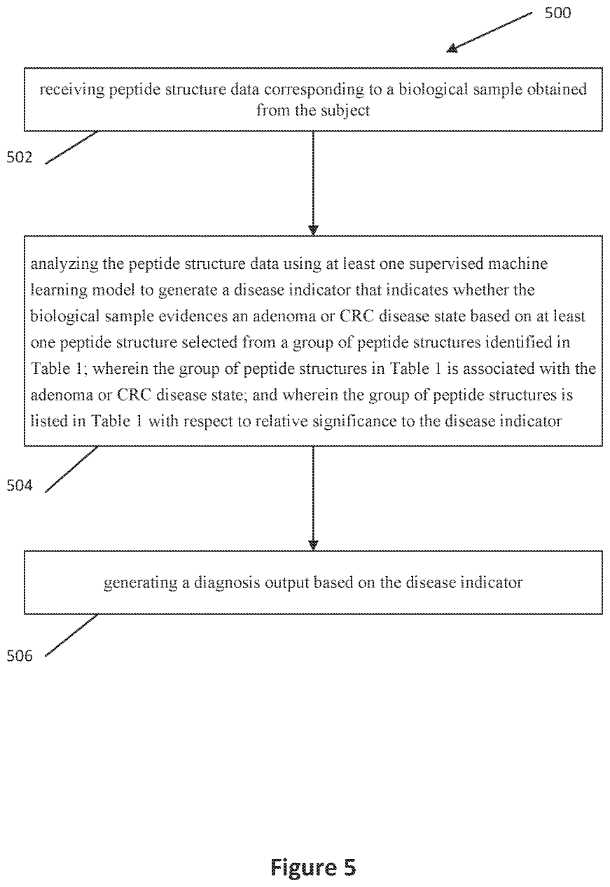

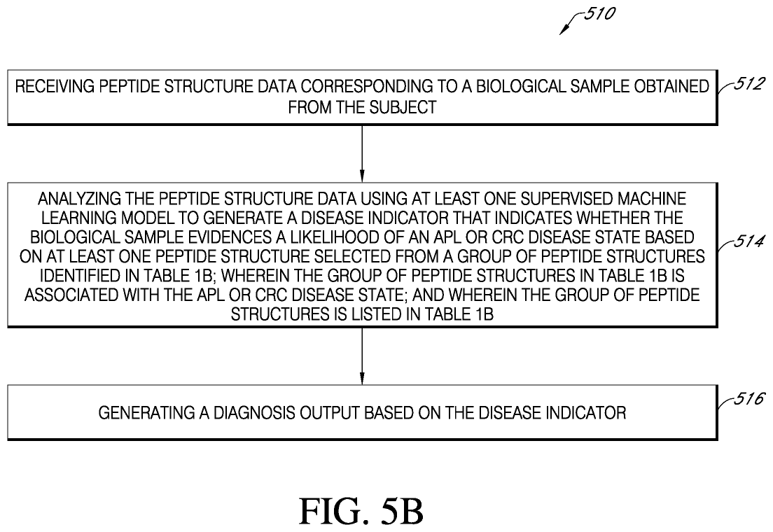

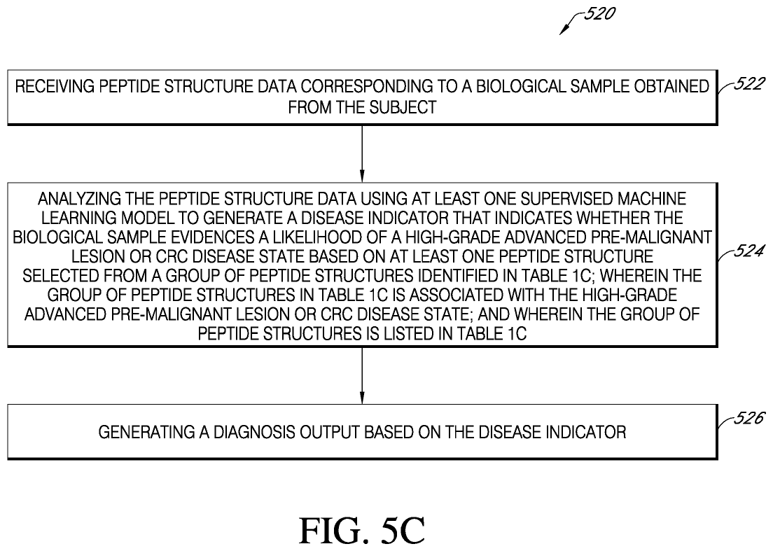

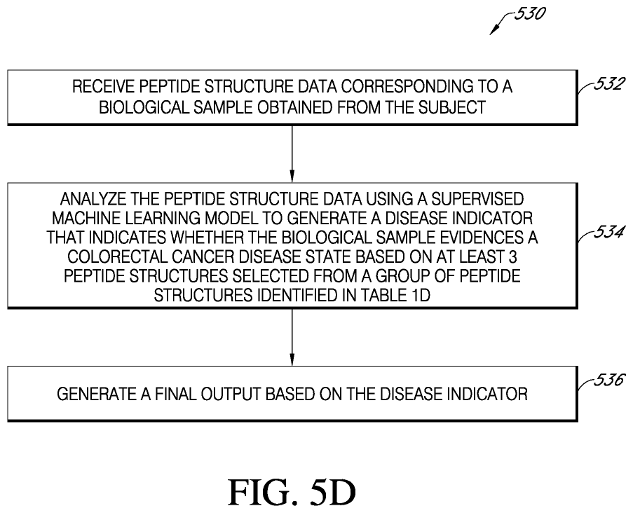

FIG. 5 is a flowchart of a process for diagnosing a subject with respect to adenoma or colorectal cancer (CRC) disease state, in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate a final output that includes at least a diagnosis output for the subject.

Step 502 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 1, with the peptide sequence being one of SEQ ID NOS: 7-12 in Table 1 below.

Step 504 includes analyzing the peptide structure data using at least one supervised machine learning model to generate a disease indicator that indicates whether the biological sample evidences an adenoma or CRC disease state based on at least one peptide structure selected from a group of peptide structures identified in Table 1. In step 504, in accordance with various embodiments, the group of peptide structures can be associated with the colorectal cancer disease state. In step 504, in accordance with various embodiments, the group of peptide structures can be associated with the adenoma or CRC disease state. In step 504, in accordance with various embodiments, the group of peptide structures can be listed in Table 1 with respect to relative significance to the disease indicator.

The group of peptide structures in Table 1 includes peptide structures that have been determined relevant to distinguishing at least between colorectal cancer (and/or adenoma) and a healthy state. For example, the group of peptide structures may be used to predict the probability of colorectal cancer (and/or adenoma) for use in clinically screening patients. In one or more embodiments, the group of peptide structures in Table 1 may also be peptide structures that have been determined relevant to distinguishing between colorectal cancer (and/or adenoma) and a healthy state.

In one or more embodiments, the at least 1 peptide structures includes at least 1, at least 2, at least 3, at least 4, at least 5, or all 6 of the peptide structures PS-1 to PS-6 in Table 1.

In one or more embodiments, step 504 may be implemented using a binary classification model (e.g., a regression model). In some examples, the regression model may be, for example, penalized multivariable regression model. In various embodiments, the disease indicator may be computed using a weight coefficient associated with each peptide structure, the weight coefficient of a corresponding peptide structure of the peptide structures may indicate the relative significance of the corresponding peptide structure to the disease indicator.

In some embodiments, step 504 may include computing a peptide structure profile for the biological sample that identifies a weighted value for each peptide structure. The weighted value for a peptide structure of the peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature—relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for the adenoma or colorectal cancer disease state and the supervised machine learning model is configured to generate an output that identifies the biological sample as either evidencing (“positive for”) the adenoma or colorectal cancer disease state when the disease indicator is greater than a selected threshold or not evidencing (“negative for”) the adenoma or colorectal cancer disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 506 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the adenoma or colorectal cancer disease state if the biological sample evidences the adenoma or colorectal cancer disease state based on the disease indicator. The diagnosis may be, for example, “negative” if the biological sample does not evidence the adenoma or colorectal cancer disease state based on the disease indicator. A negative diagnosis may mean that the biological sample has a non-colorectal cancer state. The negative diagnosis for the adenoma or colorectal cancer disease state can include at least one of a healthy state, or some other non-malignant state.

Generating the diagnosis output in step 506 may include determining that the score falls above (or at or above) a selected threshold and generating a positive diagnosis for the colorectal cancer disease state. Alternatively, step 506 can include determining that the score falls below (or at or below) a selected threshold and generating a negative diagnosis for the adenoma or colorectal cancer disease state. In some scoring systems, the score can include a probability score and the selected threshold can be 0.5. In other scoring systems, the selected threshold can fall within a range between 0.30 and 0.65.

In one or more embodiments, the final output in step 506 may include a treatment output if the diagnosis output indicates a positive diagnosis for the colorectal cancer disease state or adenoma disease state. The treatment output may include, for example, at least one of an identification of a treatment for the subject, a treatment plan for administering the treatment, or both. Treatment for colorectal cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Table 1 below lists a group of peptide structures associated with malignant colorectal cancer (and/or adenoma disease state). One or more features (e.g., relative abundance, concentration, site occupancy) of these peptide structures may be used in the supervised machine learning model described above to generate a disease indicator that predicts the probability of malignancy (e.g., in the context of screening for malignant tumors).

TABLE 1
Peptide Structures Associated with Colorectal Cancer

| | | | | | Glycos | Glyco | | |
|---|---|---|---|---|---|---|---|---|
| | | | Prot | Pep | site | site | Glycan | |
| | | | SEQ | SEQ | within | within | Struct | Mono- |
| PS-ID | | Protein | ID | ID | Peptide | Protein | GL | isotopic |
| No. | PS-NAME | Name | NO | NO | SEQ | SEQ | No. | mass |
| 1 | IC1_253_5412 | Plasma | 1 | 7 | 4 | 253 | 5412 | 4450.915382 |
| | | protease C1 | | | | | | |
| | | inhibitor | | | | | | |
| 2 | HEMO_453_5402 | Hemopexin | 2 | 8 | 7 | 453 | 5402 | 3939.644654 |
| 3 | A2MG_247_5402 | Alpha-2- | 3 | 9 | 10 | 247 | 5402 | 5938.660468 |
| | | macroglobulin | | | | | | |
| 4 | HBA_VGAHAGEYGAEALER | Hemoglobin | 4 | 10 | N/A | N/A | N/A | 1528.726982 |
| | | subunit | | | | | | |
| | | alpha | | | | | | |
| 5 | AGP12_72MC_7613 | Alpha-1- | 14 and/ | 11 | 15 | 72 | 7613 | 6266.639082 |
| | | acid | or 15 | | | | | |
| | | glycoprotein | | | | | | |
| | | 1 &/or 2 | | | | | | |
| 6 | AFAM_109_5402 | Afamin | 6 | 12 | 2 | 109 | 5402 | 3380.232408 |

### VI.A.2 Exemplary Methodology—Based on Table 1B

In another embodiment, a process 510 for diagnosing a subject that has a likelihood of having advanced precancerous lesions (APL) or a colorectal cancer (CRC) disease state can be implemented using one or more of the biomarkers listed in Table 1B (see FIG. 5B). Once it is established that there is a likelihood of having advanced precancerous lesions or colorectal cancer (CRC) disease state, a recommendation to perform a colonoscopy can be provided to a subject. If it is not established that there is a likelihood of having advanced precancerous lesions or colorectal cancer (CRC) disease state, a recommendation to not perform a colonoscopy can be provided to a subject. By using a screening test based on a blood sample that assesses the likelihood of having a condition and can potentially recommend no need to perform a colonoscopy, the subject can avoid an unnecessary colonoscopy that is unpleasant and expensive. Under certain conditions, the term likelihood may be referred to as a probability. It is worthwhile to note that a test using samples such as serum or plasma (blood based) are much more convenient than a colonoscopy procedure that will likely improve compliance in monitoring for CRC/APL. Process 510 may be implemented using, for example, at least a portion of workflow 100 as described in FIGS. 1, 2A, and 2B and/or analysis system 300 as described in FIG. 3. Process 510 may be used to generate a final output that includes at least a diagnosis output for the subject.

The method for diagnosing a subject that has a likelihood of having advanced precancerous lesions (APL) or a colorectal cancer (CRC) disease state comprises step 512 that includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 1B, with the peptide sequence being one of SEQ ID NOS: 27-41 in Table 1B below.

The method for diagnosing a subject that has a likelihood of having advanced precancerous lesions (APL) or a colorectal cancer (CRC) disease state comprises step 514 that includes analyzing the peptide structure data using at least one supervised machine learning model to generate a disease indicator that indicates whether the biological sample evidences a likelihood of an advanced precancerous lesion or CRC disease state based on at least one peptide structure selected from a group of peptide structures identified in Table 1B. In accordance with various embodiments, the group of peptide structures can be associated with the colorectal cancer disease state. In accordance with other embodiments, the group of peptide structures can be associated with the APL or CRC disease state.

The group of peptide structures in Table 1B includes peptide structures that have been determined relevant to distinguishing at least between colorectal cancer/APL and a healthy state. For example, the group of peptide structures may be used to predict the probability of colorectal cancer/APL for use in clinically screening patients. In one or more embodiments, the group of peptide structures in Table 1B may also be peptide structures that have been determined relevant to distinguishing between colorectal cancer/APL and a healthy state.

In one or more embodiments, the at least 1 peptide structures include at least 1, at least 2, at least 3, at least 4, at least 5, at least 6, at least 7, at least 8, at least 9, at least 10, at least 11, at least 12, at least 13, at least 14, or all 15 of the peptide structures PS-1 to PS-21 in Table 1B.

The method for diagnosing a subject that has a likelihood of having advanced precancerous lesions (APL) or a colorectal cancer (CRC) disease state may be implemented using a binary classification model (e.g., a regression model). In some examples, the regression model may be, for example, penalized multivariable regression model. In various embodiments, the disease indicator may be computed using a weight coefficient associated with each peptide structure, the weight coefficient of a corresponding peptide structure of the peptide structures may indicate the relative significance of the corresponding peptide structure to the disease indicator.

The method for diagnosing a subject that has a likelihood of having advanced precancerous lesions (APL) or a colorectal cancer (CRC) disease state may include computing a peptide structure profile for the biological sample that identifies a weighted value for each peptide structure. The weighted value for a peptide structure of the peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature—relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for either APL or colorectal cancer disease state and the supervised machine learning model is configured to generate an output that identifies the biological sample as either evidencing (“positive for”) the APL or colorectal cancer disease state when the disease indicator is greater than a selected threshold or not evidencing (“negative for”) the APL or colorectal cancer disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

The method for diagnosing a subject that has a likelihood of having advanced precancerous lesions (APL) or a colorectal cancer (CRC) disease state comprises a step 516 that includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the APL or colorectal cancer disease state if the biological sample evidences the APL or colorectal cancer disease state based on the disease indicator. The diagnosis may be, for example, “negative” if the biological sample does not evidence the APL or colorectal cancer disease state based on the disease indicator. A negative diagnosis may mean that the biological sample has a non-colorectal cancer state. The negative diagnosis for the APL or colorectal cancer disease state can include at least one of a healthy state, non-APL, or some other non-malignant state.

Generating the diagnosis output may include determining that the score falls above (or at or above) a selected threshold and generating a positive diagnosis for the colorectal cancer disease/APL state. Alternatively, the diagnosis output can include determining that the score falls below (or at or below) a selected threshold and generating a negative diagnosis for the APL/colorectal cancer disease state. In some scoring systems, the score can include a probability score and the selected threshold can be 0.5. In other scoring systems, the selected threshold can fall within a range between 0.30 and 0.65.

In one or more embodiments, the final output of the method may include a treatment output if the diagnosis output indicates a positive diagnosis for the APL/colorectal cancer disease state. The treatment output may include, for example, at least one of an identification of a treatment for the subject, a treatment plan for administering the treatment, or both. Treatment for colorectal cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Table B below lists a group of peptide structures associated with malignant colorectal cancer or APL. One or more features (e.g., relative abundance, concentration, site occupancy) of these peptide structures may be used in the supervised machine learning model described above to generate a disease indicator that predicts the probability of malignancy (e.g., in the context of screening for malignant tumors).

TABLE 1B
Peptide Structures Associated with Advanced Precancerous Lesions (APL) or Colorectal Cancer (CRC)

| | | | | | Glyco | Glyco | | Mono- |
|---|---|---|---|---|---|---|---|---|
| PS- | | | Prot | Pep | site | site | Glycan | isotopic |
| ID | | Protein | SEQ | SEQ | within | within | Struct | mass |
| No. | PS-NAME | Name | ID NO | ID NO | Prot SEQ | Pept SEQ | GL No. | (grams/mole) |
| 7 | A1AT_271MC_5402 | Alpha-1- | 13 | 27 | 271 | 4 | 5402 | 5744.602348 |
| | | antitrypsin | | | | | | |
| 8 | AGP1_103_7624 | Alpha-1-acid | 14 | 28 | 103 | 2 | 7624 | 4584.72581 |
| | | glycoprotein | | | | | | |
| | | 1 | | | | | | |
| 9 | AGP12_56_6503 | Alpha-1-acid | 14 &/ | 29 | 56 | 5 | 6503 | 3656.339868 |
| | | glycoprotein | or 15 | | | | | |
| | | 1&2 | | | | | | |
| 10 | ANGT_47_5402 | Angiotensinogen | 16 | 30 | 47 | 12 | 5402 | 4739.046348 |
| 11 | FETUA_176_6503 | Alpha-2-HS- | 17 | 31 | 176 | 11 | 6503 | 5225.145768 |
| | | glycoprotein | | | | | | |
| 12 | HEMO_453_6503 | Hemopexin | 2 | 32 | 453 | 7 | 6503 | 4595.872252 |
| 13 | HPT_184_7602 | Haptoglobin | 19 | 33 | 184 | 6 | 7602 | 5613.421726 |
| 14 | HRG_345_5411 | Histidine- | 20 | 34 | 345 | 6 | 5411 | 3682.464658 |
| | | rich | | | | | | |
| | | Glycoprotein | | | | | | |
| 15 | KNG1_137_1101 | Kininogen-1 | 21 | 35 | 137 | 5 | 1101 | 5411.52575 |
| 16 | ITIH4_LGVYELLLK | Inter-alpha- | 18 | 36 | n/a | n/a | n/a | 1046.637562 |
| | | trypsin | | | | | | |
| | | inhibitor | | | | | | |
| | | heavy chain | | | | | | |
| | | H4 | | | | | | |
| 17 | ANGT_SLDFTELDVAAEK | Angiotensinogen | 16 | 37 | n/a | n/a | n/a | 2111.965564 |
| 18 | FINC_SYTITGLQPGTDYK | Fibronectin | 23 | 38 | n/a | n/a | n/a | 1542.756554 |
| 19 | SEPP1_VSLATVDK | Selenoprotein | 24 | 39 | n/a | n/a | n/a | 831.470164 |
| | | P | | | | | | |
| 20 | VTNC_GQYCYELDEK | Vitronectin | 25 | 40 | n/a | n/a | n/a | 1303.539038 |
| 21 | THRB_416MC_5402 | Prothrombin | 26 | 41 | 416 | 17 | 5402 | 5375.373484 |

### VI.A.3 Exemplary Methodology—Based on Table 1C

In another embodiment, a process 520 for diagnosing a subject that has a likelihood of having high-grade advanced pre-malignant lesions or a colorectal cancer (CRC) disease state can be implemented using one or more of the biomarkers listed in Table 1C (see FIG. 5C). Once it is established that there is a likelihood of having high-grade advanced pre-malignant lesions or colorectal cancer (CRC) disease state, a recommendation to perform a colonoscopy can be provided to a subject. If it is not established that there is a likelihood of having high-grade advanced pre-malignant lesions or colorectal cancer (CRC) disease state, a recommendation to not perform a colonoscopy can be provided to a subject. By using a screening test based on a blood sample that assesses the likelihood of having a condition and can potentially recommend no need to perform a colonoscopy, the subject can avoid an unnecessary colonoscopy that is unpleasant and expensive. Under certain conditions, the term likelihood may be referred to as a probability. It is worthwhile to note that a test using samples such as serum or plasma (blood based) are much more convenient than a colonoscopy procedure that will likely improve compliance in monitoring for CRC/high-grade advanced pre-malignant lesions. Process 520 may be implemented using, for example, at least a portion of workflow 100 as described in FIGS. 1, 2A, and 2B and/or analysis system 300 as described in FIG. 3. Process 520 may be used to generate a final output that includes at least a diagnosis output for the subject.

The method for diagnosing a subject that has a likelihood of having high-grade advanced pre-malignant lesions or a colorectal cancer (CRC) disease state comprises step 522 that includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 1C, with the peptide sequence being one of SEQ ID NOS: 42-111 in Table 1C below.

The method for diagnosing a subject that has a likelihood of having high-grade advanced pre-malignant lesions or a colorectal cancer (CRC) disease state comprises step 524 that includes analyzing the peptide structure data using at least one supervised machine learning model to generate a disease indicator that indicates whether the biological sample evidences a likelihood of an high-grade advanced pre-malignant lesions or CRC disease state based on at least one peptide structure selected from a group of peptide structures identified in Table 1C. In accordance with various embodiments, the group of peptide structures can be associated with the colorectal cancer disease state. In accordance with other embodiments, the group of peptide structures can be associated with the high-grade advanced pre-malignant lesions or CRC disease state.

The group of peptide structures in Table 1C includes peptide structures that have been determined relevant to distinguishing at least between colorectal cancer/high-grade advanced pre-malignant lesions and a healthy state. For example, the group of peptide structures may be used to predict the probability of colorectal cancer/high-grade advanced pre-malignant lesions for use in clinically screening patients. In one or more embodiments, the group of peptide structures in Table 1C may also be peptide structures that have been determined relevant to distinguishing between colorectal cancer/high-grade advanced pre-malignant lesions and a healthy state.

In one or more embodiments, the at least 1 peptide structures include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, or all 91 of the peptide structures PS-1 to PS-91 in Table 1C.

The method for diagnosing a subject that has a likelihood of having high-grade advanced pre-malignant lesions or a colorectal cancer (CRC) disease state may be implemented using a binary classification model (e.g., a regression model). In some examples, the regression model may be, for example, penalized multivariable regression model. In various embodiments, the disease indicator may be computed using a weight coefficient associated with each peptide structure, the weight coefficient of a corresponding peptide structure of the peptide structures may indicate the relative significance of the corresponding peptide structure to the disease indicator.

The method for diagnosing a subject that has a likelihood of having high-grade advanced pre-malignant lesions or a colorectal cancer (CRC) disease state may include computing a peptide structure profile for the biological sample that identifies a weighted value for each peptide structure. The weighted value for a peptide structure of the peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature—relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for either high-grade advanced pre-malignant lesions or colorectal cancer disease state and the supervised machine learning model is configured to generate an output that identifies the biological sample as either evidencing (“positive for”) the high-grade advanced pre-malignant lesions or colorectal cancer disease state when the disease indicator is greater than a selected threshold or not evidencing (“negative for”) the high-grade advanced pre-malignant lesions or colorectal cancer disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

The method for diagnosing a subject that has a likelihood of having high-grade advanced pre-malignant lesions or a colorectal cancer (CRC) disease state comprises a step 526 that includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the high-grade advanced pre-malignant lesions or colorectal cancer disease state if the biological sample evidences the high-grade advanced pre-malignant lesions or colorectal cancer disease state based on the disease indicator. The diagnosis may be, for example, “negative” if the biological sample does not evidence the high-grade advanced pre-malignant lesions or colorectal cancer disease state based on the disease indicator. A negative diagnosis may mean that the biological sample has a non-colorectal cancer state. The negative diagnosis for the high-grade advanced pre-malignant lesions or colorectal cancer disease state can include at least one of a healthy state, non-high-grade advanced pre-malignant lesions, or some other non-malignant state.

Generating the diagnosis output may include determining that the score falls above (or at or above) a selected threshold and generating a positive diagnosis for the colorectal cancer disease/high-grade advanced pre-malignant lesions state. Alternatively, the diagnosis output can include determining that the score falls below (or at or below) a selected threshold and generating a negative diagnosis for the high-grade advanced pre-malignant lesions/colorectal cancer disease state. In some scoring systems, the score can include a probability score and the selected threshold can be 0.5. In other scoring systems, the selected threshold can fall within a range between 0.30 and 0.65.

In one or more embodiments, the final output of the method may include a treatment output if the diagnosis output indicates a positive diagnosis for the high-grade advanced pre-malignant lesions/colorectal cancer disease state. The treatment output may include, for example, at least one of an identification of a treatment for the subject, a treatment plan for administering the treatment, or both. Treatment for colorectal cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Table 1C below lists a group of peptide structures associated with malignant colorectal cancer or high-grade advanced pre-malignant lesions. One or more features (e.g., relative abundance, concentration, site occupancy) of these peptide structures may be used in the supervised machine learning model described above to generate a disease indicator that predicts the probability of malignancy (e.g., in the context of screening for malignant tumors).

TABLE 1C
Peptide Structures Associated with high-grade advanced pre-malignant lesions or Colorectal Cancer (CRC)

| PS- | | | Prot | Pep | Glyco site | Glyco site | Glycan | Mono- |
|---|---|---|---|---|---|---|---|---|
| ID | | Protein | SEQ | SEQ | within | within | Struct | isotopic |
| No. | PS-NAME | Name | ID NO | ID NO | Prot SEQ | Pept SEQ | GL No. | mass |
| 22 | A1AT_107_5402 | Alpha-1- | 13 | 42 | 107 | 14 | 5402 | 5895.588876 |
| | | antitrypsin | | | | | | |
| 23 | A1AT_271_5402 | Alpha-1- | 13 | 43 | 271 | 4 | 5402 | 3959.660292 |
| | | antitrypsin | | | | | | |
| 24 | A1AT_271_5412 | Alpha-1- | 13 | 44 | 271 | 4 | 5412 | 4105.718198 |
| | | antitrypsin | | | | | | |
| 25 | A1AT_70_NONGLYCOSYLATED | Alpha-1- | 13 | 45 | N/A | N/A | N/A | 3180.627698 |
| | | antitrypsin | | | | | | |
| 26 | A2GL_186_5402 | Leucine- | 112 | 46 | 186 | 8 | 5402 | 3628.627482 |
| | | richAlpha-2- | | | | | | |
| | | glycoprotein | | | | | | |
| 27 | A2MG_1424_5411 | Alpha-2- | 3 | 47 | 1424 | 3 | 5411 | 4221.908394 |
| | | macroglobulin | | | | | | |
| 28 | A2MG_247MC_5401 | Alpha-2- | 3 | 48 | 247 | 10 | 5401 | 4344.878414 |
| | | macroglobulin | | | | | | |
| 29 | A2MG_247MC_5402 | Alpha-2- | 3 | 49 | 247 | 10 | 5402 | 4635.973824 |
| | | macroglobulin | | | | | | |
| 30 | AACT_127_5402 | Alpha-1- | 113 | 50 | 127 | 3 | 5402 | 4416.822744 |
| | | antichymotrypsin | | | | | | |
| 31 | AACT_127_6502 | Alpha-1- | 113 | 51 | 127 | 3 | 6502 | 4781.954932 |
| | | antichymotrypsin | | | | | | |
| 32 | AGP1_93_7614 | Alpha-1-acid | 14 | 52 | 93 | 7 | 7614 | 5578.174858 |
| | | glycoprotein | | | | | | |
| | | 1 | | | | | | |
| 33 | AGP12_56_6503 | Alpha-1-acid | 14 and/ | 53 | 56 | 5 | 6503 | 3656.339868 |
| | | glycoprotein | or 15 | | | | | |
| | | 1or2 | | | | | | |
| 34 | AGP12_72MC_7601 | Alpha-1-acid | 14 and/ | 54 | 72 | 15 | 7601 | 5538.390356 |
| | | glycoprotein | or 15 | | | | | |
| | | 1or2 | | | | | | |
| 35 | AGP12_72MC_7602 | Alpha-1-acid | 14 and/ | 55 | 72 | 15 | 7602 | 5829.485766 |
| | | glycoprotein | or 15 | | | | | |
| | | 1or2 | | | | | | |
| 36 | ANGT_47_5401 | Angiotensinogen | 16 | 56 | 47 | 12 | 5401 | 4447.950938 |
| 37 | APOC3_74_1111 | Apolipoprotein | 114 | 57 | 94 | 14 | 1111 | 2938.301842 |
| | | C-III | | | | | | |
| 38 | APOC3_74MC_1101 | Apolipoprotein | 114 | 58 | 94 | 16 | 1101 | 3035.365836 |
| | | C-III | | | | | | |
| 39 | APOE_212_1102 | Apolipoprotein | 115 | 59 | 212 | 3 | 1102 | 2444.117676 |
| | | E | | | | | | |
| 40 | APOE_212_NONGLYCOSYLATED | Apolipoprotein | 115 | 60 | N/A | N/A | N/A | 1496.794668 |
| | | E | | | | | | |
| 41 | APOE_308_NONGLYCOSYLATED | Apolipoprotein | 115 | 61 | N/A | N/A | N/A | 1619.790308 |
| | | E | | | | | | |
| 42 | APOH_253_6503 | Beta-2- | 116 | 62 | 253 | 3 | 6503 | 4110.558324 |
| | | glycoprotein1 | | | | | | |
| 43 | APOH_253_NONGLYCOSYLATED | Beta-2- | | 63 | N/A | N/A | N/A | 1249.558334 |
| | | glycoprotein1 | 116 | | | | | |
| 44 | APOM_135_NONGLYCOSYLATED | Apolipoprotein | 117 | 64 | N/A | N/A | N/A | 2531.142012 |
| | | M | | | | | | |
| 45 | C4BPA_221_5412 | C4b-binding | 118 | 65 | 221 | 15 | 5412 | 5722.458066 |
| | | protein alpha | | | | | | |
| | | chain | | | | | | |
| 46 | CERU_397_5401 | Ceruloplasmin | 119 | 66 | 397 | 2 | 5401 | 4039.668558 |
| 47 | CERU_397_5402 | Ceruloplasmin | 119 | 67 | 397 | 2 | 5402 | 4330.763968 |
| 48 | CERU_762_5402 | Ceruloplasmin | 119 | 68 | 762 | 9 | 5402 | 4225.768996 |
| 49 | CERU_762_6513 | Ceruloplasmin | 119 | 69 | 762 | 9 | 6513 | 5028.0545 |
| 50 | CFAH_1029_5412 | Complement | 120 | 70 | 1029 | 6 | 5412 | 3774.448658 |
| | | FactorH | | | | | | |
| 51 | CFAH_1095_5402 | Complement | 120 | 71 | 1095 | 17 | 5402 | 5222.006324 |
| | | FactorH | | | | | | |
| 52 | CO3_85_5200 | Complement | 121 | 72 | 85 | 12 | 5200 | 3470.575618 |
| | | C3 | | | | | | |
| 53 | CO3_85_6200 | Complement | 121 | 73 | 85 | 12 | 6200 | 3632.628438 |
| | | C3 | | | | | | |
| 54 | CO6_855_5402 | Complement | 122 | 74 | 855 | 4 | 5402 | 2940.148656 |
| | | componentC6 | | | | | | |
| 55 | FETUA_156_5402 | Alpha-2-HS- | 17 | 75 | 156 | 12 | 5402 | 3975.611638 |
| | | glycoprotein | | | | | | |
| 56 | FETUA_176_6502 | Alpha-2-HS- | 17 | 76 | 176 | 11 | 6502 | 4934.050358 |
| | | glycoprotein | | | | | | |
| 57 | FINC_542_6502 | Fibronectin | 23 | 77 | 542 | 8 | 6502 | 4501.68705 |
| 58 | HEMO_240/246_5402 | Hemopexin | 2 | 78 | 240 or 246 | 1 or 7 | 5402 | 4055.55902 |
| 59 | HEMO_453_5402 | Hemopexin | 2 | 79 | 453 | 7 | 5402 | 3939.644654 |
| 60 | HEMO_64_5402 | Hemopexin | 2 | 80 | 64 | 15 | 5402 | 4731.839512 |
| 61 | HPT_207_121005 | Haptoglobin | 19 | 81 | 207 or 211 | 5 or 9 | 6502 or 6503 | 6888.630826 |
| 62 | HPT_241_5402 | Haptoglobin | 19 | 82 | 241 | 6 | 5402 | 3998.776322 |
| 63 | HRG_125_5402 | Histidine-rich | 20 | 83 | 125 | 5 | 5402 | 4218.740064 |
| | | Glycoprotein | | | | | | |
| 64 | IC1_238_5402 | Plasma | 1 | 84 | 238 | 5 | 5402 | 3113.207564 |
| | | protease C1 | | | | | | |
| | | inhibitor | | | | | | |
| 65 | IC1_238_5411 | Plasma | 1 | 85 | 238 | 6 | 5411 | 2968.17006 |
| | | protease C1 | | | | | | |
| | | inhibitor | | | | | | |
| 66 | IC1_352_5412 | Plasma | 1 | 86 | 352 | 9 | 5412 | 4663.188246 |
| | | protease C1 | | | | | | |
| | | inhibitor | | | | | | |
| 67 | IGA12_144MC_5400 | Immunoglobulin | 123 and/ | 87 | 144 (P01876) | 13 | 5400 | 3978.812328 |
| | | heavy | or 124 | | or 131 | | | |
| | | constant | | | (P01877) | | | |
| | | alpha 1or2 | | | | | | |
| 68 | IGA12_144MC_5500 | Immunoglobulin | 123 and/ | 88 | 144 (P01876) | 13 | 5500 | 4181.891696 |
| | | heavy | or 124 | | or 131 | | | |
| | | constant | | | (P01877) | | | |
| | | alpha 1or2 | | | | | | |
| 69 | IGA2_205_5410 | Immunoglobulin | 124 | 89 | 205 | 6 | 5410 | 2726.188952 |
| | | heavy | | | | | | |
| | | constant | | | | | | |
| | | alpha 2 | | | | | | |
| 70 | IGG1_297_3210 | Immunoglobulin | 125 | 90 | 180 | 5 | 3210 | 2226.879804 |
| | | heavy | | | | | | |
| | | constant | | | | | | |
| | | gamma 1 | | | | | | |
| 71 | IGG1_297_3410 | Immunoglobulin | 125 | 91 | 180 | 5 | 3410 | 2633.03854 |
| | | heavy | | | | | | |
| | | constant | | | | | | |
| | | gamma 1 | | | | | | |
| 72 | IGG1_297_4400 | Immunoglobulin | 125 | 92 | 180 | 5 | 4400 | 2649.033454 |
| | | heavy | | | | | | |
| | | constant | | | | | | |
| | | gamma 1 | | | | | | |
| 73 | IGG1_297_5410 | Immunoglobulin | 125 | 93 | 180 | 5 | 5410 | 2957.14418 |
| | | heavy | | | | | | |
| | | constant | | | | | | |
| | | gamma 1 | | | | | | |
| 74 | IGG1_297_5500 | Immunoglobulin | 125 | 94 | 180 | 5 | 5500 | 3014.165642 |
| | | heavy | | | | | | |
| | | constant | | | | | | |
| | | gamma 1 | | | | | | |
| 75 | IGG2_297_5400 | Immunoglobulin | 126 | 95 | 176 | 5 | 5400 | 2779.096446 |
| | | heavy | | | | | | |
| | | constant | | | | | | |
| | | gamma 2 | | | | | | |
| 76 | IGG34_297_4410 | Immunoglobulin | 127 | 96 | 227 | 5 | 4410 | 2779.096446 |
| | | heavy | | | | | | |
| | | constant | | | | | | |
| | | gamma 3 | | | | | | |
| 77 | IGG34_297_4411 | Immunoglobulin | 127 | 97 | 227 | 5 | 4411 | 3070.191856 |
| | | heavy | | | | | | |
| | | constant | | | | | | |
| | | gamma 3 | | | | | | |
| 78 | IGJ_71MC_NONGLYCOSYLATED | Immunoglobulin | 128 | 98 | N/A | N/A | N/A | 2147.169814 |
| | | J chain | | | | | | |
| 79 | IGM_209_5401 | Immunoglobulin | 129 | 99 | 209 | 7 | 5401 | 4251.745104 |
| | | heavy | | | | | | |
| | | constant mu | | | | | | |
| 80 | IGM_439_5200 | Immunoglobulin | 129 | 100 | 440 | 9 | 5200 | 3580.52053 |
| | | heavy | | | | | | |
| | | constant mu | | | | | | |
| 81 | IGM_46_4310 | Immunoglobulin | 129 | 101 | 46 | 3 | 4310 | 2687.11785 |
| | | heavy | | | | | | |
| | | constant mu | | | | | | |
| 82 | IGM_46_5412 | Immunoglobulin | 129 | 102 | 46 | 3 | 5412 | 3634.440858 |
| | | heavy | | | | | | |
| | | constant mu | | | | | | |
| 83 | IGM_46_6301 | Immunoglobulin | 129 | 103 | 46 | 3 | 6301 | 3156.260994 |
| | | heavy | | | | | | |
| | | constant mu | | | | | | |
| 84 | ITIH1_285_5402 | Inter-alpha- | 22 | 104 | 285 | 19 | 5402 | 5133.188662 |
| | | trypsin | | | | | | |
| | | inhibitor | | | | | | |
| | | heavy chain | | | | | | |
| | | H1 | | | | | | |
| 85 | KLKB1_308_5402 | Plasma | 130 | 105 | 308 | 13 | 5402 | 4187.771294 |
| | | Kallikrein | | | | | | |
| 86 | KLKB1_396_5401 | Plasma | 130 | 106 | 396 | 6 | 5401 | 4270.85736 |
| | | Kallikrein | | | | | | |
| 87 | SHBG_380_5402 | Sex | 131 | 107 | 380 | 8 | 5402 | 3247.313088 |
| | | hormone- | | | | | | |
| | | binding | | | | | | |
| | | globulin | | | | | | |
| 88 | THRB_416MC_5402 | Prothrombin | 26 | 108 | 416 | 17 | 5402 | 5375.373484 |
| 89 | TRFE_432_6502 | Serotransferrin | 132 | 109 | 432 | 12 | 6502 | 4045.648796 |
| 90 | TRFE_630_5402 | Serotransferrin | 132 | 110 | 630 | 9 | 5402 | 4718.889192 |
| 91 | VTNC_169_5402 | Vitronectin | 25 | 111 | 169 | 1 | 5402 | 3115.238472 |

### VI.A.4 Exemplary Methodology—Based on Table 1D,

In another embodiment, a process 530 for diagnosing a subject that has a likelihood of having a colorectal cancer (CRC) disease state can be implemented using one or more of the biomarkers listed in Table 1D (see FIG. 5D). Once it is established that there is a likelihood of having the colorectal cancer (CRC) disease state, a recommendation to perform a colonoscopy can be provided to a subject. If it is not established that there is a likelihood of having colorectal cancer (CRC) disease state, a recommendation to not perform a colonoscopy can be provided to a subject. By using a screening test based on a blood sample that assesses the likelihood of having a condition and can potentially recommend no need to perform a colonoscopy, the subject can avoid an unnecessary colonoscopy that is unpleasant and expensive. Under certain conditions, the term likelihood may be referred to as a probability. It is worthwhile to note that a test using samples such as serum or plasma (blood based) are much more convenient than a colonoscopy procedure that will likely improve compliance in monitoring for CRC. Process 530 may be implemented using, for example, at least a portion of workflow 100 as described in FIGS. 1, 2A, and 2B and/or analysis system 300 as described in FIG. 3. Process 530 may be used to generate a final output that includes at least a diagnosis output for the subject.

The method for diagnosing a subject that has a likelihood of having a colorectal cancer (CRC) disease state comprises step 532 that includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 1D, with the peptide sequence being one of SEQ ID NOS: 136-156 in Table 1D below.

The method for diagnosing a subject that has a likelihood of having a colorectal cancer (CRC) disease state comprises step 534 that includes analyzing the peptide structure data using at least one supervised machine learning model to generate a disease indicator that indicates whether the biological sample evidences a likelihood of a CRC disease state based on at least three peptide structure selected from a group of peptide structures identified in Table 1D. In accordance with various embodiments, the group of peptide structures can be associated with the colorectal cancer disease state.

The group of peptide structures in Table 1D includes peptide structures that have been determined relevant to distinguishing at least between colorectal cancer and a healthy state. For example, the group of peptide structures may be used to predict the probability of colorectal cancer for use in clinically screening patients. In one or more embodiments, the group of peptide structures in Table 1D may also be peptide structures that have been determined relevant to distinguishing between colorectal cancer and a healthy state.

In one or more embodiments, the at least 1 peptide structures include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or all 91 of the peptide structures PS-92 to PS-112 in Table 1D.

The method for diagnosing a subject that has a likelihood of having a colorectal cancer (CRC) disease state may be implemented using a binary classification model (e.g., a regression model). In some examples, the regression model may be, for example, penalized multivariable regression model. In various embodiments, the disease indicator may be computed using a weight coefficient associated with each peptide structure, the weight coefficient of a corresponding peptide structure of the peptide structures may indicate the relative significance of the corresponding peptide structure to the disease indicator.

The method for diagnosing a subject that has a likelihood of having a colorectal cancer (CRC) disease state may include computing a peptide structure profile for the biological sample that identifies a weighted value for each peptide structure. The weighted value for a peptide structure of the peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature—relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for either colorectal cancer disease state and the supervised machine learning model is configured to generate an output that identifies the biological sample as either evidencing (“positive for”) the colorectal cancer disease state when the disease indicator is greater than a selected threshold or not evidencing (“negative for”) the colorectal cancer disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

The method for diagnosing a subject that has a likelihood of having a colorectal cancer (CRC) disease state comprises a step 536 that includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the colorectal cancer disease state if the biological sample evidences the colorectal cancer disease state based on the disease indicator. The diagnosis may be, for example, “negative” if the biological sample does not evidence the colorectal cancer disease state based on the disease indicator. A negative diagnosis may mean that the biological sample has a non-colorectal cancer state. The negative diagnosis for the colorectal cancer disease state can include at least one of a healthy state or some other non-malignant state.

Generating the diagnosis output may include determining that the score falls above (or at or above) a selected threshold and generating a positive diagnosis for the colorectal cancer disease state. Alternatively, the diagnosis output can include determining that the score falls below (or at or below) a selected threshold and generating a negative diagnosis for colorectal cancer disease state. In some scoring systems, the score can include a probability score and the selected threshold can be 0.5. In other scoring systems, the selected threshold can fall within a range between 0.30 and 0.65.

In one or more embodiments, the final output of the method may include a treatment output if the diagnosis output indicates a positive diagnosis for the colorectal cancer disease state. The treatment output may include, for example, at least one of an identification of a treatment for the subject, a treatment plan for administering the treatment, or both. Treatment for colorectal cancer may include, for example, but is not limited to, at least one ofsurgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form oftreatment. The treatment plan may include, for example, but is not limited to, a timeline or schedule for administering the treatment, dosing information, other treatment-related information, or a combination thereof.

Table 1D below lists a group of peptide structures associated with malignant colorectal cancer. One or more features (e.g., relative abundance, concentration, site occupancy) of these peptide structures may be used in the supervised machine learning model described above to generate a disease indicator that predicts the probability of malignancy (e.g., in the context of screening for malignant tumors).

TABLE 1D
Peptide Structures Associated with Colorectal Cancer (CRC)

| PS- | | | Prot | Pep | Glycos site | Glyco site | Glycan | Mono- |
|---|---|---|---|---|---|---|---|---|
| ID | | Protein | SEQ | SEQ | within | within | Struct | isotopic |
| No. | PS-NAME | Name | ID NO | NO ID | Prot SEQ | Pept SEQ | GL No. | mass |
| 92 | AGP1_103_6503 | Alpha-1-acid | 14 | 136 | 103 | 2 | 6503 | 3636.3824 |
| | | glycoprotein | | | | | | |
| | | 1 | | | | | | |
| 93 | AGP1_103_7614 | Alpha-1-acid | 14 | 137 | 103 | 2 | 7614 | 4438.667904 |
| | | glycoprotein | | | | | | |
| | | 1 | | | | | | |
| 94 | AGP12_72_7603 | Alpha-1-acid | 14 and/ | 138 | 72 | 15 | 7603 | 5145.078652 |
| | | glycoprotein | or 15 | | | | | |
| | | 1or2 | | | | | | |
| 95 | HPT_184_5402 | Haptoglobin | 19 | 139 | 184 | 6 | 5402 | 4883.15735 |
| 96 | HPT_184_7602 | Haptoglobin | 19 | 140 | 184 | 6 | 7602 | 5613.421726 |
| 97 | HPT_207_11904 | Haptoglobin | 19 | 141 | 207 or 211 | 5 or 9 | 5402 or 6502 | 6232.403228 |
| 98 | HPT_207_11915 | Haptoglobin | 19 | 142 | 207 or 211 | 5 or 9 | 5402 or 6513 | 6669.556544 |
| 99 | HPT_207_121015 | Haptoglobin | 19 | 143 | 207 or 211 | 5 or 9 | 6502 or 6513 | 7034.688732 |
| 100 | HPT_207_5402 | Haptoglobin | 19 | 144 | 207 or 211 | 5 or 9 | 5402 | 3662.498648 |
| 101 | HPT_241_6503 | Haptoglobin | 19 | 145 | 241 | 6 | 6503 | 4655.00392 |
| 102 | HRG_271_2202 | Histidine-rich | 19 | 146 | 271 | 1 | 2202 | 2838.255284 |
| | | Glycoprotein | | | | | | |
| 103 | A2GL_DLLLPQPDLR | Leucine- | 13 | 147 | N/A | N/A | N/A | 1178.665896 |
| | | richAlpha-2- | | | | | | |
| | | glycoprotein | | | | | | |
| 104 | B2M_VNHVTLSQPK | Beta-2- | 5 | 148 | N/A | N/A | N/A | 1121.61928 |
| | | microglobulin | | | | | | |
| 105 | CO2_HAFILQDTK | Complement | 133 | 149 | N/A | N/A | N/A | 1071.57127 |
| | | C2 | | | | | | |
| 106 | CO5_GIYGTISR | Complement | 134 | 150 | N/A | N/A | N/A | 865.465746 |
| | | C5 | | | | | | |
| 107 | FETUA_AHYDLR | Alpha-2-HS- | 17 | 151 | N/A | N/A | N/A | 773.382018 |
| | | glycoprotein | | | | | | |
| 108 | IC1_GVTSVSQIFHSPDLAIR | Plasma | 1 | 152 | N/A | N/A | N/A | 1825.968604 |
| | | protease C1 | | | | | | |
| | | inhibitor | | | | | | |
| 109 | KNG1_YFIDFVAR | Kininogen-1 | 21 | 153 | N/A | N/A | N/A | 1029.528346 |
| 110 | SEPP1_VSLATVDK | Selenoprotein | 24 | 154 | N/A | N/A | N/A | 831.470164 |
| | | P | | | | | | |
| 111 | THRB_YTACETAR | Prothrombin | 26 | 155 | N/A | N/A | N/A | 970.417806 |
| 112 | ZA2G_CLAYDFYPGK | Zinc-alpha-2- | 135 | 156 | N/A | N/A | N/A | 1232.55357 |
| | | glycoprotein | | | | | | |

### VI.A.5 Additional Description of Tables 1, 1B, 1C, and 1D

Tables 1, 1B, 1C, and 1D include the Peptide Structure Identification Number (PS-ID No.), Petpide Structure Name (PS-Name), Protein Name, Protein Sequence TD Number (Prot SEQ ID No.), Peptide Sequence ID Number (Pep SEQ ID No.), Glycosylation Site within Protein Sequence (Glyco Site within Prot SEQ), Glycosylation Site within Peptide Sequence (Glyco Site within Pept SEQ), Glycan Structure GL Number (Glycan Struct GL No.), and Monoisotopic Mass. The PS-D is a reference number for a particular peptide or glycopeptide. The PS Name is a reference code for a peptide or glycopeptide. For example, the glycopeptide IC1_253_5412 (e.g., SEQ ID No 7) has a prefix portion to indicate that the peptide originated from a protein named IC1, followed by the glycan linking site position in the protein (e.g., the number 253 that is preceded by an underscore and represents a sequential amino acid position in protein IC1), and followed by the glycan structure GL number (e.g., the number 5412 that is preceded by an underscore and represents a glycan composition Hex(5)HexNAc(4)Fuc(1)NeuAc(2)). The PS-Name contains a prefix that represents an abbreviation (that may include a combination of letters and numbers) for a protein abbreviation that corresponds to the Protein Abbreviation of Tables 4, 4B, 4C, and 4D. The term Glyco Site within Prot SEQ is a number that refers to the sequential position of an amino acid of the corresponding protein in which a glycan is attached. For the Glyco Site within Prot SEQ, the amino acid position of the peptide sequence is defined by the sequentially numbered order of amino acids based on the Uniprot ID of the corresponding protein for the peptide sequence. The term Glyco Site within Pept SEQ is a number that refers to the sequential position of an amino acid of the corresponding peptide in which a glycan is attached. For the Glyco Site within Pept SEQ, the amino acid position of the peptide sequence is defined by the sequentially numbered order of amino acids for the peptide sequence that corresponds to Tables 3A, 3C, 3E, and 3G. The term Glycan Structure GL No. is a number that corresponds to a symbol structure and a composition of the glycan as indicated in Tables 5, and 5B to 5G. The term monoisotopic mass represents the mass of the glycopeptide in grams per mole.

In some embodiments, the term AGP12 (e.g, SEQ ID No. 11) represents that the glycopeptide is a fragment of either of the proteins AGP1 or AGP2. In some embodiments, the term IGA12 (SEQ ID No. 88) represents that the glycopeptide is a fragment of either of the proteins IGA1 or IGA2. For the SEQ ID NO:79 in Table 3, the identity of the glycopeptide is one of two possibilities that have the same monoisotopic mass. In the first possibility, the glycan having the Glycan GL NO 6513 is attached to the peptide with a Glycan linking site position of 5 in the peptide sequence. In the second possibility, the glycan having the Glycan GL NO 6502 is attached to the peptide with a Glycan linking site position of 9 in the peptide sequence.

In Tables 1, 1B, 1C, and 1D, if the first number subsequent to the first underscore in the PS-NAME is inconsistent with the Glyco site within Prot SEQ number, then the Glyco site within Prot SEQ number should be used for identification of the peptide. If the second number subsequent to the second underscore in the Peptide Structure (PS) NAME is inconsistent with the Glycan Structure GL NO column number, then the Glycan Structure GL NO column number should be used for identification of the glycan portion of the glycopeptide. If the PS-NAME does not contain any numbers, then the peptide is non-glycosylated. In some instances of the PS-NAME, subsequent to the prefix, there is a number noted with the notation MC that indicates that there was a missed cleavage at position in the peptide sequence as noted by the number.

VI.B.1 Training the Model to Diagnose with respect to the CRC Disease State—Table 1

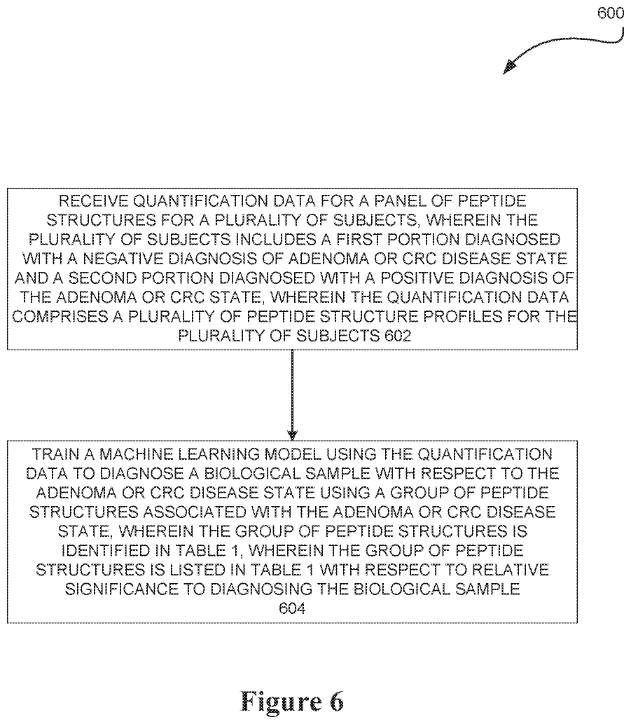

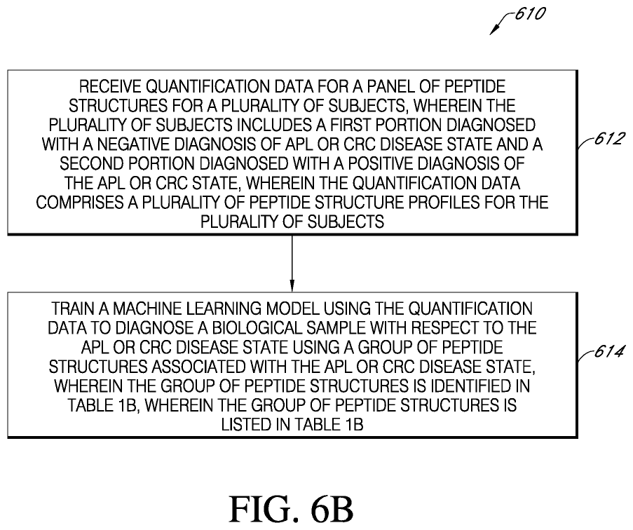

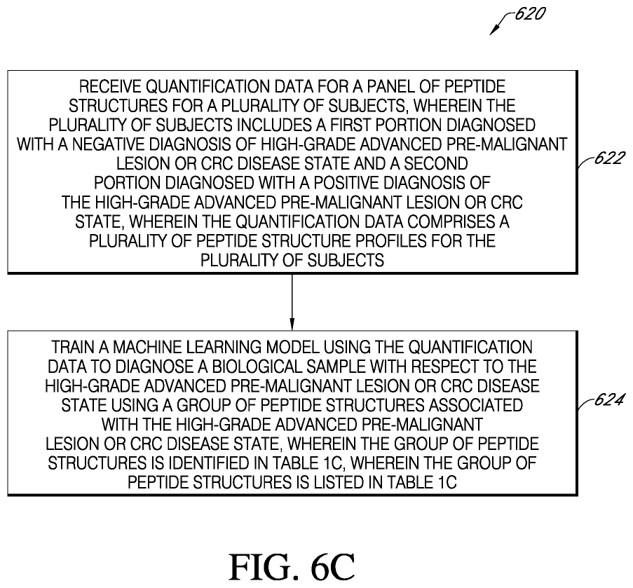

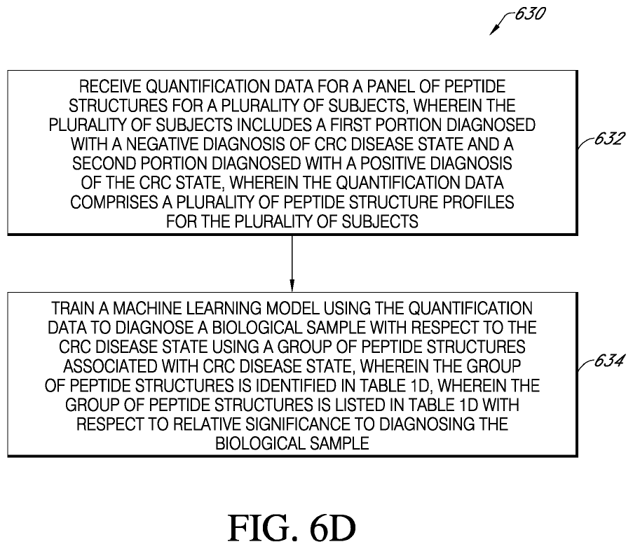

FIG. 6 is a flowchart of a process for training a model to diagnose a subject with respect to an adenoma or CRC disease state in accordance with one or more embodiments. Process 600 may be implemented using, for example, at least a portion of workflow 100 as described in FIGS. 1, 2, and/or analysis system 300 as described in FIG. 3. In some embodiments, process 600 may be one example of an implementation for training the model used in the process 500 in FIG. 5.

Step 602 includes receiving quantification data for a panel of peptide structures for a plurality of subjects. The plurality of subjects includes a first portion diagnosed with a negative diagnosis of an adenoma or CRC disease state and a second portion diagnosed with a positive diagnosis of the adenoma or CRC disease state. The quantification data comprises a plurality of peptide structure profiles for the plurality of subjects.

Step 604 includes training a machine learning model using the quantification data to diagnose a biological sample with respect to the adenoma or CRC disease state using a group of peptide structures associated with the adenoma or CRC disease state (e.g., the group of peptide structures is identified in Table 1). The group of peptide structures is listed in Table 1 with respect to relative significance to diagnosing the biological sample. Step 604 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the adenoma or CRC disease state and a negative diagnosis for any subject of the plurality of subjects determined not to have the adenoma or CRC disease state.

The machine learning model can include a binary classification model. Some binary classification models can include logistical regression models. Some logistical regression models can include LASSO regression models.

An alternative or additional step in process 600 can include performing a differential expression analysis using initial training data to compare a first portion of the plurality of subjects diagnosed with the positive diagnosis for the adenoma or CRC disease state versus a second portion of the plurality of subjects diagnosed with the negative diagnosis for the adenoma or CRC disease state.

An alternative or additional step in process 600 can include identifying a training group of peptide structures based on the differential expression analysis for use as prognostic markers for the adenoma or CRC disease state.

An alternative or additional step in process 600 can include forming the training data based on the training group of peptide structures identified.

An alternative or additional step in process 600 can include identifying a training group of peptide structures based on the differential expression analysis, wherein the training group of peptide structures is a subset of the plurality of peptide structures relevant to diagnosing the adenoma or CRC disease state. The subset may be identified based on at least one of fold-changes, false discovery rates, or p-values computed as part of the differential expression analysis.

An alternative or additional step in process 600 can include training a machine learning model, using the quantification data for the training group of peptide structures, to diagnose a subject of a biological sample with respect to the adenoma or CRC disease state using a group of peptide structures associated with the adenoma or CRC disease state. The group of peptide structures may be a subset of the training group of peptide structures and is identified in Table 1. The group of peptide structures is listed in Table 1 with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures identified in Table 1. The markers used for training of the LASSO regression model may, in one or more embodiments, additionally include one or more other peptide structure markers.

VI.B.2 Training the Model to Diagnose with respect to the CRC Disease State—Table 1B

FIG. 6B is a flowchart of a process for training a model to diagnose a subject with respect to APL or CRC disease state in accordance with one or more embodiments. Process 610 may be implemented using, for example, at least a portion of workflow 100 as described in FIGS. 1, 2, and/or analysis system 300 as described in FIG. 3. In some embodiments, process 610 may be one example of an implementation for training the model used in the process 510 in FIG. 5B.

Step 612 includes receiving quantification data for a panel of peptide structures for a plurality of subjects. The plurality of subjects includes a first portion diagnosed with a negative diagnosis of an APL or CRC disease state and a second portion diagnosed with a positive diagnosis of the APL or CRC disease state. The quantification data comprises a plurality of peptide structure profiles for the plurality of subjects.

Step 614 includes training a machine learning model using the quantification data to diagnose a biological sample with respect to the APL or CRC disease state using a group of peptide structures associated with the APL or CRC disease state (e.g., the group of peptide structures is identified in Table 1B). The group of peptide structures is listed in Table 1B with respect to relative significance to diagnosing the biological sample. Step 614 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the APL or CRC disease state and a negative diagnosis for any subject of the plurality of subjects determined not to have the APL or CRC disease state.

The machine learning model can include a binary classification model. Some binary classification models can include logistical regression models. Some logistical regression models can include LASSO regression models.

An alternative or additional step in process 610 can include performing a differential expression analysis using initial training data to compare a first portion of the plurality of subjects diagnosed with the positive diagnosis for the APL or CRC disease state versus a second portion of the plurality of subjects diagnosed with the negative diagnosis for the APL or CRC disease state.

An alternative or additional step in process 610 can include identifying a training group of peptide structures based on the differential expression analysis for use as prognostic markers for the APL or CRC disease state.

An alternative or additional step in process 610 can include forming the training data based on the training group of peptide structures identified.

An alternative or additional step in process 610 can include identifying a training group of peptide structures based on the differential expression analysis, wherein the training group of peptide structures is a subset of the plurality of peptide structures relevant to diagnosing the APL or CRC disease state. The subset may be identified based on at least one of fold-changes, false discovery rates, or p-values computed as part of the differential expression analysis.

An alternative or additional step in process 610 can include training a machine learning model, using the quantification data for the training group of peptide structures, to diagnose a subject of a biological sample with respect to the APL or CRC disease state using a group of peptide structures associated with the APL or CRC disease state. The group of peptide structures may be a subset of the training group of peptide structures and is identified in Table 1B. The group of peptide structures is listed in Table 1B with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures identified in Table 1B. The markers used for training of the LASSO regression model may, in one or more embodiments, additionally include one or more other peptide structure markers.

VI.B.3 Training the Model to Diagnose with Respect to the CRC Disease State—Table 1C

FIG. 6C is a flowchart of a process for training a model to diagnose a subject with respect to high-grade advanced pre-malignant lesion or CRC disease state in accordance with one or more embodiments. Process 620 may be implemented using, for example, at least a portion of workflow 100 as described in FIGS. 1, 2, and/or analysis system 300 as described in FIG. 3. In some embodiments, process 620 may be one example of an implementation for training the model used in the process 520 in FIG. 5C.

Step 622 includes receiving quantification data for a panel of peptide structures for a plurality of subjects. The plurality of subjects includes a first portion diagnosed with a negative diagnosis of a high-grade advanced pre-malignant lesion or CRC disease state and a second portion diagnosed with a positive diagnosis of the high-grade advanced pre-malignant lesion or CRC disease state. The quantification data comprises a plurality of peptide structure profiles for the plurality of subjects.

Step 624 includes training a machine learning model using the quantification data to diagnose a biological sample with respect to the high-grade advanced pre-malignant lesion or CRC disease state using a group of peptide structures associated with the high-grade advanced pre-malignant lesion or CRC disease state (e.g., the group of peptide structures is identified in Table 1C). The group of peptide structures is listed in Table 1C with respect to relative significance to diagnosing the biological sample. Step 624 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the high-grade advanced pre-malignant lesion or CRC disease state and a negative diagnosis for any subject of the plurality of subjects determined not to have the high-grade advanced pre-malignant lesion or CRC disease state.

The machine learning model can include a binary classification model. Some binary classification models can include logistical regression models. Some logistical regression models can include LASSO regression models.

An alternative or additional step in process 620 can include performing a differential expression analysis using initial training data to compare a first portion of the plurality of subjects diagnosed with the positive diagnosis for the high-grade advanced pre-malignant lesion or CRC disease state versus a second portion of the plurality of subjects diagnosed with the negative diagnosis for the high-grade advanced pre-malignant lesion or CRC disease state.

An alternative or additional step in process 620 can include identifying a training group of peptide structures based on the differential expression analysis for use as prognostic markers for the high-grade advanced pre-malignant lesion or CRC disease state.

An alternative or additional step in process 620 can include forming the training data based on the training group of peptide structures identified.

An alternative or additional step in process 620 can include identifying a training group of peptide structures based on the differential expression analysis, wherein the training group of peptide structures is a subset of the plurality of peptide structures relevant to diagnosing the high-grade advanced pre-malignant lesion or CRC disease state. The subset may be identified based on at least one of fold-changes, false discovery rates, or p-values computed as part of the differential expression analysis.

An alternative or additional step in process 620 can include training a machine learning model, using the quantification data for the training group of peptide structures, to diagnose a subject of a biological sample with respect to the high-grade advanced pre-malignant lesion or CRC disease state using a group of peptide structures associated with the high-grade advanced pre-malignant lesion or CRC disease state. The group of peptide structures may be a subset of the training group of peptide structures and is identified in Table 1C. The group of peptide structures is listed in Table 1C with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures identified in Table 1C. The markers used for training of the LASSO regression model may, in one or more embodiments, additionally include one or more other peptide structure markers.

VI.B.4 Training the Model to Diagnose with Respect to the CRC Disease State—Table ID

FIG. 6D is a flowchart of a process for training a model to diagnose a subject with respect to CRC disease state in accordance with one or more embodiments. Process 630 may be implemented using, for example, at least a portion of workflow 100 as described in FIGS. 1, 2, and/or analysis system 300 as described in FIG. 3. In some embodiments, process 630 may be one example of an implementation for training the model used in the process 530 in FIG. 5D.

Step 632 includes receiving quantification data for a panel of peptide structures for a plurality of subjects. The plurality of subjects includes a first portion diagnosed with a negative diagnosis of a CRC disease state and a second portion diagnosed with a positive diagnosis of the CRC disease state. The quantification data comprises a plurality of peptide structure profiles for the plurality of subjects.

Step 634 includes training a machine learning model using the quantification data to diagnose a biological sample with respect to the CRC disease state using a group of peptide structures associated with the CRC disease state (e.g., the group of peptide structures is identified in Table 1D). The group of peptide structures is listed in Table 1D with respect to relative significance to diagnosing the biological sample. Step 634 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the CRC disease state and a negative diagnosis for any subject of the plurality of subjects determined not to have the CRC disease state.

The machine learning model can include a binary classification model. Some binary classification models can include logistical regression models. Some logistical regression models can include LASSO regression models.

An alternative or additional step in process 630 can include performing a differential expression analysis using initial training data to compare a first portion of the plurality of subjects diagnosed with the positive diagnosis for the CRC disease state versus a second portion of the plurality of subjects diagnosed with the negative diagnosis for the CRC disease state.

An alternative or additional step in process 630 can include identifying a training group of peptide structures based on the differential expression analysis for use as prognostic markers for the CRC disease state.

An alternative or additional step in process 630 can include forming the training data based on the training group of peptide structures identified.

An alternative or additional step in process 630 can include identifying a training group of peptide structures based on the differential expression analysis, wherein the training group of peptide structures is a subset of the plurality of peptide structures relevant to diagnosing the CRC disease state. The subset may be identified based on at least one of fold-changes, false discovery rates, or p-values computed as part of the differential expression analysis.

An alternative or additional step in process 630 can include training a machine learning model, using the quantification data for the training group of peptide structures, to diagnose a subject of a biological sample with respect to the CRC disease state using a group of peptide structures associated with the CRC disease state. The group of peptide structures may be a subset of the training group of peptide structures and is identified in Table 1D. The group of peptide structures is listed in Table 1D with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures identified in Table 1D. The markers used for training of the LASSO regression model may, in one or more embodiments, additionally include one or more other peptide structure markers.

### VI.C.1 Monitoring a Subject for an Adenoma or Colorectal Cancer Disease State Based on Table 1

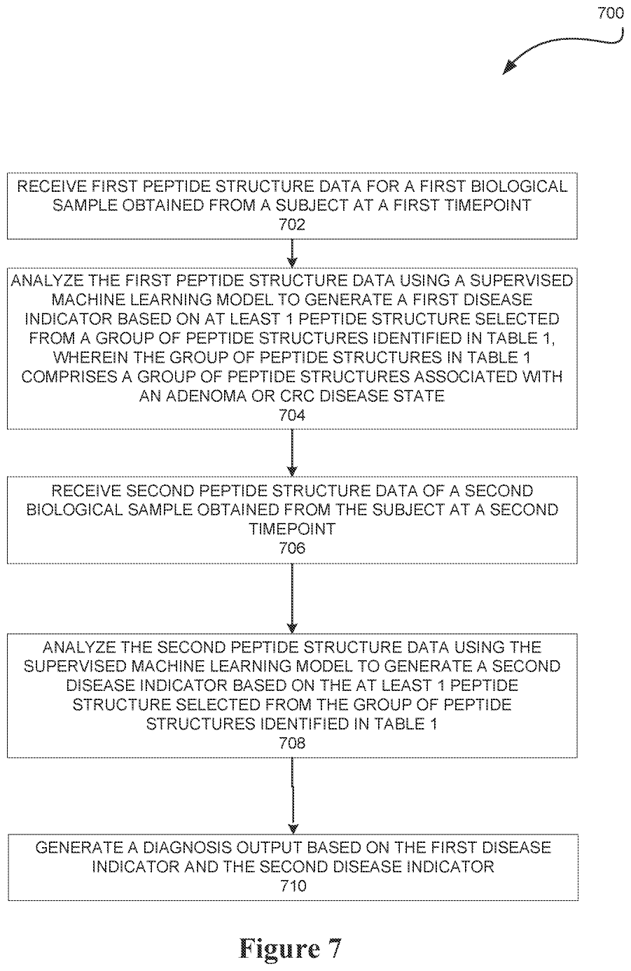

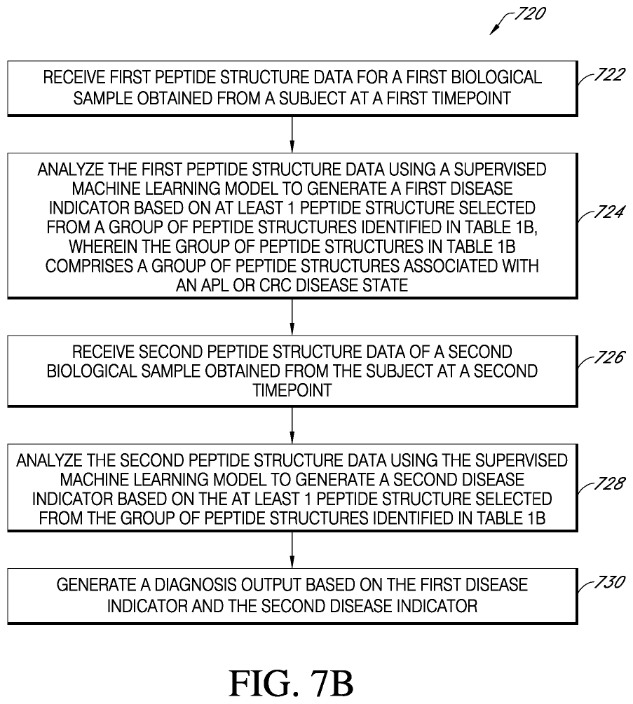

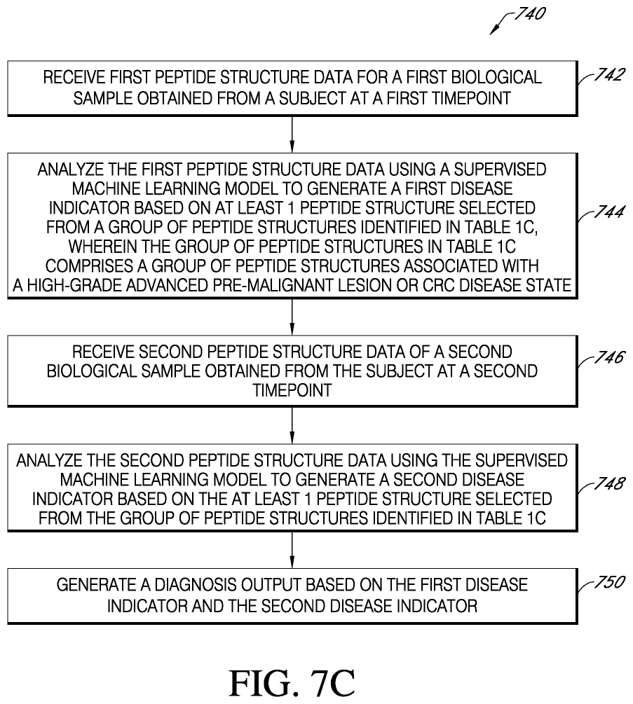

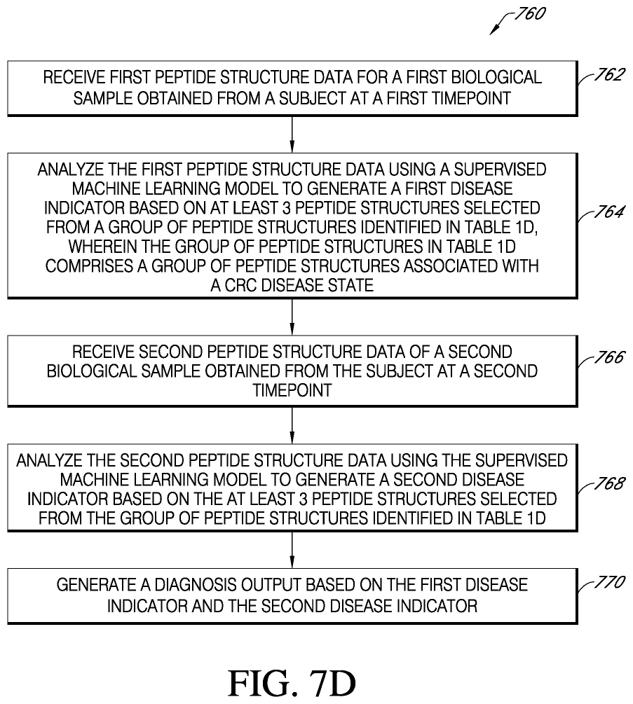

FIG. 7 is a flowchart of a process for monitoring a subject for an adenoma or Colorectal Cancer (CRC) disease state in accordance with one or more embodiments. Process 700 may be implemented using, for example, at least a portion of workflow 100 as described in FIGS. 1, 2, and/or analysis system 300 as described in FIG. 3.

Step 702 includes receiving first peptide structure data for a first biological sample obtained from a subject at a first timepoint.

Step 704 includes analyzing the first peptide structure data using a supervised machine learning model to generate a first disease indicator based on at least 1 peptide structure selected from a group of peptide structures identified in Table 1. The group of peptide structures in Table 1 includes a group of peptide structures associated with an adenoma or CRC disease state in accordance with various embodiments. The supervised machine can be a binary classification model. In some embodiments, the binary classification model can be a logistical regression model.

Step 706 includes receiving second peptide structure data of a second biological sample obtained from the subject at a second timepoint.

Step 708 includes analyzing the second peptide structure data using the supervised machine learning model to generate a second disease indicator based on the at least 1 peptide structure selected from the group of peptide structures identified in Table 1.

Step 710 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the adenoma or CRC disease state and the second biological sample evidences the positive diagnosis for the adenoma or CRC disease. In other embodiments, the diagnosis output identifies whether a non-adenoma or non-CRC disease state has progressed to the adenoma or CRC disease state, respectively, wherein the non-adenoma or non-CRC disease state includes either a healthy state, or a control state.

In accordance with various embodiments, a method is provided for identifying and managing a subject at risk of an adenoma or CRC disease state. The method can comprise receiving a biological sample from the subject, determining a quantity of at least 1 peptide structure identified in Table 1 in the biological sample, analyzing the quantity of each peptide structure using at least one machine learning model to generate a disease indicator, generating a diagnosis output based on the disease indicator that classifies the biological sample as evidencing that the subject has a risk for adenoma or CRC, and identifying a need for a colonoscopy of the subject based on the classified risk of adenoma or CRC.

In various embodiments of the method is provided for identifying and managing a subject at risk of an adenoma or CRC disease state, the disease indicator comprises a disease score.

In various embodiments, generating the diagnosis output comprises determining that the disease score falls above a selected threshold, and generating the diagnosis output based on the disease score falling above the selected threshold, wherein the diagnosis output includes a positive diagnosis for the adenoma or CRC disease state.

In various embodiments, generating the diagnosis output comprises determining that the disease score falls below a selected threshold, and generating the diagnosis output based on the disease score falling below the selected threshold, wherein the diagnosis output includes a negative diagnosis for the adenoma or CRC disease state.

In various embodiments, the method further comprises identifying a need for a colonoscopy of the subject based on the classified risk of adenoma or CRC when the disease indicator falls above a risk threshold.

In various embodiments, the method further comprises identifying a need for a colonoscopy of the subject based on the classified risk of adenoma or CRC when the disease indicator falls above the selected threshold.

In various embodiments, the disease indicator comprises a risk score, and the method further comprises identifying a need for a colonoscopy of the subject based on the classified risk of adenoma or CRC when the risk score falls above a risk threshold.

In various embodiments, the method further comprises receiving medical information for the subject, the information including at least one of: personal and family medical history for the subject, and presence of hereditary medical conditions for the subject, and analyzing (1) the quantity of each peptide structure using at least one machine learning model, and (2) the received medical information, to generate a disease indicator.

In various embodiments, the medical information for the subject includes one or more of: demographic information for the subject, coded list of medical problems for the subject, previous colonoscopy findings, and answers provided by the subject to a questionnaire. In various embodiments, the personal and family medical history for the subject includes information that identifies whether the subject or a member of the subject's family has a history of adenomatous polyps or colorectal cancer. In various embodiments, the presence of hereditary medical conditions for the subject includes information that identifies whether the subject has colorectal cancer syndrome or inflammatory bowel disease.

VLC.2 Monitoring a Subject for an adenoma or Colorectal Cancer Disease State Based on Table 1B

FIG. 7B is a flowchart of a process for monitoring a subject for an APL or Colorectal Cancer (CRC) disease state in accordance with one or more embodiments. Process 720 may be implemented using, for example, at least a portion of workflow 100 as described in FIGS. 1, 2, and/or analysis system 300 as described in FIG. 3.

Step 722 includes receiving first peptide structure data for a first biological sample obtained from a subject at a first timepoint.

Step 724 includes analyzing the first peptide structure data using a supervised machine learning model to generate a first disease indicator based on at least 1 peptide structure selected from a group of peptide structures identified in Table 1B. The group of peptide structures in Table 1B includes a group of peptide structures associated with an APL or CRC disease state in accordance with various embodiments. The supervised machine can be a binary classification model. In some embodiments, the binary classification model can be a logistical regression model.

Step 726 includes receiving second peptide structure data of a second biological sample obtained from the subject at a second timepoint.

Step 728 includes analyzing the second peptide structure data using the supervised machine learning model to generate a second disease indicator based on the at least 1 peptide structure selected from the group of peptide structures identified in Table 1B.

Step 730 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the APL or CRC disease state and the second biological sample evidences the positive diagnosis for the APL or CRC disease. In other embodiments, the diagnosis output identifies whether a non-APL or non-CRC disease state has progressed to the APL or CRC disease state, respectively, wherein the non-APL or non-CRC disease state includes either a healthy state, or a control state.

In accordance with various embodiments, a method is provided for identifying and managing a subject at risk of an APL or CRC disease state. The method can comprise receiving a biological sample from the subject, determining a quantity of at least 1 peptide structure identified in Table 1B in the biological sample, analyzing the quantity of each peptide structure using at least one machine learning model to generate a disease indicator, generating a diagnosis output based on the disease indicator that classifies the biological sample as evidencing that the subject has a risk for APL or CRC, and identifying a need for a colonoscopy of the subject based on the classified risk of APL or CRC.

In various embodiments of the method is provided for identifying and managing a subject at risk of an APL or CRC disease state, the disease indicator comprises a disease score.

In various embodiments, generating the diagnosis output comprises determining that the disease score falls above a selected threshold, and generating the diagnosis output based on the disease score falling above the selected threshold, wherein the diagnosis output includes a positive diagnosis for the APL or CRC disease state.

In various embodiments, generating the diagnosis output comprises determining that the disease score falls below a selected threshold, and generating the diagnosis output based on the disease score falling below the selected threshold, wherein the diagnosis output includes a negative diagnosis for the APL or CRC disease state.

In various embodiments, the method further comprises identifying a need for a colonoscopy of the subject based on the classified risk of APL or CRC when the disease indicator falls above a risk threshold.

In various embodiments, the method further comprises identifying a need for a colonoscopy of the subject based on the classified risk of APL or CRC when the disease indicator falls above the selected threshold.

In various embodiments, the disease indicator comprises a risk score, and the method further comprises identifying a need for a colonoscopy of the subject based on the classified risk of APL or CRC when the risk score falls above a risk threshold.

In various embodiments, the method further comprises receiving medical information for the subject, the information including at least one of: personal and family medical history for the subject, and presence of hereditary medical conditions for the subject, and analyzing (1) the quantity of each peptide structure using at least one machine learning model, and (2) the received medical information, to generate a disease indicator.

In various embodiments, the medical information for the subject includes one or more of: demographic information for the subject, coded list of medical problems for the subject, previous colonoscopy findings, and answers provided by the subject to a questionnaire. In various embodiments, the personal and family medical history for the subject includes information that identifies whether the subject or a member of the subject's family has a history of adenomatous polyps or colorectal cancer. In various embodiments, the presence of hereditary medical conditions for the subject includes information that identifies whether the subject has colorectal cancer syndrome or inflammatory bowel disease.

### VI.C.3 Monitoring a Subject for an Adenoma or Colorectal Cancer Disease State Based on Table 1C

FIG. 7C is a flowchart of a process for monitoring a subject for a high-grade advanced pre-malignant lesion or Colorectal Cancer (CRC) disease state in accordance with one or more embodiments. Process 740 may be implemented using, for example, at least a portion of workflow 100 as described in FIGS. 1, 2, and/or analysis system 300 as described in FIG. 3.

Step 742 includes receiving first peptide structure data for a first biological sample obtained from a subject at a first timepoint.

Step 744 includes analyzing the first peptide structure data using a supervised machine learning model to generate a first disease indicator based on at least 1 peptide structure selected from a group of peptide structures identified in Table 1C. The group of peptide structures in Table 1C includes a group of peptide structures associated with a high-grade advanced pre-malignant lesion or CRC disease state in accordance with various embodiments. The supervised machine can be a binary classification model. In some embodiments, the binary classification model can be a logistical regression model.

Step 746 includes receiving second peptide structure data of a second biological sample obtained from the subject at a second timepoint.

Step 748 includes analyzing the second peptide structure data using the supervised machine learning model to generate a second disease indicator based on the at least 1 peptide structure selected from the group of peptide structures identified in Table 1C.

Step 750 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the high-grade advanced pre-malignant lesion or CRC disease state and the second biological sample evidences the positive diagnosis for the high-grade advanced pre-malignant lesion or CRC disease. In other embodiments, the diagnosis output identifies whether a non-high-grade advanced pre-malignant lesion or non-CRC disease state has progressed to the high-grade advanced pre-malignant lesion or CRC disease state, respectively, wherein the non-high-grade advanced pre-malignant lesion or non-CRC disease state includes either a healthy state, or a control state.

In accordance with various embodiments, a method is provided for identifying and managing a subject at risk of an high-grade advanced pre-malignant lesion or CRC disease state. The method can comprise receiving a biological sample from the subject, determining a quantity of at least 1 peptide structure identified in Table 1C in the biological sample, analyzing the quantity of each peptide structure using at least one machine learning model to generate a disease indicator, generating a diagnosis output based on the disease indicator that classifies the biological sample as evidencing that the subject has a risk for high-grade advanced pre-malignant lesion or CRC, and identifying a need for a colonoscopy of the subject based on the classified risk of high-grade advanced pre-malignant lesion or CRC.

In various embodiments of the method is provided for identifying and managing a subject at risk of a high-grade advanced pre-malignant lesion or CRC disease state, the disease indicator comprises a disease score.

In various embodiments, generating the diagnosis output comprises determining that the disease score falls above a selected threshold, and generating the diagnosis output based on the disease score falling above the selected threshold, wherein the diagnosis output includes a positive diagnosis for the high-grade advanced pre-malignant lesion or CRC disease state.

In various embodiments, generating the diagnosis output comprises determining that the disease score falls below a selected threshold, and generating the diagnosis output based on the disease score falling below the selected threshold, wherein the diagnosis output includes a negative diagnosis for the high-grade advanced pre-malignant lesion or CRC disease state.

In various embodiments, the method further comprises identifying a need for a colonoscopy of the subject based on the classified risk of high-grade advanced pre-malignant lesion or CRC when the disease indicator falls above a risk threshold.

In various embodiments, the method further comprises identifying a need for a colonoscopy of the subject based on the classified risk of high-grade advanced pre-malignant lesion or CRC when the disease indicator falls above the selected threshold.

In various embodiments, the disease indicator comprises a risk score, and the method further comprises identifying a need for a colonoscopy of the subject based on the classified risk of high-grade advanced pre-malignant lesion or CRC when the risk score falls above a risk threshold.

In various embodiments, the method further comprises receiving medical information for the subject, the information including at least one of: personal and family medical history for the subject, and presence of hereditary medical conditions for the subject, and analyzing (1) the quantity of each peptide structure using at least one machine learning model, and (2) the received medical information, to generate a disease indicator.

In various embodiments, the medical information for the subject includes one or more of: demographic information for the subject, coded list of medical problems for the subject, previous colonoscopy findings, and answers provided by the subject to a questionnaire. In various embodiments, the personal and family medical history for the subject includes information that identifies whether the subject or a member of the subject's family has a history of adenomatous polyps or colorectal cancer. In various embodiments, the presence of hereditary medical conditions for the subject includes information that identifies whether the subject has colorectal cancer syndrome or inflammatory bowel disease.

### VI.C.4 Monitoring a Subject for Colorectal Cancer Disease State Based on Table 1D

FIG. 7D is a flowchart of a process for monitoring a subject for a Colorectal Cancer (CRC) disease state in accordance with one or more embodiments. Process 760 may be implemented using, for example, at least a portion of workflow 100 as described in FIGS. 1, 2, and/or analysis system 300 as described in FIG. 3.

Step 762 includes receiving first peptide structure data for a first biological sample obtained from a subject at a first timepoint.

Step 764 includes analyzing the first peptide structure data using a supervised machine learning model to generate a first disease indicator based on at least 3 peptide structures selected from a group of peptide structures identified in Table 1D. The group of peptide structures in Table 1D includes a group of peptide structures associated with an CRC disease state in accordance with various embodiments. The supervised machine can be a binary classification model. In some embodiments, the binary classification model can be a logistical regression model.

Step 766 includes receiving second peptide structure data of a second biological sample obtained from the subject at a second timepoint.

Step 768 includes analyzing the second peptide structure data using the supervised machine learning model to generate a second disease indicator based on the at least 1 peptide structure selected from the group of peptide structures identified in Table 1D.

Step 770 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the CRC disease state and the second biological sample evidences the positive diagnosis for the CRC disease. In other embodiments, the diagnosis output identifies whether a non-CRC disease state has progressed to the CRC disease state, wherein the non-CRC disease state includes either a healthy state, or a control state.

In accordance with various embodiments, a method is provided for identifying and managing a subject at risk of a CRC disease state. The method can comprise receiving a biological sample from the subject, determining a quantity of at least 3 peptide structures identified in Table 1D in the biological sample, analyzing the quantity of each peptide structure using at least one machine learning model to generate a disease indicator, generating a diagnosis output based on the disease indicator that classifies the biological sample as evidencing that the subject has a risk for CRC, and identifying a need for a colonoscopy of the subject based on the classified risk of CRC.

In various embodiments of the method is provided for identifying and managing a subject at risk of an CRC disease state, the disease indicator comprises a disease score.

In various embodiments, generating the diagnosis output comprises determining that the disease score falls above a selected threshold, and generating the diagnosis output based on the disease score falling above the selected threshold, wherein the diagnosis output includes a positive diagnosis for the CRC disease state.

In various embodiments, generating the diagnosis output comprises determining that the disease score falls below a selected threshold, and generating the diagnosis output based on the disease score falling below the selected threshold, wherein the diagnosis output includes a negative diagnosis for the CRC disease state.

In various embodiments, the method further comprises identifying a need for a colonoscopy of the subject based on the classified risk of CRC when the disease indicator falls above a risk threshold.

In various embodiments, the method further comprises identifying a need for a colonoscopy of the subject based on the classified risk of CRC when the disease indicator falls above the selected threshold.

In various embodiments, the disease indicator comprises a risk score, and the method further comprises identifying a need for a colonoscopy of the subject based on the classified risk of CRC when the risk score falls above a risk threshold.

In various embodiments, the method further comprises receiving medical information for the subject, the information including at least one of: personal and family medical history for the subject, and presence of hereditary medical conditions for the subject, and analyzing (1) the quantity of each peptide structure using at least one machine learning model, and (2) the received medical information, to generate a disease indicator.

In various embodiments, the medical information for the subject includes one or more of: demographic information for the subject, coded list of medical problems for the subject, previous colonoscopy findings, and answers provided by the subject to a questionnaire. In various embodiments, the personal and family medical history for the subject includes information that identifies whether the subject or a member of the subject's family has a history of adenomatous polyps or colorectal cancer. In various embodiments, the presence of hereditary medical conditions for the subject includes information that identifies whether the subject has colorectal cancer syndrome or inflammatory bowel disease.

### VII.A Peptide Structure and Product Ion Compositions, Kits and Reagents Based on Table 1

Aspects of the disclosure include compositions comprising one or more of the peptide structures listed in Table 1. In some embodiments, a composition comprises a plurality of the peptide structures listed in Table 1. In some embodiments, a composition comprises 1, 2, 3, 4, 5, or all of the peptide structures listed in Table 1. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 7-12, listed in Table 1 and/or Table 3A.

Aspects of the disclosure include compositions comprising one or more precursor ions having a defined charge and/or defined mass-to-charge (m/z) ratio, as listed in Table 2. Aspects of the disclosure include compositions comprising one or more product ions (1^{st} or 2^{nd}) having a defined mass-to-charge (m/z) ratio, which product ions are produced by converting a peptide structure described herein (e.g., a peptide structure listed in Table 1) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1). In some embodiments, a composition comprises a set of the product ions listed in Table 2, having an m/z ratio selected from the list provided for each peptide structure in Table 1.

In some embodiments, a composition comprises at least one of peptide structures PS-1, PS-2, PS-3, PS-4, PS-5, and PS-6 identified in Table 1. In one or more embodiments, a composition comprises at least 1, at least 2, at least 3, at least 4, at least 5, or all 6 of the peptide structures PS-1, PS-2, PS-3, PS-4, PS-5, and PS-6 in Table 1.

In one or more embodiments, a composition comprises at least 1, at least 2, at least 3, at least 4, at least 5, or all 6 of the peptide structures PS-1, PS-2, PS-3, PS-4, PS-5, and PS-6 in Table 2.

In some embodiments, a composition comprises a peptide structure or a product ion. The peptide structure or product ion comprises an amino acid sequence having at least 90% sequence identity to any one of SEQ ID NOS: 7-12, as identified in Table 3A, corresponding to peptide structures PS-1, PS-2, PS-3, PS-4, PS-5, and PS-6 in Table 1.

In some embodiments, the product ion is selected as one from a group consisting of product ions (1^{st} or 2^{nd}) identified in Table 2, including product ions falling within an identified m/z range of the m/z ratio identified in Table 2 and characterized as having a precursor ion having an m/z ratio within an identified m/z range of the m/z ratio identified in Table 2. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 2, and characterized as having a precursor ion having an m/z ratio that falls within at least one of first range (±0.5), a second range (±1.0), or a third range (±1.0) of the precursor ion m/z ratio identified in Table 2.

TABLE 2
Mass Spectrometry-Related Characteristics for the Peptide Structures associated
with Adenoma or Colorectal Cancer in accordance with Table 1

| | Mono- | | | | | | |
|---|---|---|---|---|---|---|---|
| | isotopic | 1^{st} | 1^{st} | 1^{st} | 2nd | | |
| PS-ID | mass | Precursor | Precursor. | Product | Prod | RT | Collision. |
| NO. | (grams/mole) | m/z | charge | m/z | m/z | (min) | Energy |
| 1 | 4450.915382 | 1114.2 | 3 | 204.1 | 1152.6 | 35.4 | 30 |
| 2 | 3939.644654 | 1314.9 | 5 | 366.1 | N/A | 30.6 | 30 |
| 3 | 5938.660468 | 1189.2 | 3 | 366.1 | N/A | 39.9 | 26 |
| 4 | 1528.726982 | 510.6 | 4 | 617.3 | 745.4 | 13.75 | 14 |
| 14 and/ | 6266.639082 | 1568.4 | 4 | 366.1 | 366.1 | 40.9 | 30 |
| or 15 | | | | | | | |
| 6 | 3380.232408 | 1128.1 | 3 | 366.1 | N/A | 4.9 | 30 |

Table 3A defines the peptide sequences for SEQ ID NOS: 7-12 from Table 1. Table 3A further identifies a corresponding protein SEQ ID NO. for each peptide sequence.

TABLE 3A
Peptide SEQ ID NOS in accordance with Table 1

| Prot | | Pept. |
|---|---|---|
| Seq ID NO. | Peptide Sequence | SEQ ID NO |
| 1 | VLSNNSDANLELINTWVAK | 7 |
| 2 | ALPQPQNVTSLLGCTH | 8 |
| 3 | IITILEEEMNVSVCGLYTYG | 9 |
| | KPVPGHVTVSICR | |
| 4 | VGAHAGEYGAEALER | 10 |
| 14 and/or 15 | SVQEIQATFFYFTPNKTEDTI | 11 |
| | FLR | |
| 6 | HNFSHCCSK | 12 |

Table 3B provides an indication of particular markers and includes the starting position of the peptide sequence within the protein sequence and the end position of the peptide sequence within the protein sequence.

TABLE 3B
Markers and Protein Positions in accordance with Table 1

| PS-ID | | | Start | End |
|---|---|---|---|---|
| No. | PS-NAME | Peptide.sequence | Position | Position |
| 1 | IC1_253_5412 | VLSNNSDANLELINTWVAK | 250 | 268 |
| 2 | HEMO_453_5402 | ALPQPQNVTSLLGCTH | 447 | 462 |
| 3 | A2MG_247_5402 | IITILEEEMNVSVCGLYTYGKP | 238 | 270 |
| | | VPGHVTVSICR | | |
| 4 | HBA_ | VGAHAGEYGAEALER | 18 | 32 |
| | VGAHAGEYGAEALER | | | |
| 5 | AGP12_72MC_7613 | SVQEIQATFFYFTPNKTEDTIF | 58 | 81 |
| | | LR | | |
| 6 | AFAM_109_5402 | HNFSHCCSK | 108 | 116 |

Table 4 identifies the proteins of SEQ ID NOS: 1-4, 6, and 14-15 from Table 1. Table 4 identifies a corresponding protein abbreviation and protein name for each of protein SEQ ID NOS: 1-4, 6, and 14-15. Further, Table 4 identifies a corresponding Uniprot ID for each of protein SEQ ID NOS: 1-4, 6, and 14-15.

TABLE 4
Protein SEQ ID NOS in accordance with Table 1

| Protein | | | Prot Seq |
|---|---|---|---|
| Abbrev. | Protein.Name | Uniprot ID | ID NO. |
| IC1 | Plasma protease C1 inhibitor | P05155 | 1 |
| HEMO | Hemopexin | P02790 | 2 |
| A2MG | Alpha-2-macroglobulin | P01023 | 3 |
| HBA | Hemoglobin subunit alpha | P69905 | 4 |
| AGP12 | Alpha-1-acid glycoprotein 1&2 | P02763 &/or | 14 and/or |
| | | P19652 | 15 |
| AFAM | Afamin | P43652 | 6 |

Table 5 identifies and defines the glycan structures included in Table 1, all of which are N-glycans. Table 5 identifies a coded representation ofthe composition for each glycan structure included in Table 1. As used herein, the 4-digit GL NO. is a designation that represents the number of hexoses, the number of HexNAcs, the number of Fucoses, and the number of Neuraminic Acids.

TABLE 5
Glycan Structure GL NOS: Compositions and Symbol Structures in
accordance with Table 1

| Glycan | | Glycan Symbol | Glycan Mass |
|---|---|---|---|
| GL NO. | Glycan Composition | Structure | (grams/mole) |
| 5412 | Hex(5)HexNAc(4)Fuc(1)NeuAc(2) | 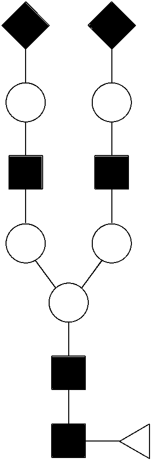 | 2368.8408 |
| 5402 | Hex(5)HexNAc(4)Fuc(0)NeuAc(2) | 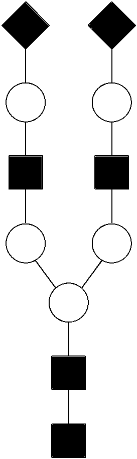 | 2222.78289 |
| 7613 | Hex(7)HexNAc(6)Fuc(1)NeuAc(3) | 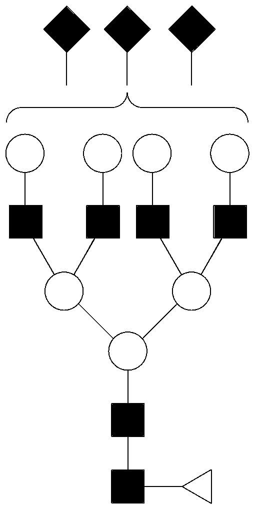 | 3390.20058 |

Legend for Table 5:

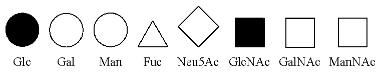

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating an adenoma or CRC disease state. A transition includes a precursor ion and at least one product ion grouping. As reviewed herein, the peptide structures in Table 1, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, adenoma or CRC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2A. The alkylation procedure may be implemented in a manner similar to, for example, alkylation procedure 204 in FIG. 2A. The digestion procedure may be implemented in a manner similar to, for example, digestion procedure 206 in FIG. 2A.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 2 or an m/z ratio within an identified m/z ratio as provided in Table 2. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VII.B Peptide Structure and Product Ion Compositions, Kits and Reagents Based on Table 1B

Aspects of the disclosure include compositions comprising one or more of the peptide structures listed in Table 1B. In some embodiments, a composition comprises a plurality of the peptide structures listed in Table 1B. In some embodiments, a composition comprises 1, 2, 3, 4, 5, 6, 7, 8, 9, 10, 11, 12, 13, 14, or all of the peptide structures listed in Table 1B and/or Table 3C. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 27-41, listed in Table 1B.

Aspects of the disclosure include compositions comprising one or more precursor ions having a defined charge and/or defined mass-to-charge (m/z) ratio, as listed in Table 2B. Aspects of the disclosure include compositions comprising one or more product ions having a defined mass-to-charge (m/z) ratio, which product ions are produced by converting a peptide structure described herein (e.g., a peptide structure listed in Table 1B and 3C) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1B). In some embodiments, a composition comprises a set of the product ions listed in Table 2B, having an m/z ratio selected from the list provided for each peptide structure in Table 1B or Table 2B.

In some embodiments, a composition comprises at least one of peptide structures PS-7, PS-8, PS-9, PS-10, PS-11, PS-12, PS-13, PS-14, PS-15, PS-16, PS-17, PS-18, PS-19, PS-20, and PS-21 identified in Table 1B. In one or more embodiments, a composition comprises at least 1, at least 2, at least 3, at least 4, at least 5, at least 6, at least 7, at least 8, at least 9, at least 10, at least 11, at least 12, at least 13, at least 14, or all 15 of the peptide structures PS-7, PS-8, PS-9, PS-10, PS-11, PS-12, PS-13, PS-14, PS-15, PS-16, PS-17, PS-18, PS-19, PS-20, and PS-21 in Table 1B.

In one or more embodiments, a composition comprises at least 1, at least 2, at least 3, at least 4, at least 5, at least 6, at least 7, at least 8, at least 9, at least 10, at least 11, at least 12, at least 13, at least 14, or all 15 of the peptide structures PS-7, PS-8, PS-9, PS-10, PS-11, PS-12, PS-13, PS-14, PS-15, PS-16, PS-17, PS-18, PS-19, PS-20, and PS-21 in Table 2B.

In some embodiments, a composition comprises a peptide structure or a product ion. The peptide structure or product ion comprises an amino acid sequence having at least 90% sequence identity to any one of SEQ ID NOS: 27-41, as identified in Table 3C, corresponding to peptide structures PS-1, PS-2, PS-3, PS-4, PS-5, and PS-6 in Table 1B and/or 3C.

In some embodiments, the product ion is selected as one from a group consisting of product ions identified in Table 2B, including product ions falling within an identified m/z range of the m/z ratio identified in Table 2B and characterized as having a precursor ion having an m/z ratio within an identified m/z range of the m/z ratio identified in Table 2B. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 2B, and characterized as having a precursor ion having an m/z ratio that falls within at least one of first range (±0.5), a second range (±1.0), or a third range (±1.0) of the precursor ion m/z ratio identified in Table 2B.

TABLE 2B
Mass Spectrometry-Related Characteristics for the Peptide Structures
associated with APL or CRC in accordance with Table 1B

| PS- | Mono- | 1^{st} | 1^{st} | 1^{st} | 2nd | | Collision |
|---|---|---|---|---|---|---|---|
| ID | isotopic | Precursor | Precursor | Product | Prod | RT | Energy - |
| No. | mass | m/z | charge | m/z | m/z | (min) | V |
| 7 | 5744.602348 | 1150.3 | 5 | 1249.3 | 366.1 | 42.4 | 28 |
| 8 | 4584.72581 | 1147.3 | 4 | 366.1 | n/a | 5.8 | 28 |
| 9 | 3656.339868 | 1220.1 | 3 | 274.1 | 999.4 | 5.4 | 35 |
| 10 | 4739.046348 | 949.2 | 5 | 274.1 | 913.5 | 25.3 | 18 |
| 11 | 5225.145768 | 1307.1 | 4 | 366.1 | n/a | 30.7 | 33 |
| 12 | 4595.872252 | 1150.5 | 4 | 274.1 | 970 | 31 | 25 |
| 13 | 5613.421726 | 1124 | 5 | 366.1 | n/a | 33.2 | 20 |
| 14 | 3682.464658 | 921.6 | 4 | 366.1 | n/a | 5.6 | 25 |
| 15 | 5411.52575 | 1083.9 | 5 | 274.1 | n/a | 38.1 | 25 |
| 16 | 1046.637562 | 524.3 | 2 | 778.5 | 934.6 | 36.1 | 15 |
| 17 | 2111.965564 | 719.4 | 3 | 316.2 | 975.5 | 30.5 | 21 |
| 18 | 1542.756554 | 772.4 | 2 | 680.3 | 978.4 | 22.7 | 22 |
| 19 | 831.470164 | 416.7 | 2 | 646.4 | 733.4 | 12.7 | 10 |
| 20 | 1303.539038 | 652.8 | 2 | 956.4 | 796.4 | 14.9 | 19 |
| 21 | 5375.373484 | 1076.5 | 5 | 274.1 | n/a | 40.5 | 20 |

Table 3C defines the peptide sequences for SEQ ID NOS: 27-41 from Table 1B. Table 4B further identifies a corresponding protein SEQ ID NO. for each peptide sequence.

TABLE 3C
Peptide SEQ ID NOS in accordance with Table 1B

| PS-ID No. | Peptide Sequence | Pept. SEQ ID NO |
|---|---|---|
| 7 | YLGNATAIFFLPDEGKLQHLENELTHDIITK | 27 |
| 8 | ENGTISR | 28 |
| 9 | NEEYNK | 29 |
| 10 | VYIHPFHLVIHNESTCEQLAK | 30 |
| 11 | AALAAFNAQNNGSNFQLEEISR | 31 |
| 12 | ALPQPQNVTSLLGCTH | 32 |
| 13 | MVSHHNLTTGATLINEQWLLTTAK | 33 |
| 14 | HSHNNNSSDLHPHK | 34 |
| 15 | FSVATQTCQITPAEGPVVTAQYDCLGCVH | 35 |
| | PISTQSPDLEPILR | |
| 16 | LGVYELLLK | 36 |
| 17 | SLDFTELDVAAEK | 37 |
| 18 | SYTITGLQPGTDYK | 38 |
| 19 | VSLATVDK | 39 |
| 20 | GQYCYELDEK | 40 |
| 21 | WVLTAAHCLLYPPWDKNFTENDLLVR | 41 |

Table 3D provides an indication of particular markers and includes the starting position of the peptide sequence within the protein sequence and the end position of the peptide sequence within the protein sequence.

TABLE 3D
Markers and Protein Positions in accordance with Table 1B

| PS- | | | | |
|---|---|---|---|---|
| ID | | | Start | End |
| No. | PS-NAME | Peptide.sequence | Position | Position |
| 7 | A1AT_271MC_5402 | YLGNATAIFFLPDEGKLQHLENEL | 268 | 298 |
| | | THDIITK | | |
| 8 | AGP1_103_7624 | ENGTISR | 102 | 108 |
| 9 | AGP12_56_6503 | NEEYNK | 52 | 57 |
| 10 | ANGT_47_5402 | VYIHPFHLVIHNESTCEQLAK | 36 | 56 |
| 11 | FETUA_176_6503 | AALAAFNAQNNGSNFQLEEISR | 166 | 187 |
| 12 | HEMO_453_6503 | ALPQPQNVTSLLGCTH | 447 | 462 |
| 13 | HPT_184_7602 | MVSHHNLTTGATLINEQWLLTT | 179 | 202 |
| | | AK | | |
| 14 | HRG_345_5411 | HSHNNNSSDLHPHK | 340 | 353 |
| 15 | KNG1_137_1101 | FSVATQTCQITPAEGPVVTAQYD | 133 | 175 |
| | | CLGCVHPISTQSPDLEPILR | | |
| 16 | ITIH4_LGVYELLLK | LGVYELLLK | 154 | 162 |
| 17 | ANGT_SLDFTELDVAAEK | SLDFTELDVAAEK | 238 | 250 |
| 18 | FINC_SYTITGLQPGTDYK | SYTITGLQPGTDYK | 1958 | 1971 |
| 19 | SEPP1_VSLATVDK | VSLATVDK | 189 | 196 |
| 20 | VTNC_GQYCYELDEK | GQYCYELDEK | 177 | 186 |
| 21 | THRB_416MC_5402 | WVLTAAHCLLYPPWDKNFTEND | 400 | 425 |
| | | LLVR | | |

Table 4B identifies the proteins of SEQ ID NOS: 2, 13-21, and 23-26 from Table 1B. Table 4B identifies a corresponding protein abbreviation and protein name for each of protein SEQ ID NOS: 2, 13-21, and 23-26. Further, Table 4B identifies a corresponding Uniprot ID for each of protein SEQ ID NOS: 2, 13-21, and 23-26.

TABLE 4B
Protein SEQ ID NOS in accordance with Table 1B

| Protein | | | Prot Seq |
|---|---|---|---|
| Abbrev. | Protein Name | Uniprot ID | ID NO. |
| A1AT | Alpha-1-antitrypsin | P01009 | 13 |
| AGP1 | Alpha-1-acid glycoprotein 1 | P02763 | 14 |
| AGP12 | Alpha-1-acid glycoprotein 1&2 | P02763&P19652 | 14 &15 |
| ANGT | Angiotensinogen | P01019 | 16 |
| FETUA | Alpha-2-HS-glycoprotein | P02765 | 17 |
| HEMO | Hemopexin | P02790 | 2 |
| HPT | Haptoglobin | P00738 | 19 |
| HRG | Histidine-rich Glycoprotein | P04196 | 20 |
| KNG1 | Kininogen-1 | P01042 | 21 |
| ITIH4 | Inter-alpha-trypsin | Q14624 | 18 |
| | inhibitor heavy chain H4 | | |
| FINC | Fibronectin | P02751 | 23 |
| SEPP1 | Selenoprotein P | P49908 | 24 |
| VTNC | Vitronectin | P04004 | 25 |
| THRB | Prothrombin | P00734 | 26 |

Tables 5B and 5C identify and define the N-glycan and O-glycan structures, respectively, that are included in Table 1B. Both Tables 5B and 5C identify a coded representation of the composition for each glycan structure included in Table 1B. As used herein, the 4-digit GL NO. is a designation that represents the number of hexoses, the number of HexNAcs, the number of Fucoses, and the number of Neuraminic Acids.

TABLE 5B
N-Glycan Structure GL NOS: Compositions and Symbol Structures
in accordance with Table 1B

| Glycan | | Glycan Symbol | Glycan Mass |
|---|---|---|---|
| GL NO | Glycan Composition | Structure | (grams/mole) |
| 5402 | Hex(5)HexNAc(4)Fuc(0)NeuAc(2) | 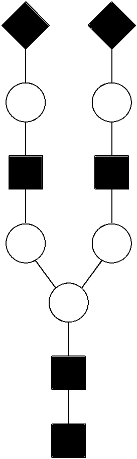 | 2204.77239 |
| 7624 | Hex(7)HexNAc(6)Fuc(2)NeuAc(4) | 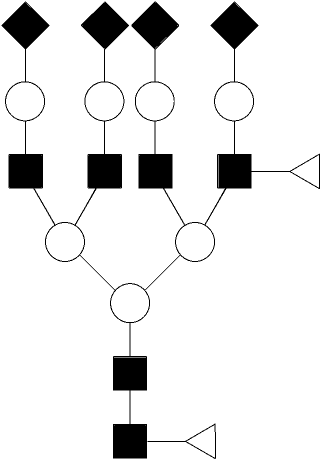 | 3809.3434 |
| 6503 | Hex(6)HexNAc(5)Fuc(0)NeuAc(3) | 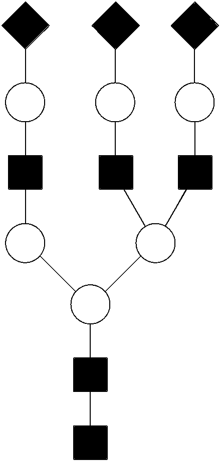 | 2860.99999 |
| 7602 | Hex(7)HexNAc(6)Fuc(0)NeuAc(2) | 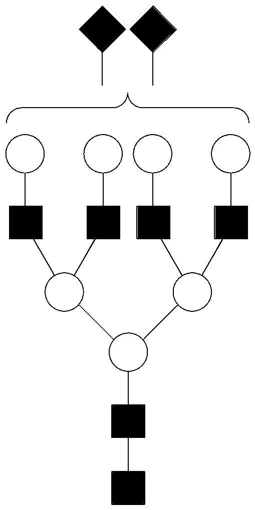 | 2935.03677 |
| 5411 | Hex)HexNAc(4)Fuc(1)NeuAc(1) | 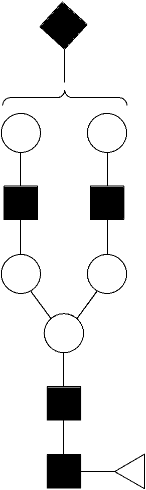 | 2059.73489 |

TABLE 5C
O-Glycan Structure GL NOS: Composition and Symbol Structures in
accordance with Table 1B

| Glycan | | Glycan Symbol | |
|---|---|---|---|
| GL NO | Glycan Composition | Structure | Glycan Mass |
| 1101 | Hex(1)HexNAc(1)Fuc(0)NeuAc(1) | 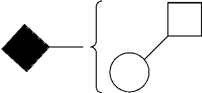 | 656.227598 |

### Legend for Tables 5B and 5C:

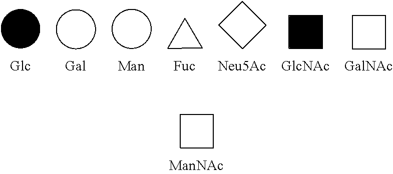

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating an APL or CRC disease state. A transition includes a precursor ion and at least one product ion grouping. As reviewed herein, the peptide structures in Table 1B, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, APL or CRC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2A. The alkylation procedure may be implemented in a manner similar to, for example, alkylation procedure 204 in FIG. 2A. The digestion procedure may be implemented in a manner similar to, for example, digestion procedure 206 in FIG. 2A.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 2B or an m/z ratio within an identified m/z ratio as provided in Table 2B. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VII.C Peptide Structure and Product Ion Compositions, Kits and Reagents Based on Table 1C

Aspects of the disclosure include compositions comprising one or more of the peptide structures listed in Table 1C. In some embodiments, a composition comprises a plurality of the peptide structures listed in Table 1C. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or all of the peptide structures listed in Table 1C. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 42-111, listed in Table 1C and/or Table 3E.

Aspects of the disclosure include compositions comprising one or more precursor ions having a defined charge and/or defined mass-to-charge (m/z) ratio, as listed in Table 2C. Aspects of the disclosure include compositions comprising one or more product ions having a defined mass-to-charge (m/z) ratio, which product ions are produced by converting a peptide structure described herein (e.g., a peptide structure listed in Table 1C and 3E) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1C). In some embodiments, a composition comprises a set of the product ions listed in Table 2C, having an m/z ratio selected from the list provided for each peptide structure in Table 1C or Table 3E.

In some embodiments, a composition comprises at least one of peptide structures of PS-ID No's. 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91 identified in Table 1C. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, or all 70 of the peptide structures of PS-ID No's. 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91 identified in Table 1C.

In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, or all 70 of the peptide structures of PS-ID No's. 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91 identified in Table 2C.

In some embodiments, a composition comprises a peptide structure or a product ion. The peptide structure or product ion comprises an amino acid sequence having at least 90% sequence identity to any one of SEQ ID NOS: 42-111, as identified in Tables 3E and/or 3F, corresponding to peptide structures PS-ID No's 22-91 in Table 1C.

In some embodiments, the product ion is selected as one from a group consisting of product ions identified in Table 2C, including product ions falling within an identified m/z range of the m/z ratio identified in Table 2C and characterized as having a precursor ion having an m/z ratio within an identified m/z range of the m/z ratio identified in Table 2C. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 2C, and characterized as having a precursor ion having an m/z ratio that falls within at least one of first range (±0.5), a second range (±1.0), or a third range (±1.0) of the precursor ion m/z ratio identified in Table 2C.

TABLE 2C
Mass Spectrometry-Related Characteristics for the Peptide Structures associated
with high-grade advanced pre-malignant lesions or CRC in accordance with Table 1C

| PS- | 1st | 1st | 2nd | 2nd | 1^{st} | 2nd | | Collision |
|---|---|---|---|---|---|---|---|---|
| ID | Precursor | Precursor | Precursor | Precursor | Product | Product | RT - | Energy - |
| No. | m/z | charge | m/z | charge | m/z | m/z | min | V |
| 22 | 1180.6 | 5 | N/A | N/A | 366.1 | 1299 | 42.9 | 29 |
| 23 | 991.2 | 4 | N/A | N/A | 366.1 | 980.5 | 38.2 | 24 |
| 24 | 1027.7 | 4 | N/A | N/A | 366.1 | 980 | 38 | 25 |
| 25 | 796.7 | 4 | N/A | N/A | 967.5 | 820.5 | 47.4 | 20 |
| 26 | 1210.9 | 3 | 908.4 | 4 | 366.1 | 366.1 | 40.2 | 32 |
| 27 | 1057 | 4 | N/A | N/A | 366.1 | 1184.1 | 43.5 | 22 |
| 28 | 1450 | 3 | N/A | N/A | 366.1 | 1318.7 | 40.165 | 30 |
| 29 | 1160.5 | 4 | N/A | N/A | 366.1 | 1318.7 | 40.665 | 20 |
| 30 | 1105.7 | 4 | N/A | N/A | 204.1 | 1208.6 | 33.9 | 30 |
| 31 | 1197 | 4 | N/A | N/A | 366.1 | 1208.6 | 33.5 | 25 |
| 32 | 1116.9 | 5 | N/A | N/A | 366.1 | 1060 | 23.8 | 25 |
| 33 | 1220.1 | 3 | N/A | N/A | 274.1 | 999.4 | 5.4 | 35 |
| 34 | 1386.1 | 4 | 1109.1 | 5 | 366.1 | 366.1 | 40 | 25 |
| 35 | 1458.9 | 4 | N/A | N/A | 366.1 | 1550.3 | 40.5 | 25 |
| 36 | 891 | 5 | N/A | N/A | 366.1 | 913.5 | 23.9 | 15 |
| 37 | 980.4 | 3 | N/A | N/A | 274.1 | N/A | 37.1 | 24 |
| 38 | 1012.8 | 3 | N/A | N/A | 274.1 | N/A | 36.95 | 24 |
| 39 | 816 | 3 | N/A | N/A | 274.1 | N/A | 19.3 | 20 |
| 40 | 749.8 | 2 | N/A | N/A | 827.4 | 642.4 | 19.1 | 22 |
| 41 | 811.4 | 2 | N/A | N/A | 569.2 | 765.4 | 13.6 | 30 |
| 42 | 1028.9 | 4 | N/A | N/A | 274.1 | 1453.6 | 19.6 | 25 |
| 43 | 626.2 | 2 | N/A | N/A | 491.2 | 780.3 | 19.9 | 20 |
| 44 | 845.3 | 3 | N/A | N/A | 1052.5 | 523.3 | 31.1 | 28 |
| 45 | 1146.1 | 5 | N/A | N/A | 366.1 | N/A | 37.1 | 25 |
| 46 | 1011.4 | 4 | N/A | N/A | 366.1 | 1165.5 | 26.7 | 20 |
| 47 | 1084.2 | 4 | N/A | N/A | 204.1 | N/A | 27.4 | 35 |
| 48 | 1057.9 | 4 | N/A | N/A | 366.1 | 1113 | 20.1 | 25 |
| 49 | 1258.5 | 4 | N/A | N/A | 274.1 | 1113 | 20.8 | 30 |
| 50 | 945.1 | 4 | N/A | N/A | 204.1 | N/A | 12 | 35 |
| 51 | 1307.3 | 4 | N/A | N/A | 366.1 | 1074.4 | 36.8 | 30 |
| 52 | 1158.2 | 3 | 1157.9 | 3 | 1230.1 | 204.1 | 27.4 | 35 |
| 53 | 1212.2 | 3 | 1211.9 | 3 | 1230.1 | 366.1 | 27.2 | 35 |
| 54 | 981.4 | 3 | N/A | N/A | 366.1 | N/A | 4.1 | 27 |
| 55 | 995.4 | 4 | N/A | N/A | 366.1 | N/A | 27.3 | 24 |
| 56 | 1234.3 | 4 | N/A | N/A | 366.1 | N/A | 30.2 | 31 |
| 57 | 1127.2 | 4 | N/A | N/A | 366.1 | N/A | 14.2 | 30 |
| 58 | 1015.2 | 4 | N/A | N/A | 366.1 | 685.6 | 7.3 | 30 |
| 59 | 1314.9 | 3 | N/A | N/A | 366.1 | N/A | 30.6 | 30 |
| 60 | 1184.5 | 4 | N/A | N/A | 204.1 | N/A | 40.5 | 35 |
| 61 | 1378.9 | 5 | N/A | N/A | 366.1 | N/A | 13.3 | 35 |
| 62 | 1001.2 | 4 | N/A | N/A | 366.1 | N/A | 30.4 | 24 |
| 63 | 1056.2 | 4 | 1407.9 | 3 | 366.1 | 366.1 | 28.4 | 25 |
| 64 | 1039.1 | 3 | N/A | N/A | 366.1 | 1112.5 | 10.2 | 25 |
| 65 | 990.7 | 3 | N/A | N/A | 366.1 | 1112.5 | 9.8 | 25 |
| 66 | 1167.3 | 4 | N/A | N/A | 366.1 | N/A | 39.4 | 30 |
| 67 | 1327.9 | 3 | N/A | N/A | 204.1 | 1281.2 | 41.66 | 25 |
| 68 | 1047 | 4 | N/A | N/A | 366.1 | 1281.2 | 41.57 | 20 |
| 69 | 909.8 | 3 | N/A | N/A | 366.1 | N/A | 12.2 | 22 |
| 70 | 1114.9 | 2 | N/A | N/A | 1392.6 | N/A | 7.9 | 35 |
| 71 | 879 | 3 | N/A | N/A | 204.1 | 1392.6 | 7.9 | 21 |
| 72 | 884.4 | 3 | N/A | N/A | 204.1 | 1392.6 | 7.9 | 21 |
| 73 | 987.1 | 3 | N/A | N/A | 366.1 | 1392.6 | 7.8 | 24 |
| 74 | 1006.1 | 3 | N/A | N/A | 204.1 | 1392.6 | 8 | 35 |
| 75 | 927.7 | 3 | N/A | N/A | 366.1 | N/A | 12.9 | 25 |
| 76 | 927.4 | 3 | N/A | N/A | 204.1 | 1376.6 | 9.9 | 15 |
| 77 | 1024.5 | 3 | N/A | N/A | 204.1 | 1376.6 | 10.5 | 25 |
| 78 | 717.2 | 3 | N/A | N/A | 912 | 608.3 | 26.8 | 20 |
| 79 | 1064.4 | 4 | N/A | N/A | 366.1 | 1271.6 | 25.4 | 30 |
| 80 | 1194.5 | 3 | N/A | N/A | 1284.7 | 204.1 | 31.3 | 25 |
| 81 | 896.7 | 3 | N/A | N/A | 204.1 | 744.4 | 6.3 | 30 |
| 82 | 909.6 | 4 | N/A | N/A | 366.1 | N/A | 6.6 | 22 |
| 83 | 1053.1 | 3 | N/A | N/A | 366.1 | N/A | 6.1 | 25 |
| 84 | 1285.1 | 4 | N/A | N/A | 366.1 | N/A | 38.05 | 30 |
| 85 | 1048.4 | 4 | N/A | N/A | 1094 | N/A | 37.8 | 18 |
| 86 | 1069.2 | 4 | N/A | N/A | 204.1 | N/A | 39.8 | 25 |
| 87 | 1083.8 | 3 | N/A | N/A | 204.1 | N/A | 16.9 | 30 |
| 88 | 1076.5 | 5 | N/A | N/A | 274.1 | N/A | 40.5 | 20 |
| 89 | 1012.7 | 4 | N/A | N/A | 366.1 | 840.4 | 26.8 | 25 |
| 90 | 1181.1 | 4 | N/A | N/A | 366.1 | N/A | 32.7 | 29 |
| 91 | 1039.4 | 3 | N/A | N/A | 366.1 | 1114.6 | 24.8 | 32 |

Table 3E defines the peptide sequences for SEQ ID NOS: 42-111 from Table 1C. Table 4C further identifies a corresponding protein SEQ ID NO. for each peptide sequence.

TABLE 3E
Peptide SEQ ID NOS in accordance with Table 1C

| PS-ID No. | Peptide.sequence | Pept. SEQ ID NO |
|---|---|---|
| 22 | ADTHDEILEGLNFNLTEIPEAQIHEGFQELLR | 42 |
| 23 | YLGNATAIFFLPDEGK | 43 |
| 24 | YLGNATAIFFLPDEGK | 44 |
| 25 | QLAHQSNSTNIFFSPVSIATAFAMLSLGTK | 45 |
| 26 | LPPGLLANFTLLR | 46 |
| 27 | VSNQTLSLFFTVLQDVPVR | 47 |
| 28 | IITILEEEMNVSVCGLYTYGK | 48 |
| 29 | IITILEEEMNVSVCGLYTYGK | 49 |
| 30 | TLNQSSDELQLSMGNAMFVK | 50 |
| 31 | TLNQSSDELQLSMGNAMFVK | 51 |
| 32 | QDQCIYNTTYLNVQR | 52 |
| 33 | NEEYNK | 53 |
| 34 | SVQEIQATFFYFTPNKTEDTIFLR | 54 |
| 35 | SVQEIQATFFYFTPNKTEDTIFLR | 55 |
| 36 | VYIHPFHLVIHNESTCEQLAK | 56 |
| 37 | FSEFWDLDPEVRPTSAVAA | 57 |
| 38 | DKFSEFWDLDPEVRPTSAVAA | 58 |
| 39 | AATVGSLAGQPLQER | 59 |
| 40 | AATVGSLAGQPLQER | 60 |
| 41 | VQAAVGTSAAPVPSDNH | 61 |
| 42 | LGNWSAMPSCK | 62 |
| 43 | LGNWSAMPSCK | 63 |
| 44 | TELFSSSCPGGIMLNETGQGYQR | 64 |
| 45 | FSLLGHASISCTVENETIGVWRPSPPTCEK | 65 |
| 46 | ENLTAPGSDSAVFFEQGTTR | 66 |
| 47 | ENLTAPGSDSAVFFEQGTTR | 67 |
| 48 | ELHHLQEQNVSNAFLDK | 68 |
| 49 | ELHHLQEQNVSNAFLDK | 69 |
| 50 | MDGASNVTCINSR | 70 |
| 51 | SPYEMFGDEEVMCLNGNWTEPPQCK | 71 |
| 52 | TVLTPATNHMGNVTFTIPANR | 72 |
| 53 | TVLTPATNHMGNVTFTIPANR | 73 |
| 54 | LSSNSTK | 74 |
| 55 | VCQDCPLLAPLNDTR | 75 |
| 56 | AALAAFNAQNNGSNFQLEEISR | 76 |
| 57 | HEEGHMLNCTCFGQGR | 77 |
| 58 | NGTGHGNSTHHGPEYMR | 78 |
| 59 | ALPQPQNVTSLLGCTH | 79 |
| 60 | CSDGWSFDATTLDDNGTMLFFK | 80 |
| 61 | NLFLNHSENATAK | 81 |
| 62 | VVLHPNYSQVDIGLIK | 82 |
| 63 | VIDFNCTTSSVSSALANTK | 83 |
| 64 | DTFVNASR | 84 |
| 65 | DTFVNASR | 85 |
| 66 | VGQLQLSHNLSLVILVPQNLK | 86 |
| 67 | PALEDLLLGSEANLTCTLTGLR | 87 |
| 68 | PALEDLLLGSEANLTCTLTGLR | 88 |
| 69 | TPLTANITK | 89 |
| 70 | EEQYNSTYR | 90 |
| 71 | EEQYNSTYR | 91 |
| 72 | EEQYNSTYR | 92 |
| 73 | EEQYNSTYR | 93 |
| 74 | EEQYNSTYR | 94 |
| 75 | EEQFNSTFR | 95 |
| 76 | EEQYNSTFR | 96 |
| 77 | EEQYNSTFR | 97 |
| 78 | IIVPLNNRENISDPTSPLR | 98 |
| 79 | GLTFQQNASSMCVPDQDTAIR | 99 |
| 80 | STGKPTLYNVSLVMSDTAGTCY | 100 |
| 81 | YKNNSDISSTR | 101 |
| 82 | YKNNSDISSTR | 102 |
| 83 | YKNNSDISSTR | 103 |
| 84 | ICDLLVANNHFAHFFAPQNLTNMNK | 104 |
| 85 | IYPGVDFGGEELNVTFVK | 105 |
| 86 | IVGGTNSSWGEWPWQVSLQVK | 106 |
| 87 | LDVDQALNR | 107 |
| 88 | WVLTAAHCLLYPPWDKNFTENDLLVR | |
| 89 | CGLVPVLAENYNK | 109 |
| 90 | QQQHLFGSNVTDCSGNFCLFR | 110 |
| 91 | NGSLFAFR | 111 |

Table 3F provides an indication of particular markers and includes the starting position of the peptide sequence within the protein sequence and the end position of the peptide sequence within the protein sequence.

TABLE 3F
Markers and Protein Positions in accordance with Table 1C

| PS- | | | | |
|---|---|---|---|---|
| ID | | | Start | End |
| No. | PS-NAME | Peptide Sequence | Position | Position |
| 22 | A1AT_107_5402 | ADTHDEILEGLNFNLTEIPEAQIHE | 94 | 125 |
| | | GFQELLR | | |
| 23 | A1AT_271_5402 | YLGNATAIFFLPDEGK | 268 | 283 |
| 24 | A1AT_271_5412 | YLGNATAIFFLPDEGK | 268 | 283 |
| 25 | A1AT_70_ | QLAHQSNSTNIFFSPVSIATAFAM | 64 | 93 |
| | NONGLYCOSYLATED | LSLGTK | | |
| 26 | A2GL_186_5402 | LPPGLLANFTLLR | 179 | 191 |
| 27 | A2MG_1424_5411 | VSNQTLSLFFTVLQDVPVR | 1422 | 1440 |
| 28 | A2MG_247MC_5401 | IITILEEEMNVSVCGLYTYGK | 238 | 258 |
| 29 | A2MG_247MC_5402 | IITILEEEMNVSVCGLYTYGK | 238 | 258 |
| 30 | AACT_127_5402 | TLNQSSDELQLSMGNAMFVK | 125 | 144 |
| 31 | AACT_127_6502 | TLNQSSDELQLSMGNAMFVK | 125 | 144 |
| 32 | AGP1_93_7614 | QDQCIYNTTYLNVQR | 87 | 101 |
| 33 | AGP12_56_6503 | NEEYNK | 52 | 57 |
| 34 | AGP12_72MC_7601 | SVQEIQATFFYFTPNKTEDTIFLR | 58 | 81 |
| 35 | AGP12_72MC_7602 | SVQEIQATFFYFTPNKTEDTIFLR | 58 | 81 |
| 36 | ANGT_47_5401 | VYIHPFHLVIHNESTCEQLAK | 36 | 56 |
| 37 | APOC3_74_1111 | FSEFWDLDPEVRPTSAVAA | 81 | 99 |
| 38 | APOC3_74MC_1101_ | DKFSEFWDLDPEVRPTSAVAA | 79 | 99 |
| | corr | | | |
| 39 | APOE_212_1102 | AATVGSLAGQPLQER | 210 | 224 |
| 40 | APOE_212_ | AATVGSLAGQPLQER | 210 | 224 |
| | NONGLYCOSYLATED | | | |
| 41 | APOE_308_ | VQAAVGTSAAPVPSDNH | 301 | 317 |
| | NONGLYCOSYLATED | | | |
| 42 | APOH_253_6503 | LGNWSAMPSCK | 251 | 261 |
| 43 | APOH_253_ | LGNWSAMPSCK | 251 | 261 |
| | NONGLYCOSYLATED | | | |
| 44 | APOM_135_ | TELFSSSCPGGIMLNETGQGYQR | 121 | 143 |
| | NONGLYCOSYLATED | | | |
| 45 | C4BPA_221_5412 | FSLLGHASISCTVENETIGVWRPSP | 207 | 236 |
| | | PTCEK | | |
| 46 | CERU_397_5401 | ENLTAPGSDSAVFFEQGTTR | 396 | 415 |
| 47 | CERU_397_5402 | ENLTAPGSDSAVFFEQGTTR | 396 | 415 |
| 48 | CERU_762_5402 | ELHHLQEQNVSNAFLDK | 754 | 770 |
| 49 | CERU_762_6513 | ELHHLQEQNVSNAFLDK | 754 | 770 |
| 50 | CFAH_1029_5412 | MDGASNVTCINSR | 1024 | 1036 |
| 51 | CFAH_1095_5402 | SPYEMFGDEEVMCLNGNWTEPP | 1079 | 1103 |
| | | QCK | | |
| 52 | CO3_85_5200 | TVLTPATNHMGNVTFTIPANR | 74 | 94 |
| 53 | CO3_85_6200 | TVLTPATNHMGNVTFTIPANR | 74 | 94 |
| 54 | CO6_855_5402 | LSSNSTK | 852 | 858 |
| 55 | FETUA_156_5402 | VCQDCPLLAPLNDTR | 145 | 159 |
| 56 | FETUA_176_6502 | AALAAFNAQNNGSNFQLEEISR | 166 | 187 |
| 57 | FINC_542_6502 | HEEGHMLNCTCFGQGR | 535 | 550 |
| 58 | HEMO_240/246_5402 | NGTGHGNSTHHGPEYMR | 240 | 256 |
| 59 | HEMO_453_5402 | ALPQPQNVTSLLGCTH | 447 | 462 |
| 60 | HEMO_64_5402 | CSDGWSFDATTLDDNGTMLFFK | 50 | 71 |
| 61 | HPT_207_121005 | NLFLNHSENATAK | 203 | 215 |
| 62 | HPT_241_5402 | VVLHPNYSQVDIGLIK | 236 | 251 |
| 63 | HRG_125_5402 | VIDFNCTTSSVSSALANTK | 121 | 139 |
| 64 | IC1_238_5402 | DTFVNASR | 234 | 241 |
| 65 | IC1_238_5411 | DTFVNASR | 234 | 241 |
| 66 | IC1_352_5412 | VGQLQLSHNLSLVILVPQNLK | 344 | 364 |
| 67 | IGA12_144MC_5400 | PALEDLLLGSEANLTCTLTGLR | 132 | 153 |
| | | | (P01876) | (P01876) |
| | | | or 119 | or 140 |
| | | | (P01877) | (P01877) |
| 68 | IGA12_144MC_5500 | PALEDLLLGSEANLTCTLTGLR | 132 | 153 |
| | | | (P01876) | (P01876) |
| | | | or 119 | or 140 |
| | | | (P01877) | (P01877) |
| 69 | IGA2_205_5410 | TPLTANITK | 200 | 208 |
| 70 | IGG1_297_3210 | EEQYNSTYR | 176 | 184 |
| 71 | IGG1_297_3410 | EEQYNSTYR | 176 | 184 |
| 72 | IGG1_297_4400 | EEQYNSTYR | 176 | 184 |
| 73 | IGG1_297_5410 | EEQYNSTYR | 176 | 184 |
| 74 | IGG1_297_5500 | EEQYNSTYR | 176 | 184 |
| 75 | IGG2_297_5400 | EEQFNSTFR | 172 | 180 |
| 76 | IGG34_297_4410 | EEQYNSTFR | 223 | 231 |
| 77 | IGG34_297_4411 | EEQYNSTFR | 223 | 231 |
| 78 | IGJ_71MC_ | IIVPLNNRENISDPTSPLR | 62 | 80 |
| | NONGLYCOSYLATED | | | |
| 79 | IGM_209_5401 | GLTFQQNASSMCVPDQDTAIR | 203 | 223 |
| 80 | IGM_439_5200 | STGKPTLYNVSLVMSDTAGTCY | 432 | 453 |
| 81 | IGM_46_4310 | YKNNSDISSTR | 44 | 54 |
| 82 | IGM_46_5412 | YKNNSDISSTR | 44 | 54 |
| 83 | IGM_46_6301 | YKNNSDISSTR | 44 | 54 |
| 84 | ITIH1_285_5402 | ICDLLVANNHFAHFFAPQNLTNM | 267 | 291 |
| | | NK | | |
| 85 | KLKB1_308_5402 | IYPGVDFGGEELNVTFVK | 296 | 313 |
| 86 | KLKB1_396_5401 | IVGGTNSSWGEWPWQVSLQVK | 391 | 411 |
| 87 | SHBG_380_5402 | LDVDQALNR | 373 | 381 |
| 88 | THRB_416MC_5402 | WVLTAAHCLLYPPWDKNFTENDL | 400 | 425 |
| | | LVR | | |
| 89 | TRFE_432_6502 | CGLVPVLAENYNK | 421 | 433 |
| 90 | TRFE_630_5402 | QQQHLFGSNVTDCSGNFCLFR | 622 | 642 |
| 91 | VTNC_169_5402 | NGSLFAFR | 169 | 176 |

Table 4C identifies the proteins of SEQ ID NOS: 1-3, 13-17, 19-20, 22, 23, 25-26, 112-132 from Table 1C. Table 4C identifies a corresponding protein abbreviation and protein name for each of protein SEQ ID NOS: 1-3, 13-17, 19-20, 22, 23, 25-26, 112-132. Further, Table 4C identifies a corresponding Uniprot TD for each of protein SEQ ID NOS: 1-3, 13-17, 19-20, 22, 23, 25-26, 112-132.

TABLE 4C
Protein SEQ ID NOS in accordance with Table 1C

| Protein | | | Prot Seq |
|---|---|---|---|
| Abbrev. | Protein Name | Uniprot ID | ID NO. |
| IC1 | Plasma protease C1 inhibitor | P05155 | 1 |
| HEMO | Hemopexin | P02790 | 2 |
| A2MG | Alpha-2-macroglobulin | P01023 | 3 |
| A1AT | Alpha-1-antitrypsin | P01009 | 13 |
| AGP1 | Alpha-1-acid glycoprotein 1 | P02763 | 14 |
| AGP2 | Alpha-1-acid glycoprotein 2 | P19652 | 15 |
| ANGT | Angiotensinogen | P01019 | 16 |
| FETUA | Alpha-2-HS-glycoprotein | P02765 | 17 |
| HPT | Haptoglobin | P00738 | 19 |
| HRG | Histidine-rich Glycoprotein | P04196 | 20 |
| ITIH1 | Inter-alpha-trypsin | P19827 | 22 |
| | inhibitor heavy chain H1 | | |
| FINC | Fibronectin | P02751 | 23 |
| VTNC | Vitronectin | P04004 | 25 |
| THRB | Prothrombin | P00734 | 26 |
| A2GL | Leucine-richAlpha-2-glycoprotein | P02750 | 112 |
| AACT | Alpha-1-antichymotrypsin | P01011 | 113 |
| APOC3 | Apolipoprotein C-III | P02656 | 114 |
| APOE | Apolipoprotein E | P02649 | 115 |
| APOH | Beta-2-glycoprotein1 | P02749 | 116 |
| APOM | Apolipoprotein M | O95445 | 117 |
| C4BPA | C4b-binding protein alpha chain | P04003 | 118 |
| CERU | Ceruloplasmin | P00450 | 119 |
| CFAH | ComplementFactorH | P08603 | 120 |
| CO3 | ComplementC3 | P01024 | 121 |
| CO6 | ComplementcomponentC6 | P13671 | 122 |
| IGA1 | Immunoglobulin heavy | P01876 | 123 |
| | constant alpha 1 | | |
| IGA2 | Immunoglobulin heavy | P01877 | 124 |
| | constant alpha 2 | | |
| IGG1 | Immunoglobulin heavy | P01857 | 125 |
| | constant gamma 1 | | |
| IGG2 | Immunoglobulin heavy | P01859 | 126 |
| | constant gamma 2 | | |
| IGG34 | Immunoglobulin heavy | P01860 | 127 |
| | constant gamma 3 | | |
| IGJ | Immunoglobulin J chain | P01591 | 128 |
| IGM | Immunoglobulin heavy constant mu | P01871 | 129 |
| KLKB1 | Plasma Kallikrein | P03952 | 130 |
| SHBG | Sex hormone-binding globulin | P04278 | 131 |
| TRFE | Serotransferrin | P02787 | 132 |

Table 5D and 5E identify and define the N-glycan and O-glycan structures, respectively, that are included in Table 1C as Glycan Structure GL No's. Both Tables 5D and 5E identify a coded representation of the composition for each glycan structure included in Table 1C. As used herein, the 4-digit GL NO. is a designation that represents the number of hexoses, the number of HexNAcs, the number of Fucoses, and the number of Neuraminic Acids.

TABLE 5D
N-Glycan Symbol Structure GL NOS: Compositions and Symbol
Structures in accordance with Table 1C

| Glycan | | Glyan Symbol | Glycan Mass |
|---|---|---|---|
| GL NO | Glycan Composition | Structure | (grams/mole) |
| 3210 | Hex(3)HexNAc(2)Fuc(1)NeuAc(0) | 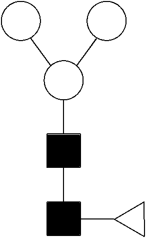 | 1038.375102 |
| 3410 | Hex(3)HexNAc(4)Fuc(1)NeuAc(0) | 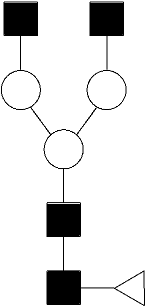 | 1444.533838 |
| 4310 | Hex(4)HexNAc(3)Fuc(1)NeuAc(0) | 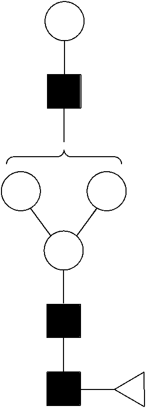 | 1403.50729 |
| 4400 | Hex(4)HexNAc(4)Fuc(0)NeuAc(0) | 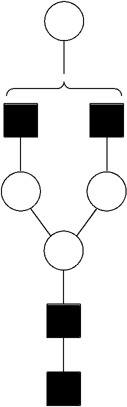 | 1460.528752 |
| 4410 | Hex(4)HexNAc(4)Fuc(1)NeuAc(0) | 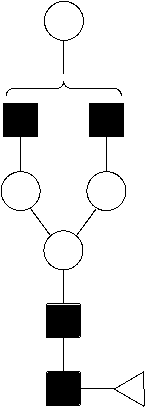 | 1606.586658 |
| 4411 | Hex(4)HexNAc(4)Fuc(1)NeuAc(1) | 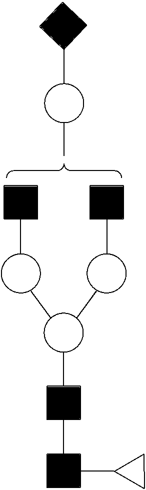 | 1897.682068 |
| 5200 | Hex(5)HexNAc(2)Fuc(0)NeuAc(0) | 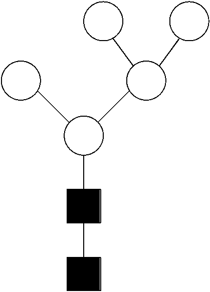 | 1216.422836 |
| 5400 | Hex(5)HexNAc(4)Fuc(0)NeuAc(0) | 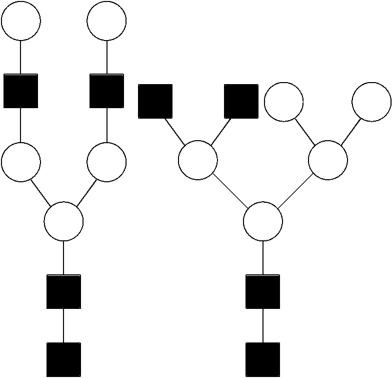 | 1622.581572 |
| 5401 | Hex(5)HexNAc(4)Fuc(0)NeuAc(1) | 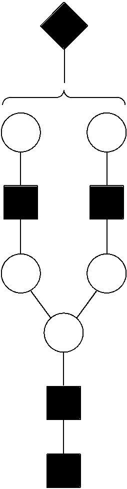 | 1913.676982 |
| 5402 | Hex(5)HexNAc(4)Fuc(0)NeuAc(2) | 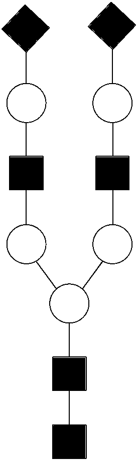 | 2204.77239 |
| 5410 | Hex(5)HexNAc(4)Fuc(1)NeuAc(0) | 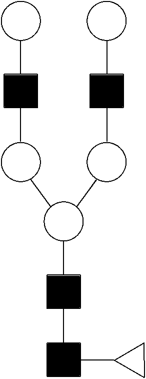 | 1768.639478 |
| 5411 | Hex(5)HexNAc(4)Fuc(1)NeuAc(1) | 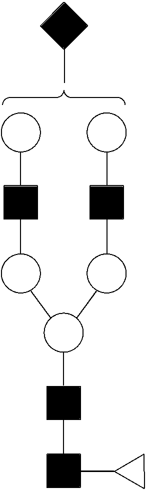 | 2059.73489 |
| 5412 | Hex(5)HexNAc(4)Fuc(1)NeuAc(2) | 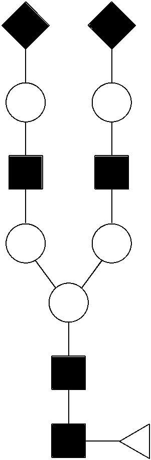 | 2350.830298 |
| 5500 | Hex(5)HexNAc(5)Fuc(0)NeuAc(0) | 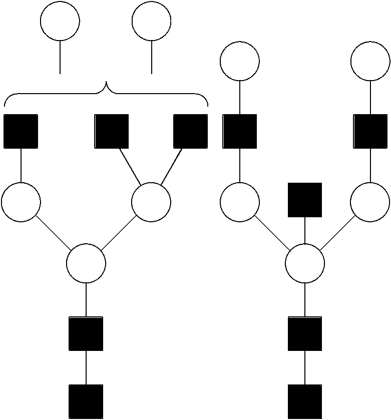 | 1825.66094 |
| 6200 | Hex(6)HexNAc(2)Fuc(0)NeuAc(0) | 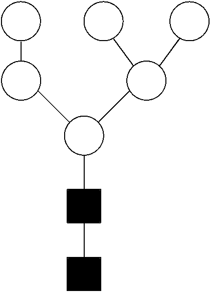 | 1378.475656 |
| 6301 | Hex(6)HexNAc(3)Fuc(0)NeuAc(1) | 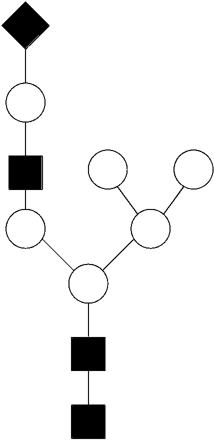 | 1872.650434 |
| 6502 | Hex(6)HexNAc(5)Fuc(0)NeuAc(2) | 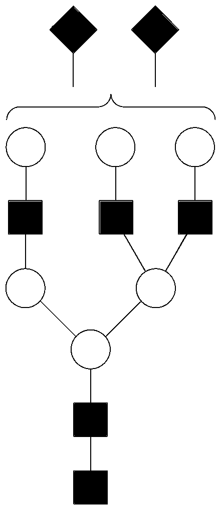 | 2569.90458 |
| 6503 | Hex(6)HexNAc(5)Fuc(0)NeuAc(3) | 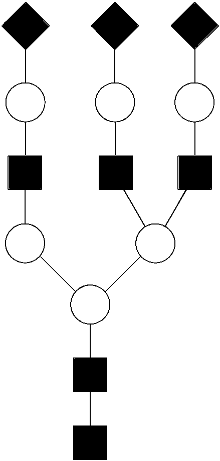 | 2860.99999 |
| 6513 | Hex(6)HexNAc(5)Fuc(1)NeuAc(3) | 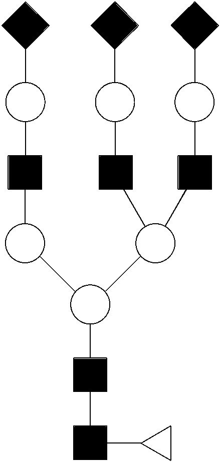 | 3007.057896 |
| 7601 | Hex(7)HexNAc(6)Fuc(0)NeuAc(1) | 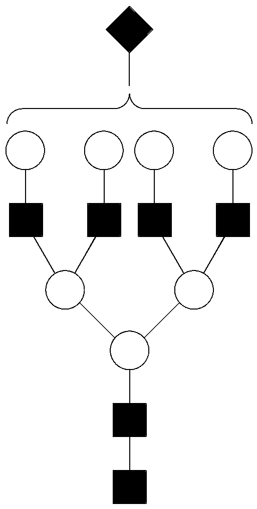 | 2643.941358 |
| 7602 | Hex(7)HexNAc(6)Fuc(0)NeuAc(2) | 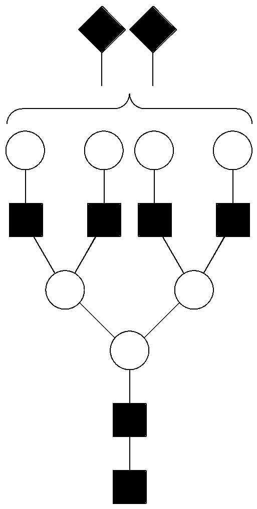 | 2935.03677 |
| 7614 | Hex(7)HexNAc(6)Fuc(1)NeuAc(4) | 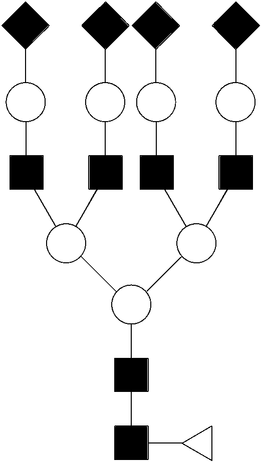 | 3663.285494 |

TABLE 5E
O-Glycan GL NOS: Compositions and Symbol Structures in
accordance with Table 1C

| Glycan | | Glycan Symbol | Glycan Mass |
|---|---|---|---|
| GL No. | Glycan Composition | Structure | (grams/mole) |
| 1101 | Hex(1)HexNAc(1)Fuc(0)NeuAc(1) | 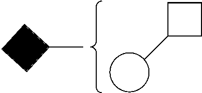 | 656.227598 |
| 1102 | Hex(1)HexNAc(1)Fuc(0)NeuAc(2) | 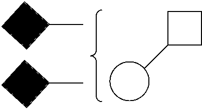 | 947.323008 |
| 1111 | Hex(1)HexNAc(1)Fuc(1)NeuAc(1) | 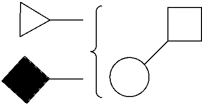 | 802.285504 |

### Legend for Tables 5D and 5E:

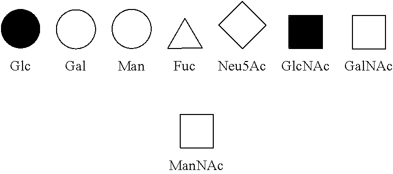

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating an high-grade advanced pre-malignant lesion or CRC disease state. A transition includes a precursor ion and at least one product ion grouping. As reviewed herein, the peptide structures in Table 1C, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, high-grade advanced pre-malignant lesion or CRC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 2C or an m/z ratio within an identified m/z ratio as provided in Table 2C. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VII.D Peptide Structure and Product Ion Compositions, Kits and Reagents Based on Table 1D

Aspects of the disclosure include compositions comprising one or more of the peptide structures listed in Table 1D. In some embodiments, a composition comprises a plurality of the peptide structures listed in Table 1D. In some embodiments, a composition comprises 1, 2, 3, 4, 5, 6, 7, 8, 9, 10, 11, 12, 13, 14, 15, 16, 17, 18, 19, 20, or all of the peptide structures listed in Table 1D. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 136-146, listed in Table 1D and/or Table 3G.

Aspects of the disclosure include compositions comprising one or more precursor ions having a defined charge and/or defined mass-to-charge (m/z) ratio, as listed in Table 2D. Aspects of the disclosure include compositions comprising one or more product ions having a defined mass-to-charge (m/z) ratio, which product ions are produced by converting a peptide structure described herein (e.g., a peptide structure listed in Table 1D and 3H) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1D). In some embodiments, a composition comprises a set of the product ions listed in Table 2D, having an m/z ratio selected from the list provided for each peptide structure in Table 1D or Table 3G.

In some embodiments, a composition comprises at least one of peptide structures of PS-ID No's. 92, 93, 94, 95, 96, 97, 98, 99, 100, 101, 102, 103, 104, 105, 106, 107, 108, 109, 110, 111, and 112 identified in Table 1D. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or all 21 of the peptide structures of PS-ID No's. 92, 93, 94, 95, 96, 97, 98, 99, 100, 101, 102, 103, 104, 105, 106, 107, 108, 109, 110, 111, and 112 identified in Table 1D.

In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or all 21 of the peptide structures of PS-ID No's. 92, 93, 94, 95, 96, 97, 98, 99, 100, 101, 102, 103, 104, 105, 106, 107, 108, 109, 110, 111, and 112 identified in Table 2D.

In some embodiments, a composition comprises a peptide structure or a product ion. The peptide structure or product ion comprises an amino acid sequence having at least 90% sequence identity to any one of SEQ ID NOS: 136-156, as identified in Tables 3G and/or 3H, corresponding to peptide structures PS-TD No's 92-112 in Table 1D.

In some embodiments, the product ion is selected as one from a group consisting of product ions identified in Table 2D, including product ions falling within an identified m/z range ofthe m/z ratio identified in Table 2D and characterized as having a precursor ion having an m/z ratio within an identified m/z range ofthe m/z ratio identified in Table 2D. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 2D, and characterized as having a precursor ion having an m/z ratio that falls within at least one of first range (±0.5), a second range (±1.0), or a third range (±1.0) of the precursor ion m/z ratio identified in Table 2D.

TABLE 2D
Mass Spectrometry-Related Characteristics for the Peptide
Structures associated with CRC in accordance with Table 1D

| PS- | 1st | 1st | 2nd | 2nd | 1^{st} | 2nd | | Collision |
|---|---|---|---|---|---|---|---|---|
| ID | Precursor | Precursor | Precursor | Precursor | Product | Product | RT - | Energy - |
| No. | m/z | charge | m/z | charge | m/z | m/z | min | V |
| 92 | 1213.3 | 3 | N/A | N/A | 366.1 | 979.5 | 5.8 | 30 |
| 93 | 1110.8 | 4 | N/A | N/A | 366.1 | 979.5 | 5.9 | 27 |
| 94 | 1287.8 | 4 | N/A | N/A | 366.1 | 1062.5 | 37.5 | 25 |
| 95 | 1222.2 | 4 | N/A | N/A | 366.1 | 1441.7 | 33.8 | 30 |
| 96 | 1124 | 5 | N/A | N/A | 366.1 | N/A | 33.2 | 20 |
| 97 | 1247.7 | 5 | N/A | N/A | 366.1 | N/A | 13.2 | 31 |
| 98 | 1335.1 | 5 | N/A | N/A | 366.1 | N/A | 13.4 | 34 |
| 99 | 1173.6 | 6 | N/A | N/A | 366.1 | N/A | 13.2 | 29 |
| 100 | 1221.8 | 3 | N/A | N/A | 366.1 | N/A | 14.7 | 30 |
| 101 | 1165 | 4 | N/A | N/A | 366.1 | N/A | 31.1 | 29 |
| 102 | 710.8 | 4 | N/A | N/A | 274.1 | 865.5 | 6.6 | 15 |
| 103 | 590.3 | 2 | N/A | N/A | 725.4 | 342.2 | 30.6 | 15 |
| 104 | 561.8 | 2 | N/A | N/A | 244.2 | 455.3 | 9.5 | 25 |
| 105 | 536.8 | 2 | N/A | N/A | 864.5 | 604.3 | 14.8 | 18 |
| 106 | 433.7 | 2 | N/A | N/A | 696.4 | 533.3 | 13.6 | 11 |
| 107 | 387.7 | 2 | N/A | N/A | 288.2 | 209.1 | 7.9 | 12 |
| 108 | 609.7 | 3 | N/A | N/A | 692.4 | 835.9 | 32.9 | 13 |
| 109 | 515.8 | 2 | N/A | N/A | 720.4 | 607.3 | 31.8 | 14 |
| 110 | 416.7 | 2 | N/A | N/A | 646.4 | 733.4 | 12.7 | 10 |
| 111 | 486.2 | 2 | N/A | N/A | 707.3 | 636.3 | 6.4 | 13 |
| 112 | 617.3 | 2 | N/A | N/A | 301.2 | 960.4 | 25.6 | 22 |

Table 3G defines the peptide sequences for SEQ ID NOS: 136-156 from Table 1D. Table 4D further identifies a corresponding protein SEQ ID NO. for each peptide sequence.

TABLE 3G
Peptide SEQ ID NOS in accordance with Table 1D

| PS- | | Pept. |
|---|---|---|
| ID No. | Peptide.sequence | SEQ ID NO |
| 92 | ENGTISR | 136 |
| 93 | ENGTISR | 137 |
| 94 | SVQEIQATFFYFTPNK | 138 |
| 95 | MVSHHNLTTGATLINEQWLLTTAK | 139 |
| 96 | MVSHHNLTTGATLINEQWLLTTAK | 140 |
| 97 | NLFLNHSENATAK | 141 |
| 98 | NLFLNHSENATAK | 142 |
| 99 | NLFLNHSENATAK | 143 |
| 100 | NLFLNHSENATAK | 144 |
| 101 | VVLHPNYSQVDIGLIK | 145 |
| 102 | SSTTKPPFKPHGSR | 146 |
| 103 | DLLLPQPDLR | 147 |
| 104 | VNHVTLSQPK | 148 |
| 105 | HAFILQDTK | 149 |
| 106 | GIYGTISR | 150 |
| 107 | AHYDLR | 151 |
| 108 | GVTSVSQIFHSPDLAIR | 152 |
| 109 | YFIDFVAR | 153 |
| 110 | VSLATVDK | 154 |
| 111 | YTACETAR | 155 |
| 112 | CLAYDFYPGK | 156 |

Table 3H provides an indication of particular markers and includes the starting position of the peptide sequence within the protein sequence and the end position of the peptide sequence within the protein sequence.

TABLE 3H
Markers and Protein Positions in accordance with Table 1D

| PS- | | | | |
|---|---|---|---|---|
| ID | | | Start | End |
| No. | PS-NAME | Peptide Sequence | Position | Position |
| 92 | AGP1_103_6503 | ENGTISR | 102 | 108 |
| 93 | AGP1_103_7614 | ENGTISR | 102 | 108 |
| 94 | AGP12_72_7603 | SVQEIQATFFYFTPNK | 58 | 73 |
| 95 | HPT_184_5402 | MVSHHNLTTGATLINEQWLLTTA | 179 | 202 |
| | | K | | |
| 96 | HPT_184_7602 | MVSHHNLTTGATLINEQWLLTTA | 179 | 202 |
| | | K | | |
| 97 | HPT_207_11904 | NLFLNHSENATAK | 203 | 215 |
| 98 | HPT_207_11915 | NLFLNHSENATAK | 203 | 215 |
| 99 | HPT_207_121015 | NLFLNHSENATAK | 203 | 215 |
| 100 | HPT_207_5402 | NLFLNHSENATAK | 203 | 215 |
| 101 | HPT_241_6503 | VVLHPNYSQVDIGLIK | 236 | 251 |
| 102 | HRG_271_2202 | SSTTKPPFKPHGSR | 271 | 284 |
| 103 | A2GL_DLLLPQPDLR | DLLLPQPDLR | 230 | 239 |
| 104 | B2M_VNHVTLSQPK | VNHVTLSQPK | 102 | 111 |
| 105 | CO2_HAFILQDTK | HAFILQDTK | 435 | 443 |
| 106 | CO5_GIYGTISR | GIYGTISR | 948 | 955 |
| 107 | FETUA_AHYDLR | AHYDLR | 312 | 317 |
| 108 | IC1_ | GVTSVSQIFHSPDLAIR | 217 | 233 |
| | GVTSVSQIFHSPDLAIR | | | |
| 109 | KNG1_YFIDFVAR | YFIDFVAR | 317 | 324 |
| 110 | SEPP1_VSLATVDK | VSLATVDK | 189 | 196 |
| 111 | THRB_YTACETAR | YTACETAR | 87 | 94 |
| 112 | ZA2G_CLAYDFYPGK | CLAYDFYPGK | 225 | 234 |

Table 4D identifies the proteins of SEQ ID NOS: 1, 5, 13, 14, 15, 17, 19, 20, 21, 24, 26, 133, 134, and 135 from Table 1D. Table 4D identifies a corresponding protein abbreviation and protein name for each of protein SEQ ID NOS: 1, 5, 13, 14, 15, 17, 19, 20, 21, 24, 26, 133, 134, and 135. Further, Table 4D identifies a corresponding Uniprot ID for each of protein SEQ ID NOS: 1, 5, 13, 14, 15, 17, 19, 20, 21, 24, 26, 133, 134, and 135.

TABLE 4D
Protein SEQ ID NOS in accordance with Table 1D

| Protein | | | Prot Seq |
|---|---|---|---|
| Abbrev. | Protein Name | Uniprot ID | ID NO. |
| AGP1 | Alpha-1-acid glycoprotein 1 | P02763 | 14 |
| AGP2 | Alpha-1-acid glycoprotein 2 | P19652 | 15 |
| HPT | Haptoglobin | P00738 | 19 |
| A2GL | Leucine-richAlpha-2-glycoprotein | P02750 | 13 |
| B2M | Beta-2-microglobulin | P61769 | 5 |
| CO2 | ComplementC2 | P06681 | 133 |
| CO5 | ComplementC5 | P01031 | 134 |
| FETUA | Alpha-2-HS-glycoprotein | P02765 | 17 |
| IC1 | Plasma protease C1 inhibitor | P05155 | 1 |
| KNG1 | Kininogen-1 | P01042 | 21 |
| SEPP1 | Selenoprotein P | P49908 | 24 |
| THRB | Prothrombin | P00734 | 26 |
| ZA2G | Zinc-alpha-2-glycoprotein | P25311 | 135 |

Table 5F and 5G identify and define the N-glycan and O-glycan structures, respectively, that are included in Table 1D as Glycan Structure GL No's. Both Tables 5F and 5G identify a coded representation of the composition for each glycan structure included in Table 1D. As used herein, the 4-digit GL NO. is a designation that represents the number of hexoses, the number of HexNAcs, the number of Fucoses, and the number of Neuraminic Acids.

TABLE 5F
N-Glycan Symbol Structure GL NOS: Compositions and Symbol
Structures in accordance with Table 1D

| Glycan | | Glycan Symbol | Glycan Mass |
|---|---|---|---|
| GL NO | Glycan Composition | Structure | (grams/mole) |
| 5402 | Hex(5)HexNAc(4)Fuc(0)NeuAc(2) | 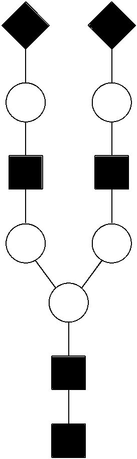 | 2204.77239 |
| 6502 | Hex(6)HexNAc(5)Fuc(0)NeuAc(2) | 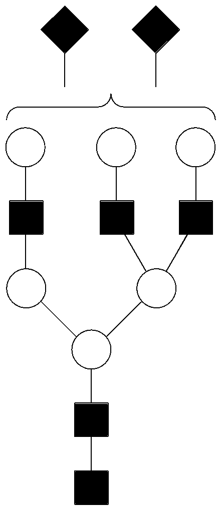 | 2569.90458 |
| 6503 | Hex(6)HexNAc(5)Fuc(0)NeuAc(3) | 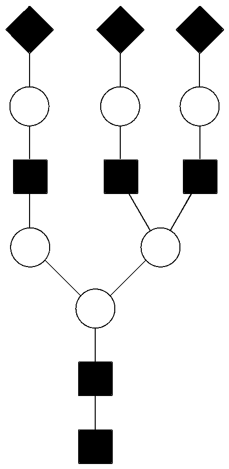 | 2860.99999 |
| 6513 | Hex(6)HexNAc(5)Fuc(1)NeuAc(3) | 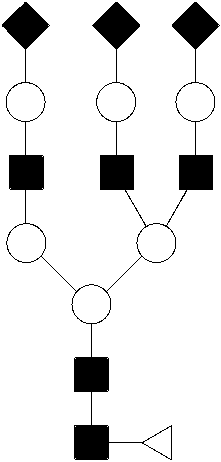 | 3007.057896 |
| 7602 | Hex(7)HexNAc(6)Fuc(0)NeuAc(2) | 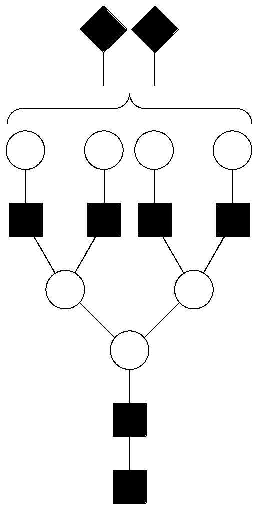 | 2935.03677 |
| 7603 | Hex(7)HexNAc(6)Fuc(0)NeuAc(3) | 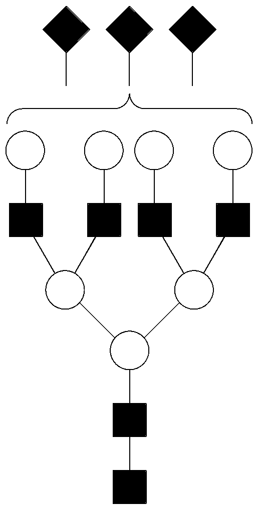 | 3226.132178 |
| 7614 | Hex(7)HexNAc(6)Fuc(1)NeuAc(4) | 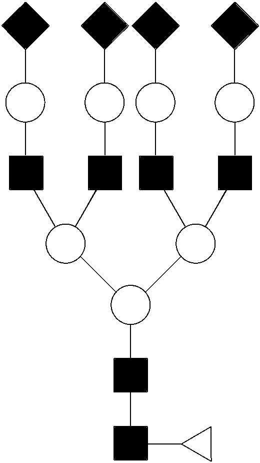 | 3663.285494 |

TABLE 5G
O-Glycan GL NOS: Compositions and Symbol Structures in
accordance with Table 1D

| Glycan | | Glycan Symbol | Glycan Mass |
|---|---|---|---|
| GL No. | Glycan Composition | Structure | (grams/mole) |
| 2202 | Hex(2)HexNAc(2)Fuc(0)NeuAc(2) | 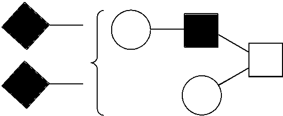 | 1312.455196 |

### Legend for Tables 5F and 5G:

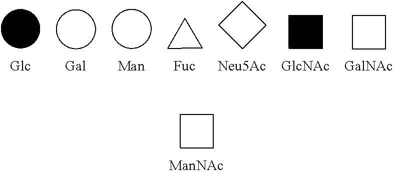

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating a CRC disease state. A transition includes a precursor ion and at least one product ion grouping. As reviewed herein, the peptide structures in Table 1D, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, CRC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 2D or an m/z ratio within an identified m/z ratio as provided in Table 2D. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data

VIII.A.1 Representative Experimental Results—Subject Sample Model & Corresponding Training of Said Model based on Table 1

To assess the association of individual peptide structures (biomarkers) with adenoma or colorectal cancer, differential expression analysis (DEA) was run on a cohort of 563 samples sourced from different biorepositories, including 247 CRC samples (mean age 65.6; 50% female), 32 adenoma samples (mean age 68.6; 53% female), 196 healthy control samples (mean age 51.7; 52% female), and 88 ulcerative colitis control samples (mean age 44.1; 47% female). Of the 563 samples, the distribution is as follows: 35% healthy; 16% UC, 6% AA, 11% CRC stage 1, 10% CRC stage 2, 13% CRC stage 3, and 10% CRC stage 4 with a mean of 57 years of age (range: 19-94). The samples were split into a training (50%) and a hold-out testing set (50%) for the development of a machine learning (ML)-based multivariable predictive model. Statistical analysis was performed on normalized data to identify biomarkers differentiating AAs and different stages of CRC from controls. The inclusion of ulcerative colitis control samples in the study served to rule out non-cancerous inflammation as the source of the differentiation.

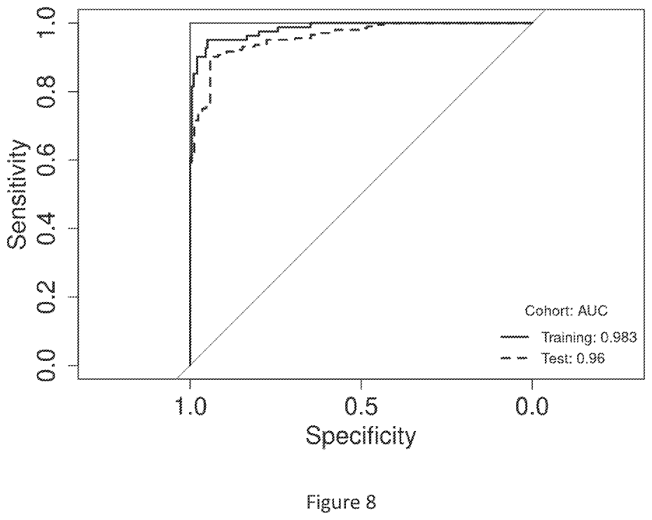

Results of the DEA are summarized below with reference to Table 6 and FIGS. 8-10. There were 250 differentially abundant (FDR<0.05) glycopeptides/peptides when comparing CRC and AA samples with healthy and UC controls. A subset was assessed, generating a six (6) biomarker ML classification model (see Table 1 for a listing of the biomarkers). The model was applied to the hold-out test and achieved an overall sensitivity of 91.4% and specificity of 91.8% for predicting AA/CRC versus healthy/UC with an area under the receiver operating characteristic of 0.962 (AuROC=0.98 for training set). AA and CRC separately were predicted with a sensitivity of 84.4% and 92.8%, respectively, relative to healthy/UC with sensitivities for CRC stage 1/2 and stage 3/4 being 91.2% and 93.2%, respectively.

FIG. 8 contains two ROC curves providing train and test performance (AUC) for a classifier model that classifies CRC and adenoma samples from the control samples.

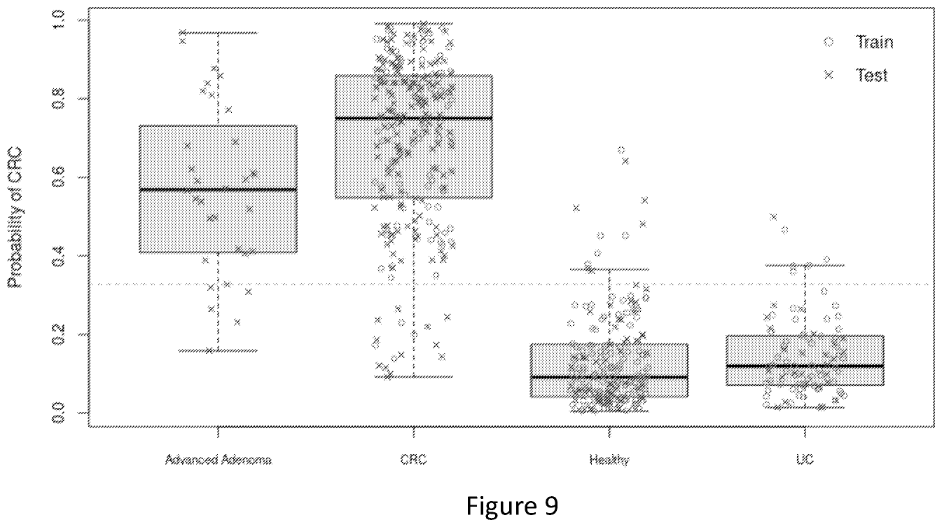

FIG. 9 demonstrates a probability of CRC or adenoma based on an examination of a Train & Test data set to determine the performance of the classifier model, utilizing samples of adenoma, ulcerative colitis control, healthy control, and colorectal cancer for a collection of stages.

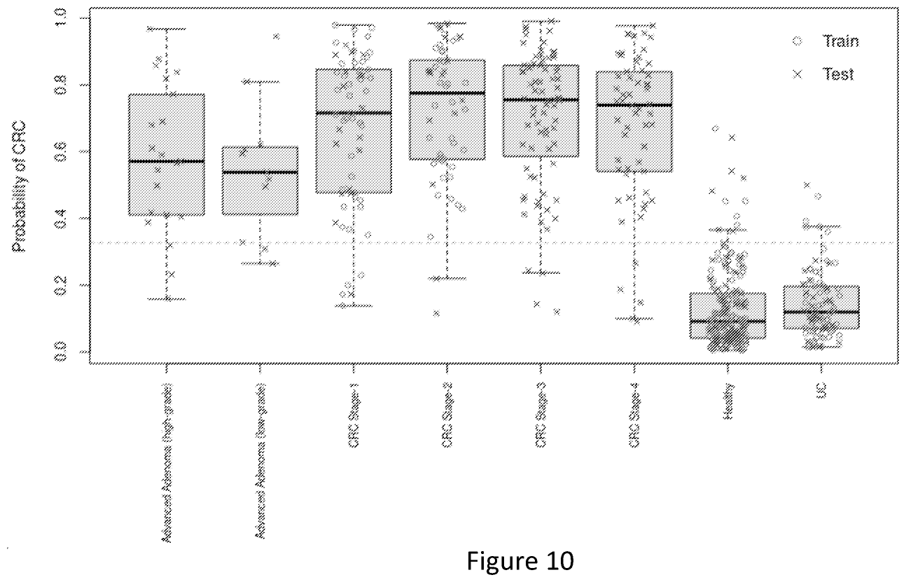

FIG. 10 demonstrates a probability of advanced adenoma (AA) or CRC based on an examination of a Train & Test data set to determine the performance of the classifier model, utilizing samples of advanced adenoma (high-grade), advanced adenoma (low-grade), respective stages 1, 2, 3, and 4 of CRC, healthy control, ulcerative colitis control. Equivalent probability distributions between training and test sets indicates a well-fit model, and application to advanced adenomas and stages 3 and 4 of CRC, exclusively considered in the test set, demonstrates a biologically-relevant score that tracks with the progression of the disease.

TABLE 6
Differential Expression Analysis (DEA)

| | | Sensi- | Speci- | | | |
|---|---|---|---|---|---|---|
| Comparison | Accuracy | tivity | ficity | PPV | NPV | F1 |
| CRC/AA vs | 0.915 | 0.914 | 0.918 | 0.963 | 0.821 | 0.938 |
| Healthy/UC | | | | | | |
| Controls | | | | | | |
| CRC vs Healthy/UC | 0.924 | 0.928 | 0.918 | 0.957 | 0.867 | 0.942 |
| Controls | | | | | | |
| AA vs Healthy/UC | 0.897 | 0.844 | 0.918 | 0.794 | 0.94 | 0.818 |
| Controls | | | | | | |
| CRC Stage 1/2 vs | 0.916 | 0.912 | 0.918 | 0.816 | 0.963 | 0.861 |
| Healthy/UC | | | | | | |
| Controls | | | | | | |
| CRC Stage 3/4 vs | 0.926 | 0.932 | 0.918 | 0.946 | 0.897 | 0.939 |
| Healthy/UC | | | | | | |
| Controls | | | | | | |

### VII.E Additional Description of Tables 2, 2B, 2C, and 2D for MRM-MS

Tables 2, 2B, 2C, and 2D show various parameters associated with the identification of the peptide and glycopeptides using LC and MRM-MS. The term monoisotopic mass represents the mass of the glycopeptide in grams per mole. The first precursor m/z represents a ratio value associated with an ionized form having a first precursor charge for the peptide or glycopeptide. Similarly, the second precursor m/z represents a ratio value associated with an ionized form having a second precursor charge for the peptide or glycopeptide. The first precursor ion is associated with a first product ion having a m/z ratio that was formed from a collision and the second precursor ion is associated with a second product ion having a m/z ratio that was formed from a collision. Under certain circumstances, the first precursor and the second precursor may be the same, but the associated first and second product m/z ratios are different. The retention time (RT) represents the amount of time in minutes for the peptide elute from the chromatography column. The collision energy represents the energy applied to the peptide for creating fragments (i.e., product ions) such as, for example, in the 2nd quadrupole of the triple quadrupole MS.

### VII.F Additional Description of Tables 5, 5B to 5H for Glycans

Tables 5, 5B to 5H illustrate the Glycan GL No., composition, symbol structure, and glycan mass of detected glycan moieties that correspond to glycopeptides of Tables 1, 1B, 1C, and 1D based on the Glycan GL No. It should be noted that Tables 5, 5B, 5D, and 5F represent N-linked glycans and Tables 5C, 5E, and 5G represent O-linked glycans.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these clasess are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. For example, it should be noted that the Glycan Structure GL NOs. 1102 is an O-linked glycan (see SEQ ID No 59 in Table 5E). For reference, N-linked glycans have a glycan attached to the amino acid asparagine and O-linked glycans have a glycan attached to either a serine or a threonine.

The identity of the various monosaccharides is illustrated by the Legend section located at the end of Tables 5, 5C, 5E, and 5G. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

Referring back to Table 5D, for some entries, there are two symbol structures provided for one Glycan Structure GL No such as, for example, Glycan Structure GL No 5400 or 5500. Thus, the identify of a peptide that references a Glycan Structure GL NO that has two symbol structures could be one of two possibilities based on the MRM of the LC-MS analysis. In some instances, a bracket symbol is used as part of the Symbol Structure to indicate that the precise bonding linkage is not exactly known, but that the linking line segment is attached to one of the plurality of adjacent carbohydrates immediately adjacent to the bracket

### VI.F Sequence of Amino Acids for Proteins Corresponding to Tables 1, 1B, IC, and 1D

Table 14B lists the SEQ ID NO, Protein Abbreviations, Protein Name, Uniprot ID, and Protein sequence for each of the proteins listed Tables 2, 2B, 2C, and 2D.

TABLE 14B
Sequence of Amino Acids for Proteins Corresponding to Tables 2, 2B, 2C, and
2D

| SEQ | | | | |
|---|---|---|---|---|
| ID | Protein | | Uniprot | |
| NO | Abbreviation | Protein Name | ID | Protein Sequence |
| 1 | IC1 | Plasma | P05155 | MASRLTLLTLLLLLLAGDRASSNPNATSSSSQDPESLQD |
| | | protease C1 | | RGEGKVATTVISKMLFVEPILEVSSLPTTNSTTNSATKIT |
| | | inhibitor | | ANTTDEPTTQPTTEPTTQPTIQPTQPTTQLPTDSPTQPTTG |
| | | | | SFCPGPVTLCSDLESHSTEAVLGDALVDFSLKLYHAFSA |
| | | | | MKKVETNMAFSPFSIASLLTQVLLGAGENTKTNLESILS |
| | | | | YPKDFTCVHQALKGFTTKGVTSVSQIFHSPDLAIRDTFV |
| | | | | NASRTLYSSSPRVLSNNSDANLELINTWVAKNTNNKISR |
| | | | | LLDSLPSDTRLVLLNAIYLSAKWKTTFDPKKTRMEPFHF |
| | | | | KNSVIKVPMMNSKKYPVAHFIDQTLKAKVGQLQLSHN |
| | | | | LSLVILVPQNLKHRLEDMEQALSPSVFKAIMEKLEMSKF |
| | | | | QPTLLTLPRIKVTTSQDMLSIMEKLEFFDFSYDLNLCGLT |
| | | | | EDPDLQVSAMQHQTVLELTETGVEAAAASAISVARTLL |
| | | | | VFEVQQPFLFVLWDQQHKFPVFMGRVYDPRA |
| 2 | HEMO | Hemopexin | P02790 | MARVLGAPVA LGLWSLCWSL AIATPLPPTS AHGNVAEGET |
| | | | | KPDPDVTERC SDGWSFDATT |
| | | | | LDDNGTMLFF KGEFVWKSHK WDRELISERW KNFPSPVDAA |
| | | | | FRQGHNSVFL IKGDKVWVYP |
| | | | | PEKKEKGYPK LLQDEFPGIP SPLDAAVECH RGECQAEGVL |
| | | | | FFQGDREWFW DLATGTMKER |
| | | | | SWPAVGNCSS ALRWLGRYYC FQGNQFLRFD PVRGEVPPRY |
| | | | | PRDVRDYFMP CPGRGHGHRN |
| | | | | GTGHGNSTHH GPEYMRCSPH LVLSALTSDN HGATYAFSGT |
| | | | | HYWRLDTSRD GWHSWPIAHQ |
| | | | | WPQGPSAVDA AFSWEEKLYL VQGTQVYVFL TKGGYTLVSG |
| | | | | YPKRLEKEVG TPHGIILDSV |
| | | | | DAAFICPGSS RLHIMAGRRL WWLDLKSGAQ ATWTELPWPH |
| | | | | EKVDGALCME KSLGPNSCSA |
| | | | | NGPGLYLIHG PNLYCYSDVE KLNAAKALPQ |
| | | | | PQNVTSLLGC TH |
| 3 | A2MG | Alpha-2- | P01023 | MGKNKLLHPSLVLLLLVLLPTDASVSGKPQYMVLVPSL |
| | | macroglobulin | | LHTETTEKGCVLLSYLNETVTVSASLESVRGNRSLFTDL |
| | | | | EAENDVLHCVAFAVPKSSSNEEVMFLTVQVKGPTQEFK |
| | | | | KRTTVMVKNEDSLVFVQTDKSIYKPGQTVKFRVVSMD |
| | | | | ENFHPLNELIPLVYIQDPKGNRIAQWQSFQLEGGLKQFS |
| | | | | FPLSSEPFQGSYKVVVQKKSGGRTEHPFTVEEFVLPKFE |
| | | | | VQVTVPKIITILEEEMNVSVCGLYTYGKPVPGHVTVSIC |
| | | | | RKYSDASDCHGEDSQAFCEKFSGQLNSHGCFYQQVKT |
| | | | | KVFQLKRKEYEMKLHTEAQIQEEGTVVELTGRQSSEITR |
| | | | | TITKLSFVKVDSHFRQGIPFFGQVRLVDGKGVPIPNKVIF |
| | | | | IRGNEANYYSNATTDEHGLVQFSINTTNVMGTSLTVRV |
| | | | | NYKDRSPCYGYQWVSEEHEEAHHTAYLVFSPSKSFVHL |
| | | | | EPMSHELPCGHTQTVQAHYILNGGTLLGLKKLSFYYLI |
| | | | | MAKGGIVRTGTHGLLVKQEDMKGHFSISIPVKSDIAPVA |
| | | | | RLLIYAVLPTGDVIGDSAKYDVENCLANKVDLSFSPSQS |
| | | | | LPASHAHLRVTAAPQSVCALRAVDQSVLLMKPDAELSA |
| | | | | SSVYNLLPEKDLTGFPGPLNDQDNEDCINRHNVYINGIT |
| | | | | YTPVSSTNEKDMYSFLEDMGLKAFTNSKIRKPKMCPQL |
| | | | | QQYEMHGPEGLRVGFYESDVMGRGHARLVHVEEPHTE |
| | | | | TVRKYFPETWIWDLVVVNSAGVAEVGVTVPDTITEWK |
| | | | | AGAFCLSEDAGLGISSTASLRAFQPFFVELTMPYSVIRGE |
| | | | | AFTLKATVLNYLPKCIRVSVQLEASPAFLAVPVEKEQAP |
| | | | | HCICANGRQTVSWAVTPKSLGNVNFTVSAEALESQELC |
| | | | | GTEVPSVPEHGRKDTVIKPLLVEPEGLEKETTFNSLLCPS |
| | | | | GGEVSEELSLKLPPNVVEESARASVSVLGDILGSAMQNT |
| | | | | QNLLQMPYGCGEQNMVLFAPNIYVLDYLNETQQLTPEI |
| | | | | KSKAIGYLNTGYQRQLNYKHYDGSYSTFGERYGRNQG |
| | | | | NTWLTAFVLKTFAQARAYIFIDEAHITQALIWLSQRQKD |
| | | | | NGCFRSSGSLLNNAIKGGVEDEVTLSAYITIALLEIPLTV |
| | | | | THPVVRNALFCLESAWKTAQEGDHGSHVYTKALLAYA |
| | | | | FALAGNQDKRKEVLKSLNEEAVKKDNSVHWERPQKPK |
| | | | | APVGHFYEPQAPSAEVEMTSYVLLAYLTAQPAPTSEDL |
| | | | | TSATNIVKWITKQQNAQGGFSSTQDTVVALHALSKYGA |
| | | | | ATFTRTGKAAQVTIQSSGTFSSKFQVDNNNRLLLQQVSL |
| | | | | PELPGEYSMKVTGEGCVYLQTSLKYNILPEKEEFPFALG |
| | | | | VQTLPQTCDEPKAHTSFQISLSVSYTGSRSASNMAIVDV |
| | | | | KMVSGFIPLKPTVKMLERSNHVSRTEVSSNHVLIYLDKV |
| | | | | SNQTLSLFFTVLQDVPVRDLKPAIVKVYDYYETDEFAIA |
| | | | | EYNAPCSKDLGNA |
| 4 | HBA | Hemoglobin | P69905 | MVLSPADKTNVKAAWGKVGAHAGEYGAEALERMFLS |
| | | subunit alpha | | FPTTKTYFPHFDLSHGSAQVKGHGKKVADALTNAVAH |
| | | | | VDDMPNALSALSDLHAHKLRVDPVNFKLLSHCLLVTLA |
| | | | | AHLPAEFTPAVHASLDKFLASVSTVLTSKYR |
| 5 | B2M | Beta-2- | P61769 | MSRSVALAVLALLSLSGLEAIQRTPKIQVYSRHPAENGK |
| | | microglobulin | | SNFLNCYVSGFHPSDIEVDLLKNGERIEKVEHSDLSFSK |
| | | | | DWSFYLLYYTEFTPTEKDEYACRVNHVTLSQPKIVKWD |
| | | | | RDM |
| 6 | AFAM | Afamin | P43652 | MKLLKLTGFIFFLFFLTESLTLPTQPRDIENFNSTQKFIED |
| | | | | NIEYITIIAFAQYVQEATFEEMEKLVKDMVEYKDRCMA |
| | | | | DKTLPECSKLPNNVLQEKICAMEGLPQKHNFSHCCSKV |
| | | | | DAQRRLCFFYNKKSDVGFLPPFPTLDPEEKCQAYESNRE |
| | | | | SLLNHFLYEVARRNPFVFAPTLLTVAVHFEEVAKSCCEE |
| | | | | QNKVNCLQTRAIPVTQYLKAFSSYQKHVCGALLKFGTK |
| | | | | VVHFIYIAILSQKFPKIEFKELISLVEDVSSNYDGCCEGD |
| | | | | VVQCIRDTSKVMNHICSKQDSISSKIKECCEKKIPERGQC |
| | | | | IINSNKDDRPKDLSLREGKFTDSENVCQERDADPDTFFA |
| | | | | KFTFEYSRRHPDLSIPELLRIVQIYKDLLRNCCNTENPPG |
| | | | | CYRYAEDKFNETTEKSLKMVQQECKHFQNLGKDGLKY |
| | | | | HYLIRLTKIAPQLSTEELVSLGEKMVTAFTTCCTLSEEFA |
| | | | | CVDNLADLVFGELCGVNENRTINPAVDHCCKTNFAFRR |
| | | | | PCFESLKADKTYVPPPFSQDLFTFHADMCQSQNEELQR |
| | | | | KTDRFLVNLVKLKHELTDEELQSLFTNFANVVDKCCKA |
| | | | | ESPEVCFNEESPKIGN |
| 13 | A2GL | Leucine- | P02750 | MSSWSRQRPKSPGGIQPHVSRTLFLLLLLAASAWGVTLS |
| | | richAlpha-2- | | PKDCQVFRSDHGSSISCQPPAEIPGYLPADTVHLAVEFFN |
| | | glycoprotein | | LTHLPANLLQGASKLQELHLSSNGLESLSPEFLRPVPQL |
| | | | | RVLDLTRNALTGLPPGLFQASATLDTLVLKENQLEVLE |
| | | | | VSWLHGLKALGHLDLSGNRLRKLPPGLLANFTLLRTLD |
| | | | | LGENQLETLPPDLLRGPLQLERLHLEGNKLQVLGKDLLL |
| | | | | PQPDLRYLFLNGNKLARVAAGAFQGLRQLDMLDLSNN |
| | | | | SLASVPEGLWASLGQPNWDMRDGFDISGNPWICDQNLS |
| | | | | DLYRWLQAQKDKMFSQNDTRCAGPEAVKGQTLLAVA |
| | | | | KSQ |
| 14 | AGP1 | Alpha-1-acid | P02763 | MALSWVLTVLSLLPLLEAQIPLCANLVPVPITNATLDRIT |
| | | glycoprotein 1 | | GKWFYIASAFRNEEYNKSVQEIQATFFYFTPNKTEDTIFL |
| | | | | REYQTRQDQCIYNTTYLNVQRENGTISRYVGGQEHFAH |
| | | | | LLILRDTKTYMLAFDVNDEKNWGLSVYADKPETTKEQL |
| | | | | GEFYEALDCLRIPKSDVVYTDWKKDKCEPLEKQHEKER |
| | | | | KQEEGES |
| 15 | AGP2 | Alpha-1-acid | P19652 | MALSWVLTVLSLLPLLEAQIPLCANLVPVPITNATLDRIT |
| | | glycoprotein 1 | | GKWFYIASAFRNEEYNKSVQEIQATFFYFTPNKTEDTIFL |
| | | | | REYQTRQNQCFYNSSYLNVQRENGTVSRYEGGREHVA |
| | | | | HLLFLRDTKTLMFGSYLDDEKNWGLSFYADKPETTKEQ |
| | | | | LGEFYEALDCLCIPRSDVMYTDWKKDKCEPLEKQHEKE |
| | | | | RKQEEGES |
| 16 | ANGT | Angiotensinogen | P01019 | MRKRAPQSEMAPAGVSLRATILCLLAWAGLAAGDRVY |
| | | | | IHPFHLVIHNESTCEQLAKANAGKPKDPTFIPAPIQAKTS |
| | | | | PVDEKALQDQLVLVAAKLDTEDKLRAAMVGMLANFL |
| | | | | GFRIYGMHSELWGVVHGATVLSPTAVFGTLASLYLGAL |
| | | | | DHTADRLQAILGVPWKDKNCTSRLDAHKVLSALQAVQ |
| | | | | GLLVAQGRADSQAQLLLSTVVGVFTAPGLHLKQPFVQG |
| | | | | LALYTPVVLPRSLDFTELDVAAEKIDRFMQAVTGWKTG |
| | | | | CSLMGASVDSTLAFNTYVHFQGKMKGFSLLAEPQEFW |
| | | | | VDNSTSVSVPMLSGMGTFQHWSDIQDNFSVTQVPFTES |
| | | | | ACLLLIQPHYASDLDKVEGLTFQQNSLNWMKKLSPRTI |
| | | | | HLTMPQLVLQGSYDLQDLLAQAELPAILHTELNLQKLS |
| | | | | NDRIRVGEVLNSIFFELEADEREPTESTQQLNKPEVLEVT |
| | | | | LNRPFLFAVYDQSATALHFLGRVANPLSTA |
| 17 | FETUA | Alpha-2-HS- | P02765 | MKSLVLLLCLAQLWGCHSAPHGPGLIYRQPNCDDPETE |
| | | glycoprotein | | EAALVAIDYINQNLPWGYKHTLNQIDEVKVWPQQPSGE |
| | | | | LFEIEIDTLETTCHVLDPTPVARCSVRQLKEHAVEGDCD |
| | | | | FQLLKLDGKFSVVYAKCDSSPDSAEDVRKVCQDCPLLA |
| | | | | PLNDTRVVHAAKAALAAFNAQNNGSNFQLEEISRAQLV |
| | | | | PLPPSTYVEFTVSGTDCVAKEATEAAKCNLLAEKQYGF |
| | | | | CKATLSEKLGGAEVAVTCMVFQTQPVSSQPQPEGANEA |
| | | | | VPTPVVDPDAPPSPPLGAPGLPPAGSPPDSHVLLAAPPG |
| | | | | HQLHRAHYDLRHTFMGVVSLGSPSGEVSHPRKTRTVV |
| | | | | QPSVGAAAGPVVPPCPGRIRHFKV |
| 18 | ITIH4 | Inter-alpha- | Q14624 | MKPPRPVRTCSKVLVLLSLLAIHQTTTAEKNGIDIYSLTV |
| | | trypsin | | DSRVSSRFAHTVVTSRVVNRANTVQEATFQMELPKKAF |
| | | inhibitor heavy | | ITNFSMIIDGMTYPGIIKEKAEAQAQYSAAVAKGKSAGL |
| | | chain H4 | | VKATGRNMEQFQVSVSVAPNAKITFELVYEELLKRRLG |
| | | | | VYELLLKVRPQQLVKHLQMDIHIFEPQGISFLETESTFMT |
| | | | | NQLVDALTTWQNKTKAHIRFKPTLSQQQKSPEQQETVL |
| | | | | DGNLIIRYDVDRAISGGSIQIENGYFVHYFAPEGLTTMPK |
| | | | | NVVFVIDKSGSMSGRKIQQTREALIKILDDLSPRDQFNLI |
| | | | | VFSTEATQWRPSLVPASAENVNKARSFAAGIQALGGTNI |
| | | | | NDAMLMAVQLLDSSNQEERLPEGSVSLIILLTDGDPTVG |
| | | | | ETNPRSIQNNVREAVSGRYSLFCLGFGFDVSYAFLEKLA |
| | | | | LDNGGLARRIHEDSDSALQLQDFYQEVANPLLTAVTFE |
| | | | | YPSNAVEEVTQNNFRLLFKGSEMVVAGKLQDRGPDVL |
| | | | | TATVSGKLPTQNITFQTESSVAEQEAEFQSPKYIFHNFME |
| | | | | RLWAYLTIQQLLEQTVSASDADQQALRNQALNLSLAYS |
| | | | | FVTPLTSMVVTKPDDQEQSQVAEKPMEGESRNRNVHS |
| | | | | GSTFFKYYLQGAKIPKPEASFSPRRGWNRQAGAAGSRM |
| | | | | NFRPGVLSSRQLGLPGPPDVPDHAAYHPFRRLAILPASA |
| | | | | PPATSNPDPAVSRVMNMKIEETTMTTQTPAPIQAPSAILP |
| | | | | LPGQSVERLCVDPRHRQGPVNLLSDPEQGVEVTGQYER |
| | | | | EKAGFSWIEVTFKNPLVWVHASPEHVVVTRNRRSSAYK |
| | | | | WKETLFSVMPGLKMTMDKTGLLLLSDPDKVTIGLLFW |
| | | | | DGRGEGLRLLLRDTDRFSSHVGGTLGQFYQEVLWGSPA |
| | | | | ASDDGRRTLRVQGNDHSATRERRLDYQEGPPGVEISCW |
| | | | | SVEL |
| 19 | HPT | Haptoglobin | P00738 | MSALGAVIALLLWGQLFAVDSGNDVTDIADDGCPKPPE |
| | | | | IAHGYVEHSVRYQCKNYYKLRTEGDGVYTLNDKKQWI |
| | | | | NKAVGDKLPECEADDGCPKPPEIAHGYVEHSVRYQCK |
| | | | | NYYKLRTEGDGVYTLNNEKQWINKAVGDKLPECEAVC |
| | | | | GKPKNPANPVQRILGGHLDAKGSFPWQAKMVSHHNLT |
| | | | | TGATLINEQWLLTTAKNLFLNHSENATAKDIAPTLTLYV |
| | | | | GKKQLVEIEKVVLHPNYSQVDIGLIKLKQKVSVNERVM |
| | | | | PICLPSKDYAEVGRVGYVSGWGRNANFKFTDHLKYVM |
| | | | | LPVADQDQCIRHYEGSTVPEKKTPKSPVGVQPILNEHTF |
| | | | | CAGMSKYQEDTCYGDAGSAFAVHDLEEDTWYATGILS |
| | | | | FDKSCAVAEYGVYVKVTSIQDWVQKTIAEN |
| 20 | HRG | Histidine-rich | P04196 | MKALIAALLLITLQYSCAVSPTDCSAVEPEAEKALDLIN |
| | | Glycoprotein | | KRRRDGYLFQLLRIADAHLDRVENTTVYYLVLDVQESD |
| | | | | CSVLSRKYWNDCEPPDSRRPSEIVIGQCKVIATRHSHES |
| | | | | QDLRVIDFNCTTSSVSSALANTKDSPVLIDFFEDTERYRK |
| | | | | QANKALEKYKEENDDFASFRVDRIERVARVRGGEGTG |
| | | | | YFVDFSVRNCPRHHFPRHPNVFGFCRADLFYDVEALDL |
| | | | | ESPKNLVINCEVFDPQEHENINGVPPHLGHPFHWGGHER |
| | | | | SSTTKPPFKPHGSRDHHHPHKPHEHGPPPPPDERDHSHG |
| | | | | PPLPQGPPPLLPMSCSSCQHATFGTNGAQRHSHNNNSSD |
| | | | | LHPHKHHSHEQHPHGHHPHAHHPHEHDTHRQHPHGHH |
| | | | | PHGHHPHGHHPHGHHPHGHHPHCHDFQDYGPCDPPPH |
| | | | | NQGHCCHGHGPPPGHLRRRGPGKGPRPFHCRQIGSVYR |
| | | | | LPPLRKGEVLPLPEANFPSFPLPHHKHPLKPDNQPFPQSV |
| | | | | SESCPGKFKSGFPQVSMFFTHTFPK |
| 21 | KNG1 | Kininogen-1 | P01042 | MKLITILFLCSRLLLSLTQESQSEEIDCNDKDLFKAVDAA |
| | | | | LKKYNSQNQSNNQFVLYRITEATKTVGSDTFYSFKYEIK |
| | | | | EGDCPVQSGKTWQDCEYKDAAKAATGECTATVGKRSS |
| | | | | TKFSVATQTCQITPAEGPVVTAQYDCLGCVHPISTQSPD |
| | | | | LEPILRHGIQYFNNNTQHSSLFMLNEVKRAQRQVVAGL |
| | | | | NFRITYSIVQTNCSKENFLFLTPDCKSLWNGDTGECTDN |
| | | | | AYIDIQLRIASFSQNCDIYPGKDFVQPPTKICVGCPRDIPT |
| | | | | NSPELEETLTHTITKLNAENNATFYFKIDNVKKARVQVV |
| | | | | AGKKYFIDFVARETTCSKESNEELTESCETKKLGQSLDC |
| | | | | NAEVYVVPWEKKIYPTVNCQPLGMISLMKRPPGFSPFRS |
| | | | | SRIGEIKEETTVSPPHTSMAPAQDEERDSGKEQGHTRRH |
| | | | | DWGHEKQRKHNLGHGHKHERDQGHGHQRGHGLGHG |
| | | | | HEQQHGLGHGHKFKLDDDLEHQGGHVLDHGHKHKHG |
| | | | | HGHGKHKNKGKKNGKHNGWKTEHLASSSEDSTTPSAQ |
| | | | | TQEKTEGPTPIPSLAKPGVTVTFSDFQDSDLIATMMPPIS |
| | | | | PAPIQSDDDWIPDIQIDPNGLSFNPISDFPDTTSPKCPGRP |
| | | | | WKSVSEINPTTQMKESYYFDLTDGLS |
| 22 | ITIH1 | Inter-alpha- | P19827 | MDGAMGPRGLLLCMYLVSLLILQAMPALGSATGRSKSS |
| | | trypsin | | EKRQAVDTAVDGVFIRSLKVNCKVTSRFAHYVVTSQV |
| | | inhibitor heavy | | VNTANEAREVAFDLEIPKTAFISDFAVTADGNAFIGDIK |
| | | chain H1 | | DKVTAWKQYRKAAISGENAGLVRASGRTMEQFTIHLT |
| | | | | VNPQSKVTFQLTYEEVLKRNHMQYEIVIKVKPKQLVHH |
| | | | | FEIDVDIFEPQGISKLDAQASFLPKELAAQTIKKSFSGKK |
| | | | | GHVLFRPTVSQQQSCPTCSTSLLNGHFKVTYDVSRDKIC |
| | | | | DLLVANNHFAHFFAPQNLTNMNKNVVFVIDISGSMRGQ |
| | | | | KVKQTKEALLKILGDMQPGDYFDLVLFGTRVQSWKGS |
| | | | | LVQASEANLQAAQDFVRGFSLDEATNLNGGLLRGIEILN |
| | | | | QVQESLPELSNHASILIMLTDGDPTEGVTDRSQILKNVR |
| | | | | NAIRGRFPLYNLGFGHNVDFNFLEVMSMENNGRAQRIY |
| | | | | EDHDATQQLQGFYSQVAKPLLVDVDLQYPQDAVLALT |
| | | | | QNHHKQYYEGSEIVVAGRIADNKQSSFKADVQAHGEG |
| | | | | QEFSITCLVDEEEMKKLLRERGHMLENHVERLWAYLTI |
| | | | | QELLAKRMKVDREERANLSSQALQMSLDYGFVTPLTS |
| | | | | MSIRGMADQDGLKPTIDKPSEDSPPLEMLGPRRTFVLSA |
| | | | | LQPSPTHSSSNTQRLPDRVTGVDTDPHFIIHVPQKEDTLC |
| | | | | FNINEEPGVILSLVQDPNTGFSVNGQLIGNKARSPGQHD |
| | | | | GTYFGRLGIANPATDFQLEVTPQNITLNPGFGGPVFSWR |
| | | | | DQAVLRQDGVVVTINKKRNLVVSVDDGGTFEVVLHRV |
| | | | | WKGSSVHQDFLGFYVLDSHRMSARTHGLLGQFFHPIGF |
| | | | | EVSDIHPGSDPTKPDATMVVRNRRLTVTRGLQKDYSKD |
| | | | | PWHGAEVSCWFIHNNGAGLIDGAYTDYIVPDIF |
| 23 | FINC | Fibronectin | P02751 | MLRGPGPGLLLLAVQCLGTAVPSTGASKSKRQAQQMV |
| | | | | QPQSPVAVSQSKPGCYDNGKHYQINQQWERTYLGNAL |
| | | | | VCTCYGGSRGFNCESKPEAEETCFDKYTGNTYRVGDTY |
| | | | | ERPKDSMIWDCTCIGAGRGRISCTIANRCHEGGQSYKIG |
| | | | | DTWRRPHETGGYMLECVCLGNGKGEWTCKPIAEKCFD |
| | | | | HAAGTSYVVGETWEKPYQGWMMVDCTCLGEGSGRIT |
| | | | | CTSRNRCNDQDTRTSYRIGDTWSKKDNRGNLLQCICTG |
| | | | | NGRGEWKCERHTSVQTTSSGSGPFTDVRAAVYQPQPHP |
| | | | | QPPPYGHCVTDSGVVYSVGMQWLKTQGNKQMLCTCL |
| | | | | GNGVSCQETAVTQTYGGNSNGEPCVLPFTYNGRTFYSC |
| | | | | TTEGRQDGHLWCSTTSNYEQDQKYSFCTDHTVLVQTR |
| | | | | GGNSNGALCHFPFLYNNHNYTDCTSEGRRDNMKWCGT |
| | | | | TQNYDADQKFGFCPMAAHEEICTTNEGVMYRIGDQWD |
| | | | | KQHDMGHMMRCTCVGNGRGEWTCIAYSQLRDQCIVD |
| | | | | DITYNVNDTFHKRHEEGHMLNCTCFGQGRGRWKCDPV |
| | | | | DQCQDSETGTFYQIGDSWEKYVHGVRYQCYCYGRGIG |
| | | | | EWHCQPLQTYPSSSGPVEVFITETPSQPNSHPIQWNAPQP |
| | | | | SHISKYILRWRPKNSVGRWKEATIPGHLNSYTIKGLKPG |
| | | | | VVYEGQLISIQQYGHQEVTRFDFTTTSTSTPVTSNTVTGE |
| | | | | TTPFSPLVATSESVTEITASSFVVSWVSASDTVSGFRVEY |
| | | | | ELSEEGDEPQYLDLPSTATSVNIPDLLPGRKYIVNVYQIS |
| | | | | EDGEQSLILSTSQTTAPDAPPDTTVDQVDDTSIVVRWSR |
| | | | | PQAPITGYRIVYSPSVEGSSTELNLPETANSVTLSDLQPG |
| | | | | VQYNITIYAVEENQESTPVVIQQETTGTPRSDTVPSPRDL |
| | | | | QFVEVTDVKVTIMWTPPESAVTGYRVDVIPVNLPGEHG |
| | | | | QRLPISRNTFAEVTGLSPGVTYYFKVFAVSHGRESKPLT |
| | | | | AQQTTKLDAPTNLQFVNETDSTVLVRWTPPRAQITGYR |
| | | | | LTVGLTRRGQPRQYNVGPSVSKYPLRNLQPASEYTVSL |
| | | | | VAIKGNQESPKATGVFTTLQPGSSIPPYNTEVTETTIVIT |
| | | | | WTPAPRIGFKLGVRPSQGGEAPREVTSDSGSIVVSGLTP |
| | | | | GVEYVYTIQVLRDGQERDAPIVNKVVTPLSPPTNLHLEA |
| | | | | NPDTGVLTVSWERSTTPDITGYRITTTPTNGQQGNSLEE |
| | | | | VVHADQSSCTFDNLSPGLEYNVSVYTVKDDKESVPISD |
| | | | | TIIPEVPQLTDLSFVDITDSSIGLRWTPLNSSTIIGYRITVV |
| | | | | AAGEGIPIFEDFVDSSVGYYTVTGLEPGIDYDISVITLING |
| | | | | GESAPTTLTQQTAVPPPTDLRFTNIGPDTMRVTWAPPPSI |
| | | | | DLTNFLVRYSPVKNEEDVAELSISPSDNAVVLTNLLPGT |
| | | | | EYVVSVSSVYEQHESTPLRGRQKTGLDSPTGIDFSDITA |
| | | | | NSFTVHWIAPRATITGYRIRHHPEHFSGRPREDRVPHSR |
| | | | | NSITLTNLTPGTEYVVSIVALNGREESPLLIGQQSTVSDV |
| | | | | PRDLEVVAATPTSLLISWDAPAVTVRYYRITYGETGGNS |
| | | | | PVQEFTVPGSKSTATISGLKPGVDYTITVYAVTGRGDSP |
| | | | | ASSKPISINYRTEIDKPSQMQVTDVQDNSISVKWLPSSSP |
| | | | | VTGYRVTTTPKNGPGPTKTKTAGPDQTEMTIEGLQPTV |
| | | | | EYVVSVYAQNPSGESQPLVQTAVTNIDRPKGLAFTDVD |
| | | | | VDSIKIAWESPQGQVSRYRVTYSSPEDGIHELFPAPDGEE |
| | | | | DTAELQGLRPGSEYTVSVVALHDDMESQPLIGTQSTAIP |
| | | | | APTDLKFTQVTPTSLSAQWTPPNVQLTGYRVRVTPKEK |
| | | | | TGPMKEINLAPDSSSVVVSGLMVATKYEVSVYALKDTL |
| | | | | TSRPAQGVVTTLENVSPPRRARVTDATETTITISWRTKT |
| | | | | ETITGFQVDAVPANGQTPIQRTIKPDVRSYTITGLQPGTD |
| | | | | YKIYLYTLNDNARSSPVVIDASTAIDAPSNLRFLATTPNS |
| | | | | LLVSWQPPRARITGYIIKYEKPGSPPREVVPRPRPGVTEA |
| | | | | TITGLEPGTEYTIYVIALKNNQKSEPLIGRKKTDELPQLV |
| | | | | TLPHPNLHGPEILDVPSTVQKTPFVTHPGYDTGNGIQLP |
| | | | | GTSGQQPSVGQQMIFEEHGFRRTTPPTTATPIRHRPRPYP |
| | | | | PNVGEEIQIGHIPREDVDYHLYPHGPGLNPNASTGQEAL |
| | | | | SQTTISWAPFQDTSEYIISCHPVGTDEEPLQFRVPGTSTS |
| | | | | ATLTGLTRGATYNVIVEALKDQQRHKVREEVVTVGNS |
| | | | | VNEGLNQPTDDSCFDPYTVSHYAVGDEWERMSESGFK |
| | | | | LLCQCLGFGSGHFRCDSSRWCHDNGVNYKIGEKWDRQ |
| | | | | GENGQMMSCTCLGNGKGEFKCDPHEATCYDDGKTYH |
| | | | | VGEQWQKEYLGAICSCTCFGGQRGWRCDNCRRPGGEP |
| | | | | SPEGTTGQSYNQYSQRYHQRTNTNVNCPIECFMPLDVQ |
| | | | | ADREDSRE |
| 24 | SEPP1 | Selenoprotein | P49908 | MWRSLGLALALCLLPSGGTESQDQSSLCKQPPAWSIRD |
| | | P | | QDPMLNSNGSVTVVALLQASUYLCILQASKLEDLRVKL |
| | | | | KKEGYSNISYIVVNHQGISSRLKYTHLKNKVSEHIPVYQ |
| | | | | QEENQTDVWTLLNGSKDDFLIYDRCGRLVYHLGLPFSF |
| | | | | LTFPYVEEAIKIAYCEKKCGNCSLTTLKDEDFCKRVSLA |
| | | | | TVDKTVETPSPHYHHEHHHNHGHQHLGSSELSENQQPG |
| | | | | APNAPTHPAPPGLHHHHKHKGQHRQGHPENRDMPASE |
| | | | | DLQDLQKKLCRKRCINQLLCKLPTDSELAPRSUCCHCR |
| | | | | HLIFEKTGSAITUQCKENLPSLCSUQGLRAEENITESCQU |
| | | | | RLPPAAUQISQQLIPTEASASURUKNQAKKUEUPSN |
| 25 | VTNC | Vitronectin | P04004 | MAPLRPLLILALLAWVALADQESCKGRCTEGFNVDKKC |
| | | | | QCDELCSYYQSCCTDYTAECKPQVTRGDVFTMPEDEYT |
| | | | | VYDDGEEKNNATVHEQVGGPSLTSDLQAQSKGNPEQTP |
| | | | | VLKPEEEAPAPEVGASKPEGIDSRPETLHPGRPQPPAEEE |
| | | | | LCSGKPFDAFTDLKNGSLFAFRGQYCYELDEKAVRPGY |
| | | | | PKLIRDVWGIEGPIDAAFTRINCQGKTYLFKGSQYWRFE |
| | | | | DGVLDPDYPRNISDGFDGIPDNVDAALALPAHSYSGRE |
| | | | | RVYFFKGKQYWEYQFQHQPSQEECEGSSLSAVFEHFAM |
| | | | | MQRDSWEDIFELLFWGRTSAGTRQPQFISRDWHGVPGQ |
| | | | | VDAAMAGRIYISGMAPRPSLAKKQRFRHRNRKGYRSQ |
| | | | | RGHSRGRNQNSRRPSRATWLSLFSSEESNLGANNYDDY |
| | | | | RMDWLVPATCEPIQSVFFFSGDKYYRVNLRTRRVDTVD |
| | | | | PPYPRSIAQYWLGCPAPGHL |
| 26 | THRB | Prothrombin | P00734 | MAHVRGLQLPGCLALAALCSLVHSQHVFLAPQQARSLL |
| | | | | QRVRRANTFLEEVRKGNLERECVEETCSYEEAFEALESS |
| | | | | TATDVFWAKYTACETARTPRDKLAACLEGNCAEGLGT |
| | | | | NYRGHVNITRSGIECQLWRSRYPHKPEINSTTHPGADLQ |
| | | | | ENFCRNPDSSTTGPWCYTTDPTVRRQECSIPVCGQDQVT |
| | | | | VAMTPRSEGSSVNLSPPLEQCVPDRGQQYQGRLAVTTH |
| | | | | GLPCLAWASAQAKALSKHQDENSAVQLVENFCRNPDG |
| | | | | DEEGVWCYVAGKPGDFGYCDLNYCEEAVEEETGDGLD |
| | | | | EDSDRAIEGRTATSEYQTFFNPRTFGSGEADCGLRPLFE |
| | | | | KKSLEDKTERELLESYIDGRIVEGSDAEIGMSPWQVMLF |
| | | | | RKSPQELLCGASLISDRWVLTAAHCLLYPPWDKNFTEN |
| | | | | DLLVRIGKHSRTRYERNIEKISMLEKIYIHPRYNWRENL |
| | | | | DRDIALMKLKKPVAFSDYIHPVCLPDRETAASLLQAGY |
| | | | | KGRVTGWGNLKETWTANVGKGQPSVLQVVNLPIVERP |
| | | | | VCKDSTRIRITDNMFCAGYKPDEGKRGDACEGDSGGPF |
| | | | | VMKSPFNNRWYQMGIVSWGEGCDRDGKYGFYTHVFR |
| | | | | LKKWIQKVIDQFGE |
| 112 | A2GL | Leucine- | P02750 | MSSWSRQRPKSPGGIQPHVSRTLFLLLLLAASAWGVTLS |
| | | richAlpha-2- | | PKDCQVFRSDHGSSISCQPPAEIPGYLPADTVHLAVEFFN |
| | | glycoprotein | | LTHLPANLLQGASKLQELHLSSNGLESLSPEFLRPVPQL |
| | | | | RVLDLTRNALTGLPPGLFQASATLDTLVLKENQLEVLE |
| | | | | VSWLHGLKALGHLDLSGNRLRKLPPGLLANFTLLRTLD |
| | | | | LGENQLETLPPDLLRGPLQLERLHLEGNKLQVLGKDLLL |
| | | | | PQPDLRYLFLNGNKLARVAAGAFQGLRQLDMLDLSNN |
| | | | | SLASVPEGLWASLGQPNWDMRDGFDISGNPWICDQNLS |
| | | | | DLYRWLQAQKDKMFSQNDTRCAGPEAVKGQTLLAVA |
| | | | | KSQ |
| 113 | AACT | Alpha-1- | P01011 | MERMLPLLALGLLAAGFCPAVLCHPNSPLDEENLTQEN |
| | | antichymotrypsin | | QDRGTHVDLGLASANVDFAFSLYKQLVLKAPDKNVIFS |
| | | | | PLSISTALAFLSLGAHNTTLTEILKGLKFNLTETSEAEIHQ |
| | | | | SFQHLLRTLNQSSDELQLSMGNAMFVKEQLSLLDRFTE |
| | | | | DAKRLYGSEAFATDFQDSAAAKKLINDYVKNGTRGKIT |
| | | | | DLIKDLDSQTMMVLVNYIFFKAKWEMPFDPQDTHQSRF |
| | | | | YLSKKKWVMVPMMSLHHLTIPYFRDEELSCTVVELKYT |
| | | | | GNASALFILPDQDKMEEVEAMLLPETLKRWRDSLEFREI |
| | | | | GELYLPKFSISRDYNLNDILLQLGIEEAFTSKADLSGITG |
| | | | | ARNLAVSQVVHKAVLDVFEEGTEASAATAVKITLLSAL |
| | | | | VETRTIVRFNRPFLMIIVPTDTQNIFFMSKVTNPKQA |
| 114 | APOC3 | Apolipoprotein | P02656 | MQPRVLLVVALLALLASARASEAEDASLLSFMQGYMK |
| | | C-III | | HATKTAKDALSSVQESQVAQQARGWVTDGFSSLKDYW |
| | | | | STVKDKFSEFWDLDPEVRPTSAVAA |
| 115 | APOE | Apolipoprotein | P02649 | MKVLWAALLVTFLAGCQAKVEQAVETEPEPELRQQTE |
| | | E | | WQSGQRWELALGRFWDYLRWVQTLSEQVQEELLSSQV |
| | | | | TQELRALMDETMKELKAYKSELEEQLTPVAEETRARLS |
| | | | | KELQAAQARLGADMEDVCGRLVQYRGEVQAMLGQST |
| | | | | EELRVRLASHLRKLRKRLLRDADDLQKRLAVYQAGAR |
| | | | | EGAERGLSAIRERLGPLVEQGRVRAATVGSLAGQPLQE |
| | | | | RAQAWGERLRARMEEMGSRTRDRLDEVKEQVAEVRA |
| | | | | KLEEQAQQIRLQAEAFQARLKSWFEPLVEDMQRQWAG |
| | | | | LVEKVQAAVGTSAAPVPSDNH |
| 116 | APOH | Beta-2- | P02749 | MISPVLILFSSFLCHVAIAGRTCPKPDDLPFSTVVPLKTFY |
| | | glycoprotein1 | | EPGEEITYSCKPGYVSRGGMRKFICPLTGLWPINTLKCTP |
| | | | | RVCPFAGILENGAVRYTTFEYPNTISFSCNTGFYLNGAD |
| | | | | SAKCTEEGKWSPELPVCAPIICPPPSIPTFATLRVYKPSAG |
| | | | | NNSLYRDTAVFECLPQHAMFGNDTITCTTHGNWTKLPE |
| | | | | CREVKCPFPSRPDNGFVNYPAKPTLYYKDKATFGCHDG |
| | | | | YSLDGPEEIECTKLGNWSAMPSCKASCKVPVKKATVVY |
| | | | | QGERVKIQEKFKNGMLHGDKVSFFCKNKEKKCSYTED |
| | | | | AQCIDGTIEVPKCFKEHSSLAFWKTDASDVKPC |
| 117 | APOM | Apolipoprotein | 095445 | MFHQIWAALLYFYGIILNSIYQCPEHSQLTTLGVDGKEF |
| | | M | | PEVHLGQWYFIAGAAPTKEELATFDPVDNIVENMAAGS |
| | | | | APMQLHLRATIRMKDGLCVPRKWIYHLTEGSTDLRTEG |
| | | | | RPDMKTELFSSSCPGGIMLNETGQGYQRFLLYNRSPHPP |
| | | | | EKCVEEFKSLTSCLDSKAFLLTPRNQEACELSNN |
| 118 | C4BPA | C4b-binding | P04003 | MHPPKTPSGALHRKRKMAAWPFSRLWKVSDPILFQMT |
| | | protein alpha | | LIAALLPAVLGNCGPPPTLSFAAPMDITLTETRFKTGTTL |
| | | chain | | KYTCLPGYVRSHSTQTLTCNSDGEWVYNTFCIYKRCRH |
| | | | | PGELRNGQVEIKTDLSFGSQIEFSCSEGFFLIGSTTSRCEV |
| | | | | QDRGVGWSHPLPQCEIVKCKPPPDIRNGRHSGEENFYA |
| | | | | YGFSVTYSCDPRFSLLGHASISCTVENETIGVWRPSPPTC |
| | | | | EKITCRKPDVSHGEMVSGFGPIYNYKDTIVFKCQKGFVL |
| | | | | RGSSVIHCDADSKWNPSPPACEPNSCINLPDIPHASWET |
| | | | | YPRPTKEDVYVVGTVLRYRCHPGYKPTTDEPTTVICQK |
| | | | | NLRWTPYQGCEALCCPEPKLNNGEITQHRKSRPANHCV |
| | | | | YFYGDEISFSCHETSRFSAICQGDGTWSPRTPSCGDICNF |
| | | | | PPKIAHGHYKQSSSYSFFKEEIIYECDKGYILVGQAKLSC |
| | | | | SYSHWSAPAPQCKALCRKPELVNGRLSVDKDQYVEPE |
| | | | | NVTIQCDSGYGVVGPQSITCSGNRTWYPEVPKCEWETP |
| | | | | EGCEQVLTGKRLMQCLPNPEDVKMALEVYKLSLEIEQL |
| | | | | ELQRDSARQSTLDKEL |
| 119 | CERU | Ceruloplasmin | P00450 | MKILILGIFLFLCSTPAWAKEKHYYIGIIETTWDYASDHG |
| | | | | EKKLISVDTEHSNIYLQNGPDRIGRLYKKALYLQYTDET |
| | | | | FRTTIEKPVWLGFLGPIIKAETGDKVYVHLKNLASRPYT |
| | | | | FHSHGITYYKEHEGAIYPDNTTDFQRADDKVYPGEQYT |
| | | | | YMLLATEEQSPGEGDGNCVTRIYHSHIDAPKDIASGLIG |
| | | | | PLIICKKDSLDKEKEKHIDREFVVMFSVVDENFSWYLED |
| | | | | NIKTYCSEPEKVDKDNEDFQESNRMYSVNGYTFGSLPG |
| | | | | LSMCAEDRVKWYLFGMGNEVDVHAAFFHGQALTNKN |
| | | | | YRIDTINLFPATLFDAYMVAQNPGEWMLSCQNLNHLKA |
| | | | | GLQAFFQVQECNKSSSKDNIRGKHVRHYYIAAEEIIWNY |
| | | | | APSGIDIFTKENLTAPGSDSAVFFEQGTTRIGGSYKKLVY |
| | | | | REYTDASFTNRKERGPEEEHLGILGPVIWAEVGDTIRVT |
| | | | | FHNKGAYPLSIEPIGVRFNKNNEGTYYSPNYNPQSRSVP |
| | | | | PSASHVAPTETFTYEWTVPKEVGPTNADPVCLAKMYYS |
| | | | | AVDPTKDIFTGLIGPMKICKKGSLHANGRQKDVDKEFY |
| | | | | LFPTVFDENESLLLEDNIRMFTTAPDQVDKEDEDFQESN |
| | | | | KMHSMNGFMYGNQPGLTMCKGDSVVWYLFSAGNEAD |
| | | | | VHGIYFSGNTYLWRGERRDTANLFPQTSLTLHMWPDTE |
| | | | | GTFNVECLTTDHYTGGMKQKYTVNQCRRQSEDSTFYL |
| | | | | GERTYYIAAVEVEWDYSPQREWEKELHHLQEQNVSNA |
| | | | | FLDKGEFYIGSKYKKVVYRQYTDSTFRVPVERKAEEEH |
| | | | | LGILGPQLHADVGDKVKIIFKNMATRPYSIHAHGVQTES |
| | | | | STVTPTLPGETLTYVWKIPERSGAGTEDSACIPWAYYST |
| | | | | VDQVKDLYSGLIGPLIVCRRPYLKVFNPRRKLEFALLFL |
| | | | | VFDENESWYLDDNIKTYSDHPEKVNKDDEEFIESNKMH |
| | | | | AINGRMFGNLQGLTMHVGDEVNWYLMGMGNEIDLHT |
| | | | | VHFHGHSFQYKHRGVYSSDVFDIFPGTYQTLEMFPRTP |
| | | | | GIWLLHCHVTDHIHAGMETTYTVLQNEDTKSG |
| 120 | CFAH | ComplementFactorH | P08603 | MRLLAKIICLMLWAICVAEDCNELPPRRNTEILTGSWSD |
| | | | | QTYPEGTQAIYKCRPGYRSLGNVIMVCRKGEWVALNPL |
| | | | | RKCQKRPCGHPGDTPFGTFTLTGGNVFEYGVKAVYTCN |
| | | | | EGYQLLGEINYRECDTDGWTNDIPICEVVKCLPVTAPEN |
| | | | | GKIVSSAMEPDREYHFGQAVRFVCNSGYKIEGDEEMHC |
| | | | | SDDGFWSKEKPKCVEISCKSPDVINGSPISQKIIYKENER |
| | | | | FQYKCNMGYEYSERGDAVCTESGWRPLPSCEEKSCDNP |
| | | | | YIPNGDYSPLRIKHRTGDEITYQCRNGFYPATRGNTAKC |
| | | | | TSTGWIPAPRCTLKPCDYPDIKHGGLYHENMRRPYFPV |
| | | | | AVGKYYSYYCDEHFETPSGSYWDHIHCTQDGWSPAVP |
| | | | | CLRKCYFPYLENGYNQNYGRKFVQGKSIDVACHPGYA |
| | | | | LPKAQTTVTCMENGWSPTPRCIRVKTCSKSSIDIENGFIS |
| | | | | ESQYTYALKEKAKYQCKLGYVTADGETSGSITCGKDG |
| | | | | WSAQPTCIKSCDIPVFMNARTKNDFTWFKLNDTLDYEC |
| | | | | HDGYESNTGSTTGSIVCGYNGWSDLPICYERECELPKID |
| | | | | VHLVPDRKKDQYKVGEVLKFSCKPGFTIVGPNSVQCYH |
| | | | | FGLSPDLPICKEQVQSCGPPPELLNGNVKEKTKEEYGHS |
| | | | | EVVEYYCNPRFLMKGPNKIQCVDGEWTTLPVCIVEEST |
| | | | | CGDIPELEHGWAQLSSPPYYYGDSVEFNCSESFTMIGHR |
| | | | | SITCIHGVWTQLPQCVAIDKLKKCKSSNLIILEEHLKNKK |
| | | | | EFDHNSNIRYRCRGKEGWIHTVCINGRWDPEVNCSMAQ |
| | | | | IQLCPPPPQIPNSHNMTTTLNYRDGEKVSVLCQENYLIQ |
| | | | | EGEEITCKDGRWQSIPLCVEKIPCSQPPQIEHGTINSSRSS |
| | | | | QESYAHGTKLSYTCEGGFRISEENETTCYMGKWSSPPQ |
| | | | | CEGLPCKSPPEISHGVVAHMSDSYQYGEEVTYKCFEGF |
| | | | | GIDGPAIAKCLGEKWSHPPSCIKTDCLSLPSFENAIPMGE |
| | | | | KKDVYKAGEQVTYTCATYYKMDGASNVTCINSRWTG |
| | | | | RPTCRDTSCVNPPTVQNAYIVSRQMSKYPSGERVRYQC |
| | | | | RSPYEMFGDEEVMCLNGNWTEPPQCKDSTGKCGPPPPI |
| | | | | DNGDITSFPLSVYAPASSVEYQCQNLYQLEGNKRITCRN |
| | | | | GQWSEPPKCLHPCVISREIMENYNIALRWTAKQKLYSRT |
| | | | | GESVEFVCKRGYRLSSRSHTLRTTCWDGKLEYPTCAKR |
| 121 | CO3 | ComplementC3 | P01024 | MGPTSGPSLLLLLLTHLPLALGSPMYSIITPNILRLESEET |
| | | | | MVLEAHDAQGDVPVTVTVHDFPGKKLVLSSEKTVLTP |
| | | | | ATNHMGNVTFTIPANREFKSEKGRNKFVTVQATFGTQV |
| | | | | VEKVVLVSLQSGYLFIQTDKTIYTPGSTVLYRIFTVNHKL |
| | | | | LPVGRTVMVNIENPEGIPVKQDSLSSQNQLGVLPLSWDI |
| | | | | PELVNMGQWKIRAYYENSPQQVFSTEFEVKEYVLPSFE |
| | | | | VIVEPTEKFYYIYNEKGLEVTITARFLYGKKVEGTAFVIF |
| | | | | GIQDGEQRISLPESLKRIPIEDGSGEVVLSRKVLLDGVQN |
| | | | | PRAEDLVGKSLYVSATVILHSGSDMVQAERSGIPIVTSP |
| | | | | YQIHFTKTPKYFKPGMPFDLMVFVTNPDGSPAYRVPVA |
| | | | | VQGEDTVQSLTQGDGVAKLSINTHPSQKPLSITVRTKKQ |
| | | | | ELSEAEQATRTMQALPYSTVGNSNNYLHLSVLRTELRP |
| | | | | GETLNVNFLLRMDRAHEAKIRYYTYLIMNKGRLLKAGR |
| | | | | QVREPGQDLVVLPLSITTDFIPSFRLVAYYTLIGASGQRE |
| | | | | VVADSVWVDVKDSCVGSLVVKSGQSEDRQPVPGQQM |
| | | | | TLKIEGDHGARVVLVAVDKGVFVLNKKNKLTQSKIWD |
| | | | | VVEKADIGCTPGSGKDYAGVFSDAGLTFTSSSGQQTAQ |
| | | | | RAELQCPQPAARRRRSVQLTEKRMDKVGKYPKELRKC |
| | | | | CEDGMRENPMRFSCQRRTRFISLGEACKKVELDCCNYIT |
| | | | | ELRRQHARASHLGLARSNLDEDIIAEENIVSRSEFPESWL |
| | | | | WNVEDLKEPPKNGISTKLMNIFLKDSITTWEILAVSMSD |
| | | | | KKGICVADPFEVTVMQDFFIDLRLPYSVVRNEQVEIRAV |
| | | | | LYNYRQNQELKVRVELLHNPAFCSLATTKRRHQQTVTI |
| | | | | PPKSSLSVPYVIVPLKTGLQEVEVKAAVYHHFISDGVRK |
| | | | | SLKVVPEGIRMNKTVAVRTLDPERLGREGVQKEDIPPA |
| | | | | DLSDQVPDTESETRILLQGTPVAQMTEDAVDAERLKHLI |
| | | | | VTPSGCGEQNMIGMTPTVIAVHYLDETEQWEKFGLEKR |
| | | | | QGALELIKKGYTQQLAFRQPSSAFAAFVKRAPSTWLTA |
| | | | | YVVKVFSLAVNLIAIDSQVLCGAVKWLILEKQKPDGVF |
| | | | | QEDAPVIHQEMIGGLRNNNEKDMALTAFVLISLQEAKDI |
| | | | | CEEQVNSLPGSITKAGDFLEANYMNLQRSYTVAIAGYA |
| | | | | LAQMGRLKGPLLNKFLTTAKDKNRWEDPGKQLYNVEA |
| | | | | TSYALLALLQLKDFDFVPPVVRWLNEQRYYGGGYGST |
| | | | | QATFMVFQALAQYQKDAPDHQELNLDVSLQLPSRSSKI |
| | | | | THRIHWESASLLRSEETKENEGFTVTAEGKGQGTLSVVT |
| | | | | MYHAKAKDQLTCNKFDLKVTIKPAPETEKRPQDAKNT |
| | | | | MILEICTRYRGDQDATMSILDISMMTGFAPDTDDLKQLA |
| | | | | NGVDRYISKYELDKAFSDRNTLIIYLDKVSHSEDDCLAF |
| | | | | KVHQYFNVELIQPGAVKVYAYYNLEESCTRFYHPEKED |
| | | | | GKLNKLCRDELCRCAEENCFIQKSDDKVTLEERLDKAC |
| | | | | EPGVDYVYKTRLVKVQLSNDFDEYIMAIEQTIKSGSDEV |
| | | | | QVGQQRTFISPIKCREALKLEEKKHYLMWGLSSDFWGE |
| | | | | KPNLSYIIGKDTWVEHWPEEDECQDEENQKQCQDLGAF |
| | | | | TESMVVFGCPN |
| 122 | CO6 | Complement- | P13671 | MARRSVLYFILLNALINKGQACFCDHYAWTQWTSCSKT |
| | | componentC6 | | CNSGTQSRHRQIVVDKYYQENFCEQICSKQETRECNWQ |
| | | | | RCPINCLLGDFGPWSDCDPCIEKQSKVRSVLRPSQFGGQ |
| | | | | PCTAPLVAFQPCIPSKLCKIEEADCKNKFRCDSGRCIARK |
| | | | | LECNGENDCGDNSDERDCGRTKAVCTRKYNPIPSVQLM |
| | | | | GNGFHFLAGEPRGEVLDNSFTGGICKTVKSSRTSNPYRV |
| | | | | PANLENVGFEVQTAEDDLKTDFYKDLTSLGHNENQQGS |
| | | | | FSSQGGSSFSVPIFYSSKRSENINHNSAFKQAIQASHKKD |
| | | | | SSFIRIHKVMKVLNFTTKAKDLHLSDVFLKALNHLPLEY |
| | | | | NSALYSRIFDDFGTHYFTSGSLGGVYDLLYQFSSEELKN |
| | | | | SGLTEEEAKHCVRIETKKRVLFAKKTKVEHRCTTNKLSE |
| | | | | KHEGSFIQGAEKSISLIRGGRSEYGAALAWEKGSSGLEE |
| | | | | KTFSEWLESVKENPAVIDFELAPIVDLVRNIPCAVTKRN |
| | | | | NLRKALQEYAAKFDPCQCAPCPNNGRPTLSGTECLCVC |
| | | | | QSGTYGENCEKQSPDYKSNAVDGQWGCWSSWSTCDA |
| | | | | TYKRSRTRECNNPAPQRGGKRCEGEKRQEEDCTFSIME |
| | | | | NNGQPCINDDEEMKEVDLPEIEADSGCPQPVPPENGFIR |
| | | | | NEKQLYLVGEDVEISCLTGFETVGYQYFRCLPDGTWRQ |
| | | | | GDVECQRTECIKPVVQEVLTITPFQRLYRIGESIELTCPK |
| | | | | GFVVAGPSRYTCQGNSWTPPISNSLTCEKDTLTKLKGH |
| | | | | CQLGQKQSGSECICMSPEEDCSHHSEDLCVFDTDSNDYF |
| | | | | TSPACKFLAEKCLNNQQLHFLHIGSCQDGRQLEWGLER |
| | | | | TRLSSNSTKKESCGYDTCYDWEKCSASTSKCVCLLPPQ |
| | | | | CFKGGNQLYCVKMGSSTSEKTLNICEVGTIRCANRKME |
| | | | | ILHPGKCLA |
| 123 | IGA1 | Immunoglobulin | P01876 | ASPTSPKVFPLSLCSTQPDGNVVIACLVQGFFPQEPLSVT |
| | | heavy | | WSESGQGVTARNFPPSQDASGDLYTTSSQLTLPATQCL |
| | | constant alpha | | AGKSVTCHVKHYTNPSQDVTVPCPVPSTPPTPSPSTPPTP |
| | | 1 | | SPSCCHPRLSLHRPALEDLLLGSEANLTCTLTGLRDASG |
| | | | | VTFTWTPSSGKSAVQGPPERDLCGCYSVSSVLPGCAEP |
| | | | | WNHGKTFTCTAAYPESKTPLTATLSKSGNTFRPEVHLLP |
| | | | | PPSEELALNELVTLTCLARGFSPKDVLVRWLQGSQELPR |
| | | | | EKYLTWASRQEPSQGTTTFAVTSILRVAAEDWKKGDTF |
| | | | | SCMVGHEALPLAFTQKTIDRLAGKPTHVNVSVVMAEV |
| | | | | DGTCY |
| 124 | IGA2 | Immunoglobulin | P01877 | ASPTSPKVFPLSLDSTPQDGNVVVACLVQGFFPQEPLSV |
| | | heavy | | TWSESGQNVTARNFPPSQDASGDLYTTSSQLTLPATQCP |
| | | constant alpha | | DGKSVTCHVKHYTNSSQDVTVPCRVPPPPPCCHPRLSL |
| | | 2 | | HRPALEDLLLGSEANLTCTLTGLRDASGATFTWTPSSGK |
| | | | | SAVQGPPERDLCGCYSVSSVLPGCAQPWNHGETFTCTA |
| | | | | AHPELKTPLTANITKSGNTFRPEVHLLPPPSEELALNELV |
| | | | | TLTCLARGFSPKDVLVRWLQGSQELPREKYLTWASRQE |
| | | | | PSQGTTTYAVTSILRVAAEDWKKGETFSCMVGHEALPL |
| | | | | AFTQKTIDRMAGKPTHINVSVVMAEADGTCY |
| 125 | IGG1 | Immunoglobulin | P01857 | ASTKGPSVFPLAPSSKSTSGGTAALGCLVKDYFPEPVTV |
| | | heavy | | SWNSGALTSGVHTFPAVLQSSGLYSLSSVVTVPSSSLGT |
| | | constant | | QTYICNVNHKPSNTKVDKKVEPKSCDKTHTCPPCPAPE |
| | | gamma 1 | | LLGGPSVFLFPPKPKDTLMISRTPEVTCVVVDVSHEDPE |
| | | | | VKFNWYVDGVEVHNAKTKPREEQYNSTYRVVSVLTVL |
| | | | | HQDWLNGKEYKCKVSNKALPAPIEKTISKAKGQPREPQ |
| | | | | VYTLPPSRDELTKNQVSLTCLVKGFYPSDIAVEWESNG |
| | | | | QPENNYKTTPPVLDSDGSFFLYSKLTVDKSRWQQGNVF |
| | | | | SCSVMHEALHNHYTQKSLSLSPGK |
| 126 | IGG2 | Immunoglobulin | P01859 | ASTKGPSVFPLAPCSRSTSESTAALGCLVKDYFPEPVTVS |
| | | heavy | | WNSGALTSGVHTFPAVLQSSGLYSLSSVVTVPSSNFGTQ |
| | | constant | | TYTCNVDHKPSNTKVDKTVERKCCVECPPCPAPPVAGP |
| | | gamma 2 | | SVFLFPPKPKDTLMISRTPEVTCVVVDVSHEDPEVQFNW |
| | | | | YVDGVEVHNAKTKPREEQFNSTFRVVSVLTVVHQDWL |
| | | | | NGKEYKCKVSNKGLPAPIEKTISKTKGQPREPQVYTLPP |
| | | | | SREEMTKNQVSLTCLVKGFYPSDISVEWESNGQPENNY |
| | | | | KTTPPMLDSDGSFFLYSKLTVDKSRWQQGNVFSCSVMH |
| | | | | EALHNHYTQKSLSLSPGK |
| 127 | IGG34 | Immunoglobulin | P01860 | ASTKGPSVFPLAPCSRSTSGGTAALGCLVKDYFPEPVTV |
| | | heavy | | SWNSGALTSGVHTFPAVLQSSGLYSLSSVVTVPSSSLGT |
| | | constant | | QTYTCNVNHKPSNTKVDKRVELKTPLGDTTHTCPRCPE |
| | | gamma 3 | | PKSCDTPPPCPRCPEPKSCDTPPPCPRCPEPKSCDTPPPCP |
| | | | | RCPAPELLGGPSVFLFPPKPKDTLMISRTPEVTCVVVDV |
| | | | | SHEDPEVQFKWYVDGVEVHNAKTKPREEQYNSTFRVV |
| | | | | SVLTVLHQDWLNGKEYKCKVSNKALPAPIEKTISKTKG |
| | | | | QPREPQVYTLPPSREEMTKNQVSLTCLVKGFYPSDIAVE |
| | | | | WESSGQPENNYNTTPPMLDSDGSFFLYSKLTVDKSRWQ |
| | | | | QGNIFSCSVMHEALHNRFTQKSLSLSPGK |
| 128 | IGJ | Immunoglobulin | P01591 | MKNHLLFWGVLAVFIKAVHVKAQEDERIVLVDNKCKC |
| | | J chain | | ARITSRIIRSSEDPNEDIVERNIRIIVPLNNRENISDPTSPLR |
| | | | | TRFVYHLSDLCKKCDPTEVELDNQIVTATQSNICDEDSA |
| | | | | TETCYTYDRNKCYTAVVPLVYGGETKMVETALTPDAC |
| | | | | YPD |
| 129 | IGM | Immunoglobulin | P01871 | GSASAPTLFPLVSCENSPSDTSSVAVGCLAQDFLPDSITF |
| | | heavy | | SWKYKNNSDISSTRGFPSVLRGGKYAATSQVLLPSKDV |
| | | constant mu | | MQGTDEHVVCKVQHPNGNKEKNVPLPVIAELPPKVSVF |
| | | | | VPPRDGFFGNPRKSKLICQATGFSPRQIQVSWLREGKQV |
| | | | | GSGVTTDQVQAEAKESGPTTYKVTSTLTIKESDWLGQS |
| | | | | MFTCRVDHRGLTFQQNASSMCVPDQDTAIRVFAIPPSFA |
| | | | | SIFLTKSTKLTCLVTDLTTYDSVTISWTRQNGEAVKTHT |
| | | | | NISESHPNATFSAVGEASICEDDWNSGERFTCTVTHTDL |
| | | | | PSPLKQTISRPKGVALHRPDVYLLPPAREQLNLRESATIT |
| | | | | CLVTGFSPADVFVQWMQRGQPLSPEKYVTSAPMPEPQA |
| | | | | PGRYFAHSILTVSEEEWNTGETYTCVVAHEALPNRVTE |
| | | | | RTVDKSTGKPTLYNVSLVMSDTAGTCY |
| 130 | KLKB1 | Plasma | P03952 | MILFKQATYFISLFATVSCGCLTQLYENAFFRGGDVASM |
| | | Kallikrein | | YTPNAQYCQMRCTFHPRCLLFSFLPASSINDMEKRFGCF |
| | | | | LKDSVTGTLPKVHRTGAVSGHSLKQCGHQISACHRDIY |
| | | | | KGVDMRGVNFNVSKVSSVEECQKRCTNNIRCQFFSYAT |
| | | | | QTFHKAEYRNNCLLKYSPGGTPTAIKVLSNVESGFSLKP |
| | | | | CALSEIGCHMNIFQHLAFSDVDVARVLTPDAFVCRTICT |
| | | | | YHPNCLFFTFYTNVWKIESQRNVCLLKTSESGTPSSSTP |
| | | | | QENTISGYSLLTCKRTLPEPCHSKIYPGVDFGGEELNVTF |
| | | | | VKGVNVCQETCTKMIRCQFFTYSLLPEDCKEEKCKCFL |
| | | | | RLSMDGSPTRIAYGTQGSSGYSLRLCNTGDNSVCTTKTS |
| | | | | TRIVGGTNSSWGEWPWQVSLQVKLTAQRHLCGGSLIGH |
| | | | | QWVLTAAHCFDGLPLQDVWRIYSGILNLSDITKDTPFSQ |
| | | | | IKEIIIHQNYKVSEGNHDIALIKLQAPLNYTEFQKPICLPS |
| | | | | KGDTSTIYTNCWVTGWGFSKEKGEIQNILQKVNIPLVTN |
| | | | | EECQKRYQDYKITQRMVCAGYKEGGKDACKGDSGGPL |
| | | | | VCKHNGMWRLVGITSWGEGCARREQPGVYTKVAEYM |
| | | | | DWILEKTQSSDGKAQMQSPA |
| 131 | SHBG | Sex hormone- | P04278 | MESRGPLATSRLLLLLLLLLLRHTRQGWALRPVLPTQSA |
| | | binding | | HDPPAVHLSNGPGQEPIAVMTFDLTKITKTSSSFEVRTW |
| | | globulin | | DPEGVIFYGDTNPKDDWFMLGLRDGRPEIQLHNHWAQ |
| | | | | LTVGAGPRLDDGRWHQVEVKMEGDSVLLEVDGEEVLR |
| | | | | LRQVSGPLTSKRHPIMRIALGGLLFPASNLRLPLVPALD |
| | | | | GCLRRDSWLDKQAEISASAPTSLRSCDVESNPGIFLPPGT |
| | | | | QAEFNLRDIPQPHAEPWAFSLDLGLKQAAGSGHLLALG |
| | | | | TPENPSWLSLHLQDQKVVLSSGSGPGLDLPLVLGLPLQL |
| | | | | KLSMSRVVLSQGSKMKALALPPLGLAPLLNLWAKPQG |
| | | | | RLFLGALPGEDSSTSFCLNGLWAQGQRLDVDQALNRSH |
| | | | | EIWTHSCPQSPGNGTDASH |
| 132 | TRFE | Serotransferrin | P02787 | MRLAVGALLVCAVLGLCLAVPDKTVRWCAVSEHEATK |
| | | | | CQSFRDHMKSVIPSDGPSVACVKKASYLDCIRAIAANEA |
| | | | | DAVTLDAGLVYDAYLAPNNLKPVVAEFYGSKEDPQTF |
| | | | | YYAVAVVKKDSGFQMNQLRGKKSCHTGLGRSAGWNIP |
| | | | | IGLLYCDLPEPRKPLEKAVANFFSGSCAPCADGTDFPQL |
| | | | | CQLCPGCGCSTLNQYFGYSGAFKCLKDGAGDVAFVKH |
| | | | | STIFENLANKADRDQYELLCLDNTRKPVDEYKDCHLAQ |
| | | | | VPSHTVVARSMGGKEDLIWELLNQAQEHFGKDKSKEF |
| | | | | QLFSSPHGKDLLFKDSAHGFLKVPPRMDAKMYLGYEY |
| | | | | VTAIRNLREGTCPEAPTDECKPVKWCALSHHERLKCDE |
| | | | | WSVNSVGKIECVSAETTEDCIAKIMNGEADAMSLDGGF |
| | | | | VYIAGKCGLVPVLAENYNKSDNCEDTPEAGYFAVAVV |
| | | | | KKSASDLTWDNLKGKKSCHTAVGRTAGWNIPMGLLYN |
| | | | | KINHCRFDEFFSEGCAPGSKKDSSLCKLCMGSGLNLCEP |
| | | | | NNKEGYYGYTGAFRCLVEKGDVAFVKHQTVPQNTGGK |
| | | | | NPDPWAKNLNEKDYELLCLDGTRKPVEEYANCHLARA |
| | | | | PNHAVVTRKDKEACVHKILRQQQHLFGSNVTDCSGNFC |
| | | | | LFRSETKDLLFRDDTVCLAKLHDRNTYEKYLGEEYVKA |
| | | | | VGNLRKCSTSSLLEACTFRRP |
| 133 | CO2 | ComplementC2 | P06681 | MGPLMVLFCLLFLYPGLADSAPSCPQNVNISGGTFTLSH |
| | | | | GWAPGSLLTYSCPQGLYPSPASRLCKSSGQWQTPGATR |
| | | | | SLSKAVCKPVRCPAPVSFENGIYTPRLGSYPVGGNVSFE |
| | | | | CEDGFILRGSPVRQCRPNGMWDGETAVCDNGAGHCPN |
| | | | | PGISLGAVRTGFRFGHGDKVRYRCSSNLVLTGSSERECQ |
| | | | | GNGVWSGTEPICRQPYSYDFPEDVAPALGTSFSHMLGA |
| | | | | TNPTQKTKESLGRKIQIQRSGHLNLYLLLDCSQSVSEND |
| | | | | FLIFKESASLMVDRIFSFEINVSVAIITFASEPKVLMSVLN |
| | | | | DNSRDMTEVISSLENANYKDHENGTGTNTYAALNSVYL |
| | | | | MMNNQMRLLGMETMAWQEIRHAIILLTDGKSNMGGSP |
| | | | | KTAVDHIREILNINQKRNDYLDIYAIGVGKLDVDWREL |
| | | | | NELGSKKDGERHAFILQDTKALHQVFEHMLDVSKLTDT |
| | | | | ICGVGNMSANASDQERTPWHVTIKPKSQETCRGALISD |
| | | | | QWVLTAAHCFRDGNDHSLWRVNVGDPKSQWGKEFLIE |
| | | | | KAVISPGFDVFAKKNQGILEFYGDDIALLKLAQKVKMS |
| | | | | THARPICLPCTMEANLALRRPQGSTCRDHENELLNKQS |
| | | | | VPAHFVALNGSKLNINLKMGVEWTSCAEVVSQEKTMF |
| | | | | PNLTDVREVVTDQFLCSGTQEDESPCKGESGGAVFLER |
| | | | | RFRFFQVGLVSWGLYNPCLGSADKNSRKRAPRSKVPPP |
| | | | | RDFHINLFRMQPWLRQHLGDVLNFLPL |
| 134 | CO5 | ComplementC5 | P01031 | MGLLGILCFLIFLGKTWGQEQTYVISAPKIFRVGASENIV |
| | | | | IQVYGYTEAFDATISIKSYPDKKFSYSSGHVHLSSENKFQ |
| | | | | NSAILTIQPKQLPGGQNPVSYVYLEVVSKHFSKSKRMPI |
| | | | | TYDNGFLFIHTDKPVYTPDQSVKVRVYSLNDDLKPAKR |
| | | | | ETVLTFIDPEGSEVDMVEEIDHIGIISFPDFKIPSNPRYGM |
| | | | | WTIKAKYKEDFSTTGTAYFEVKEYVLPHFSVSIEPEYNFI |
| | | | | GYKNFKNFEITIKARYFYNKVVTEADVYITFGIREDLKD |
| | | | | DQKEMMQTAMQNTMLINGIAQVTFDSETAVKELSYYS |
| | | | | LEDLNNKYLYIAVTVIESTGGFSEEAEIPGIKYVLSPYKL |
| | | | | NLVATPLFLKPGIPYPIKVQVKDSLDQLVGGVPVTLNAQ |
| | | | | TIDVNQETSDLDPSKSVTRVDDGVASFVLNLPSGVTVLE |
| | | | | FNVKTDAPDLPEENQAREGYRAIAYSSLSQSYLYIDWTD |
| | | | | NHKALLVGEHLNIIVTPKSPYIDKITHYNYLILSKGKIIHF |
| | | | | GTREKFSDASYQSINIPVTQNMVPSSRLLVYYIVTGEQT |
| | | | | AELVSDSVWLNIEEKCGNQLQVHLSPDADAYSPGQTVS |
| | | | | LNMATGMDSWVALAAVDSAVYGVQRGAKKPLERVFQ |
| | | | | FLEKSDLGCGAGGGLNNANVFHLAGLTFLTNANADDS |
| | | | | QENDEPCKEILRPRRTLQKKIEEIAAKYKHSVVKKCCYD |
| | | | | GACVNNDETCEQRAARISLGPRCIKAFTECCVVASQLR |
| | | | | ANISHKDMQLGRLHMKTLLPVSKPEIRSYFPESWLWEV |
| | | | | HLVPRRKQLQFALPDSLTTWEIQGVGISNTGICVADTVK |
| | | | | AKVFKDVFLEMNIPYSVVRGEQIQLKGTVYNYRTSGMQ |
| | | | | FCVKMSAVEGICTSESPVIDHQGTKSSKCVRQKVEGSSS |
| | | | | HLVTFTVLPLEIGLHNINFSLETWFGKEILVKTLRVVPEG |
| | | | | VKRESYSGVTLDPRGIYGTISRRKEFPYRIPLDLVPKTEI |
| | | | | KRILSVKGLLVGEILSAVLSQEGINILTHLPKGSAEAELM |
| | | | | SVVPVFYVFHYLETGNHWNIFHSDPLIEKQKLKKKLKE |
| | | | | GMLSIMSYRNADYSYSVWKGGSASTWLTAFALRVLGQ |
| | | | | VNKYVEQNQNSICNSLLWLVENYQLDNGSFKENSQYQP |
| | | | | IKLQGTLPVEARENSLYLTAFTVIGIRKAFDICPLVKIDTA |
| | | | | LIKADNFLLENTLPAQSTFTLAISAYALSLGDKTHPQFRS |
| | | | | IVSALKREALVKGNPPIYRFWKDNLQHKDSSVPNTGTA |
| | | | | RMVETTAYALLTSLNLKDINYVNPVIKWLSEEQRYGGG |
| | | | | FYSTQDTINAIEGLTEYSLLVKQLRLSMDIDVSYKHKGA |
| | | | | LHNYKMTDKNFLGRPVEVLLNDDLIVSTGFGSGLATVH |
| | | | | VTTVVHKTSTSEEVCSFYLKIDTQDIEASHYRGYGNSDY |
| | | | | KRIVACASYKPSREESSSGSSHAVMDISLPTGISANEEDL |
| | | | | KALVEGVDQLFTDYQIKDGHVILQLNSIPSSDFLCVRFRI |
| | | | | FELFEVGFLSPATFTVYEYHRPDKQCTMFYSTSNIKIQK |
| | | | | VCEGAACKCVEADCGQMQEELDLTISAETRKQTACKPE |
| | | | | IAYAYKVSITSITVENVFVKYKATLLDIYKTGEAVAEKD |
| | | | | SEITFIKKVTCTNAELVKGRQYLIMGKEALQIKYNFSFR |
| | | | | YIYPLDSLTWIEYWPRDTTCSSCQAFLANLDEFAEDIFLN |
| | | | | GC |
| 135 | ZA2G | Zinc-alpha-2- | P25311 | MVRMVPVLLSLLLLLGPAVPQENQDGRYSLTYIYTGLS |
| | | glycoprotein | | KHVEDVPAFQALGSLNDLQFFRYNSKDRKSQPMGLWR |
| | | | | QVEGMEDWKQDSQLQKAREDIFMETLKDIVEYYNDSN |
| | | | | GSHVLQGRFGCEIENNRSSGAFWKYYYDGKDYIEFNKE |
| | | | | IPAWVPFDPAAQITKQKWEAEPVYVQRAKAYLEEECPA |
| | | | | TLRKYLKYSKNILDRQDPPSVVVTSHQAPGEKKKLKCL |
| | | | | AYDFYPGKIDVHWTRAGEVQEPELRGDVLHNGNGTYQ |
| | | | | SWVVVAVPPQDTAPYSCHVQHSSLAQPLVVPWEAS |

VIII.A.2 Representative Experimental Results—Subject Sample Model & Corresponding Training of Said Model based on Table 1B

To assess the association of individual peptide structures (biomarkers) with APL or colorectal cancer, differential expression analysis (DEA) was run using the Wilcoxon test on a cohort of 787 samples sourced from different biorepositories, including 427 CRC samples, 180 APL samples, 99 non-APL samples, and 81 healthy control samples, adjusting for age and sex.

In some aspects, a subject was classified with APL if there was one or more of the following clinical conditions such as adenomas ≥10 mm in diameter; sessile serrated lesions ≥10 mm in diameter; or adenomas <10 mm in diameter if it contains at least 25% villous features, high-grade dysplasia, or carcinoma. A subject was classified with non-advanced precancerous lesions (non-APL) if there was one or more of the following clinical conditions such as adenomas <10 mm in diameter (including <25% villous features, no high-grade dysplasia, no carcinoma); serrated adenomas <10 mm in diameter; hyperplastic polyps; or inflammatory polyps (or pseudo-polyps). Under certain circumstances, APL may be referred to as precancerous and non-APL may be referred to as non-precancerous.

The data set was split into three categories, which were train (60%), validation (15%) and a hold-out test (25%) and were set stratified randomly by the sex, age quartiles, institution and disease indication of the samples. Table 7 displays distribution of the number of subjects for each condition in the train/validation/test set.

TABLE 7

| | | Healthy | | |
|---|---|---|---|---|
| | APL | Controls | CRC | Non-APL |
| Train (N = 476) | 110 | 49 | 255 | 62 |
| Validation (N = 113) | 26 | 11 | 62 | 14 |
| Test (N = 198) | 44 | 21 | 110 | 23 |

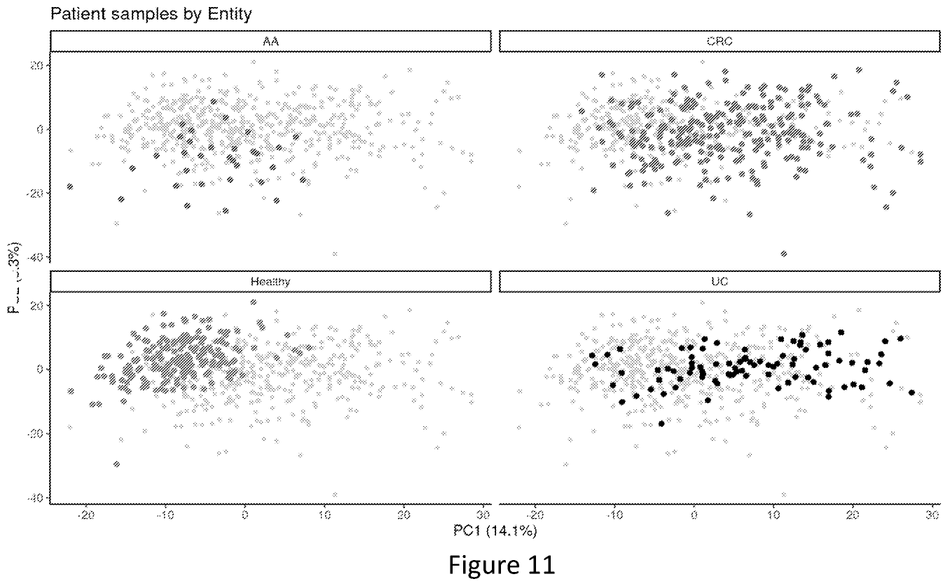

The results of the DEA are summarized below with reference to Tables 7-8 and FIGS. 11-14. Table 8 shows the p values (<0.05) and the false discovery rates for the biomarkers PS-TD No. 7-21. The DEA output based on the training data of Table 7 is shown in Table 8 that compares the cohort of control/non-APL vs the cohort of APL/CRC.

TABLE 8

| | | Control/Non-APL | Control/Non-APL |
|---|---|---|---|
| PS-ID | | vs. APL/CRC | vs. APL/CRC |
| No. | Marker | P-value | FDR |
| 7 | A1AT_271MC_5402 | 5.70E−15 | 2.17E−12 |
| 8 | AGP1_103_7624 | 1.31E−06 | 3.12E−05 |
| 9 | AGP12_56_6503 | 1.68E−13 | 2.50E−12 |
| 10 | ANGT_47_5402 | 8.53E−16 | 1.82E−14 |
| 11 | FETUA_176_6503 | 3.05E−07 | 1.06E−05 |
| 12 | HEMO_453_6503 | 3.14E−06 | 5.55E−05 |
| 13 | HPT_184_7602 | 3.35E−06 | 5.55E−05 |
| 14 | HRG_345_5411 | 1.08E−21 | 8.98E−20 |
| 15 | KNG1_137_1101 | 6.61E−16 | 1.51E−14 |
| 16 | ITIH4_LGVYELLLK | 1.11E−14 | 2.07E−13 |
| 17 | ANGT_SLDFTELDVAAEK | 5.72E−18 | 2.00E−16 |
| 18 | FINC_SYTITGLOPGTDYK | 6.15E−25 | 3.67E−22 |
| 19 | SEPP1_VSLATVDK | 2.51E−14 | 4.15E−13 |
| 20 | VTNC_GQYCYELDEK | 1.42E−23 | 2.83E−21 |
| 21 | THRB_416MC_5402 | 3.63E−23 | 5.41E−21 |

Table 9 shows the model performance metrics of accuracy, sensitivity, and specificity for the validation based on 113 subjects.

TABLE 9

| Comparison | Accuracy | Sensitivity | Specificity |
|---|---|---|---|
| APL/CRC vs Non-APL/Ctrl | 0.8938 | 0.8864 | 0.92 |
| APL vs Non-APL/Ctrl | 0.8824 | 0.8462 | 0.92 |
| CRC vs Non-APL/Ctrl | 0.9080 | 0.9032 | 0.92 |
| CRC½ vs Non-APL/Ctrl | 0.9211 | 0.9216 | 0.92 |
| CRC¾ vs Non-APL/Ctrl | 0.8889 | 0.8182 | 0.92 |

Table 10 shows the model performance metrics of accuracy, sensitivity, and specificity for the test set based on 198 subjects. For both of Tables 9 and 10, the model performance metrics were evaluated for comparing the cohorts of the combination of APL and CRC vs the combination of non-APL and control (Ctrl); APL vs the combination of non-APL and control; CRC vs the combination of non-APL and control; the combination of CRC1 and CRC2 vs the combination of non-APL and control; and the combination of CRC3 and CRC4 vs the combination of non-APL and control. It should be noted that that CRC1, CRC2, CRC3, and CRC4 represent stages 1, 2, 3, and 4 of CRC, respectively. CRC1/2 represents the combination of stages 1 and 2 of CRC and may be referred to as early stage CRC. CRC3/4 represents the combination of stages 3 and 4 of CRC and may be referred to as late stage CRC. It is worthwhile to note that the sensitivity of APL vs Non-APL/Ctrl was 0.84 and 0.85 for Tables 8 and 9, respectively, that corresponds to unmatched sensitivity for this condition compared to a commercial screening assay for CRC.

TABLE 10

| Comparison | Accuracy | Sensitivity | Specificity |
|---|---|---|---|
| APL/CRC vs Non-APL/Ctrl | 0.8939 | 0.8896 | 0.9091 |
| APL vs Non-APL/Ctrl | 0.8750 | 0.8409 | 0.9091 |
| CRC vs Non-APL/Ctrl | 0.9091 | 0.9091 | 0.9091 |
| CRC½ vs Non-APL/Ctrl | 0.9248 | 0.9326 | 0.9091 |
| CRC¾ vs Non-APL/Ctrl | 0.8769 | 0.8095 | 0.9091 |

FIG. 11 shows a ROC curve providing test, train, and validation performance for a classifier model that classifies CRC and APL samples from the control and non-APL samples. The ROC curve of FIG. 11 corresponds to the data for the comparison of APL/CRC vs Non-APL/Ctrl.

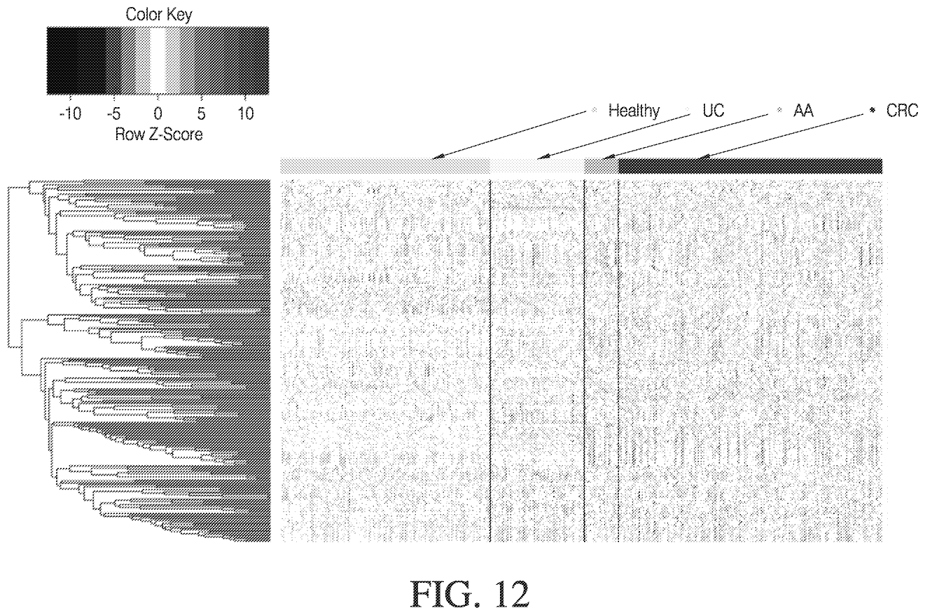

FIG. 12 demonstrates a support vector machine (SVM) score for classifying a sample as being CRC/APL or control/non-APL based on the training data set to determine the performance of the classifier model, utilizing samples of healthy controls, non-APL, APL, CRC stage 1/2, and CRC stage 3/4.

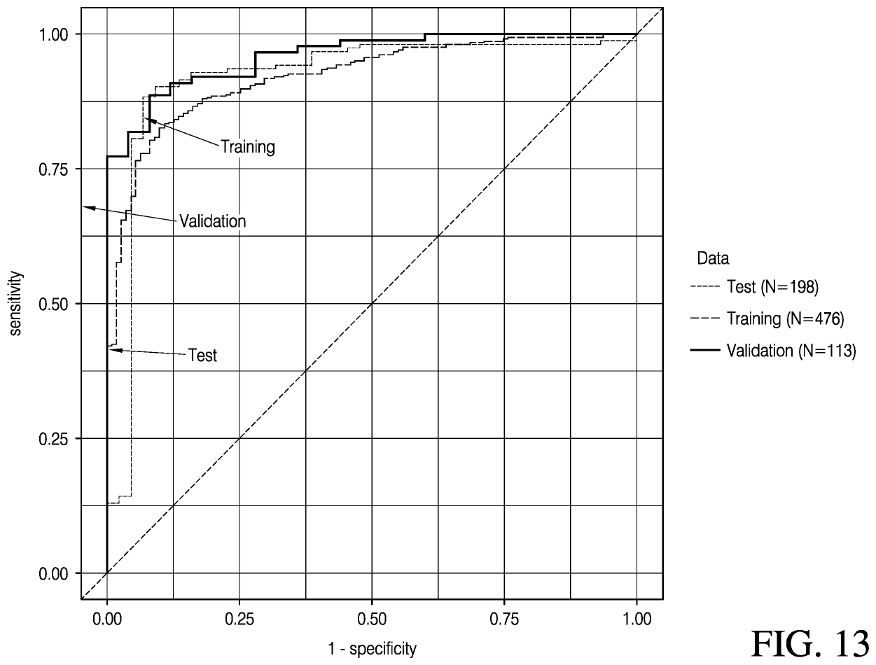

FIG. 13 demonstrates a support vector machine (SVM) score for classifying a sample as being either CRC/APL or control/non-APL based on the validation data set to determine the performance of the classifier model, utilizing samples of healthy controls, non-APL, APL, CRC stage 1/2, and CRC stage 3/4. For the validation data set, the median SVM scores of the controls and non-APL cohorts are negative values and the median SVM scores of the APL, CRC stage 1/2, and CRC stage 3/4 cohorts are positive values indicating that the model can classify a sample between controls/non-APL and APL/CRC stages 1-4.

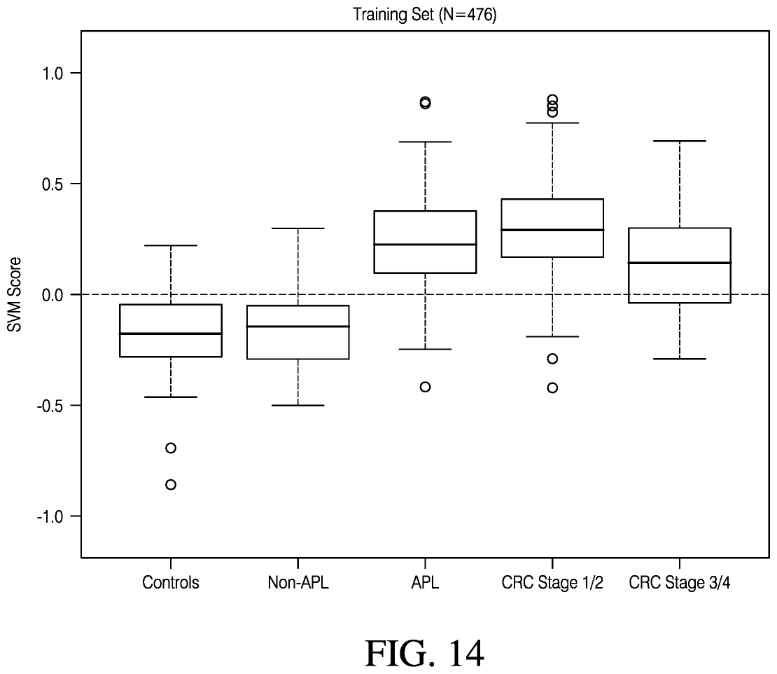

FIG. 14 demonstrates a support vector machine (SVM) score for classifying a sample as being CRC/APL or control/non-APL based on the test data set to determine the performance of the classifier model, utilizing samples of healthy controls, non-APL, APL, CRC stage 1/2, and CRC stage 3/4. For the test data set, the median SVM scores of the controls and non-APL cohorts are negative values and the median SVM scores of the APL, CRC stage 1/2, and CRC stage 3/4 cohorts are positive values indicating that the model can classify a sample between controls/non-APL and APL/CRC stages 1-4.

VIII.A.3 Representative Experimental Results—Subject Sample Model & Corresponding Training of Said Model based on Table 1C

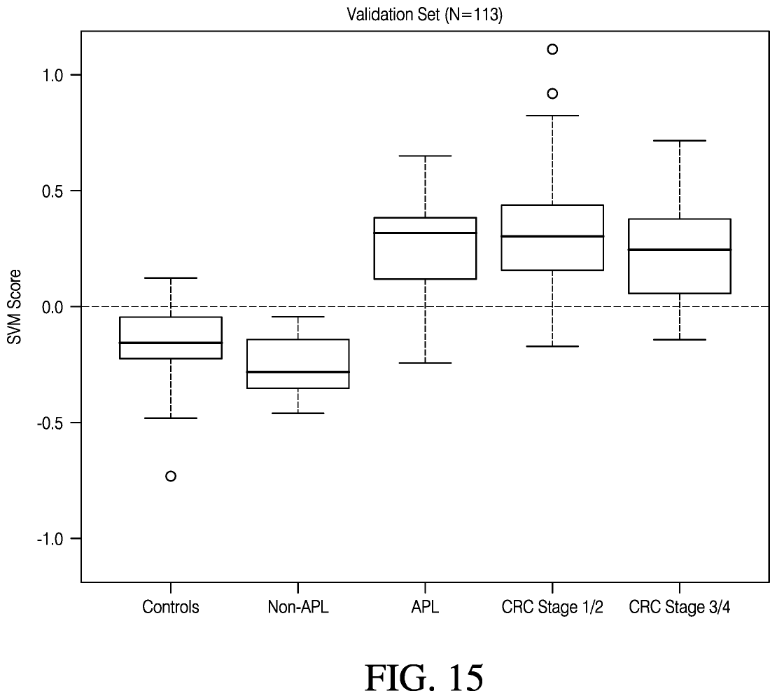

To assess the association of individual peptide structures (biomarkers) with high-grade advanced pre-malignant lesions or colorectal cancer, differential expression analysis (DEA) was run using the Wilcoxon test on a cohort of 2092 samples sourced from different biorepositories, including 533 CRC samples, 296 advanced adenoma samples, 622 samples with benign polyps, and 641 healthy control samples, adjusting for age and sex. Low-grade adenomas (e.g., benign polyps) are adenomas 10-14 mm with no dysplasia and high-grade advanced pre-malignant lesions are adenomas 15 mm or larger or adenomas of any size with high-grade dysplasia. Using the biomarkers of Table 1C, a model was developed that had biomarker weights as shown in Table 11 based on the relative abundance values measured for the biomarkers. The performance metrics of this model were shown in FIG. 15 that has a 35% sensitivity for low-grade adenoma, a 74% sensitivity for high-grade advanced pre-malignant lesions, a sensitivity for CRC stages 1 & 2, and a 92% specificity for CRC stages 1 & 2.

TABLE 11
Coefficients for each marker used in a model for classifying healthy
control vs high-grade advanced pre-malignant lesions/ CRC.

| PS-ID | | Pep SEQ | |
|---|---|---|---|
| No. | PS-NAME | ID NO | Coefficients |
| 22 | A1AT_107_5402 | 42 | 3.197599606 |
| 23 | A1AT_271_5402 | 43 | 14.23622102 |
| 24 | A1AT_271_5412 | 44 | 367.3167681 |
| 25 | A1AT_70_NONGLYCOSYLATED | 45 | −1163.409438 |
| 26 | A2GL_186_5402 | 46 | −0.299136471 |
| 27 | A2MG_1424_5411 | 47 | 4.656282923 |
| 28 | A2MG_247MC_5401 | 48 | 9.042603538 |
| 29 | A2MG_247MC_5402 | 49 | 12.89906073 |
| 30 | AACT_127_5402 | 50 | −29.59234284 |
| 31 | AACT_127_6502 | 51 | −0.385609481 |
| 32 | AGP1_93_7614 | 52 | 0.319029211 |
| 33 | AGP12_56_6503 | 53 | −142.2395549 |
| 34 | AGP12_72MC_7601 | 54 | −49.77500372 |
| 35 | AGP12_72MC_7602 | 55 | −111.3635181 |
| 36 | ANGT_47_5401 | 56 | −3.735967203 |
| 37 | APOC3_74_1111 | 57 | −13.53802108 |
| 38 | APOC3_74MC_1101_corr | 58 | −2.730413236 |
| 39 | APOE_212_1102 | 59 | −21.6388563 |
| 40 | APOE_212_NONGLYCOSYLATED | 60 | −12.18738381 |
| 41 | APOE_308_NONGLYCOSYLATED | 61 | −15.20903398 |
| 42 | APOH_253_6503 | 62 | −39.44188258 |
| 43 | APOH_253_NONGLYCOSYLATED | 63 | −80.28230898 |
| 44 | APOM_135_NONGLYCOSYLATED | 64 | −2.523989465 |
| 45 | C4BPA_221_5412 | 65 | −6.182930434 |
| 46 | CERU_397_5401 | 66 | −56.5443109 |
| 47 | CERU_397_5402 | 67 | −8.241893988 |
| 48 | CERU_762_5402 | 68 | −5.095401257 |
| 49 | CERU_762_6513 | 69 | 22.88355968 |
| 50 | CFAH_1029_5412 | 70 | 1.947469991 |
| 51 | CFAH_1095_5402 | 71 | 4.956918551 |
| 52 | CO3_85_5200 | 72 | −4.264680935 |
| 53 | CO3_85_6200 | 73 | −2.401796199 |
| 54 | CO6_855_5402 | 74 | 0.700855838 |
| 55 | FETUA_156_5402 | 75 | 0.759669257 |
| 56 | FETUA_176_6502 | 76 | 126.9683249 |
| 57 | FINC_542_6502 | 77 | 8.712744295 |
| 58 | HEMO_240/246_5402 | 78 | −0.012009746 |
| 59 | HEMO_453_5402 | 79 | −9.57056986 |
| 60 | HEMO_64_5402 | 80 | 124.7193917 |
| 61 | HPT_207_121005 | 81 | 93.17652476 |
| 62 | HPT_241_5402 | 82 | 2.735083935 |
| 63 | HRG_125_5402 | 83 | 0.273311843 |
| 64 | IC1_238_5402 | 84 | 76.83326169 |
| 65 | IC1_238_5411 | 85 | −8.981792833 |
| 66 | IC1_352_5412 | 86 | −25.5605439 |
| 67 | IGA12_144MC_5400 | 87 | −1117.400186 |
| 68 | IGA12_144MC_5500 | 88 | −55.90224867 |
| 69 | IGA2_205_5410 | 89 | −3.25168817 |
| 70 | IGG1_297_3210 | 90 | 718.2713381 |
| 71 | IGG1_297_3410 | 91 | 3.443070623 |
| 72 | IGG1_297_4400 | 92 | 0.490189709 |
| 73 | IGG1_297_5410 | 93 | −2.254818684 |
| 74 | IGG1_297_5500 | 94 | 35.85570875 |
| 75 | IGG2_297_5400 | 95 | 4.027503707 |
| 76 | IGG34_297_4410 | 96 | 0.000144478 |
| 77 | IGG34_297_4411 | 97 | 11.11650088 |
| 78 | IGJ_71MC_NONGLYCOSYLATED | 98 | 0.760192852 |
| 79 | IGM_209_5401 | 99 | −59.71829501 |
| 80 | IGM_439_5200 | 100 | −13.94003749 |
| 81 | IGM_46_4310 | 101 | −153.6247407 |
| 82 | IGM_46_5412 | 102 | 17.22860615 |
| 83 | IGM_46_6301 | 103 | −37.07557601 |
| 84 | ITIH1_285_5402 | 104 | 7.797795311 |
| 85 | KLKB1_308_5402 | 105 | 0.408791555 |
| 86 | KLKB1_396_5401 | 106 | 7.108131807 |
| 87 | SHBG_380_5402 | 107 | −0.489636431 |
| 88 | THRB_416MC_5402 | 108 | −4.367450884 |
| 89 | TRFE_432_6502 | 109 | −24.73061612 |
| 90 | TRFE_630_5402 | 110 | −20.65882019 |
| 91 | VTNC_169_5402 | 111 | 3.979370643 |

In various embodiments, using the values of Table 11, a probability can be determined by summing together the product of the concentration (or relative abundance) of each biomarker in the sample and the respective coefficient and then adding the summation and the intercept to yield the logit of a probability score. For example, the logit of the probability, to which the inverse logit function can be applied, is equal to:

$${\sum }_{i = 42}^{i = 111} [ ( {Concentration}_{SEQ ID NO : i} ) \times ( {Coefficient}_{SEQ ID NO : i} ) ] + Intercept$$

VIII.A.4 Representative Experimental Results—Subject Sample Model & Corresponding Training of Said Model based on Table 1D

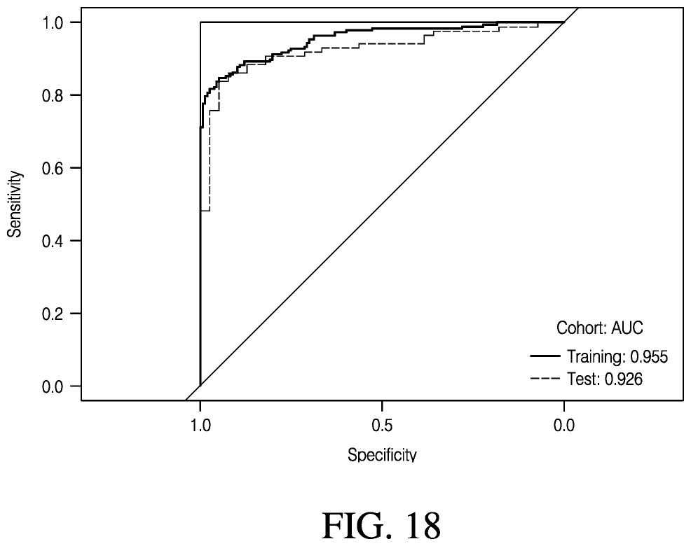

FIG. 18 is an illustration of the sensitivity and specificity of the methods disclosed herein for classifying colorectal cancer and advanced colon adenomas from healthy control samples using the biomarkers in Table 1D.

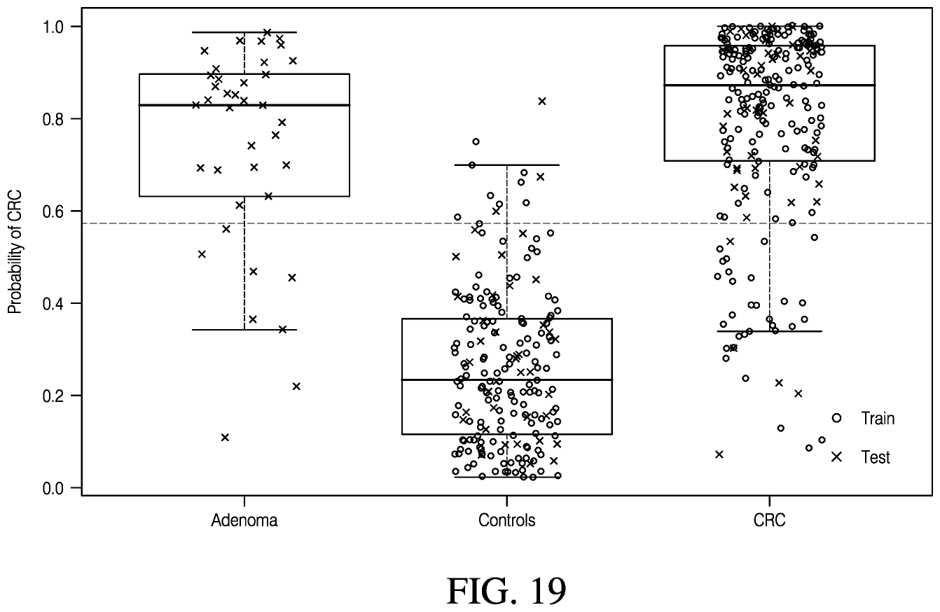

FIG. 19 is an illustration of the resultant distribution of predicted probabilities indicating a well-trained model, and application to blinded healthy patients and those with advanced colon adenoma and/or colorectal cancer.

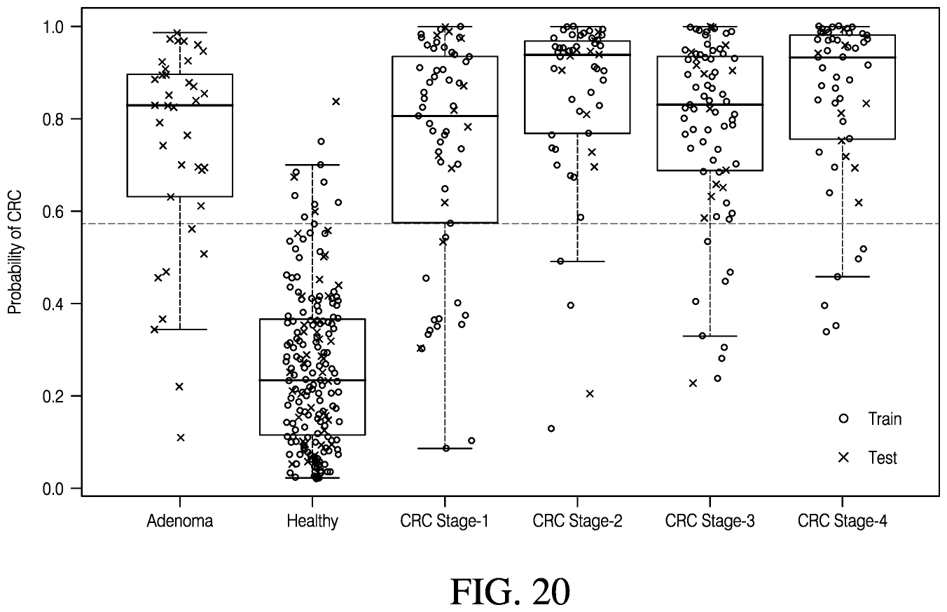

FIG. 20 is an illustration of the resultant distribution of predicted probabilities indicating a well-trained model, and application to blinded healthy patients and increasing severity with disease progression indicating a link to the biology of colorectal cancer.

### VIII.A.5 Representative Experimental Embodiments for MS-HPLC-

Samples were injected into a HPLC/triple quadrupole mass spectrometer. At 0-3 minutes the eluent was diverted to waste, 3-47.8 minutes the eluent was passed to the MS instrument, and 47.8-49 minutes the eluent was diverted to waste. During this time course, a constant solvent gradient was completed. The aqueous mobile phase A was 0.1% formic acid in water (vol:vol), and the organic mobile phase B was 0.1% formic acid in acetonitrile (vol:vol). Separation of peptides and glycopeptides was performed using a binary gradient of 0.0-9.0 min, 1-10% B; 9.0-36.0 min, 10-25% B; 36.0-48.0 min, 25-44% B; 48.0-48.1 min, 44-1% B; 48.1-49.0 min, 1% B. The liquid chromatography system was an Agilent 1290 Infinity II UHPLC system that used a 20 μL loop volume, 4 μL injection volume, Waters ACQUITY UPLC Peptide HSS T3 Column, 100 Å port volume, 1.8 μm particle size, 2.1 mm×150 mm (diameter×length) with HSS T3 guard column, 2.1 mm×5 mm. The output of the chromatography column was either outputted to a waste channel or to the mass spectrometer via an electrospray ionization unit using a microprocessor controlled valve depending on the time of the chromatography run (see Table 1).

The mass spectrometry system was an Agilent 6495C triple quadrupole mass spectrometer. Samples were introduced into the mass spectrometer using an electrospray ionization (ESI) source operated in the positive ion mode. Nitrogen drying and sheath gas temperatures were set at 290° C. and 300° C., respectively. Drying and sheath gas flow rates were set at 11 L/min and 12 L/min, respectively. The nebulizer pressure was set to 30 psi. Data acquired from the UHPLC/QqQ-MS was collected using Agilent MassHunter Workstation LC/MS Data Acquisition B10.1.67. Sample analysis was performed using a dynamic multiple reaction monitoring (dMRM) method. Collision induced dissociation was used for fragmentation.

### IX. Exemplary Embodiments for Adenoma or Colorectal Cancer Diagnosis and Treatment

The present disclosure concerns embodiments for systems, methods, and compositions related to identification of adenoma or colorectal cancer (CRC), or risk thereof, in an individual. The embodiments concern classifying biological samples, measuring for one or more certain markers from a biological sample, assaying for one or more certain markers from a biological sample, determining the presence of one or more certain markers from a biological sample, and so forth. The embodiments of the disclosure utilize models that accurately either identify that an individual has an adenoma or CRC or that has a higher risk for adenoma or CRC over the general population based on the presence of one or more markers in sample(s) from the individual. The individual may or may not be at a higher risk for adenoma or CRC based on one or more risk factors. An individual may be at risk for CRC based on family or personal history; age (e.g., 50 or older); having one or more genetic markers associated with CRC; having inflammatory bowel disease such as Crohn's disease or ulcerative colitis; having a genetic syndrome such as familial adenomatous polyposis (FAP) or hereditary non-polyposis colorectal cancer (Lynch syndrome); having lack of regular physical activity; having a diet low in fruits and vegetables; having a low-fiber and/or high-fat diet; being overweight or obese; high alcohol consumption, and/or tobacco use. An individual may be at risk for adenomas based on age, body weight, waist circumference, blood lipid, and/or blood glucose levels.

In various embodiments of the disclosure, an individual is in need of identifying whether or not they have adenoma or CRC, or a risk thereof. The individual may be subjected to measuring or testing for one or more markers encompassed herein as a matter of routine health maintenance or because of a specific concern, for example, such as the presence of one or more risk factors and/or one or more symptoms of adenoma or CRC. The individual may be in need of such identification based on any one of the risk factors noted above, or the individual may be in need of such identification based on having one or more symptoms of adenoma or CRC.

In some cases, the analysis of the sample of the individual as described herein is the sole test utilized for identifying adenoma or CRC, whereas in other cases a medical provider may utilize one or more other tests, such as ultrasound; magnetic resonance imaging; CT scan; biopsy; a combination thereof, colonoscopy, and so forth. In particular embodiments, measuring for one or more peptide structure markers as in Table 1 are utilized alone or in conjunction with one or more of these tests.

In some cases, the analysis of the sample of the individual as described herein is the sole test utilized for identifying APL or CRC, whereas in other cases a medical provider may utilize one or more other tests, such as ultrasound; magnetic resonance imaging; CT scan; biopsy; a combination thereof, colonoscopy, and so forth. In particular embodiments, measuring for one or more peptide structure markers as in Table 1B are utilized alone or in conjunction with one or more of these tests.

In some cases, the analysis of the sample of the individual as described herein is the sole test utilized for identifying high-grade advanced pre-malignant lesion or CRC, whereas in other cases a medical provider may utilize one or more other tests, such as ultrasound; magnetic resonance imaging; CT scan; biopsy; a combination thereof, colonoscopy, and so forth. In particular embodiments, measuring for one or more peptide structure markers as in Table 1C are utilized alone or in conjunction with one or more of these tests.

In some cases, the analysis of the sample of the individual as described herein is the sole test utilized for identifying CRC, whereas in other cases a medical provider may utilize one or more other tests, such as ultrasound; magnetic resonance imaging; CT scan; biopsy; a combination thereof, colonoscopy, and so forth. In particular embodiments, measuring for one or more peptide structure markers as in Table 1D are utilized alone or in conjunction with one or more of these tests.

The systems, methods, and compositions encompassed herein are sufficiently specific to utilize markers that distinguish between control and adenoma or CRC. In some embodiments, the markers are accurate regardless of the status of one or more characteristics of the individual: biological sex, sample source, sample collection, smoker status, or age, as examples.

In some embodiments, the individual is suspected of having adenoma or CRC or is at risk for adenoma or CRC and is in need of diagnosis thereof in addition to identification whether it is a particular stage of CRC. In various embodiments, the individual is known to have CRC and is in need of determining whether it is early stage CRC or late stage CRC, such as to determine a treatment regimen for the cancer. In specific embodiments, the same test that identifies whether an individual has CRC determines whether the CRC is early stage or late stage or a particular stage.

In various embodiments, the sample for analysis for adenoma or CRC identification may be a solid or fluid from the individual, such as stool, peripheral blood, serum, and/or plasma from the individual. The present disclosure provides for measuring for one or more circulating glycoproteins, glycopeptides, or non-glycosylated peptides in stool, blood, serum, or plasma to diagnose or identify the presence of adenoma or CRC and/or to identify early stage or late stage CRC in an individual. In various embodiments, the sample is measured for 1, 2, 3, 4, 5, or all 6 of the peptides of Table 1.

Embodiments of the disclosure include methods of classifying samples, including stool, peripheral blood, serum, or plasma samples, from an individual suspected of having, known to have, or at risk for having adenoma or CRC by measuring from the sample for one or more glycopeptides and/or non-glycosylated peptides encompassed herein. The methods encompass whether or not adenoma or CRC is identified in the individual. In some cases, the measuring identifies the individual as not having adenoma or CRC or as having adenoma or CRC. In various embodiments, in cases wherein the individual has one or more glycopeptides and/or non-glycosylated peptides of Table 1, or certain levels thereof compared to control or healthy individuals, the individual may be determined to have adenoma or CRC. In various embodiments, in cases wherein the individual lacks the glycopeptides and/or non-glycosylated peptides of Table 1, or has certain levels thereof compared to control or healthy individuals, the individual may be determined not to have adenoma or CRC. The measuring may identify the individual as having a particular stage of CRC, including at least early stage or late stage. In specific cases, the measuring comprises successive or concomitant steps of identifying that the individual has adenoma or CRC and whether the individual has early stage or late stage CRC.

In various embodiments, an individual at risk for having adenoma or CRC is subjected to methods of the disclosure to identify, or not, the presence of adenoma or CRC. Such methods also measure for one or more glycopeptides and/or non-glycosylated peptides encompassed herein. In various embodiments, in cases wherein the individual has one or more glycopeptides and/or non-glycosylated peptides of Table 1, the individual may be determined to have adenoma or CRC. In various embodiments, in cases wherein the individual lacks the glycopeptides and/or non-glycosylated peptides of Table 1, the individual may be determined not to have adenoma or CRC and is not treated for adenoma or CRC. The individual may be of any kind, although in specific cases individual at risk for having adenomas and/or colorectal cancer has a family history or one or more other risk factors.

Embodiments of the disclosure include methods of predicting that an individual will have adenoma or CRC, including early stage or late stage CRC, or identifying early stage or late stage CRC in an individual, by measuring for one or more glycopeptides or non-glycosylated peptides from Table 1 in one or more samples from the individual. The individual may be known to have adenoma or CRC or may be suspected of having adenoma or CRC In various embodiments, the sample is measured for 1, 2, 3, 4, 5, or all 6 of the peptides of Table 1.

In embodiments wherein the measuring identifies the individual as having CRC, the individual may be recommended to take action to treat the CRC, such as with at least one of radiation therapy, chemotherapy or drug therapy (Bevacizumab, evacizumab, Irinotecan Hydrochloride, Capecitabine, Cetuximab, Ramucirumab, Oxaliplatin, Cetuximab, 5-FU, Ipilimumab, Irinotecan Hydrochloride, Pembrolizumab, Leucovorin Calcium, Trifluridine and Tipiracil Hydrochloride, Nivolumab, Nivolumab, Oxaliplatin. Panitumumab, Pembrolizumab, Ramucirumab, Regorafenib, Regorafenib, Panitumumab, Ziv-Aflibercept), chemoradiotherapy, surgery, hormone therapy and/or a targeted drug therapy, as examples.

Embodiments of the disclosure include methods of treating adenoma or CRC in a subject, the method comprising: receiving a biological sample from the subject; determining a quantity of at least 1 peptide structure identified in Table 1 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has adenoma or CRC; and administering a therapeutically effective amount of the treatment for adenoma or CRC. The treatment may be of any kind, including at least one or more of biopsy, radiation therapy, chemotherapy, chemoradiotherapy, surgery, or a targeted drug therapy. In specific embodiments, the method further comprises preparing the biological sample to form a prepared sample comprising a set of peptide structures; and inputting the prepared sample into the MRM-MS system using a liquid chromatography system. The method may also be further defined as determining a quantity of at least 1 peptide structure identified in Table 1 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has adenoma or CRC; and administering a therapeutically effective amount of the treatment for adenoma or CRC.

Certain embodiments of the disclosure encompass methods of designing a treatment for a subject diagnosed with adenoma or CRC state, the method comprising: designing a therapeutic regimen for treating the subject in response to measuring that a biological sample obtained from the subject evidences the state using part or all of any method encompassed herein, including identifying one or more peptide structures of Table 1. Various embodiments include methods of planning a treatment for a subject diagnosed with an adenoma or CRC state, the method comprising: generating a treatment plan for treating the subject in response to measuring that a biological sample obtained from the subject evidences the state using part or all of any method encompassed herein, including identifying one or more peptide structures of Table 1.

Embodiments of the disclosure include methods of treating a subject diagnosed with adenoma or CRC state, the method comprising: administering to the subject a therapeutically effective amount of one or more therapeutics or treatments to treat the subject based on measuring that a biological sample obtained from the subject evidences the state using part or all of any method encompassed herein, including that identifies one or more peptide structures of Table 1.

Embodiments of the disclosure include methods of treating a subject diagnosed with APL or CRC state, the method comprising: administering to the subject a therapeutically effective amount of one or more therapeutics or treatments to treat the subject based on measuring that a biological sample obtained from the subject evidences the state using part or all of any method encompassed herein, including that identifies one or more peptide structures of Table 1B.

Embodiments of the disclosure include methods of treating a subject diagnosed with high-grade advanced pre-malignant lesion or CRC state, the method comprising: administering to the subject a therapeutically effective amount of one or more therapeutics or treatments to treat the subject based on measuring that a biological sample obtained from the subject evidences the state using part or all of any method encompassed herein, including that identifies one or more peptide structures of Table 1C.

Embodiments of the disclosure include methods of treating a subject diagnosed with the CRC state, the method comprising: administering to the subject a therapeutically effective amount of one or more therapeutics or treatments to treat the subject based on measuring that a biological sample obtained from the subject evidences the state using part or all of any method encompassed herein, including that identifies one or more peptide structures of Table 1D.

In various embodiments, methods of treating a subject diagnosed with adenoma or CRC state are encompassed herein, the method comprising: selecting a therapeutic or treatment to treat the subject based on determining that the subject is responsive to the therapeutic using any method encompassed herein, including that identifies one or more peptide structures of Table 1.

In various embodiments, methods are included for classifying a sample from an individual suspected of having, known to have, or at risk for adenoma or CRC, comprising the step of measuring from the sample for one or more glycopeptides and/or non-glycosylated peptides in Table 1. In specific embodiments, the measuring identifies the individual as not having adenoma or CRC or as having adenoma or CRC. The measuring may identify the individual as having early stage or late stage CRC, in specific embodiments, and the detection of early stage malignancy is useful such that a treatment path may be determined as soon as possible. In certain embodiments, the measuring comprises successive or concomitant steps of identifying that the individual has adenoma or CRC and/or that the individual has early stage or late stage CRC. The individual may or may not be at risk for adenoma or CRC. In specific cases, when the measuring identifies the individual as having adenoma or CRC, the individual is administered an effective amount of at least one of biopsy, radiation therapy, chemotherapy, chemoradiotherapy, surgery, or a targeted drug therapy. In various embodiments, the sample is measured for 1, 2, 3, 4, 5, or all 6 of the glycopeptides and/or non-glycosylated peptides of Table 1.

Embodiments of the disclosure include methods of diagnosing adenoma or CRC in an individual, comprising the step of identifying 1, 2, 3, 4, 5, or all 6 of the peptide structures identified in Table 1 from a sample from the individual.

In various embodiments, an individual is measured for 1, 2, 3, 4, 5, or all 6 of the peptide structures identified in Table 1 from a sample from the individual for the purpose of identification of adenoma or CRC. In specific embodiments, when 1, 2, 3, 4, 5, or all 6 of the peptide structures identified in Table 1 are measured in a sample from the individual, the individual is determined either to have adenoma, to have CRC, or to require further testing to definitively determine whether the individual has adenoma or CRC. In specific cases, the individual is subject to further testing of any kind and is determined either to have adenoma or CRC, based on the presence of cancerous cells in the sample, for an example. Such further testing may or may not include colonoscopy, biopsy, biomarker testing of the cells, blood tests, CT scan, MRI, or a combination thereof.

In various embodiments, the disclosure relates to a method of screening a subject to identify and quantify risk of adenoma or CRC, and thereby identify subjects suitable for further invasive investigation such as a colonoscopy. The method measures for certain one or more glycosylated or aglycosylated peptides that are shown to correlate with adenoma or CRC and involves assaying a biological sample from the subject for one or a combination of biomarkers selected from PS-1 to PS-6, where the one or combination of biomarkers is chosen such that their detection correlates to at least an increased risk over the general population of the subject being positive for adenoma or CRC. Detection of one or all of the combination of biomarkers indicates that the subject should undergo at least colonoscopy. In doing so, if one or more polyps and/or lesions are detected they may be removed for further analysis.

Subjects for which the systems and methods and compositions of the present disclosure may be subjected to may follow recommendations of The American Cancer Society that people at average risk of CRC start regular screening at age 45. An individual at average risk is considered one who has not had a personal history of colorectal cancer or certain types of polyps; a family history of colorectal cancer; a personal history of inflammatory bowel disease (ulcerative colitis or Crohn's disease); a confirmed or suspected hereditary colorectal cancer syndrome, such as familial adenomatous polyposis (FAP) or Lynch syndrome (hereditary non-polyposis colon cancer or HNPCC); or a personal history of getting radiation to the abdomen (belly) or pelvic area to treat a prior cancer. In some cases, the subject may also be subjected to a stool-based test that looks for signs of cancer in a person's stool or with a visual exam that looks at the colon and rectum.

Subjects who are in good health and with a life expectancy of more than 10 years may be subjected to systems, methods and compositions of the present disclosure through the age of 75. Subjects aged 76 through 85 may be subjected to the systems, methods, and compositions of the present disclosure based on the subject's preferences, life expectancy, overall health, and prior screening history.

### X. Glycopeptide Biomarker Discovery For Diagnosing Colorectal Cancer

Provided herein are methods useful for diagnosing colorectal cancer (CRC) based upon one or more biomarkers. These methods are particularly useful because CRC is often asymptomatic until it has metastasized and has become life threatening, limiting possible therapeutic options. Thus, early diagnosis of CRC is key for effective treatment outcomes. In some embodiments, the diagnosis is based upon the presence, absence, and/or amount of one or more peptide structures comprising a sequence set forth in SEQ ID NOs: 168-198. In some embodiments, the diagnosis is based upon the presence, absence, and/or amount of one or more peptide structures comprising a sequence set forth in SEQ ID NOs: 168, 171, 172, 176, 181, 184, 187, 192, and 194. In some embodiments, the biomarkers are used to identify a person at risk for developing CRC and recommend a follow up procedure for a definitive diagnosis. In some embodiments, following a determination that an individual is at risk for developing CRC based upon the biomarkers provided herein, the individual is recommended to receive an endoscopy.

In some embodiments, the present methods are able to diagnosis an individual as at risk for developing colorectal cancer (CRC) based upon the presence, absence, and/or amount of one or more peptide structures comprising a sequence set forth in SEQ ID NOs: 168-198. In some embodiments, the present methods are able to predict the likelihood or risk that an individual will develop CRC based upon the presence, absence, and/or amount of one or more peptide structures comprising a sequence set forth in SEQ ID NOs: 168-198.

### X.I. Definitions

As used herein, the term “plurality” is more than 1 and may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list is required to be included. The item may be a particular object, thing, step, operation, process, or category. In other words, “at least one of” means any combination of items or number of items may be used from the list, but not all of the items in the list may be required. For example, without limitation, “at least one of item A, item B, and item C” intends and includes any of item A; item A and item B; item B; item A, item B, and item C; item B and item C; item C; and item A and C. It is understood that “at least one of” includes instance where more than one of any listed item is present. For example, and without limitation, at least one of item A, item B, and item C include an embodiment in which two of item A is present, one of item B is present, and ten of item C is present.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance.

The term “amino acid,” as used herein, generally refers to any organic compound that includes an amino group (e.g., —NH₂), a carboxyl group (—COOH), and a side chain group (R) which varies based on a specific amino acid. Thus, “amino acid” includes organic compounds of the formula NH₂—CH(R)—COOH where R represents an amino acid side chain group. In some instance R represents the side chain of a natural amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation. N-linked glycosylation can include a glycan attached to an asparagine. O-linked glycosylation can include a glycan attached to either a serine or a threonine.

The term “biomarker,” as used herein, generally refers to any measurable substance taken as a sample from a subject whose presence, absence and/or amount is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a disease state, a health state, an asymptomatic state, a symptomatic state, etc.). The term “biomarker” can be used interchangeably with the term “marker.” Biomarkers include peptide structures such as those listed in Table 13A.

The term “denaturation,” as used herein, generally refers to protein unfolding. Non-limiting examples include proteins or nucleic acids being exposed to an external compound or environmental condition such as acid, base, elevated temperature, pressure, radiation, etc.

The term “denatured protein,” as used herein, generally refers to a protein that loses quaternary structure, tertiary structure, and secondary structure which is present in its native state.

The terms “digestion” or “enzymatic digestion,” or “proteolytic digest,” as used herein, generally refer to breaking apart a polymer (e.g., cutting a polypeptide at a cut site). Proteins may be digested in preparation for mass spectrometry using trypsin digestion protocols. Proteins may be digested using other proteases in preparation for mass spectrometry if access is limited to cleavage sites.

The term “disease progression,” as used herein, refers to a progression of a disease from no disease or a less advanced form of disease to a more advanced (e.g., severe) form of the disease. A disease progression may include any number of stages of the disease.

The term “disease state” as used herein, generally refers to a condition that affects the structure or function of an organism. Disease states can include, for example, stages of a disease progression. Disease states can include any state of a disease whether symptomatic or asymptomatic. Disease states can cause minor, moderate, or severe disruptions in the structure or function of a subject. Disease state includes colorectal cancer (CRC), early-stage CRC, late-stage CRC, severe CRC, disposition or likelihood of CRC, or normal or healthy state with respect to CRC.

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycoprotein” or “glycopolypeptide” as used herein, generally refers to a protein having at least one glycan residue bonded thereto. In some examples, a glycoprotein is a protein with at least one oligosaccharide chain covalently bonded thereto. Examples of glycoproteins, include but are not limited SEQ ID NOs: 13 and 19.

The term “glycopeptide” as used herein, refers to a fragment of a glycoprotein, unless specified otherwise to the contrary. In various embodiments, glycopeptides comprise carbohydrate moieties (e.g., one or more glycans) covalently attached to a side chain (i.e., R group) of an amino acid residue. Examples of glycopeptides, include but are not limited to the glycopeptides provided in Table 13A. Examples of glycopeptides, include but are not limited to the glycopeptides provided in Table 13B. Examples of glycopeptides, include but are not limited to SEQ ID NOs: 168-198.

The term “liquid chromatography,” as used herein, generally refers to a technique used to separate a sample into parts. Liquid chromatography can be used to separate, identify, and quantify components.

The term “mass spectrometry” as used herein, generally refers to an analytical technique used to identify molecules by measuring mass-to-charge (m/z) ratios along with corresponding abundance values. In various embodiments described herein, mass spectrometry can be involved in characterization and sequencing of proteins as well as to determine the presence, absence and/or abundance or peptides or proteins.

The term “m/z” or “mass-to-charge ratio” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “peptide,” as used herein, refers to amino acids linked by peptide bonds less than 50 amino acids in length. Peptides can include amino acid chains shorter than 10 residues, including, oligopeptides, dipeptides, tripeptides, and tetrapeptides. Peptides include glycopeptides, which are peptides that contain at least one glycan residue bonded thereto. For example, peptides include peptides comprising, consisting of, or consisting essentially of the peptide structures provided in Table 13A and Table 13B.

The terms “protein” or “polypeptide” may be used interchangeably herein and refer to a polymer in which the monomers are amino acid residues that are joined together through amide bonds of at least 50 amino acid residues in length. Proteins may be digested in preparation for mass spectrometry using trypsin digestion protocols. Proteins may be digested using other proteases in preparation for mass spectrometry if access is limited to cleavage sites. Proteins include glycoproteins, which are proteins that contain at least one glycan residue bonded thereto.

The term “peptide structure,” as used herein, generally refers to peptides or a portion thereof or glycopeptides or a portion thereof. In various embodiments described herein, a peptide structure can include any molecule comprising at least two amino acids in sequence. A peptide structure of a glycopeptide includes description of the peptide amino acids sequence as well as the location and identity of the associated glycan.

The term “reduction,” as used herein, generally refers to the gain of an electron by a substance. In various embodiments, reduction may be used to break disulfide bonds between two cysteines.

The term “sample” and “biological sample” as used herein, generally refers to a sample obtained from a subject of interest. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates.

The term “subject” or “individual” as used herein, refer to a human. A subject can include a healthy or asymptomatic individual, an individual that has or is suspected of having a disease (e.g., CRC) or a pre-disposition to the disease, and/or an individual that needs therapy or suspected of needing therapy. A subject can be a patient.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry. For example, a quadrupole mass analyzer of a mass spectrometer can be configured to filter a preselected m/z value that corresponds to a target glycopeptide analyte in an ionized state.

As used herein, a “peptide data set,” may be used interchangeably with “peptide structure data” and can refer to any data of or relating to a peptide presence or abundance. For example, peptide data set or peptide structure data can be based upon a mass spectrometry run, an ELISA, or western blot. A peptide data set can comprise data obtained from a sample or biological sample using mass spectrometry. A peptide dataset can comprise data relating to a non-glycosylated endogenous peptide (NGEP) external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “non-glycosylated endogenous peptide” (“NGEP”), which may also be referred to as an aglycosylated peptide, may refer to a peptide structure that does not comprise a glycan molecule. In various embodiments, an NGEP and a target glycopeptide analyte can originate from the same subject. In various embodiments, an NGEP can be labeled with an isotope in preparation for mass spectrometry analysis.

As used herein, a “transition,” may refer to or identify a peptide structure. In some embodiments, a transition can refer to the specific pair of m/z values associated with a precursor ion and a product or fragment ion.

As used herein, an “abundance value” may refer to “abundance” or a quantitative value associated with abundance.

As used herein, “abundance,” may refer to a quantitative value generated using mass spectrometry. In various embodiments, the quantitative value may relate to an amount of a particular peptide structure (e.g., biomarker) present in a biological sample. In some embodiments, the amount may be in relation to other structures present in the sample (e.g., relative abundance). In some embodiments, the quantitative value may comprise an amount of an ion produced using mass spectrometry. In some embodiments, the quantitative value may be associated with an m/z value (e.g., abundance on y-axis and m/z on x-axis). In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot. In some embodiments, the relative abundance can be proportional to the total number of peptide spectrum matches (PSMs) for one peptide structure where the term all of the measured peptide structures can be determined by a filtering criteria (e.g., pGlyco3 false discovery rate (FDR)<0.1%).

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled. In some instances, the term internal standard can be referred to with the abbreviation ISTD.

“Likelihood of developing CRC” means the probability, based upon one or more criteria, that a subject will develop CRC.

“Healthy” or “normal” as used herein refers to an individual who does not have CRC and/or has a low risk of CRC. The individual may have other diseases, disorders, and/or conditions, which may or may not relate to CRC. For example, an individual who does not have CRC but does have irritable bowel disease is considered healthy or normal as used herein.

“Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease.

### X.II. Exemplary Workflow

FIG. 1 is a schematic diagram of an exemplary workflow 100 for the detection of peptide structures associated with a disease state for use in diagnosis and/or treatment in accordance with one or more embodiments. Workflow 100 may include various operations including, for example, sample collection 102, sample intake 104, sample preparation and processing 106, data analysis 108, and output generation 130.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be plasma, serum, blood, or stool collected that can be collected into a vial with a septum cap. Biological sample 112 may be obtained in any of a number of different ways. In various embodiments, biological sample 112 includes whole blood sample 116 obtained via a blood draw. In other embodiments, biological sample 112 includes a set of aliquoted samples 118 that includes, for example, a serum sample, a plasma sample, a blood cell (e.g., white blood cell (WBC), red blood cell (RBC) sample, another type of sample, or a combination thereof. Biological sample 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance values (e.g., abundance or raw abundance) for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes plasma or serum sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. Peptide structure analysis can include determining the composition and the associated quantity for the various peptides and glycopeptides present in the sample by processing the output of a mass spectrometer. In other embodiments, output generation 110 may be considered a separate operation from data analysis 108. Output generation 110 may include, for example, generating final output 128 based on the results of peptide structure analysis 126. In various embodiments, final output 128 may be used for determining the research, diagnosis, and/or treatment of a state associated with CRC.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, the report can comprise a target glycopeptide analyte concentration as a function of the NGEP concentration value and the normalized abundance value. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of, for example, CRC.

### X.III. Detection and Quantification of Peptide Structures

FIG. 2A and FIG. 2B are schematic diagrams of a workflow for sample preparation and processing 106 in accordance with one or more embodiments. FIG. 2A and FIG. 2B are described with continuing reference to FIG. 1. Sample preparation and processing 106 may include, for example, preparation workflow 200 shown in FIG. 2A and data acquisition 124 shown in FIG. 2B.

### X.III.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent (e.g. heating the sample to about 90° C. to about 100° C. for about 1 to about 10 minutes. The thermal denaturation can disrupt ionic bonding, hydrophobic interactions, and/or hydrogen bonding.

In one or more embodiments, the denaturation procedure may include using one or more denaturing agents, temperature (e.g., heat), or both. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The acetamide group or alkylation group that attaches to the protein or peptide results in a different form that is not naturally occurring in nature. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards. In some embodiments, post-digestion procedure 207 further includes a procedure for enrichment of glycopeptides in the digested sample. The enrichment procedure may include, for example, using a Hydrophilic Interaction Liquid Chromatography (HILIC) concentration phase.

Although preparation workflow 200 has been described with respect to a sample created or taken from biological sample 112, such as a blood-based sample 116 (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptide structures 122.

### X.II.B. Peptide Structure Identification and Quantification

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, quantification 208 of peptides and glycopeptides can incorporate use of liquid chromatography-mass spectrometry LC-MS instrumentation. For example, LC-MS/MS, or tandem MS may be used. In general, LC-MS (e.g., LC-MS/MS) can combine the physical separation capabilities of liquid chromatography (LC) with the mass analysis capabilities of mass spectrometry (MS). According to some embodiments described herein, this technique allows for the separation of digested peptides to be fed from the LC column into the MS ion source through an interface. In various embodiments, quantification 208 is targeted quantification.

In various embodiments, any LC-MS device can be incorporated into the workflow described herein. In various embodiments, an instrument or instrument system suited for identification and quantification 208 may include, for example, a LC-MS/MS (such as an Orbitrap). In various embodiment, the mass spectrometry comprises atmospheric pressure mass spectrometry. In various embodiments, the mass spectrometry comprises field asymmetric Ion mobility spectrometry (FAIMS). In various embodiments, quantification 208 is performed using data dependent acquisition (DDA) mass spectrometry. DDA-MS is a mass spectrometry method in which the most abundant ions within a certain m/z range (MS1) are individually selected, fragmented and analyzed in a second stage (MS2) of tandem mass spectrometry.

In various embodiments, an instrument or instrument system suited for identification and quantification 208 may include, for example, a Triple Quadrupole LC-MS. In various embodiments, quantification 208 is performed using multiple reaction monitoring mass spectrometry (MRM-MS). MRM is a mass spectrometry method in which a precursor ion of a particular m/z (e.g., peptide analyte) is selected in the first quadrupole (Q1) and transmitted to the second quadrupole (Q2) for fragmentation. The resulting product ions are then transmitted to the third quadrupole (Q3), which detects only product ions with selected predefined m/z values. The particular m/z value set for the first quadrupole (Q1) and the selected predefined m/z values of the third quadrupole have a mass range that ranges within +/−1, +/−0.5, or +/−0.1 m/z values.

In various embodiments described herein, identification of a particular protein or peptide and an associated quantity can be assessed. In various embodiments described herein, identification of a particular glycopeptide and an associated quantity can be assessed. In various embodiments described herein, identification of a particular glycan and an associated quantity can be assessed. In various embodiments described herein, particular glycans can be matched to a glycosylation site on a protein or peptide and the abundance values measured. In various embodiments, a glycopeptide of any of SEQ ID NOs: 168-198 and an associated quantity is assessed. In various embodiments, a glycopeptide provided in Table 13A and an associated quantity is assessed. In various embodiments, a glycopeptide of any of SEQ ID NOs: 168, 171, 172, 176, 181, 184, 187, 192, and 194 and an associated quantity is assessed. In various embodiments, a glycopeptide provided in Table 13B and an associated quantity is assessed. The glycan portion of the glycopeptide is provided in Table 15 that indicate the corresponding symbol structure and composition for the glycopeptides of Tables 13A and 13B.

In some cases, quantification 208 includes using a specific collision energy associated for the appropriate fragmentation to consistently see an abundant product ion. Glycopeptides may have a lower collision energy than aglycosylated peptide structures. When analyzing a sample that includes glycopeptides, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

In some embodiments, the presence, absence, and/or amount of at least one peptide structures is determined by a method other than mass spectrometry, for example by ELISA or immunoblotting (such as western blot). In some embodiments, the presence, absence/and or amount of a peptide structure set forth in Table 13A is determined by a method other than mass spectrometry, for example by ELISA or immunoblotting (such as western blot). In some embodiments, the presence, absence and/or amount of a peptide structure comprising a sequence set forth in SEQ ID NOs: 168-198 or SEQ ID NOs: 168, 171, 172, 176, 181, 184, 187, 192, and 194 is determined by a method other than mass spectrometry, for example by ELISA or immunoblotting (such as western blot).

In some embodiments, the presence, absence, and/or amount of at least one peptide structures is determined by a method other than mass spectrometry, for example by ELISA or immunoblotting (such as western blot). In some embodiments, the presence, absence/and or amount of a peptide structure set forth in Table 13B is determined by a method other than mass spectrometry, for example by ELISA or immunoblotting (such as western blot). In some embodiments, the presence, absence and/or amount of a peptide structure comprising a sequence set forth in SEQ ID NOs: 168, 171, 172, 176, 181, 184, 187, 192, and 194 is determined by a method other than mass spectrometry, for example by ELISA or immunoblotting (such as western blot).

It is worthwhile to note that Table 13A and Table 13B includes the term Peptide Structure (PS) Name that refers to a reference name for a peptide or glycopeptide. The Peptide Structure (PS) Name of Table 13A and Table 13B contains a prefix that represents an acronym for a protein abbreviation that corresponds to the Protein Abbreviation of Table 14. The term Peptide Sequence lists the order of amino acids in a series of single letter abbreviations. The term Linking Site Pos. in Protein Sequence is a number that refers to the position of an amino acid in which a glycan is attached. For the Linking Site Pos. in Protein Sequence, the amino acid position of the peptide sequence is defined by the numbered order of amino acids based on the UniProt ID of the corresponding protein for the peptide sequence. The term Linking Site Pos. in Peptide Sequence is a number that refers to the position of an amino acid in which a glycan is attached. For the Linking Site Pos. in peptide Sequence, the amino acid position of the peptide sequence is defined by the numbered order of amino acids (from left to right) for the peptide sequence. The term Glycan Structure GL No. is a number that corresponds to a symbol structure and a composition of the glycans as indicated in Table 15.

In some embodiments, the at least one peptide structure comprises a peptide sequence and a glycan structure in accordance with Table 13A or 13B. In some embodiments, the glycan structure is attached to a linking site position in the peptide sequence in accordance with Table 13A or 13B. For example, glycopeptide HPT (241)-4310 set forth by SEQ ID NO:19 describes the primary structure of the peptide listed under the Peptide Sequence column, wherein the Glycan Structure GL No 4310 is attached to the peptide at position 241 with respect to the position on the protein HPT in accordance with Table 13A or 13B. In this example, the Glycan Structure GL No 4310 is attached at position 6 (Asparagine 6, Asn6) of the peptide sequence listed in accordance with Table 13A or 13B.

Referring to Table 15, the term Symbol Structure illustrates a geometric linking structure of the carbohydrates where the bottommost carbohydrate is bound to the amino acid. The identity of the various monosaccharides is illustrated by the Legend section located at the end of Table 15. The abbreviations of the Legend section are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading. The term Composition refers to the number of various classes of carbohydrates that make up the glycan. The quantity for each class of carbohydrate is depicted as a number in parenthesis to the right of an abbreviation that corresponds to the class of the carbohydrate. These abbreviations are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine.

In some embodiments, the glycan structure of the peptide sequence comprises a glycan structure GL number in accordance with Table 13A or Table 13B, wherein the glycan structure comprises a composition in accordance with the glycan structure GL number and Table 15. For example, glycopeptide HPT (241)—4310 set forth by SEQ ID NO:19 describes the Glycan Structure GL No 4310 attached to the peptide at position 241 with respect to the position on the protein HPT (or position 6 of the listing peptide sequence), wherein the Glycan Structure GL No 4310 refers to Hex(4)HexNAc(3)Fuc(1)NeuAc(0) in accordance with Table 15.

In some embodiments, the glycan structure of the peptide sequence comprises a glycan structure GL number in accordance with Table 13A or Table 13B, wherein the glycan structure comprises a symbol structure in accordance with the glycan structure GL number and Table 15. For example, glycopeptide HPT (241)—4310 set forth by SEQ ID NO:19 describes the Glycan Structure GL No 4310 attached to the peptide at position 241 with respect to the position on the protein HPT (or position 6 of the listing peptide sequence moving from left to right), wherein the Glycan Structure GL No 4310 refers to the symbol structure provided in Table 15.

### X.IV. Methods of Sample Preparation and Analysis for Obtaining Biomarkers for Colorectal Cancer (CRC)

In some embodiments, the method of identifying one or more glycopeptide biomarkers associated with colorectal cancer (CRC) comprises obtaining a biological sample from a first set of one or more individuals with CRC and a second control biological sample from a second set of one or more individuals who do not have CRC. The biological samples may each be subsequently digested, enriched, and analyzed for quantification of at least one glycopeptide.

In some embodiments, the method of identifying one or more glycopeptide biomarker associated with colorectal cancer comprises obtaining a first set of biological samples from one or more individuals with colorectal cancer and a second set of control biological samples from one or more individuals who do not have colorectal cancer. In some embodiments, the method comprises digesting the first set of biological samples and the second set of control biological samples with a protease. In some embodiments, the method comprises enriching the first set of biological samples and the second set of control biological samples for at least one glycopeptide. In some embodiments, the enriching the first set of biological samples and the second set of control biological samples for the at least one glycopeptide is performed after the digesting the biological sample and the control sample with the protease. In some embodiments, the enriching the first set of biological samples or the second set of control biological samples for the at least one glycopeptide is performed after the digesting the biological sample or the control sample with the protease. In some embodiments, the method comprises performing liquid chromatography mass spectrometry (LC/MS) on the first set of biological samples and the second set of control biological samples to identify glycopeptides present in the first set of biological samples and second set of control samples. In some embodiments, the method comprises determining which glycopeptides are present in the first set of biological samples and are not present in the second set of control samples, and thereby identifying one or more glycopeptide biomarker associated with colorectal cancer.

In some embodiments, the first set of biological samples and second set of control biological samples each comprise biological samples from at least three individuals. In some embodiments, the one or more glycopeptide biomarkers associated with colorectal cancer are present in biological samples from at least three individuals with colorectal cancer. In some embodiments, the first set of biological samples and second set of control biological samples each comprise biological samples from at least four individuals. In some embodiments, the one or more glycopeptide biomarkers associated with colorectal cancer are present in biological samples from at least four individuals with colorectal cancer. In some embodiments, the first set of biological samples and second set of control biological samples each comprise biological samples from at least five individuals. In some embodiments, the one or more glycopeptide biomarkers associated with colorectal cancer are present in biological samples from at least five individuals with colorectal cancer.

In some embodiments, the one or more glycopeptide biomarkers associated with colorectal cancer are present in at least 30%, at least 40%, at least 50%, at least 60%, at least 70%, at least 80%, or at least 90% of the first set of biological samples from the individuals with colorectal cancer. In some embodiments, the one or more glycopeptide biomarkers associated with colorectal cancer are present in about 30%, 40%, 50%, 60%, 70%, 80%, or 90% of the first set of biological samples from the individuals with colorectal cancer. In some embodiments, the one or more glycopeptide biomarkers associated with colorectal cancer are present in at least 30% of the first set of biological samples from the individuals with colorectal cancer. In some embodiments, the one or more glycopeptide biomarkers associated with colorectal cancer are present in at least 50% of the first set of biological samples from the individuals with colorectal cancer. In some embodiments, the one or more glycopeptide biomarkers associated with colorectal cancer are present in at least 70% of the first set of biological samples from the individuals with colorectal cancer. In some embodiments, the one or more glycopeptide biomarkers associated with colorectal cancer are present in at least 90% of the first set of biological samples from the individuals with colorectal cancer.

In some embodiments, the one or more glycopeptide biomarkers associated with colorectal cancer are present in less than 50%, less than 40%, less than 30%, less than 20%, less than 15%, less than 10%, less than 5%, or less than 1% of the second set of control biological samples from the individuals who do not have colorectal cancer. In some embodiments, the one or more glycopeptide biomarkers associated with colorectal cancer are present in about 50%, 40%, 30%, 20%, 15%, 10%, 5%, or 1% of the second set of control biological samples from the individuals who do not have colorectal cancer. In some embodiments, the one or more glycopeptide biomarkers associated with colorectal cancer are present in less than 30% of the second set of control biological samples from the individuals who do not have colorectal cancer. In some embodiments, the one or more glycopeptide biomarkers associated with colorectal cancer are present in less than 20% of the second set of control biological samples from the individuals who do not have colorectal cancer. In some embodiments, the one or more glycopeptide biomarkers associated with colorectal cancer are present in less than 10% of the second set of control biological samples from the individuals who do not have colorectal cancer. In some embodiments, the one or more glycopeptide biomarkers associated with colorectal cancer are present in less than 5% of the second set of control biological samples from the individuals who do not have colorectal cancer. In some embodiments, the one or more glycopeptide biomarkers associated with colorectal cancer are undetectable in the second set of control biological samples from the individuals who do not have colorectal cancer.

In some embodiments, the method further comprises denaturing the first set of biological samples and the second set of control biological samples prior to digesting first set of biological samples and the second set of control biological samples. In some embodiments, the denaturing the first set of biological samples and the second set of control biological samples comprises heating the first set of biological samples and the second set of control biological samples to at least 100° C. In some embodiments, the method further comprises reducing the first set of biological samples and the second set of control biological samples after denaturing the first set of biological samples and the second set of control biological samples prior to digesting the first set of biological samples and the second set of control biological samples. In some embodiments, the reducing the first set of biological samples and the second set of control biological samples comprises incubating the first set of biological samples and the second set of control biological samples with a reducing agent. In some embodiments, the reducing agent is dithiothreitol (DTT). In some embodiments, the method further comprises incubating the first set of biological samples and the second set of control biological samples with an alkylating agent following reducing the first set of biological samples and the second set of control biological samples, and then, quenching a remaining portion of the alkylating agent with DTT for both the first set of biological samples and the second set of control biological samples prior to digesting the first set of biological samples and the second set of control biological samples.

In some embodiments, digestion of a biological sample comprises digestion with one or more proteases. In some embodiments, one or more of the proteases are serine proteases. In some embodiments, the one or more proteases are chosen from the group comprising trypsin and endoproteinase LysC. In some embodiments, digestion of a biological sample is quenched and then halted by mixing an acid with the protease to form a proteolytic digest. In some embodiments, digestion of a biological sample is preceded by denaturing the biological sample. In some embodiments, the denaturation comprises heating the biological sample to at least 70° C., 80° C., 90° C., or 100° C. In some embodiments, the denaturation comprises heating the biological sample to at least 100° C. In some embodiments, the denaturation comprises heating the biological sample for at least 5, at least 10, at least 15, at least 20, at least 25, or at least 30 minutes. In some embodiments, the denaturation comprises heating the biological sample for at least 5 minutes. In some embodiments, denaturation further comprises the step of centrifuging the denatured biological sample. In some embodiments, the biological sample is reduced with one or more reducing agents after denaturation and prior to digestion. In some embodiments, the one or more reducing agents comprise dithiothreitol (DTT), 2-mercaptoethanol, and 2-mercaptoethylamine-HCl. In some embodiments, the biological sample is alkylated via incubation with one or more alkylating agents after reduction and prior to digestion. In some embodiments, the one or more alkylating agents comprises iodoacetamide (IAA) and iodoacetate. In some embodiments, the biological samples are incubated with one or more alkylating agents for at least 30 minutes, at least 1 hour, at least 2, hours, or at least 4 hours. In some embodiments, the biological samples are incubated with one or more alkylating agents for at least 30 minutes. In some embodiments, the alkylation of the biological sample is quenched with DTT.

In some embodiments, the method further comprises enriching for glycopeptides comprises loading the proteolytic digest onto a HILIC (hydrophilic interaction liquid chromatography) column, washing the HILIC column with a wash liquid, and eluting an enriched glycopeptide eluate from the HILIC column with an eluting liquid. In some embodiments, the HILIC sorbent material is HILICON-iSPE. In some embodiments, the enriching the first set of biological samples and the second set of control biological samples for the at least one glycopeptide is performed after the digesting the first set of biological samples and the second set of control biological samples with the protease.

In some embodiments, a glycopeptide concentration for a glycopeptide derived from the proteolytic digest sample is enriched by a factor of at least 5, at least 10, at least 15, at least 20, at least 25, at least 30, at least 40, at least 50, or greater with respect to a peptide concentration where the peptide concentration represents an amount of a peptide that is associated with the same protein as the glycopeptide. In some embodiments, a glycopeptide concentration for a glycopeptide derived from the proteolytic digest sample is enriched by a factor of between 5 and 100, 10 and 90, 20 and 80, 30 and 70, or 40 and 60 greater with respect to a peptide concentration where the peptide concentration represents an amount of a peptide that is associated with the same protein as the glycopeptide. In some embodiments, a glycopeptide concentration for a glycopeptide derived from the proteolytic digest sample is enriched by a factor of at least 30 with respect to a peptide concentration where the peptide concentration represents an amount of a peptide that is associated with the same protein as the glycopeptide. In some embodiments, a glycopeptide concentration for a glycopeptide derived from the proteolytic digest sample is enriched by a factor of 30 or greater with respect to a peptide concentration where the peptide concentration represents an amount of a peptide that is associated with the same protein as the glycopeptide.

In some embodiments, the performing liquid chromatography mass spectrometry (LC/MS). In some embodiments, the performing liquid chromatography mass spectrometry (LC/MS) uses an ion trap mass analyzer. In some embodiments, the ion trap mass analyzer comprising an outer barrel-like electrode and a coaxial inner spindle-like electrode. In some embodiments, the ion trap mass analyzer is configured to trap ions in an orbital motion around the spindle.

In some embodiments, the at least one glycopeptide that is enriched from a digested biological sample may be used to diagnose an individual having colorectal cancer (CRC). Sample processing and enrichment of a biological sample according the methods described herein precede sample analysis of the biological sample to determine the presence and/or amount of at least one glycopeptide. In some embodiments, a control sample is a sample from one or more individuals who do not have colorectal cancer. In some embodiments, the control sample is processed and enriched in the same way as the biological sample for comparison of the presence and/or amount of at least one glycopeptide. In some embodiments, the at least one glycopeptide is a glycopeptide structures from Table 13A or Table 13B. In some embodiments, the at least one glycopeptide is a glycopeptide comprising the amino acid sequence of SEQ ID NOs: 168-198. In some embodiments, the at least one glycopeptide is a glycopeptide comprising the amino acid sequence of SEQ ID NOs: 168, 171, 172, 176, 181, 184, 187, 192, and 194.

In some embodiments, the presence and/or amount of at least one glycopeptide that is enriched from the digested biological sample and the control sample may be used to diagnose an individual having colorectal cancer (CRC). In some embodiments, the presence and/or amount of at least one glycopeptide that is enriched from the digested biological sample and the control sample may be used to diagnose an individual suspected of having colorectal cancer (CRC). In some embodiments, the presence and/or amount of at least one glycopeptide that is enriched from the digested biological sample and the control sample may be used to diagnose an individual having not had an endoscopy, structural exam or a stool-based test within the past 6-12 months. In some embodiments, the presence of at least one glycopeptide in the biological sample and the absence of the same glycopeptide in the control sample may be used to diagnose an individual having or suspected of having CRC.

### X.V. Methods of Diagnosing

In some embodiments, the methods provided herein are useful for diagnosing CRC. In some embodiments the method comprises determining a risk of developing CRC. In some embodiments, a diagnosis of CRC is provided, for example, where an individual is determined to have early-stage CRC, late-stage CRC, or severe CRC.

In some embodiments, the diagnosis is based upon presence and/or amount of at least one, at least two, at least three, at least four, at least five, at least six, at least seven, at least eight, at least nine, at least 10, at least 15, at least 20, at least 25, at least 30, or 31 peptide structures from Table 13A. In some embodiments, the presence and/or amount of the peptide is determined using mass spectrometry. In some embodiments, the diagnosis is based upon the presence and/or amount of one or more peptides comprising the amino acid sequence of SEQ ID NOs: 168-198. In some embodiments, the diagnosis is based upon the presence and/or amount of two or more peptides comprising the amino acid sequence of SEQ ID NOs: 168-198. In some embodiments, the diagnosis is based upon the presence and/or amount of three or more peptides comprising the amino acid sequence of SEQ ID NOs: 168-198. In some embodiments, the diagnosis is based upon the presence and/or amount of four or more peptides comprising the amino acid sequence of SEQ ID NOs: 168-198. In some embodiments, the diagnosis is based upon the presence and/or amount of five or more peptides comprising the amino acid sequence of SEQ ID NOs: 168-198. In some embodiments, the diagnosis is based upon the presence and/or amount of six or more peptides comprising the amino acid sequence of SEQ ID NOs: 168-198. In some embodiments, the diagnosis is based upon the presence and/or amount of seven or more peptides comprising the amino acid sequence of SEQ ID NOs: 168-198. In some embodiments, the diagnosis is based upon the presence and/or amount of eight or more peptides comprising the amino acid sequence of SEQ ID NOs: 168-198. In some embodiments, the diagnosis is based upon the presence and/or amount of nine or more peptides comprising the amino acid sequence of SEQ ID NOs: 168-198. In some embodiments, the diagnosis is based upon the presence and/or amount of ten or more peptides comprising the amino acid sequence of SEQ ID NOs: 168-198. In some embodiments, the diagnosis is based upon the presence and/or amount of fifteen or more peptides comprising the amino acid sequence of SEQ ID NOs: 168-198. In some embodiments, the diagnosis is based upon the presence and/or amount of twenty or more peptides comprising the amino acid sequence of SEQ ID NOs: 168-198. In some embodiments, the diagnosis is based upon the presence and/or amount of twenty-five or more peptides comprising the amino acid sequence of SEQ ID NOs: 168-198. In some embodiments, the diagnosis is based upon the presence and/or amount of thirty or more peptides comprising the amino acid sequence of SEQ ID NOs: 168-198. In some embodiments, the diagnosis is based upon the presence and/or amount of each of the peptides comprising the amino acid sequence of SEQ ID NOs: 168-198. In some embodiments, the presence and/or amount of the peptide is determined using mass spectrometry.

In some embodiments, the risk of CRC is determined based upon presence and/or amount of at least one, at least two, at least three, at least four, at least five, at least six, at least seven, at least eight, at least nine, at least 10, at least 15, at least 20, at least 25, at least 30, or 31 peptide structures from Table 13A. In some embodiments, the presence and/or amount of the peptide is determined using mass spectrometry. In some embodiments, the risk is determined based upon the presence and/or amount of one or more peptides comprising the amino acid sequence of SEQ ID NOs: 168-198. In some embodiments, the risk is determined based upon the presence and/or amount of two or more peptides comprising the amino acid sequence of SEQ ID NOs: 168-198. In some embodiments, the risk is determined based upon the presence and/or amount of three or more peptides comprising the amino acid sequence of SEQ ID NOs: 168-198. In some embodiments, the risk is determined based upon the presence and/or amount of four or more peptides comprising the amino acid sequence of SEQ ID NOs: 168-198. In some embodiments, the risk is determined based upon the presence and/or amount of five or more peptides comprising the amino acid sequence of SEQ ID NOs: 168-198. In some embodiments, the risk is determined based upon the presence and/or amount of six or more peptides comprising the amino acid sequence of SEQ ID NOs: 168-198. In some embodiments, the risk is determined based upon the presence and/or amount of seven or more peptides comprising the amino acid sequence of SEQ ID NOs: 168-198. In some embodiments, the risk is determined based upon the presence and/or amount of eight or more peptides comprising the amino acid sequence of SEQ ID NOs: 168-198. In some embodiments, the risk is determined based upon the presence and/or amount of nine or more peptides comprising the amino acid sequence of SEQ ID NOs: 168-198. In some embodiments, the risk is determined based upon the presence and/or amount of ten or more peptides comprising the amino acid sequence of SEQ ID NOs: 168-198. In some embodiments, the risk is determined based upon the presence and/or amount of fifteen or more peptides comprising the amino acid sequence of SEQ ID NOs: 168-198. In some embodiments, the risk is determined based upon the presence and/or amount of twenty or more peptides comprising the amino acid sequence of SEQ ID NOs: 168-198. In some embodiments, the risk is determined based upon the presence and/or amount of twenty-five or more peptides comprising the amino acid sequence of SEQ ID NOs: 168-198. In some embodiments, the risk is determined based upon the presence and/or amount of thirty or more peptides comprising the amino acid sequence of SEQ ID NOs: 168-198. In some embodiments, the risk is determined based upon the presence and/or amount of each of the peptides comprising the amino acid sequence of SEQ ID NOs: 168-198. In some embodiments, the presence and/or amount of the peptide is determined using mass spectrometry. In some embodiments, the risk for CRC is determined to be low or high, or on a spectrum of low to high. In some embodiments, if the individual is determined to be at high risk for CRC an endoscopy is recommended. In some embodiments, if the individual's risk is above a set threshold, an endoscopy is recommended.

In some embodiments, the diagnosis is based upon presence and/or amount of at least one, at least two, at least three, at least four, at least five, at least six, at least seven, at least eight, or nine peptide structures from Table 13B. In some embodiments, the presence and/or amount of the peptide is determined using mass spectrometry. In some embodiments, the diagnosis is based upon the presence and/or amount of one or more peptides comprising the amino acid sequence of SEQ ID NOs: 168, 171, 172, 176, 181, 184, 187, 192, and 194. In some embodiments, the diagnosis is based upon the presence and/or amount of two or more peptides comprising the amino acid sequence of SEQ ID NOs: 168, 171, 172, 176, 181, 184, 187, 192, and 194. In some embodiments, the diagnosis is based upon the presence and/or amount of three or more peptides comprising the amino acid sequence of SEQ ID NOs: 168, 171, 172, 176, 181, 184, 187, 192, and 194. In some embodiments, the diagnosis is based upon the presence and/or amount of four or more peptides comprising the amino acid sequence of SEQ ID NOs: 168, 171, 172, 176, 181, 184, 187, 192, and 194. In some embodiments, the diagnosis is based upon the presence and/or amount of five or more peptides comprising the amino acid sequence of SEQ ID NOs: 168, 171, 172, 176, 181, 184, 187, 192, and 194. In some embodiments, the diagnosis is based upon the presence and/or amount of six or more peptides comprising the amino acid sequence of SEQ ID NOs: 168, 171, 172, 176, 181, 184, 187, 192, and 194. In some embodiments, the diagnosis is based upon the presence and/or amount of seven or more peptides comprising the amino acid sequence of SEQ ID NOs: 168, 171, 172, 176, 181, 184, 187, 192, and 194. In some embodiments, the diagnosis is based upon the presence and/or amount of eight or more peptides comprising the amino acid sequence of SEQ ID NOs: 168, 171, 172, 176, 181, 184, 187, 192, and 194. In some embodiments, the diagnosis is based upon the presence and/or amount of each of the peptides comprising the amino acid sequence of SEQ ID NOs: 168, 171, 172, 176, 181, 184, 187, 192, and 194. In some embodiments, the presence and/or amount of the peptide is determined using mass spectrometry.

In some embodiments, the diagnosis is based upon the presence and/or amount of one or more glycoproteins comprising Haptoglobin (HPT), Alpha-1-antitrypsin (A1AT), Alpha-2-macroglobulin (A2MG), Complement C5 (CO5), Polymeric immunoglobulin receptor (PIGR), Immunoglobulin heavy constant gamma 1 (IGHG1), Immunoglobulin heavy constant gamma 2 (IGHG2), Immunoglobulin heavy constant gamma 4 (IGHG4), Immunoglobulin heavy constant alpha 1 (IGHA1), Immunoglobulin heavy constant alpha 2 (IGHA2), Serum amyloid P-component (SAMP), Complement component C9 (C09), Serotransferrin (TRFE), Apolipoprotein B-100 (APOB), Complement C4-A (CO4A), Clusterin (CLUS), Complement component C6 (CO6), and Inter-alpha-trypsin inhibitor heavy chain H4 (ITIH4). In some embodiments, the diagnosis is based upon the presence and/or amount of one or more glycosylated proteins comprising HPT, A1AT, A2MG, CO5, PIGR, IGHG1, IGHG2, IGHG4, IGHA1, IGHA2, SAMP, CO9, TRFE, APOB, CO4A, CLUS, CO6, and ITIH4. In some embodiments, the diagnosis is based upon the presence and/or amount of one or more glycoprotein set forth in SEQ ID NOs: 3, 13, 18, 19, 122, 132, 134, 157, 158, 159, 160, 161, 162, 163, 164, 165, 166, and 167. In some embodiments, the diagnosis is based upon the presence and/or amount of one or more glycopeptides originating from one or more glycosylated proteins. In some embodiments, the diagnosis is based upon the presence and/or amount of one or more of SEQ ID NOs: 168-198. In some embodiments, the diagnosis is based upon the presence and/or amount of one or more of SEQ ID NOs: 168, 171, 172, 176, 181, 184, 187, 192, and 194.

In some embodiments, the diagnosis is based upon the presence and/or amount of one or more glycopeptides originating from one or more of HPT, A1AT, A2MG, IGHG1, IGHG2, or CO4A. In some embodiments, the diagnosis is based upon the presence and/or amount of one or more glycopeptides originating from HPT. In some embodiments, the diagnosis is based upon the presence and/or amount of one or more of SEQ ID NOs: 168-172. In some embodiments, the diagnosis is based upon the presence and/or amount of one or more glycopeptides originating from A1AT. In some embodiments, the diagnosis is based upon the presence and/or amount of one or more of SEQ ID NOs: 173-177. In some embodiments, the diagnosis is based upon the presence and/or amount of one or more glycopeptides originating from A2MG. In some embodiments, the diagnosis is based upon the presence and/or amount of one or more of SEQ ID NOs: 178-180. In some embodiments, the diagnosis is based upon the presence and/or amount of one or more glycopeptides originating from IGHG1. In some embodiments, the diagnosis is based upon the presence and/or amount of one or more of SEQ ID NOs: 183-184. In some embodiments, the diagnosis is based upon the presence and/or amount of one or more glycopeptides originating from IGHG2. In some embodiments, the diagnosis is based upon the presence and/or amount of one or more of SEQ ID NOs: 185-186. In some embodiments, the diagnosis is based upon the presence and/or amount of one or more glycopeptides originating from CO4A. In some embodiments, the diagnosis is based upon the presence and/or amount of one or more of SEQ ID NOs: 194-195.

In some embodiments, the diagnosis is based upon the presence and/or amount of one or more glycopeptides originating from one or more related glycoproteins. In some embodiments, the diagnosis is based upon the presence and/or amount of one or more glycopeptides originating from IGHG1, IGHG2, IGHG4, IGHA1, or IGA2. In some embodiments, the diagnosis is based upon the presence and/or amount of one or more of SEQ ID NOs:183-189. In some embodiments, the diagnosis is based upon the presence and/or amount of one or more glycopeptides originating from CO5, CO9, CO4A, or CO6. In some embodiments, the diagnosis is based upon the presence and/or amount of one or more of SEQ ID NOs:181, 188, 194, 195, and 197.

In some embodiments, the diagnosis is based upon presence and/or amount of at least one, at least two, at least three, at least four, at least five, at least six, at least seven, at least eight, at least nine, at least 10, at least 15, at least 20, at least 25, at least 30, or 31 peptide structures from Table 13A. In some embodiments, the diagnosis is based upon the presence and/or amount of one or more peptides consisting of the amino acid sequence of SEQ ID NOs: 168-198. In some embodiments, the diagnosis is based upon the presence and/or amount of two or more peptides consisting of the amino acid sequence of SEQ ID NOs: 168-198. In some embodiments, the diagnosis is based upon the presence and/or amount of three or more peptides consisting of the amino acid sequence of SEQ ID NOs: 168-198. In some embodiments, the diagnosis is based upon the presence and/or amount of four or more peptides consisting of the amino acid sequence of SEQ ID NOs: 168-198. In some embodiments, the diagnosis is based upon the presence and/or amount of five or more peptides consisting of the amino acid sequence of SEQ ID NOs: 168-198. In some embodiments, the diagnosis is based upon the presence and/or amount of six or more peptides consisting of the amino acid sequence of SEQ ID NOs: 168-198. In some embodiments, the diagnosis is based upon the presence and/or amount of seven or more peptides consisting of the amino acid sequence of SEQ ID NOs: 168-198. In some embodiments, the diagnosis is based upon the presence and/or amount of eight or more peptides consisting of the amino acid sequence of SEQ ID NOs: 168-198. In some embodiments, the diagnosis is based upon the presence and/or amount of nine or more peptides consisting of the amino acid sequence of SEQ ID NOs: 168-198. In some embodiments, the diagnosis is based upon the presence and/or amount of ten or more peptides consisting of the amino acid sequence of SEQ ID NOs: 168-198. In some embodiments, the diagnosis is based upon the presence and/or amount of fifteen or more peptides consisting of the amino acid sequence of SEQ ID NOs: 168-198. In some embodiments, the diagnosis is based upon the presence and/or amount of twenty or more peptides consisting of the amino acid sequence of SEQ ID NOs: 168-198. In some embodiments, the diagnosis is based upon the presence and/or amount of twenty-five or more peptides consisting of the amino acid sequence of SEQ ID NOs: 168-198. In some embodiments, the diagnosis is based upon the presence and/or amount of thirty or more peptides consisting of the amino acid sequence of SEQ ID NOs: 168-198. In some embodiments, the diagnosis is based upon the presence and/or amount of each of the peptides consisting of the amino acid sequence of SEQ ID NOs: 168-198. In some embodiments, the presence and/or amount of the peptide is determined using mass spectrometry.

In some embodiments, the diagnosis is based upon presence and/or amount of at least one, at least two, at least three, at least four, at least five, at least six, at least seven, at least eight, or nine peptide structures from Table 13B. In some embodiments, the diagnosis is based upon the presence and/or amount of one or more peptides consisting of the amino acid sequence of SEQ ID NOs: 168, 171, 172, 176, 181, 184, 187, 192, and 194. In some embodiments, the diagnosis is based upon the presence and/or amount of two or more peptides consisting of the amino acid sequence of SEQ ID NOs: 168, 171, 172, 176, 181, 184, 187, 192, and 194. In some embodiments, the diagnosis is based upon the presence and/or amount of three or more peptides consisting of the amino acid sequence of SEQ ID NOs: 168, 171, 172, 176, 181, 184, 187, 192, and 194. In some embodiments, the diagnosis is based upon the presence and/or amount of four or more peptides consisting of the amino acid sequence of SEQ ID NOs: 168, 171, 172, 176, 181, 184, 187, 192, and 194. In some embodiments, the diagnosis is based upon the presence and/or amount of five or more peptides consisting of the amino acid sequence of SEQ ID NOs: 168, 171, 172, 176, 181, 184, 187, 192, and 194. In some embodiments, the diagnosis is based upon the presence and/or amount of six or more peptides consisting of the amino acid sequence of SEQ ID NOs: 168, 171, 172, 176, 181, 184, 187, 192, and 194. In some embodiments, the diagnosis is based upon the presence and/or amount of seven or more peptides consisting of the amino acid sequence of SEQ ID NOs: 168, 171, 172, 176, 181, 184, 187, 192, and 194. In some embodiments, the diagnosis is based upon the presence and/or amount of eight or more peptides consisting of the amino acid sequence of SEQ ID NOs: 168, 171, 172, 176, 181, 184, 187, 192, and 194. In some embodiments, the diagnosis is based upon the presence and/or amount of each of the peptides consisting of the amino acid sequence of SEQ ID NOs: 168, 171, 172, 176, 181, 184, 187, 192, and 194. In some embodiments, the presence and/or amount of the peptide is determined using mass spectrometry.

In some embodiments, the risk of CRC is determined based upon presence and/or amount of at least one, at least two, at least three, at least four, at least five, at least six, at least seven, at least eight, or nine peptide structures from Table 13B. In some embodiments, the risk is determined based upon the presence and/or amount of one or more peptides consisting of the amino acid sequence of SEQ ID NOs: 168, 171, 172, 176, 181, 184, 187, 192, and 194. In some embodiments, the risk is determined based upon the presence and/or amount of two or more peptides consisting of the amino acid sequence of SEQ ID NOs: 168, 171, 172, 176, 181, 184, 187, 192, and 194. In some embodiments, the risk is determined based upon the presence and/or amount of three or more peptides consisting of the amino acid sequence of SEQ ID NOs: 168, 171, 172, 176, 181, 184, 187, 192, and 194. In some embodiments, the risk is determined based upon the presence and/or amount of four or more peptides consisting of the amino acid sequence of SEQ ID NOs: 168, 171, 172, 176, 181, 184, 187, 192, and 194. In some embodiments, the risk is determined based upon the presence and/or amount of five or more peptides consisting of the amino acid sequence of SEQ ID NOs: 168, 171, 172, 176, 181, 184, 187, 192, and 194. In some embodiments, the risk is determined based upon the presence and/or amount of six or more peptides consisting of the amino acid sequence of SEQ ID NOs: 168, 171, 172, 176, 181, 184, 187, 192, and 194. In some embodiments, the risk is determined based upon the presence and/or amount of seven or more peptides consisting of the amino acid sequence of SEQ ID NOs: 168, 171, 172, 176, 181, 184, 187, 192, and 194. In some embodiments, the risk is determined based upon the presence and/or amount of eight or more peptides consisting of the amino acid sequence of SEQ ID NOs: 168, 171, 172, 176, 181, 184, 187, 192, and 194. In some embodiments, the risk is determined based upon the presence and/or amount of each of the peptides consisting of the amino acid sequence of SEQ ID NOs: 168, 171, 172, 176, 181, 184, 187, 192, and 194. In some embodiments, the presence and/or amount of the peptide is determined using mass spectrometry. In some embodiments, the risk for CRC is determined to be low or high, or on a spectrum of low to high. In some embodiments, if the individual is determined to be at high risk for CRC an endoscopy is recommended. In some embodiments, if the individual's risk is above a set threshold, an endoscopy is recommended.

In some embodiments, the diagnosis is based upon presence and/or amount of at least one, at least two, at least three, at least four, at least five, at least six, at least seven, at least eight, or nine peptide structures from Table 13B. In some embodiments, the diagnosis is based upon the presence and/or amount of one or more peptides consisting of the amino acid sequence of SEQ ID NOs: 168, 171, 172, 176, 181, 184, 187, 192, and 194. In some embodiments, the diagnosis is based upon the presence and/or amount of two or more peptides consisting of the amino acid sequence of SEQ ID NOs: 168, 171, 172, 176, 181, 184, 187, 192, and 194. In some embodiments, the diagnosis is based upon the presence and/or amount of three or more peptides comprising of the amino acid sequence of SEQ ID NOs: 168, 171, and 172. In some embodiments, the diagnosis is based upon the presence and/or amount of three or more peptides comprising of the amino acid sequence of SEQ ID NOs: 176, 181, and 184. In some embodiments, the diagnosis is based upon the presence and/or amount of three or more peptides comprising of the amino acid sequence of SEQ ID NOs: 187, 192, and 194. In some embodiments, the diagnosis is based upon the presence and/or amount of three or more peptides comprising of the amino acid sequence of SEQ ID NOs:168, 176, and 187. In some embodiments, the diagnosis is based upon the presence and/or amount of three or more peptides comprising of the amino acid sequence of SEQ ID NOs: 168, 171, and 176. In some embodiments, the diagnosis is based upon the presence and/or amount of three or more peptides comprising of the amino acid sequence of SEQ ID NOs:168, 171, and 181. In some embodiments, the diagnosis is based upon the presence and/or amount of three or more peptides comprising of the amino acid sequence of SEQ ID NOs:168, 171, and 184. In some embodiments, the diagnosis is based upon the presence and/or amount of three or more peptides comprising of the amino acid sequence of SEQ ID NOs: 168, 171, and 187. In some embodiments, the diagnosis is based upon the presence and/or amount of three or more peptides comprising of the amino acid sequence of SEQ ID NOs: 168, 171, and 192. In some embodiments, the diagnosis is based upon the presence and/or amount of three or more peptides comprising of the amino acid sequence of SEQ ID NOs: 168, 171, and 194. In some embodiments, the diagnosis is based upon the presence and/or amount of three or more peptides comprising of the amino acid sequence of SEQ ID NOs: 172, 176, and 181. In some embodiments, the diagnosis is based upon the presence and/or amount of three or more peptides comprising of the amino acid sequence of SEQ ID NOs: 172, 176, and 184. In some embodiments, the diagnosis is based upon the presence and/or amount of three or more peptides comprising of the amino acid sequence of SEQ ID NOs: 172, 176, and 187. In some embodiments, the diagnosis is based upon the presence and/or amount of three or more peptides comprising of the amino acid sequence of SEQ ID NOs: 172, 176, and 192. In some embodiments, the diagnosis is based upon the presence and/or amount of three or more peptides comprising of the amino acid sequence of SEQ ID NOs: 172, 176, and 194. In some embodiments, the diagnosis is based upon the presence and/or amount of three or more peptides comprising of the amino acid sequence of SEQ ID NOs: 181, 184 and 187. In some embodiments, the diagnosis is based upon the presence and/or amount of three or more peptides comprising of the amino acid sequence of SEQ ID NOs: 181, 184, and 192. In some embodiments, the diagnosis is based upon the presence and/or amount of three or more peptides comprising of the amino acid sequence of SEQ ID NOs: 181, 184, and 194. In some embodiments, the diagnosis is based upon the presence and/or amount of three or more peptides comprising of the amino acid sequence of SEQ ID NOs:168, 192, and 194. In some embodiments, the diagnosis is based upon the presence and/or amount of three or more peptides comprising of the amino acid sequence of SEQ ID NOs: 171, 192, and 194. In some embodiments, the diagnosis is based upon the presence and/or amount of three or more peptides comprising of the amino acid sequence of SEQ ID NOs: 172, 192, and 194. In some embodiments, the diagnosis is based upon the presence and/or amount of three or more peptides comprising of the amino acid sequence of SEQ ID NOs:176, 192, and 194. In some embodiments, the diagnosis is based upon the presence and/or amount of three or more peptides comprising of the amino acid sequence of SEQ ID NOs: 181, 192, and 194. In some embodiments, the diagnosis is based upon the presence and/or amount of three or more peptides comprising of the amino acid sequence of SEQ ID NOs: 184, 192, and 194. In some embodiments, the presence and/or amount of the peptide is determined using mass spectrometry.

In some embodiments, the risk of CRC is determined based upon presence and/or amount of at least one, at least two, at least three, at least four, at least five, at least six, at least seven, at least eight, or nine peptide structures from Table 13B. In some embodiments, the risk is determined based upon the presence and/or amount of one or more peptides consisting of the amino acid sequence of SEQ ID NOs: 168, 171, 172, 176, 181, 184, 187, 192, and 194. In some embodiments, the risk is determined based upon the presence and/or amount of two or more peptides consisting of the amino acid sequence of SEQ ID NOs: 168, 171, 172, 176, 181, 184, 187, 192, and 194. In some embodiments, the risk is determined based upon the presence and/or amount of three or more peptides comprising of the amino acid sequence of SEQ ID NOs: 168, 171, 172. In some embodiments, the risk is determined based upon the presence and/or amount of three or more peptides comprising of the amino acid sequence of SEQ ID NOs: 176, 181, and 184. In some embodiments, the risk is determined based upon the presence and/or amount of three or more peptides comprising of the amino acid sequence of SEQ ID NOs: 187, 192, and 194. In some embodiments, the risk is determined based upon the presence and/or amount of three or more peptides comprising of the amino acid sequence of SEQ ID NOs:168, 176, and 187. In some embodiments, the risk is determined based upon the presence and/or amount of three or more peptides comprising of the amino acid sequence of SEQ ID NOs: 168, 171, and 176. In some embodiments, the risk is determined based upon the presence and/or amount of three or more peptides comprising of the amino acid sequence of SEQ ID NOs: 168, 171, and 181. In some embodiments, the risk is determined based upon the presence and/or amount of three or more peptides comprising of the amino acid sequence of SEQ ID NOs: 168, 171, and 184. In some embodiments, the risk is determined based upon the presence and/or amount of three or more peptides comprising of the amino acid sequence of SEQ ID NOs: 168, 171, and 187. In some embodiments, the risk is determined based upon the presence and/or amount of three or more peptides comprising of the amino acid sequence of SEQ ID NOs: 168, 171, and 192. In some embodiments, the risk is determined based upon the presence and/or amount of three or more peptides comprising of the amino acid sequence of SEQ ID NOs: 168, 171, and 194. In some embodiments, the risk is determined based upon the presence and/or amount of three or more peptides comprising of the amino acid sequence of SEQ ID NOs: 172, 176, and. In some embodiments, the risk is determined based upon the presence and/or amount of three or more peptides comprising of the amino acid sequence of SEQ ID NOs: 172, 176, and 184. In some embodiments, the risk is determined based upon the presence and/or amount of three or more peptides comprising of the amino acid sequence of SEQ ID NOs: 172, 176, and 187. In some embodiments, the risk is determined based upon the presence and/or amount of three or more peptides comprising of the amino acid sequence of SEQ ID NOs: 172, 176, and 192. In some embodiments, the risk is determined based upon the presence and/or amount of three or more peptides comprising of the amino acid sequence of SEQ ID NOs: 172, 176, and 194. In some embodiments, the risk is determined based upon the presence and/or amount of three or more peptides comprising of the amino acid sequence of SEQ ID NOs: 181, 184, and 187. In some embodiments, the risk is determined based upon the presence and/or amount of three or more peptides comprising of the amino acid sequence of SEQ ID NOs: 181, 184, and 192. In some embodiments, the risk is determined based upon the presence and/or amount of three or more peptides comprising of the amino acid sequence of SEQ ID NOs: 181, 184, and 194. In some embodiments, the risk is determined based upon the presence and/or amount of three or more peptides comprising of the amino acid sequence of SEQ ID NOs: 168, 192, and 194. In some embodiments, the risk is determined based upon the presence and/or amount of three or more peptides comprising of the amino acid sequence of SEQ ID NOs: 171, 192, and 194. In some embodiments, the risk is determined based upon the presence and/or amount of three or more peptides comprising of the amino acid sequence of SEQ ID NOs: 172, 192, and 194. In some embodiments, the risk is determined based upon the presence and/or amount of three or more peptides comprising of the amino acid sequence of SEQ ID NOs: 176, 192, and 194. In some embodiments, the risk is determined based upon the presence and/or amount of three or more peptides comprising of the amino acid sequence of SEQ ID NOs: 181, 192, and 194. In some embodiments, the risk is determined based upon the presence and/or amount of three or more peptides comprising of the amino acid sequence of SEQ ID NOs: 184, 192, and 194. In some embodiments, the presence and/or amount of the peptide is determined using mass spectrometry. In some embodiments, the risk for CRC is determined to be low or high, or on a spectrum of low to high. In some embodiments, if the individual is determined to be at high risk for CRC an endoscopy is recommended. In some embodiments, if the individual's risk is above a set threshold, an endoscopy is recommended.

In some embodiments, the method further comprises collecting a biological sample. In some embodiments, the method comprises collecting a blood sample. In some embodiments, the method comprises collecting a serum sample. In some embodiments, the method comprises collecting a serum sample. In some embodiments, the method comprises collecting a stool sample.

For example, in certain embodiments, the presence or amount of the at least one peptide structure is detected using mass spectrometry, ELISA, MRM mass spectrometry, or data dependent acquisition (DDA)-MS. In one embodiment, the at least one peptide structure is none, or below a detection limit. In one embodiment, the colorectal cancer (CRC) is early-stage CRC. In one embodiment, the CRC is late-stage CRC. In one embodiment, the CRC is severe CRC. In one embodiment, the at least one peptide structure comprises three or more peptide structures identified in Table 13A. In one embodiment, the at least one peptide structure comprises three or more peptide structures identified in Table 13B.

In certain embodiments, the present methods comprise assessing one or more risk factors or clinical indicators of the colorectal cancer (CRC), in which a clinical indicator of CRC is selected from the group consisting of changes in bowel habits, bloody stool, diarrhea, constipation, persistent abdominal pain, persistent abdominal cramps, and unexplained weight loss. In certain embodiments, the risk factor for CRC is selected from the group consisting of age, irritable bowel disease, type 2 diabetes, a family history of CRC, a genetic syndrome (e.g., Lynch syndrome), obesity, smoking, tobacco use, alcohol consumption, dietary choices, and limited physical activity. In some embodiments, the individual at risk of developing CRC is at least 35, 40, 45, 50, 55, 60, 65, or 70 years of age. In some embodiments, the individual at risk of developing CRC is at least 35 years of age. In some embodiments, the individual at risk of developing CRC is at least 50 years of age. In some embodiments, the individual at risk of developing CRC has a genetic syndrome, wherein the genetic syndrome comprises familial adenomatous polyposis (FAP) or hereditary non-polyposis colorectal cancer (Lynch syndrome). In some embodiments, the individual at risk of developing CRC consumes an abundance of red or processed meat and/or an limited amount of vegetables and fiber. In certain embodiments, the individual is determined have a healthy state, in which a healthy state may include the absence of CRC and/or a low risk for CRC.

### X.VI. Methods of Treatment

In some embodiments, provided herein are methods of treating colorectal cancer (CRC) based upon the presence and/or amount of one or more biomarkers provided herein. In some embodiments, the method further comprises administering an effective amount of a therapy for CRC. In some embodiments, the method further comprises selecting a particular therapy based upon the disease indicator.

In some embodiments, provided herein are methods of determining a risk of an individual for developing colorectal cancer (CRC) based upon the presence and/or amount of one or more biomarkers provided in Table 13A or Table 13B. In some embodiments, a specific treatment is selected based upon a determine risk for an individual suspected of having colorectal cancer (CRC). In some embodiments, a determined risk corresponding to a higher risk of developing CRC results in selection of a therapy for treating CRC. In some embodiments, a determined risk corresponding to a lower risk of developing CRC results in selection of no therapy for treating CRC.

In some embodiments, provided herein is a method of diagnosing and/or treating colorectal cancer (CRC) comprising detecting the presence and/or amount of at least one peptide structure from Table 13A and selecting a CRC therapy. In some embodiments, the diagnosis and/or treatment is based upon the presence and/or amount of at least one, at least two, at least three, at least four, at least five, at least six, at least seven, at least eight, at least nine, at least 10, at least 15, at least 20, at least 25, at least 30, or 31 peptide structures from Table 13A. In some embodiments, method of diagnosing and/or treating CRC further comprises administering an effective amount of a CRC therapy to the individual based upon the presence and/or amount of at least one peptide structure from Table 13A.

In some embodiments, provided herein is a method of diagnosing and/or treating colorectal cancer (CRC) comprising detecting the presence and/or amount of at least one peptide structure from Table 13B and selecting a CRC therapy. In some embodiments, the diagnosis and/or treatment is based upon the presence and/or amount of at least one, at least two, at least three, at least four, at least five, at least six, at least seven, at least eight, or nine peptide structures from Table 13B. In some embodiments, method of diagnosing and/or treating CRC further comprises administering an effective amount of a CRC therapy to the individual based upon the presence and/or amount of at least one peptide structure from Table 13B.

In some embodiments, provided herein is a method of treating colorectal cancer (CRC) comprising detecting the presence and/or amount of at least one peptide structure from Table 13A and selecting a CRC therapy. In some embodiments, method of treating CRC further comprises administering an effective amount of a CRC therapy to the individual based upon the presence and/or amount of at least one peptide structure from Table 13A. In some embodiments, the diagnosis and/or treatment is based upon the presence and/or amount of at least two, at least three, at least four, at least five, at least 10, at least 15, at least 20, at least 25, at least 30, or 31 peptide structures from Table 13A.

In some embodiments, the method of treating colorectal cancer (CRC) comprises detecting the presence and/or amount of at least one peptide structure from Table 13B and selecting a CRC therapy. In some embodiments, method of treating CRC further comprises administering an effective amount of a CRC therapy to the individual based upon the presence and/or amount of at least one peptide structure from Table 13B. In some embodiments, the diagnosis and/or treatment is based upon the presence and/or amount of at least two, at least three, at least four, at least five, at least six, at least seven, at least eight, or nine peptide structures from Table 13B.

### X.VI.A. Treatments for Colorectal Cancer (CRC)

In some embodiments, the method comprises selecting a therapy to treat colorectal cancer (CRC). In some embodiments, the therapy is selected based upon presence and/or amount of at least one, at least two, at least three, at least four, at least five, at least ten, at least 15, at least 20, at least 25, at least 30, or 31 peptide structures from Table 13A. In some embodiments, the therapy is selected based upon presence and/or amount of at least one, at least two, at least three, at least four, at least five, at least ten, at least 15, at least 20, at least 25, at least 30, or 31 peptides and/or glycopeptides comprising the amino acid sequence of SEQ ID NOs: 168-198 along with the associated glycan set forth in Table 13A. In some embodiments, the therapy is selected based upon presence and/or amount of at least one, at least two, at least three, at least four, at least five, at least six, at least seven, at least eight, or nine peptide structures from Table 13B. In some embodiments, the therapy is selected based upon presence and/or amount of at least one, at least two, at least three, at least four, at least five, at least six, at least seven, at least eight, or nine peptides and/or glycopeptides comprising the amino acid sequence of SEQ ID NOs: 168, 171, 172, 176, 181, 184, 187, 192, and 194 along with the associated glycan set forth in Table 13B. In some embodiments, the presence, absence, and/or amount of one or more peptides and/or glycopeptide is determined by LC-MS. In some embodiments, the therapy is selected on the basis of the stage of CRC. In some embodiments, the therapy is selected on the basis of one or more colorectal cancer (CRC) risk factor in combination with the presence, absence, and or amount of one or more peptides or glycopeptides provided herein. In some embodiments, the therapy for CRC is selected from the group comprising a surgery, a chemotherapeutic therapy, a patient-specific therapy, a targeted immunotherapy, a radiation procedure, a radiofrequency ablation (RFA) procedure, or a combination thereof. In some embodiments, the surgery comprises the removal of one or more parts of the colon and/or the lower intestine. In some embodiments, the surgery comprises a cryosurgery. In some embodiments, the chemotherapeutic therapy comprises one or more chemotherapeutics. In some embodiments, the targeted immunotherapy comprises one or more antibody directed towards an immune system checkpoint protein including but not limited to PD-1, PD-L1, and CTLA-4. In some embodiments, the therapy for CRC comprises a combination of one or more antibody that targets PD-1, PD-L1, and CTLA-4. In some embodiments, the targeted therapy comprises one or more patient-specific therapy agent selected based on patient-specific changes in tumor cell gene expression. In some embodiments, the patient-specific therapy is an inhibitor of an oncogene. In some embodiments, the patient-specific therapy is an inhibitor of one or more of VEGF, EGFR, BRAF, and MEK. In some embodiments, the radiation procedure comprises the use of high-energy rays or particles to treat CRC. In some embodiments, the internal radiation therapy (brachytherapy) comprises the placement of radioactive material in or adjacent to the tumor in the colon (e.g., rectal cavity).

In some embodiments, the method comprises administering a therapy to treat colorectal cancer (CRC). In some embodiments, the therapy is administered based upon presence and/or amount of at least one, at least two, at least three, at least four, at least five, at least ten, at least 15, at least 20, at least 25, at least 30, or 31 peptide structures from Table 13A. In some embodiments, the therapy is administered based upon presence and/or amount of at least one, at least two, at least three, at least four, at least five, at least ten, at least 15, at least 20, at least 25, at least 30, or 31 peptides and/or glycopeptides comprising the amino acid sequence of SEQ ID NOs: 168-198 along with the associated glycan set forth in Table 13A. In some embodiments, the therapy is administered based upon presence and/or amount of at least one, at least two, at least three, at least four, at least five, at least six, at least seven, at least eight, or nine peptide structures from Table 13B. In some embodiments, the therapy is administered based upon presence and/or amount of at least one, at least two, at least three, at least four, at least five, at least six, at least seven, at least eight, or nine peptides and/or glycopeptides comprising the amino acid sequence of SEQ ID NOs: 168, 171, 172, 176, 181, 184, 187, 192, and 194 along with the associated glycan set forth in Table 13B. In some embodiments, the presence, absence, and/or amount of one or more peptides and/or glycopeptide is determined by LC-MS. In some embodiments, the therapy is administered on the basis of the stage of CRC. In some embodiments, the therapy is administered on the basis of one or more CRC risk factor in combination with the presence, absence, and or amount of one or more peptides or glycopeptides provided herein. In some embodiments, the therapy for CRC is administered from the group comprising a surgery, a chemotherapeutic therapy, a patient-specific therapy, a targeted immunotherapy, a radiation procedure, a radiofrequency ablation (RFA) procedure, or a combination thereof. In some embodiments, the surgery comprises the removal of one or more parts of the colon and and/or lower intestine. In some embodiments, the surgery comprises a cryosurgery. In some embodiments, the chemotherapeutic therapy comprises one or more chemotherapeutics. In some embodiments, the targeted immunotherapy comprises one or more antibody directed towards an immune system checkpoint protein including but not limited to PD-1, PD-L1, and CTLA-4. In some embodiments, the therapy for CRC comprises a combination of one or more antibody that targets PD-1, PD-L1, and CTLA-4. In some embodiments, the targeted therapy comprises one or more patient-specific therapy agent administered based on patient-specific changes in tumor cell gene expression. In some embodiments, the patient-specific therapy is an inhibitor of an oncogene. In some embodiments, the patient-specific therapy is an inhibitor of one or more of VEGF, EGFR, BRAF, and MEK. In some embodiments, the radiation procedure comprises the use of high-energy rays or particles to treat CRC. In some embodiments, the brachytherapy comprises the placement of radioactive material in or adjacent to the tumor in the colon (e.g., rectal cavity).

In some embodiments, the method comprises administering a therapy to treat colorectal cancer (CRC). In some embodiments, the therapy is administered based upon presence and/or amount of at least one, at least two, at least three, at least four, at least five, at least ten, at least 15, at least 20, at least 25, at least 30, or 31 peptide structures from Table 13A. In some embodiments, the set of peptide structures comprising one or more, two or more, three or more, four or more, five or more, 10 or more, 15 or more, 20 or more, 25 or more, 30 or more, or each of the peptides and/or glycopeptides consisting of the amino acid sequence of SEQ ID NOs: 168-198. In some embodiments, the peptide structures are detected using LC-MS. In some embodiments, the LC-MS comprises LC-MS/MS or DDA-MS. In some embodiments, the therapy for CRC is selected from the group comprising a surgery, a chemotherapeutic therapy, a patient-specific therapy, a targeted immunotherapy, a radiation procedure, a radiofrequency ablation (RFA) procedure, or a combination thereof. In some embodiments, the surgery to treat colorectal cancer (CRC) comprises the removal of one or more parts of the colon. In some embodiments, the therapy comprises a polypectomy, a local excision, a transanal excision (TAE), lymph node removal, a transanal endoscopic microsurgery (TEM), a low anterior resection (LAR), a proctectomy with colo-anal anastomosis, an abdominoperineal resection (APR), a pelvic exenteration, or a diverting colostomy. In some embodiments, the surgery may comprise cryosurgery. In some embodiments, the peptide structure data comprises one or more peptide structure provided in Table 13A and/or Table 13B. In some embodiments, the presence, absence, and/or amount of one or more peptides and/or glycopeptides is determined by LC-MS. In some embodiments, the method comprises selecting a particular therapy described herein based upon the presence and/or amount of one or more biomarkers comprising the peptide structures provided in Table 13A or Table 13B. In some embodiments, the method comprises administering a particular therapy described herein based upon the presence and/or amount of one or more biomarkers comprising the peptide structures provided in Table 13A or Table 13B.

In some embodiments, the chemotherapeutic therapy to treat colorectal cancer (CRC) comprises 5-fluorouracil, capecitabine, oxaliplatin, irinotecan, trifluridine and tipiracil, or a combination thereof 5-fluorouracil can be dosed to a human subject with a range of about 0.4 g/m² per day to about 3 g/m² per day. Capecitabine can be dosed to a human subject at about 1250 mg/m² BID×2 weeks, followed by 1-week rest period, given as 3-week cycles. Oxaliplatin can be dosed to a human subject with a range of about 85 g/m² per day to about 600 mg/m² per day. Irinotecan can be dosed to a human subject with a range of about 125 mg/m² per day to about 350 mg/m² per day. Trifluridine/tipiracil can be dosed to a human subject with a range of about 35 mg/m² PO BID to about a not to exceed 80 mg. It should be noted that m² can refer to the approximate surface area of the human subject, PO can mean per oral or by mouth, and BID can refer bis in die or twice a day. In some embodiments, the presence, absence, and/or amount of one or more peptides and/or glycopeptides is determined by LC-MS. In some embodiments, the method comprises selecting a particular therapy described herein based upon the presence, and/or amount of one or more biomarkers comprising the peptide structures provided in Table 13A or Table 13B. In some embodiments, the method comprises selecting a particular therapy described herein based upon the presence and/or amount of one or more biomarkers comprising the glycopeptides provided in Table 13A or Table 13B. In some embodiments, the method comprises administering a particular therapy described herein based upon the presence and/or amount of one or more biomarkers comprising the peptide structures provided in Table 13A or Table 13B. In some embodiments, the method comprises administering a particular therapy described herein based upon the presence and/or amount of one or more biomarkers comprising the glycopeptides provided in Table 13A or Table 13B.

In some embodiments, the targeted immunotherapy to treat colorectal cancer (CRC) comprises one or more antibody directed towards an immune system checkpoint protein including but not limited to PD-1, PD-L1, and CTLA-4. In some embodiments, the antibody targeting PD-1 comprises nivolumab (Opdivo), pembrolizumab (Keytruda), and cemiplimab (Libtayo). In some embodiments, the antibody targeting PD-L1 comprises atezolizumab (Tecentriq), durvalumab (Imfinzi), and avelumab (Bavencio). In some embodiments, the antibody targeting CTLA-4 comprises ipilimumab (Yervoy). In some embodiments, the therapy for CRC comprises a combination of one or more antibody that targets PD-1, PD-L1, and CTLA-4. In some embodiments, the presence, absence, and/or amount of one or more peptides and/or glycopeptide is determined by LC-MS. In some embodiments, the method comprises selecting a particular therapy described herein based upon the presence and/or amount of one or more biomarkers comprising the peptide structures provided in Table 13A or Table 13B. In some embodiments, the method comprises selecting a particular therapy described herein based upon the presence and/or amount of one or more biomarkers comprising the glycopeptides provided in Table 13A or Table 13B. In some embodiments, the method comprises administering a particular therapy described herein based upon the presence and/or amount of one or more biomarkers comprising the peptide structures provided in Table 13A or Table 13B. In some embodiments, the method comprises administering a particular therapy described herein based upon the presence and/or amount of one or more biomarkers comprising the glycopeptides provided in Table 13A or Table 13B.

In some embodiments, the therapy to treat colorectal cancer (CRC) comprises one or more patient-specific therapy agent selected based on patient-specific changes in tumor cell gene expression including but not limited to changes in VEGF, EGFR, BRAF, and MEK genes. In some embodiments, the patient-specific therapy is an inhibitor of an oncogene. In some embodiments, the patient-specific therapy is an inhibitor of one or more of VEGF, EGFR, BRAF, and MEK. In some embodiments, the patient-specific therapy comprises aflibercept, cetuximab, panitumumab, encorafenib, and combinations thereof. In some embodiments the patient-specific therapy comprises an angiogenesis inhibitor. In some embodiments, the angiogenesis inhibitor comprises one of bevacizumab (Avastin, BEV) and ramucirumab (Cyramza, RAM). In some embodiments, the therapy for CRC comprises a combination of one or more patient-specific therapy agents. In some embodiments, the presence, absence, and/or amount of one or more peptides and/or glycopeptide is determined by DDA-MS. In some embodiments, the method comprises selecting a particular therapy described herein based upon the presence and/or amount of one or more biomarkers comprising the peptide structures provided in Table 13A or Table 13B. In some embodiments, the method comprises selecting a particular therapy described herein based upon the presence and/or amount of one or more biomarkers comprising the glycopeptides provided in Table 13A or Table 13B. In some embodiments, the method comprises administering a particular therapy described herein based upon the presence and/or amount of one or more biomarkers comprising the peptide structures provided in Table 13A or Table 13B. In some embodiments, the method comprises administering a particular therapy described herein based upon the presence and/or amount of one or more biomarkers comprising the glycopeptides provided in Table 13A or Table 13B.

In some embodiments, the radiation procedure comprises the use of high-energy rays or particles to treat colorectal cancer (CRC). In some embodiments, the radiation procedure comprises external beam radiation therapy (EBRT) and internal radiation therapy (also referred to as brachytherapy). In some embodiments, the EBRT comprises one or more of stereotactic ablative radiotherapy (SABR), three-dimensional conformal radiation therapy (3D-CRT), intensity modulated radiation therapy (IMRT), stereotactic body radiation therapy (SBRT) stereotactic radiosurgery (SRS) or a combination thereof. In some embodiments, the brachytherapy comprises the placement of radioactive material in or adjacent to the tumor in the colon (e.g., rectal cavity). In some embodiments, the presence, absence, and/or amount of one or more peptides and/or glycopeptide is determined by DDA-MS. In some embodiments, the method comprises selecting a particular therapy described herein based upon the presence and/or amount of one or more biomarkers comprising the peptide structures provided in Table 13A or Table 13B. In some embodiments, the method comprises selecting a particular therapy described herein based upon the presence and/or amount of one or more biomarkers comprising the glycopeptides provided in Table 13A or Table 13B. In some embodiments, the method comprises administering a particular therapy described herein based upon the presence and/or amount of one or more biomarkers comprising the peptide structures provided in Table 13A or Table 13B. In some embodiments, the method comprises administering a particular therapy described herein based upon the presence and/or amount of one or more biomarkers comprising the glycopeptides provided in Table 13A or Table 13B.

In some embodiments, the method comprises providing a recommendation to undergo an endoscopy or structural examination for colorectal cancer (CRC). In some embodiments, the endoscopy comprises a sigmoidoscopy or a colonoscopy. In some embodiments, the endoscopy is a sigmoidoscopy. In some embodiments, the endoscopy is a colonoscopy. In some embodiments, the structural examination is a computed tomography (CT) colonoscopy. In some embodiments, the recommendation to undergo an endoscopy or structural exam is based upon the determined risk of an individual having CRC. In some embodiments, the recommendation to undergo an endoscopy or structural examination is based upon the determined risk of an individual suspected of having CRC. In some embodiments, the recommendation to undergo an endoscopy or structural examination is based upon the determined risk of an individual having not received an endoscopy or structural examination within the past 3 months to 15 months. In some embodiments, the recommendation to undergo an endoscopy or structural examination is based upon the determined risk of an individual having not received an endoscopy or structural examination within the past 3 months, 6 months, 9 months, 12 months, or 15 months. In some embodiments, the recommendation to undergo an endoscopy or structural examination is based upon the determined risk of an individual having never received an endoscopy or structural examination. In some embodiments, the method comprises providing a recommendation to undergo an endoscopy or structural examination described herein based upon the presence and/or amount of one or more biomarkers comprising the peptide structures provided in Table 13A or Table 13B. In some embodiments, the method comprises providing a recommendation to undergo an endoscopy or structural examination described herein based upon the presence and/or amount of one or more biomarkers comprising the glycopeptide structures provided in Table 13A or Table 13B. In some embodiments, the presence, absence, and/or amount of one or more peptides and/or glycopeptide is determined by LC-MS, for example, DDA-MS.

In some embodiments, the method further comprises performing an endoscopy or structural examination on the individual to diagnose colorectal cancer (CRC). In some embodiments, the endoscopy comprises a sigmoidoscopy or a colonoscopy. In some embodiments, the endoscopy is a sigmoidoscopy. In some embodiments, the endoscopy is a colonoscopy. In some embodiments, the structural examination is a computed tomography (CT) colonoscopy. In some embodiments, the method further comprises performing an endoscopy or structural examination described herein based upon the presence and/or amount of one or more biomarkers comprising the peptide structures provided in Table 13A or Table 13B. In some embodiments, the method comprises performing an endoscopy or structural examination described herein based upon the presence and/or amount of one or more biomarkers comprising the glycopeptide structures provided in Table 13A or Table 13B. In some embodiments, the presence, absence, and/or amount of one or more peptides and/or glycopeptide is determined by LC-MS, for example, DDA-MS.

In some embodiments, the method further comprises performing additional bodily tests to diagnose colorectal cancer (CRC). In some embodiments, the method further comprises performing a proctoscopy to diagnose colorectal cancer (CRC). In some embodiments, the proctoscopy comprises close examination of the suspected tumor to confirm a tumor is present, obtain measurements, and define its location within the body. In some embodiments, the method further comprises collecting a biopsy sample to diagnose colorectal cancer (CRC). In some embodiments, the biopsy sample is used for detailed tissue inspection and/or CRC staging (e.g., early-stage CRC or late-stage CRC). In some embodiments, the method further comprises performing lab tests to diagnose colorectal cancer (CRC). In some embodiments, a gene analysis is used to determine if the CRC has metastasized and/or may be susceptible to a particular therapy described herein. In some embodiments, the method further comprises imaging tests to diagnose colorectal cancer (CRC). In some embodiments, the imaging test is a computed tomography (CT) scan, an abdominal ultrasound, an magnetic resonance imaging (MRI) scan, a chest X-ray, a position emission tomography (PET) scan, or an angiography. In some embodiments, the method further comprises performing additional bodily tests described herein based upon the presence and/or amount of one or more biomarkers comprising the peptide structures provided in Table 13A or Table 13B. In some embodiments, the method comprises performing additional bodily tests described herein based upon the presence and/or amount of one or more biomarkers comprising the glycopeptide structures provided in Table 13A or Table 13B. In some embodiments, the presence, absence, and/or amount of one or more peptides and/or glycopeptide is determined by LC-MS, for example, MRM-MS.

In some embodiments, the method comprises performing an endoscopy or structural examination as described herein based upon the presence and/or amount of one or more biomarkers comprising the peptide structures provided in Table 13A. In some embodiments, the method comprises administering a particular therapy described herein based upon the presence and/or amount of one or more biomarkers comprising the glycopeptides provided in Table 13B. In some embodiments, the presence, absence, and/or amount of one or more peptides and/or glycopeptide is determined by LC-MS, for example, MRM-MS. In some embodiments, the results of the endoscopy can be used to select a particular therapy described herein for treating CRC. In some embodiments, the particular therapy for treating CRC may comprise a surgery, a chemotherapeutic therapy, a patient-specific therapy, a targeted immunotherapy, a radiation procedure, a radiofrequency ablation (RFA) procedure, or a combination thereof.

In some embodiments, the method involves monitoring of the individual for progression of colorectal cancer (CRC). In some embodiments, the presence, absence, and/or amount of one or more peptides and/or glycopeptide is determined by DDA-MS. In some embodiments, the peptide structure data comprises one or more glycopeptide structure provided in Table 13A and/or Table 13B. In some embodiments, the method involving monitoring further comprises selecting a particular therapy based upon the disease indicator. In some embodiments, the method involving monitoring further comprises administering an effective amount of a therapy for CRC.

In some embodiments, the diagnosis results in further monitoring of the patient for progression of colorectal cancer (CRC). In some embodiments, the diagnosis results in providing a recommendation to the individual to undergo an endoscopy or structural examination. In some embodiments, the endoscopy comprises a sigmoidoscopy or a colonoscopy. In some embodiments, the structural examination comprises a computed tomography (CT) colonoscopy. In some embodiments, the diagnosis results in providing a recommendation to the individual to undergo routine endoscopy or structural examinations. In some embodiments, an endoscopy or structural examination is performed every 3-15 months to monitor progress of the CRC. In some embodiments, an endoscopy or structural examination is performed about every 3 months to 15 months, 4 months to 14 months, 5 months to 13 months, 6 months to 12 months, 7 months to 11 months, or 8 months to 10 months to monitor progress of the CRC. In some embodiments, an endoscopy or structural examination is performed about every 3, 4, 5, 6, 7, 8, 9, 10, 11, 12, 13, 14, or 15 months to monitor progress of the CRC. In some embodiments, the individual is admitted to the hospital for monitoring.

In some embodiments, the method further comprises assessing one or more risk factors associated with colorectal cancer (CRC) or clinical indicators of CRC to provide a diagnosis. In some embodiments, the risk factor for CRC is selected from a group consisting of: age, irritable bowel disease, type 2 diabetes, a family history of CRC, a genetic syndrome (e.g., Lynch syndrome), obesity, smoking, tobacco use, alcohol consumption, dietary choices, limited physical activity, and combinations thereof. In some embodiments, the individual at risk of developing CRC is at least 35, 40, 45, 50, 55, 60, 65, or 70 years of age. In some embodiments, the individual at risk of developing CRC is at least 35 years of age. In some embodiments, the individual at risk of developing CRC is at least 50 years of age. In some embodiments, the individual at risk of developing CRC has a body mass index (BMI) ≥35 kg/m. In some embodiments, the individual at risk of developing CRC has a genetic syndrome, wherein the genetic syndrome comprises familial adenomatous polyposis (FAP) or hereditary non-polyposis colorectal cancer (Lynch syndrome). In some embodiments, the individual at risk of developing CRC consumes an abundance of red or processed meat and/or an limited amount of vegetables and fiber. In some embodiments, the individual has 1, 2, 3, 4, 5, 6, or more risk factors for CRC. In some embodiments, the clinical indicator for CRC is selected from a group consisting of changes in bowel habits, bloody stool, diarrhea, constipation, persistent abdominal pain, persistent abdominal cramps, unexplained weight loss, and combinations thereof.

Also provided herein is a method of preventing and/or reducing the risk of colorectal cancer (CRC) in an individual determined to have a risk of developing CRC. In some embodiments, the method comprises providing a recommendation for making lifestyle changes comprising increasing physical activity, reducing consumption of alcohol and/or use of tobacco products, and consuming more vegetables and fiber. In some embodiments, the method results in a delayed progression of CRC. In some embodiments, the method results in decreased severity of CRC.

### X.VI.B. Patient Populations

Provided herein is a method of diagnosis and treatment for an individual having colorectal cancer (CRC). Further provided herein is a method of diagnosis and treatment for an individual with one or more risk factors associated with CRC. In some embodiments, the method comprises measuring the amount/presence or absence of one or more peptides structures from Table 13A or Table 13B in an individual with one or more risk factors associated with CRC. In some embodiments, the method involves diagnosing an individual based upon presence and/or amount of one or more peptide structures from Table 13A or Table 13B. In some embodiments, the method involves diagnosing an individual based upon presence and/or amount of one or more glycopeptides from Table 13A or Table 13B. In some embodiments, the diagnosis is based upon the presence and/or amount of one or more glycopeptides comprising the amino acid sequence of SEQ ID NOs: 168-198 set forth in Table 13A. In some embodiments, the diagnosis is based upon the presence and/or amount of one or more glycopeptides comprising the amino acid sequence of SEQ ID NOs: 168, 171, 172, 176, 181, 184, 187, 192, and 194 set forth in Table 13B. In some embodiments, the diagnosis is based upon the presence and/or amount of one or more glycopeptides consisting of the amino acid sequence of SEQ ID NOs: 168-198 along with the associated glycan set forth in Table 13A. In some embodiments, the diagnosis is based upon the presence and/or amount of one or more glycopeptides consisting of the amino acid sequence of SEQ ID NOs: 168, 171, 172, 176, 181, 184, 187, 192, and 194 along with the associated glycan set forth in Table 13B. In some embodiments, the individual diagnosed with CRC is administered one or more CRC therapies described herein, based on the diagnosis and determined risk. In some embodiments, the individual diagnosed with CRC is provided a recommendation to undergo an endoscopy or structural examination based upon the determined risk. In some embodiments, the endoscopy comprises a sigmoidoscopy or a colonoscopy. In some embodiments, the endoscopy is a sigmoidoscopy. In some embodiments, the endoscopy is a colonoscopy. In some embodiments, the structural examination is a computed tomography (CT) colonoscopy. In some embodiments, the individual diagnosed with CRC is provided a recommendation to undergo routine endoscopy or structural examinations to further monitor risk of developing CRC. In some embodiments, the individual is administered one or more CRC therapies described herein, based on the diagnosis and determined risk. In some embodiments, the individual confirmed to have CRC is treated based on the diagnosis and determined risk.

In some embodiments, the individual is diagnosed with colorectal cancer (CRC) when the presence or amount one or more peptide structures from Table 13A are detected and are present at a level that is different from a healthy control sample, a set of healthy control samples, or data previous obtained from a set of healthy control samples. In some embodiments, the individual is diagnosed with CRC if one or more glycopeptides comprising the amino acid sequence of SEQ ID NOs: 168-198 are detected and present at a level that is different from a healthy control sample. In some embodiments, the amount of at least one glycopeptide structure is none, or below a detection limit, for example in the healthy control sample. In some embodiments, the amount of at least one glycopeptide structure from Table 13A is none, or below a detection limit, for example in the healthy control sample. In some embodiments, the amount of at least one glycopeptide structure comprising the amino acid sequence of SEQ ID NOs: 168-198 set forth in Table 13A is none, or below a detection limit, for example in the healthy control sample. In some embodiments, the amount of at least one glycopeptide structure is significantly higher than a control sample from a healthy individual. In some embodiments, the amount of at least one glycopeptide structure from Table 13A is significantly higher than a control sample from a healthy individual. In some embodiments, the amount of at least one glycopeptide structure comprising the amino acid sequence of SEQ ID NOs: 168-198 set forth in Table 13A is significantly higher than a control sample from a healthy individual. In some embodiments, the individual is diagnosed and treated according to the presence and/or amount of one or more glycopeptide structures from Table 13A. In some embodiments, the individual is diagnosed and treated according to the presence and/or amount of one or more glycopeptide structures comprising the amino acid sequence of SEQ ID NOs: 168-198 along with the associated glycan set forth in Table 13A.

In some embodiments, the individual is diagnosed with colorectal cancer (CRC) when the presence or amount one or more peptide structures from Table 13B are detected and are present at a level that is different from a healthy control sample, a set of healthy control samples, or data previous obtained from a set of healthy control samples. In some embodiments, the individual is diagnosed with CRC if one or more glycopeptides comprising the amino acid sequence of SEQ ID NOs: 168, 171, 172, 176, 181, 184, 187, 192, and 194 are detected and are present at a level that is different from a healthy control sample. In some embodiments, the amount of at least one glycopeptide structure is none, or below a detection limit, for example in the healthy control sample. In some embodiments, the amount of at least one glycopeptide structure from Table 13B is none, or below a detection limit, for example in the healthy control sample. In some embodiments, the amount of at least one glycopeptide structure comprising the amino acid sequence of SEQ ID NOs: 168, 171, 172, 176, 181, 184, 187, 192, and 194 set forth in Table 13B is none, or below a detection limit, for example in the healthy control sample. In some embodiments, the amount of at least one glycopeptide structure is significantly higher than a control sample from a healthy individual. In some embodiments, the amount of at least one glycopeptide structure from Table 13B is significantly higher than a control sample from a healthy individual. In some embodiments, the amount of at least one glycopeptide structure comprising the amino acid sequence of SEQ ID NOs: 168, 171, 172, 176, 181, 184, 187, 192, and 194 set forth in Table 13B is significantly higher than a control sample from a healthy individual. In some embodiments, the individual is diagnosed and treated according to the presence and/or amount of one or more glycopeptide structures from Table 13B. In some embodiments, the individual is diagnosed and treated according to the presence and/or amount of one or more glycopeptide structures comprising the amino acid sequence of SEQ ID NOs: 168, 171, 172, 176, 181, 184, 187, 192, and 194 along with the associated glycan set forth in Table 13B.

In some embodiments, the individual has colorectal cancer (CRC). In some embodiments, the individual has CRC when the presence or amount one or more peptide structures from Table 13A or Table 13B are detected and are present at a level that is different from a healthy control sample, a set of healthy control samples, or data previous obtained from a set of healthy control samples. In some embodiments, the individual has stage 0, stage I, stage II, stage III, or stage IV CRC. In some embodiments, the individual has stage IVA CRC or stage IVB CRC. In some embodiments, the individual has stage IVA CRC and the cancer has spread to one organ distant from the colon. In some embodiments, the individual has stage IVB CRC and the cancer has spread to two or more organ distant from the colon. In some embodiments, the organ distal from the colon comprises the liver, a lung, an ovary, or a distant lymph node. In some embodiments, the individual has early-stage CRC. In some embodiments, the individual has late-stage CRC or advanced CRC. In some embodiments, the individual has CRC that has not spread from the site of origination. In some embodiments, the individual has CRC that has spread locally to the surrounding tissue. In some embodiments, the individual has CRC that has spread beyond the original tumor and/or the local tumor environment. In some embodiments, the individual has CRC that has spread to one or more organs beyond the colon. In some embodiments, the individual has metastatic CRC. In some embodiments, the individual has CRC and has relapsed and/or progressed. In some embodiments, the presence, absence, and/or amount of one or more peptides and/or glycopeptide is determined by DDA-MS. In some embodiments, the method comprises selecting a particular therapy described herein based upon the presence and/or amount, of one or more biomarkers comprising the glycopeptides provided in Table 13A or Table 13B. In some embodiments, the method comprises administering a particular therapy described herein based upon the presence and/or amount of one or more biomarkers comprising the glycopeptides provided in Table 13A or Table 13B. In some embodiments, the individual diagnosed with CRC is provided a recommendation to undergo an endoscopy or structural examination based upon the determined risk. In some embodiments, the individual diagnosed with CRC is provided a recommendation to undergo routine endoscopy or structural examinations to further monitor the CRC.

In some embodiments, the colon cancer is staged based on the TNM (tumor, lymph node, metastasis) staging system. In some embodiments, the system considers factors comprising the primary tumor (T), regional lymph nodes (N), and distant metastases (M). In some embodiments, the T factor refers to how large the original tumor is and whether the cancer has grown into the wall of the colon or spread to adjacent organs or structures. In some embodiments, the N factor refers to whether cancer cells have spread to nearby lymph nodes. In some embodiments, the M factor refers to whether cancer has metastasized from the colon to other parts of the body. In some aspects, the cancer has metastasized to distant parts of the body, including but not limited to the liver, the lungs, the ovaries, or one or more distant lymph nodes.

In some embodiments, the individual is suspected of having colorectal cancer (CRC). In some embodiments, the individual has not been diagnosed with CRC. In some embodiments, the individual is suspected of having CRC when the presence or amount one or more peptide structures from Table 13A or Table 13B are detected and are present at a level that is different from a healthy control sample, a set of healthy control samples, or data previous obtained from a set of healthy control samples. In some embodiments, individual is suspected of having CRC based on the presence, absence, and/or amount of one or more glycopeptide from Table 13A or Table 13B. In some embodiments, the individual is suspected of having CRC based upon presence and/or amount of at least one, at least two, at least three, at least four, at least five, at least ten, at least 15, at least 20, at least 25, at least 30, or 31 peptides and/or glycopeptides comprising the amino acid sequence of SEQ ID NOs: 168-198 along with the associated glycan set forth in Table 13A. In some embodiments, the individual is suspected of having CRC based upon presence and/or amount of at least one, at least two, at least three, at least four, at least five, at least six, at least seven, at least eight, or nine peptides and/or glycopeptides comprising the amino acid sequence of SEQ ID NOs: 168, 171, 172, 176, 181, 184, 187, 192, and 194 along with the associated glycan set forth in Table 13B. In some embodiments, the presence, absence, and/or amount of one or more glycopeptide is determined by DDA-MS. In some embodiments, the individual has not received an endoscopy or a structural examination for diagnosing CRC. In some embodiments, the individual has not received an endoscopy or a structural examination for diagnosing CRC in the past 3, 4, 5, 6, 7, 8, 9, 10, 11, 12, 13, 14, or 15 months. In some embodiments, the individual has not received an endoscopy or a structural examination for diagnosing CRC in the past 3 months 6 months, 1 year, 2 years, 3 years, 4 years, 5 years, 6 years, 7 years, 8 years, 9 years, or 10 years. In some embodiments, the individual has not received an endoscopy or a structural examination for diagnosing CRC for at least 10 or more years. In some embodiments, the individual has never received an endoscopy or a structural examination for diagnosing CRC.

In some embodiments, the individual is suspected of having colorectal cancer (CRC). In some embodiments, the individual has not been diagnosed with CRC. In some embodiments, the individual is suspected of having CRC when the presence or amount one or more peptide structures from Table 13A or Table 13B are detected and are present at a level that is different from a healthy control sample, a set of healthy control samples, or data previous obtained from a set of healthy control samples. In some embodiments, individual is suspected of having CRC based on the presence, absence, and/or amount of one or more glycopeptide from Table 13A or Table 13B. In some embodiments, the individual is suspected of having CRC based upon presence and/or amount of at least one, at least two, at least three, at least four, at least five, at least ten, at least 15, at least 20, at least 25, at least 30, or 31 peptides and/or glycopeptides comprising the amino acid sequence of SEQ ID NOs: 168-198 along with the associated glycan set forth in Table 13A. In some embodiments, the individual is suspected of having CRC based upon presence and/or amount of at least one, at least two, at least three, at least four, at least five, at least six, at least seven, at least eight, or nine peptides and/or glycopeptides comprising the amino acid sequence of SEQ ID NOs: 168, 171, 172, 176, 181, 184, 187, 192, and 194 along with the associated glycan set forth in Table 13B. In some embodiments, the presence, absence, and/or amount of one or more glycopeptide is determined by DDA-MS. In some embodiments, the individual has not received a non-invasive test for diagnosing CRC (e.g., a stool-based test). In some embodiments, the individual has not received a non-invasive test for diagnosing CRC in the past 3, 4, 5, 6, 7, 8, 9, 10, 11, 12, 13, 14, or 15 months. In some embodiments, the individual has not received a non-invasive test for diagnosing CRC in the past 3 months 6 months, 1 year, 2 years, 3 years, 4 years, 5 years, 6 years, 7 years, 8 years, 9 years, or 10 years. In some embodiments, the individual has not received a non-invasive test for diagnosing CRC for at least 10 or more years. In some embodiments, the individual has never received a non-invasive test for diagnosing CRC.

In some embodiments, the individual has had prior lines of therapy for treating colorectal cancer (CRC). In some embodiments, the individual has had at least 1, at least 2, or at least 3 prior lines of therapy for treating CRC. In some embodiments, the individual has had no more than 1, no more than 2, or no more than 3 prior lines of therapy for treating CRC. In some embodiments, the individual has not had prior therapy for treating CRC.

In some embodiments, the individual has altered gene expression relevant for colorectal cancer (CRC) treatment. In some embodiments, the individual has altered oncogene expression. In some embodiments, the individual has altered tumor cell gene expression. In some embodiments, the altered gene expression comprises altered gene expression of one or more of VEGF, EGFR, BRAF, and MEK. In some embodiments, the altered gene expression comprises altered gene expression of one or more immune system checkpoint proteins PD-1, PD-L1, and CTLA-4. In some embodiments, the individual having altered gene expression relevant for CRC treatment may benefit from a therapy comprising one or more antibody that targets PD-1, PD-L1, and CTLA-4, or a combination thereof.

In some embodiments, the individual is at risk of developing colorectal cancer (CRC). In some embodiments, the risk of CRC is determined based upon presence and/or amount of at least one peptide structures from Table 13A or Table 13B. In some embodiments, the risk of CRC is determined based upon the presence and/or amount of one or more peptides comprising the amino acid sequence of SEQ ID NOs: 168-198. In some embodiments, the individual is positive for one or more risk factor that increases the chances of developing CRC. In some embodiments, the one or more risk factor is selected from a group consisting of age, irritable bowel disease, type 2 diabetes, a family history of CRC, a genetic syndrome (e.g., Lynch syndrome), obesity, smoking, tobacco use, alcohol consumption, dietary choices, and limited physical activity. In some embodiments, the individual has at least 1, at least 2, at least 3, at least 4, at least 5, or at least 6 risk factors for CRC.

In some embodiments, the individual is positive for one or more risk factor that increases the chances of developing colorectal cancer (CRC). In some embodiments, the one or more risk factor comprises the age of the individual. In some embodiments, the individual is at least 30, at least 35, at least 40, at least 45, at least 50, at least 55, at least 60, at least 65, at least 70, at least 75, at least 80, at least 85, or at least 90 years old. In some embodiments, the individual is at least 30 years old. In some embodiments, the individual is at least 40 years old. In some embodiments, the individual is at least 50 years old. In some embodiments, the individual is at least 60 years old.

In some embodiments, the individual at risk of developing colorectal cancer (CRC) is overweight or obese. In some embodiments, the individual at risk of developing CRC has a body mass index (BMI) ≥30 kg/m. In some embodiments, the individual at risk of developing CRC has a BMI; ≥35 kg/m. In some embodiments, the individual at risk of developing CRC has a BMI≥40 kg/m. In some embodiments, the individual is considered extremely obese.

In some embodiments, the individual at risk of developing colorectal cancer (CRC) has a genetic syndrome. In some embodiments, the genetic syndrome comprises familial adenomatous polyposis (FAP) or hereditary non-polyposis colorectal cancer (Lynch syndrome).

In some embodiments, the individual at risk of developing colorectal cancer (CRC) consumes foods that may increase the risk of CRC. In some embodiments, the individual consumes an abundance of red or processed meat. In some embodiments, the individual at risk of developing CRC does not consume foods that may decrease the risk of CRC. In some embodiments, the individual consumes a limited amount of vegetables and fiber.

In some embodiments, the individual at risk of developing colorectal cancer (CRC) is a smoker or consumer of tobacco products. In some embodiments, the individual smokes cigarettes, cigars, pipes, and other tobacco-based products. In some embodiments, the individual is a smoker. In some embodiments, the individual uses tobacco-containing products.

In some embodiments, the individual is positive for one or more clinical indicators of colorectal cancer (CRC) described herein. In some embodiments, the one or more clinical indicators of CRC comprise a changes in bowel habits, bloody stool, diarrhea, constipation, persistent abdominal pain, persistent abdominal cramps, and unexplained weight loss. In some embodiments, the individual has at least 1, at least 2, at least 3, at least 4, at least 5, or at least 6 clinical indicators of CRC. In some embodiments, the individual has any combination of clinical indicators of CRC described herein.

### X.VII. Compositions and Kits

In some embodiments, provided herein is a composition comprising one or more peptide structures from Table 13A. In some embodiments, provided herein is a composition comprising two or more peptide structures from Table 13A. In some embodiments, provided herein is a composition comprising three or more peptide structures from Table 13A. In some embodiments, provided herein is a composition comprising four or more peptide structures from Table 13A. In some embodiments, provided herein is a composition comprising five or more peptide structures from Table 13A. In some embodiments, provided herein is a composition comprising six or more peptide structures from Table 13A. In some embodiments, provided herein is a composition comprising seven or more peptide structures from Table 13A. In some embodiments, provided herein is a composition comprising eight or more peptide structures from Table 13A. In some embodiments, provided herein is a composition comprising nine or more peptide structures from Table 13A. In some embodiments, provided herein is a composition comprising ten or more peptide structures from Table 13A. In some embodiments, provided herein is a composition comprising fifteen or more peptide structures from Table 13A. In some embodiments, provided herein is a composition comprising twenty or more peptide structures from Table 13A. In some embodiments, provided herein is a composition comprising twenty-five or more peptide structures from Table 13A. In some embodiments, provided herein is a composition comprising thirty or more peptide structures from Table 13A. In some embodiments, provided herein is a composition comprising thirty-one peptide structures from Table 13A. In some embodiments, the composition is from a biological sample. In some embodiments, the composition comprises one or more purified peptide structures. In some embodiments, the composition comprises enzymatically digested peptide fragments, such as those in Table 13A. In some embodiments, the composition comprises one, two, three, four, five, six, seven, eight, nine, ten, fifteen, twenty, twenty-five, thirty, or thirty-one peptides comprising a sequence set forth in SEQ ID NOs: 168-198.

In some embodiments, provided herein is a composition comprising one or more peptide structures from Table 13B. In some embodiments, provided herein is a composition comprising two or more peptide structures from Table 13B. In some embodiments, provided herein is a composition comprising three or more peptide structures from Table 13B. In some embodiments, provided herein is a composition comprising four or more peptide structures from Table 13B. In some embodiments, provided herein is a composition comprising five or more peptide structures from Table 13B. In some embodiments, provided herein is a composition comprising six or more peptide structures from Table 13B. In some embodiments, provided herein is a composition comprising seven or more peptide structures from Table 13B. In some embodiments, provided herein is a composition comprising eight or more peptide structures from Table 13B. In some embodiments, provided herein is a composition comprising nine peptide structures from Table 13B. In some embodiments, the composition is from a biological sample. In some embodiments, the composition comprises one or more purified peptide structures. In some embodiments, the composition comprises enzymatically digested peptide fragments, such as those in Table 13B. In some embodiments, the composition comprises one, two, three, four, five, six, seven, eight, or nine peptides comprising a sequence set forth in SEQ ID NOs: 168, 171, 172, 176, 181, 184, 187, 192, and 194.

In some embodiments, provided herein is a composition comprising at least one peptide comprising a sequence set forth in SEQ ID NOs: 168-198. In some embodiments, provided herein is a composition comprising at least two peptides comprising a sequence set forth in SEQ ID NOs: 168-198. In some embodiments, provided herein is a composition comprising at least three peptides comprising a sequence set forth in SEQ ID NOs: 168-198. In some embodiments, provided herein is a composition comprising at least four peptides comprising a sequence set forth in SEQ ID NOs: 168-198. In some embodiments, provided herein is a composition comprising at least five peptides comprising a sequence set forth in SEQ ID NOs: 168-198. In some embodiments, provided herein is a composition comprising at least six peptides comprising a sequence set forth in SEQ ID NOs: 168-198. In some embodiments, provided herein is a composition comprising at least seven peptides comprising a sequence set forth in SEQ ID NOs: 168-198. In some embodiments, provided herein is a composition comprising at least eight peptides comprising a sequence set forth in SEQ ID NOs: 168-198. In some embodiments, provided herein is a composition comprising at least nine peptides comprising a sequence set forth in SEQ ID NOs: 168-198. In some embodiments, provided herein is a composition comprising at least ten peptides comprising a sequence set forth in SEQ ID NOs: 168-198. In some embodiments, provided herein is a composition comprising at least fifteen peptides comprising a sequence set forth in SEQ ID NOs: 168-198. In some embodiments, provided herein is a composition comprising at least twenty peptides comprising a sequence set forth in SEQ ID NOs: 168-198. In some embodiments, provided herein is a composition comprising at least twenty-five peptides comprising a sequence set forth in SEQ ID NOs: 168-198. In some embodiments, provided herein is a composition comprising at least thirty peptides comprising a sequence set forth in SEQ ID NOs: 168-198. In some embodiments, provided herein is a composition comprising thirty-one peptides comprising a sequence set forth in SEQ ID NOs: 168-198.

In some embodiments, provided herein is a composition comprising at least one peptide comprising a sequence set forth in SEQ ID NOs: 168, 171, 172, 176, 181, 184, 187, 192, and 194. In some embodiments, provided herein is a composition comprising at least two peptides comprising a sequence set forth in SEQ ID NOs: 168, 171, 172, 176, 181, 184, 187, 192, and 194. In some embodiments, provided herein is a composition comprising at least three peptides comprising a sequence set forth in SEQ ID NOs: 168, 171, 172, 176, 181, 184, 187, 192, and 194. In some embodiments, provided herein is a composition comprising at least four peptides comprising a sequence set forth in SEQ ID NOs: 168, 171, 172, 176, 181, 184, 187, 192, and 194. In some embodiments, provided herein is a composition comprising at least five peptides comprising a sequence set forth in SEQ ID NOs: 168, 171, 172, 176, 181, 184, 187, 192, and 194. In some embodiments, provided herein is a composition comprising at least six peptides comprising a sequence set forth in SEQ ID NOs: 168, 171, 172, 176, 181, 184, 187, 192, and 194. In some embodiments, provided herein is a composition comprising at least seven peptides comprising a sequence set forth in SEQ ID NOs: 168, 171, 172, 176, 181, 184, 187, 192, and 194. In some embodiments, provided herein is a composition comprising at least eight peptides comprising a sequence set forth in SEQ ID NOs: 168, 171, 172, 176, 181, 184, 187, 192, and 194. In some embodiments, provided herein is a composition comprising nine peptides comprising a sequence set forth in SEQ ID NOs: 168, 171, 172, 176, 181, 184, 187, 192, and 194.

In some embodiments, provided herein are peptides set forth in Table 13A. In some embodiments, provided herein are peptides comprising a sequence set forth in SEQ ID NOs: 168-198. In some embodiments, provided herein are peptides set forth in Table 13B. In some embodiments, provided herein are peptides comprising a sequence set forth in SEQ ID NOs: 168, 171, 172, 176, 181, 184, 187, 192, and 194.

In some embodiments, a kit is provided, the kit comprising at least one agent for quantifying at least one peptide structure identified in Table 13A to carry out part or all of any one or more of the methods disclosed herein. In some embodiments, a kit is provided, the kit comprising at least one agent for quantifying at least one peptide structure identified in Table 13B to carry out part or all of any one or more of the methods disclosed herein.

In some embodiments, a kit is provided, the kit comprising at least one of a glycopeptide standard, a buffer, or a set of peptide sequences to carry out part or all of any one or more of the methods disclosed herein.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Digestion of Samples Prior to Enrichment and Analysis

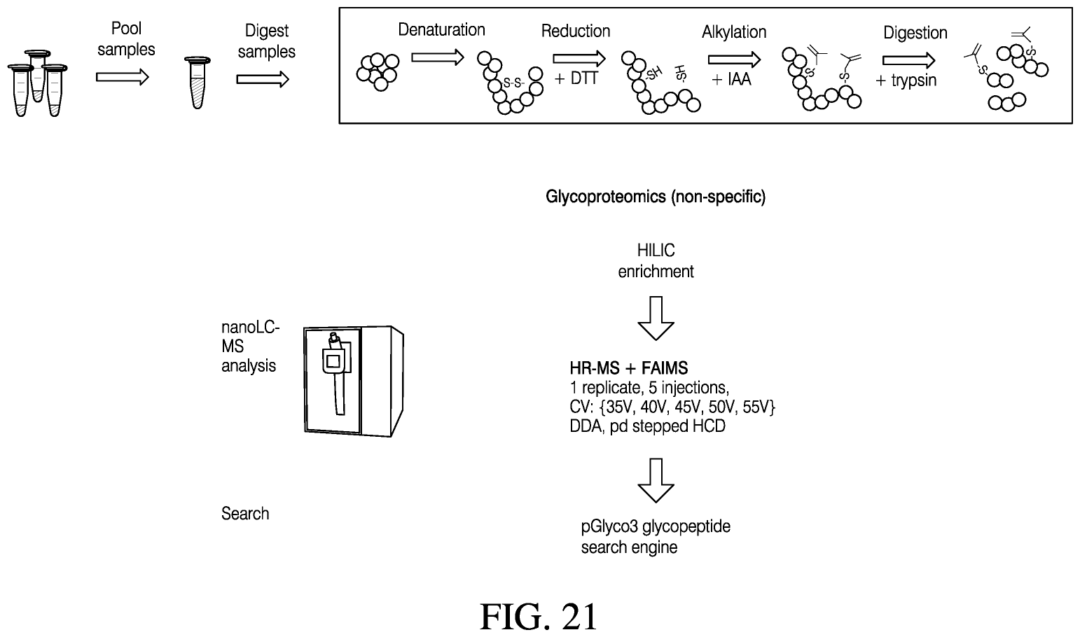

A schematic for the overall workflow for sample preparation and analysis is given in FIG. 21 for identifying new glycoproteins and glycoforms that are suitable for use as biomarkers for diagnosing colorectal cancer (CRC). A summary of the sample population used for the experiments is provided in Table 12A. The sample set consisted of human serum samples from 10 healthy subjects who were not diagnosed with colorectal cancer, and human serum samples from 9 subjects that were diagnosed with CRC. Of the 9 subjects having CRC, 5 subjects were assessed as having early-stage CRC (e.g., Stage I CRC). The remaining 4 subjects were assessed as having late-stage CRC (e.g., Stage IV CRC). Of the subjects having late-stage CRC, 3 subjects had stage IVA and 1 subject had stage IVB. For Stage I CRC, the cancer has grown through the mucosa and has invaded the muscular layer of the colon or rectum. The cancer has not spread into nearby tissue or lymph node. For Stage IVA, the cancer has spread to a single organ or tissue distant from the colon, such as the liver or lungs. For Stage IVB, the cancer has spread to two or more organs or tissues distant from the colon.

Clinical diagnosis of subjects with CRC was determined by using a colonoscopy where a scope was inserted into the interior of the colon for visual examination. Colonoscopy uses a long, flexible and slender tube attached to a video camera and monitor to view the interior of the colon and rectum for tumors or polyps. Colon cancer is typically staged based on the TNM (tumor, lymph node, metastasis) staging system. The system considers the following factors: the primary tumor (T), regional lymph nodes (N), and distant metastases (M). T refers to how large the original tumor is and whether cancer has grown into the wall of the colon or spread to adjacent organs or structures. N refers to whether cancer cells have spread to nearby lymph nodes. M refers to whether cancer has metastasized (spread) from the colon to other parts of the body, like the lungs or liver.

TABLE 12A
Summary of the biological samples and control samples tested

| | Number of | |
|---|---|---|
| Sample Type | Subjects | Comments |
| Early-stage colorectal | 5 | |
| cancer (Stage I) | | |
| Late-stage colorectal | 4 | 3 subjects at stage IVA, |
| cancer (Stage IV) | | 1 subject at stage IVB |
| Healthy Control | 10 | 5 males and 5 females; |
| | | no colorectal cancer or |
| | | advanced adenoma |
| Healthy Control - serum | 1 | Mixed serum sample |
| from Sigma | | from healthy donors |
| Healthy Control - plasma | 1 | Mixed plasma sample |
| from Sigma | | from healthy donors |

Pooled human serum for assay normalization and calibration purposes, dithiothreitol (DTT), and iodoacetamide (IAA) were purchased from Millipore Sigma (St. Louis, MO). Sequencing grade trypsin was purchased from Promega (Madison, WI). Acetonitrile (LC-MS grade) was purchased from Honeywell (Muskegon, MI). All other reagents used were procured from Millipore Sigma, VWR, and Fisher Scientific.

In the first step described for the method herein, ammonium bicarbonate (50 mM) and dithiothreitol (DTT) (50 mM) solutions were freshly prepared. The ammonium bicarbonate solution was used to make the DTT solution. Immediately prior to transfer, each biological sample and control was gently vortexed for 10 seconds. Using a single channel pipette, 10 μL of biological sample or control (e.g., plasma or serum) was transferred into a deep-well digestion plate, wherein the plate is compatible with thermal cycling. To this, the 35 μL of 50 mM ammonium bicarbonate solution was added. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

The sample plate containing the sample was incubated in a thermal cycler for 5 minutes, wherein the thermal cycler was set to 100° C. with a lid temperature of 105° C. All heated plates were allowed to cool to room temperature before removing from the respective heat source and spinning at 370×g for 1 minute. After the spin, the plate seals were removed.

After protein denaturation, all samples were reduced by adding 20 μL of the 50 mM DTT solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 60° C. water bath for 50 minutes. Plates were then removed from the water bath and centrifuged at 4,800×g for 1 minute before removing the plate seals.

Prior to the completion of this reduction incubation, a fresh 90 mM iodoacetamide (IAA) solution was prepared, and the container with the IAA solution was covered in foil. When ready, samples were alkylated by adding 20 μL of the 90 mM IAA solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in the dark at room temperature for 30 minutes. After the incubation, plate seals were removed and 10 μL of the 50 mM DTT solution was added to quench any remaining IAA in solution. The plates were then sealed with a foil heat seal using a plate sealer and vortexed at 1400 RPM for 1 minute on a microplate mixer. Plates were centrifuged at 370×g for 1 minute and the plate seals were removed.

Prior to the completion of this alkylation incubation, fresh protease solutions were prepared that were a combination of trypsin/LysC. For example, for the trypsin/LysC solution, trypsin/LysC powder was dissolved in the 50 mM ammonium bicarbonate solution for a final concentration of 0.333 μg/μL trypsin/LysC solution. To the quenched biological samples and controls, 60 μL of the 0.333 μg/μL trypsin/LysC solution was added to each well where the sample was plasma. When the sample was serum, 60 μL of the 0.333 μg/μL trypsin solution was added to each well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 37° C. water bath for 18 hours. Plates were then removed from the water bath and centrifuged at 4,800×g for 1 minute before removing the plate seals. [00152]20 μL of freshly prepared 9% formic acid solution was added to each well containing the proteolytic digested samples to stop the enzyme reaction and form the tryptically digested samples. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

### Example 2. Enrichment of Digested Samples

Serum samples from subjects having colorectal cancer (CRC) and from healthy subjects not having CRC (e.g., healthy control) were tryptically digested as described in Example 1. Digested samples were enriched for glycopeptides using a hydrophilic interaction liquid chromatography (HILIC) concentration phase. The HILIC sorbent material used in this example was the Agilent GlykoPrep Cleanup (CU) Cartridges on the Agilent Bravo Platform for AssayMAP (liquid handler). This enrichment process increased the proportion of glycopeptides with respect to the both of the peptides and glycopeptides together in the sample (e.g., >50% glycopeptides/glycopeptide+peptide) by increasing the interactions between the glycans and the sorbent material. These interactions were dominated by H-bonding between the glycan hydroxyl groups or sialic acid carboxylic acid group, and the sorbent functional groups. In some embodiments, a glycopeptide concentration for a glycopeptide derived from the proteolytic digest sample is enriched by a factor of 30 or greater with respect to a peptide concentration where the peptide concentration represents an amount of a peptide that is associated with the same protein as the glycopeptide.

170 μL of serum digest was collected and dried with a SpeedVac evaporator until the sample was dry. Next, the dry sample was reconstituted with 220 μL of 1% trifluoroacetic acid (TFA) and 80% acetonitrile (ACN). The plate was centrifuged at 1300 RPM at 4° C. for 1 minute. Following centrifugation, 200 μL of the reconstituted solution was loaded onto a Agilent GlykoPrep Cleanup (CU) Cartridge at a 3 μL/min flow rate. The flow-through from the cartridge during the loading process was discarded. The cartridge was washed with 200 μL Wash Buffer (1% TFA, 96% ACN in deionized water) at a 3 μL/min flow rate. After washing, the cartridge was eluted with 100 μL of an elution buffer (0.1% TFA in deionized water) at a 3 L/min flow rate. The eluate was collected and then dried with a SpeedVac evaporator to form the enriched sample. 50 μL of 0.1% formic acid and 3% ACN in water was added to each of the dried samples to reconstitute the sample prior to injection onto a LC-MS system.

### Example 3. Mass Spectrometry of the Enriched Samples

The HILIC enriched samples were analyzed with LC-MS. More specifically, samples were delivered using the UltiMate 3000 LC System (Thermo Scientific) with a Acclaim™ PepMap™ 100 C18 IPLC Columns (0.075 mm×150 mm) (Thermo Scientific) coupled to a FAIMS Pro device (Thermo Scientific) and Orbitrap Exploris 480 mass spectrometer (Thermo Scientific).

The LC parameters were as follows: samples were separated and delivered into the FAIMS-MS system at a flow rate of 0.4 L/min with a gradient from 99% buffer A (water containing 0.1% formic acid) and 1% buffer B (ACN containing 0.1% formic acid) to 66% buffer A and 34% buffer B in 68 minutes for colorectal cancer (CRC) samples, healthy control samples and plasma samples from Sigma. For serum samples from Sigma, different LC gradient conditions were used to increase analytical sensitivity, including gradient from 99% buffer A (water containing 0.1% formic acid) and 1% buffer B (ACN containing 0.1% formic acid) to 66% buffer A and 34% buffer B in 68/102/136/170 minutes.

The MS parameters were as follows. Each sample was acquired using a product dependent data dependent acquisition (pd-DDA) method with FAIMS operated at five different compensation voltage (CV) values of −35V, −40V, −45V, −50V, −55 V. MS parameters were as follows: spray voltage of 2.5 kV; ion transfer capillary temperature of 300° C.; MS1 resolution (FWHM) at m/z 200 set to 120,000; custom MS1 automatic gain control set to 300%; MS maximum injection time mode set to auto; MS/MS resolution (FWHM) at m/z 200 set to 60,000; custom MS/MS automatic gain control at 300%; MS/MS maximum injection time mode set to auto; isolation width of 1.6. Each precursor was first fragmented with HCD-MS/MS of NCE (normalized collisional energies) of 45 and subjected to an online product-ion screening: if three out of four glycopeptide diagnostic ions of “138.055, 168.066, 186.076, 204,067” were observed in NCE45-HCD-MS/MS spectrum, a second round of HCD-MS/MS was triggered for the same precursor with NCE of 15, 20, 34, 37, 40, or SCE-HCD-MS/MS (stepped collisional energy HCD-MS/MS).

### Example 4. Glycopeptide Identification

RAW MS data was first converted into “mzML” open format by MSConvert (https://proteowizard.sourceforge.io). In-house scripts were developed to filter product-ion trigger spectra. In the “mzML”, the collisional energy information was included in the scan header of each spectrum. “HCD45” for HCD-MS/MS of NCE45 and “HCD29” for SCE-HCD-MS/MS (29 being the numerical average of 15, 20, 34, 37, 40). All spectra that have “HCD29” were triggered glycopeptide spectra and were retained for further analysis.

All retained spectra having HCD29 were analyzed using pGlyco3 glycopeptide search engine (https://www.nature.com/articles/s41592-021-01306-0) to determine glycopeptide-spectrum matches. In some instances, a glycopeptide spectrum match may be referred to as peptide spectrum match or by the acronym PSM. The pGlyco3 search parameters were as follows: mass tolerance for precursors and fragment ions were set as ±5 p.p.m. and ±20 p.p.m., respectively. The protein databases were from Swiss-Prot, human proteome version 21.12. The enzyme was full-trypsin. Maximal missed cleavage was 3. Fixed modification was carbamidomethylation on all Cys residues (C +57.022 Da). Variable modifications contained oxidation on methionine (Met, M+15.995 Da) and acetylation on protein N-terminus (+42.011 Da). The N-glycosylation sequon (N—X—S/T, X P) was modified by changing ‘N’ to ‘J’ (the two shared the same mass). The glycan database was extracted from GlycomeDB (www.glycome-db.org), total entries of N-glycans were 7884 by considering NeuGc. The false positive rate was set to 0.1% for identifying a glycopeptide-spectrum match.

The identified glycopeptide-spectrum-matches that passed the false-positive filtering criteria were further filtered by in-house developed QC steps based on precursor isotope patterns. The weight of the first and second monoisotopic peaks (intensity one peak VS intensity of all monoisotopic peaks) was calculated in RAW MS data and in the theoretical glycopeptide pattern. If the weight difference of the first and second monoisotopic peaks was more than 5% (absolute value) off the theoretical glycopeptide pattern, corresponding glycopeptide-spectrum-matching were excluded from further analysis.

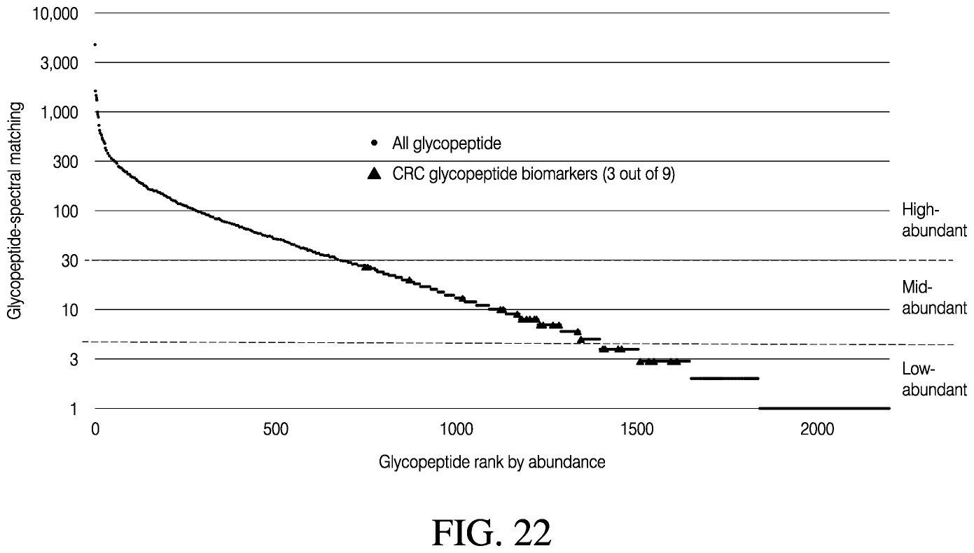

After processing the RAW MS data with the steps described herein, a large number of unique N-glycopeptides were identified in each sample (Table 12B). Here “unique N-glycopeptide” means a non-redundant combination of “Peptide sequence+N-glycan”. Each N-glycopeptide may be identified multiple times (or corresponding to multiple glycopeptide-spectral matching). The number of spectral matching for a unique N-glycopeptide was used as an indicator of N-glycopeptide abundance, as shown in FIG. 22, which ranks all of the identified glycopeptides and the CRC glycopeptide biomarkers by the number of glycopeptide spectral matches. In FIG. 22, the most abundant third (˜33%) of unique glycopeptides were defined as “high abundant glycopeptide” (spectral matches per glycopeptide is greater than 30); the middle third of unique glycopeptides were defined as “mid abundant glycopeptide” (spectral matches per glycopeptide greater than 5 and less than or equal to 30); and the lowest third of unique glycopeptide were defined as “low abundant glycopeptide” (spectral matches per glycopeptide less than or equal to 5).

TABLE 12B
Summary of the unique N-glycopeptides identified

| | No. N- | |
|---|---|---|
| Sample | glycopeptides | Dataset |
| Healthy Control - serum | 3,194 | #1 |
| from Sigma | | |
| Healthy Control - plasma | 1,987 | #2 |
| from Sigma | | |
| Healthy Control (10 | 1,544 | #3 |
| subjects) | | |
| Colorectal cancer (all 9 | 2,201 | #4 |
| patients) | | |
| Colorectal cancer | 1,203 | #4.1 |
| (individual patient) | 1,218 | #4.2 |
| | 1,175 | #4.3 |
| | 1,250 | #4.4 |
| | 1,356 | #4.5 |
| | 1,111 | #4.6 |
| | 1,157 | #4.7 |
| | 1,188 | #4.8 |
| | 1,204 | #4.9 |

For this investigation, an N-glycopeptide observed in colorectal cancer (CRC) sample (#4) and not observed in any other control sample (samples #1, #2 and #3) was considered as a CRC glycopeptide biomarker. This filter yielded 212 unique N-glycopeptides only observed in CRC subjects. This list of 212 CRC glycopeptide biomarkers was further distilled by requiring that an N-glycopeptide must be present in at least 3 of the 9 subjects (#4.1-4.9 in Table 12B). Of the 212 unique CRC glycopeptide biomarkers identified, 32 CRC glycopeptide biomarkers were observed in at least 3 individual CRC subjects. The 32 CRC glycopeptide biomarkers found in at least 3 individual CRC subjects are listed in Table 13A. To improve disease identification and patient diagnostic power, the biomarker list was further distilled by requiring a CRC glycopeptide to be present in at least 5 of the 9 subjects (#4.1-4.9 in Table 12B). This analysis yielded a subset of 9 CRC biomarkers, which are listed in Table 13B. Table 14 provides protein information that corresponds to the various glycopeptides listed in Table 13A and Table 13B.

TABLE 14
Glycoproteins associated with healthy control and colorectal (CRC)
samples

| SEQ | | | | |
|---|---|---|---|---|
| ID | Protein | Protein | Uniprot | |
| NO | Abbreviation | Name | ID | Protein Sequence |
| 19 | HPT | Haptoglobin | P00738 | MSALGAVIALLLWGQLFAVDSGNDVTDIADDGCPKPPEI |
| | | | | AHGYVEHSVRYQCKNYYKLRTEGDGVYTLNDKKQWIN |
| | | | | KAVGDKLPECEADDGCPKPPEIAHGYVEHSVRYQCKNY |
| | | | | YKLRTEGDGVYTLNNEKQWINKAVGDKLPECEAVCGKP |
| | | | | KNPANPVQRILGGHLDAKGSFPWQAKMVSHHNLTTGA |
| | | | | TLINEQWLLTTAKNLFLNHSENATAKDIAPTLTLYVGKK |
| | | | | QLVEIEKVVLHPNYSQVDIGLIKLKQKVSVNERVMPICLP |
| | | | | SKDYAEVGRVGYVSGWGRNANFKFTDHLKYVMLPVAD |
| | | | | QDQCIRHYEGSTVPEKKTPKSPVGVQPILNEHTFCAGMS |
| | | | | KYQEDTCYGDAGSAFAVHDLEEDTWYATGILSFDKSCA |
| | | | | VAEYGVYVKVTSIQDWVQKTIAEN |
| 13 | A1AT | Alpha-1- | P01009 | MPSSVSWGILLLAGLCCLVPVSLAEDPQGDAAQKTDTSH |
| | | antitrypsin | | HDQDHPTFNKITPNLAEFAFSLYRQLAHQSNSTNIFFSPVS |
| | | | | IATAFAMLSLGTKADTHDEILEGLNFNLTEIPEAQIHEGFQ |
| | | | | ELLRTLNQPDSQLQLTTGNGLFLSEGLKLVDKFLEDVKKL |
| | | | | YHSEAFTVNFGDTEEAKKQINDYVEKGTQGKIVDLVKEL |
| | | | | DRDTVFALVNYIFFKGKWERPFEVKDTEEEDFHVDQVTT |
| | | | | VKVPMMKRLGMFNIQHCKKLSSWVLLMKYLGNATAIFF |
| | | | | LPDEGKLQHLENELTHDIITKFLENEDRRSASLHLPKLSIT |
| | | | | GTYDLKSVLGQLGITKVFSNGADLSGVTEEAPLKLSKAVH |
| | | | | KAVLTIDEKGTEAAGAMFLEAIPMSIPPEVKFNKPFVFLM |
| | | | | IEQNTKSPLFMGKVVNPTQK |
| 3 | A2MG | Alpha-2- | P01023 | MGKNKLLHPSLVLLLLVLLPTDASVSGKPQYMVLVPSLLH |
| | | macroglobulin | | TETTEKGCVLLSYLNETVTVSASLESVRGNRSLFTDLEAEN |
| | | | | DVLHCVAFAVPKSSSNEEVMFLTVQVKGPTQEFKKRTTV |
| | | | | MVKNEDSLVFVQTDKSIYKPGQTVKFRVVSMDENFHPL |
| | | | | NELIPLVYIQDPKGNRIAQWQSFQLEGGLKQFSFPLSSEPF |
| | | | | QGSYKVVVQKKSGGRTEHPFTVEEFVLPKFEVQVTVPKII |
| | | | | TILEEEMNVSVCGLYTYGKPVPGHVTVSICRKYSDASDCH |
| | | | | GEDSQAFCEKFSGQLNSHGCFYQQVKTKVFQLKRKEYEM |
| | | | | KLHTEAQIQEEGTVVELTGRQSSEITRTITKLSFVKVDSHF |
| | | | | RQGIPFFGQVRLVDGKGVPIPNKVIFIRGNEANYYSNATT |
| | | | | DEHGLVQFSINTTNVMGTSLTVRVNYKDRSPCYGYQWV |
| | | | | SEEHEEAHHTAYLVFSPSKSFVHLEPMSHELPCGHTQTV |
| | | | | QAHYILNGGTLLGLKKLSFYYLIMAKGGIVRTGTHGLLVK |
| | | | | QEDMKGHFSISIPVKSDIAPVARLLIYAVLPTGDVIGDSAK |
| | | | | YDVENCLANKVDLSFSPSQSLPASHAHLRVTAAPQSVCA |
| | | | | LRAVDQSVLLMKPDAELSASSVYNLLPEKDLTGFPGPLND |
| | | | | QDNEDCINRHNVYINGITYTPVSSTNEKDMYSFLEDMGL |
| | | | | KAFTNSKIRKPKMCPQLQQYEMHGPEGLRVGFYESDVM |
| | | | | GRGHARLVHVEEPHTETVRKYFPETWIWDLVVVNSAGV |
| | | | | AEVGVTVPDTITEWKAGAFCLSEDAGLGISSTASLRAFQP |
| | | | | FFVELTMPYSVIRGEAFTLKATVLNYLPKCIRVSVQLEASP |
| | | | | AFLAVPVEKEQAPHCICANGRQTVSWAVTPKSLGNVNF |
| | | | | TVSAEALESQELCGTEVPSVPEHGRKDTVIKPLLVEPEGLE |
| | | | | KETTFNSLLCPSGGEVSEELSLKLPPNVVEESARASVSVLG |
| | | | | DILGSAMQNTQNLLQMPYGCGEQNMVLFAPNIYVLDYL |
| | | | | NETQQLTPEIKSKAIGYLNTGYQRQLNYKHYDGSYSTFGE |
| | | | | RYGRNQGNTWLTAFVLKTFAQARAYIFIDEAHITQALIW |
| | | | | LSQRQKDNGCFRSSGSLLNNAIKGGVEDEVTLSAYITIALL |
| | | | | EIPLTVTHPVVRNALFCLESAWKTAQEGDHGSHVYTKAL |
| | | | | LAYAFALAGNQDKRKEVLKSLNEEAVKKDNSVHWERPQ |
| | | | | KPKAPVGHFYEPQAPSAEVEMTSYVLLAYLTAQPAPTSE |
| | | | | DLTSATNIVKWITKQQNAQGGFSSTQDTVVALHALSKYG |
| | | | | AATFTRTGKAAQVTIQSSGTFSSKFQVDNNNRLLLQQVSL |
| | | | | PELPGEYSMKVTGEGCVYLQTSLKYNILPEKEEFPFALGV |
| | | | | QTLPQTCDEPKAHTSFQISLSVSYTGSRSASNMAIVDVKM |
| | | | | VSGFIPLKPTVKMLERSNHVSRTEVSSNHVLIYLDKVSNQ |
| | | | | TLSLFFTVLQDVPVRDLKPAIVKVYDYYETDEFAIAEYNA |
| | | | | PCSKDLGNA |
| 134 | CO5 | Complement | P01031 | MGLLGILCFLIFLGKTWGQEQTYVISAPKIFRVGASENIVI |
| | | C5 | | QVYGYTEAFDATISIKSYPDKKFSYSSGHVHLSSENKFQNS |
| | | | | AILTIQPKQLPGGQNPVSYVYLEVVSKHFSKSKRMPITYD |
| | | | | NGFLFIHTDKPVYTPDQSVKVRVYSLNDDLKPAKRETVL |
| | | | | TFIDPEGSEVDMVEEIDHIGIISFPDFKIPSNPRYGMWTIK |
| | | | | AKYKEDFSTTGTAYFEVKEYVLPHFSVSIEPEYNFIGYKNF |
| | | | | KNFEITIKARYFYNKVVTEADVYITFGIREDLKDDQKEM |
| | | | | MQTAMQNTMLINGIAQVTFDSETAVKELSYYSLEDLNNK |
| | | | | YLYIAVTVIESTGGFSEEAEIPGIKYVLSPYKLNLVATPLFL |
| | | | | KPGIPYPIKVQVKDSLDQLVGGVPVTLNAQTIDVNQETS |
| | | | | DLDPSKSVTRVDDGVASFVLNLPSGVTVLEFNVKTDAPD |
| | | | | LPEENQAREGYRAIAYSSLSQSYLYIDWTDNHKALLVGE |
| | | | | HLNIIVTPKSPYIDKITHYNYLILSKGKIIHFGTREKFSDASY |
| | | | | QSINIPVTQNMVPSSRLLVYYIVTGEQTAELVSDSVWLNI |
| | | | | EEKCGNQLQVHLSPDADAYSPGQTVSLNMATGMDSWV |
| | | | | ALAAVDSAVYGVQRGAKKPLERVFQFLEKSDLGCGAGG |
| | | | | GLNNANVFHLAGLTFLTNANADDSQENDEPCKEILRPRR |
| | | | | TLQKKIEEIAAKYKHSVVKKCCYDGACVNNDETCEQRAA |
| | | | | RISLGPRCIKAFTECCVVASQLRANISHKDMQLGRLHMKT |
| | | | | LLPVSKPEIRSYFPESWLWEVHLVPRRKQLQFALPDSLTT |
| | | | | WEIQGVGISNTGICVADTVKAKVFKDVFLEMNIPYSVVR |
| | | | | GEQIQLKGTVYNYRTSGMQFCVKMSAVEGICTSESPVID |
| | | | | HQGTKSSKCVRQKVEGSSSHLVTFTVLPLEIGLHNINFSLE |
| | | | | TWFGKEILVKTLRVVPEGVKRESYSGVTLDPRGIYGTISR |
| | | | | RKEFPYRIPLDLVPKTEIKRILSVKGLLVGEILSAVLSQEGI |
| | | | | NILTHLPKGSAEAELMSVVPVFYVFHYLETGNHWNIFHS |
| | | | | DPLIEKQKLKKKLKEGMLSIMSYRNADYSYSVWKGGSAS |
| | | | | TWLTAFALRVLGQVNKYVEQNQNSICNSLLWLVENYQL |
| | | | | DNGSFKENSQYQPIKLQGTLPVEARENSLYLTAFTVIGIRK |
| | | | | AFDICPLVKIDTALIKADNFLLENTLPAQSTFTLAISAYALS |
| | | | | LGDKTHPQFRSIVSALKREALVKGNPPIYRFWKDNLQHK |
| | | | | DSSVPNTGTARMVETTAYALLTSLNLKDINYVNPVIKWL |
| | | | | SEEQRYGGGFYSTQDTINAIEGLTEYSLLVKQLRLSMDID |
| | | | | VSYKHKGALHNYKMTDKNFLGRPVEVLLNDDLIVSTGFG |
| | | | | SGLATVHVTTVVHKTSTSEEVCSFYLKIDTQDIEASHYRG |
| | | | | YGNSDYKRIVACASYKPSREESSSGSSHAVMDISLPTGISA |
| | | | | NEEDLKALVEGVDQLFTDYQIKDGHVILQLNSIPSSDFLC |
| | | | | VRFRIFELFEVGFLSPATFTVYEYHRPDKQCTMFYSTSNIK |
| | | | | IQKVCEGAACKCVEADCGQMQEELDLTISAETRKQTACK |
| | | | | PEIAYAYKVSITSITVENVFVKYKATLLDIYKTGEAVAEKD |
| | | | | SEITFIKKVTCTNAELVKGRQYLIMGKEALQIKYNFSFRYI |
| | | | | YPLDSLTWIEYWPRDTTCSSCQAFLANLDEFAEDIFLNGC |
| 157 | PIGR | Polymeric | P01833 | MLLFVLTCLLAVFPAISTKSPIFGPEEVNSVEGNSVSITCYY |
| | | immunoglobulin | | PPTSVNRHTRKYWCRQGARGGCITLISSEGYVSSKYAGR |
| | | receptor | | ANLTNFPENGTFVVNIAQLSQDDSGRYKCGLGINSRGLSF |
| | | | | DVSLEVSQGPGLLNDTKVYTVDLGRTVTINCPFKTENAQ |
| | | | | KRKSLYKQIGLYPVLVIDSSGYVNPNYTGRIRLDIQGTGQ |
| | | | | LLFSVVINQLRLSDAGQYLCQAGDDSNSNKKNADLQVLK |
| | | | | PEPELVYEDLRGSVTFHCALGPEVANVAKFLCRQSSGENC |
| | | | | DVVVNTLGKRAPAFEGRILLNPQDKDGSFSVVITGLRKED |
| | | | | AGRYLCGAHSDGQLQEGSPIQAWQLFVNEESTIPRSPTV |
| | | | | VKGVAGGSVAVLCPYNRKESKSIKYWCLWEGAQNGRCP |
| | | | | LLVDSEGWVKAQYEGRLSLLEEPGNGTFTVILNQLTSRDA |
| | | | | GFYWCLTNGDTLWRTTVEIKIIEGEPNLKVPGNVTAVLG |
| | | | | ETLKVPCHFPCKFSSYEKYWCKWNNTGCQALPSQDEGPS |
| | | | | KAFVNCDENSRLVSLTLNLVTRADEGWYWCGVKQGHF |
| | | | | YGETAAVYVAVEERKAAGSRDVSLAKADAAPDEKVLDS |
| | | | | GFREIENKAIQDPRLFAEEKAVADTRDQADGSRASVDSGS |
| | | | | SEEQGGSSRALVSTLVPLGLVLAVGAVAVGVARARHRKN |
| | | | | VDRVSIRSYRTDISMSDFENSREFGANDNMGASSITQETSL |
| | | | | GGKEEFVATTESTTETKEPKKAKRSSKEEAEMAYKDFLLQ |
| | | | | SSTVAAEAQDGPQEA |
| 158 | IGHG1 | Immunoglobulin | P01857 | ASTKGPSVFPLAPSSKSTSGGTAALGCLVKDYFPEPVTVS |
| | | heavy | | WNSGALTSGVHTFPAVLQSSGLYSLSSVVTVPSSSLGTQT |
| | | constant | | YICNVNHKPSNTKVDKKVEPKSCDKTHTCPPCPAPELLG |
| | | gamma 1 | | GPSVFLFPPKPKDTLMISRTPEVTCVVVDVSHEDPEVKFN |
| | | | | WYVDGVEVHNAKTKPREEQYNSTYRVVSVLTVLHQDW |
| | | | | LNGKEYKCKVSNKALPAPIEKTISKAKGQPREPQVYTLPP |
| | | | | SRDELTKNQVSLTCLVKGFYPSDIAVEWESNGQPENNYK |
| | | | | TTPPVLDSDGSFFLYSKLTVDKSRWQQGNVFSCSVMHEA |
| | | | | LHNHYTQKSLSLSPGK |
| 159 | IGHG2 | Immunoglobulin | P01859 | ASTKGPSVFPLAPCSRSTSESTAALGCLVKDYFPEPVTVSW |
| | | heavy | | NSGALTSGVHTFPAVLQSSGLYSLSSVVTVPSSNFGTQTYT |
| | | constant | | CNVDHKPSNTKVDKTVERKCCVECPPCPAPPVAGPSVFL |
| | | gamma 2 | | FPPKPKDTLMISRTPEVTCVVVDVSHEDPEVQFNWYVD |
| | | | | GVEVHNAKTKPREEQFNSTFRVVSVLTVVHQDWLNGKE |
| | | | | YKCKVSNKGLPAPIEKTISKTKGQPREPQVYTLPPSREEM |
| | | | | TKNQVSLTCLVKGFYPSDISVEWESNGQPENNYKTTPPM |
| | | | | LDSDGSFFLYSKLTVDKSRWQQGNVFSCSVMHEALHNH |
| | | | | YTQKSLSLSPGK |
| 160 | IGHG4 | Immunoglobulin | P01861 | ASTKGPSVFPLAPCSRSTSESTAALGCLVKDYFPEPVTVSW |
| | | heavy | | NSGALTSGVHTFPAVLQSSGLYSLSSVVTVPSSSLGTKTYT |
| | | constant | | CNVDHKPSNTKVDKRVESKYGPPCPSCPAPEFLGGPSVFL |
| | | gamma 4 | | FPPKPKDTLMISRTPEVTCVVVDVSQEDPEVQFNWYVD |
| | | | | GVEVHNAKTKPREEQFNSTYRVVSVLTVLHQDWLNGKE |
| | | | | YKCKVSNKGLPSSIEKTISKAKGQPREPQVYTLPPSQEEMT |
| | | | | KNQVSLTCLVKGFYPSDIAVEWESNGQPENNYKTTPPVL |
| | | | | DSDGSFFLYSRLTVDKSRWQEGNVFSCSVMHEALHNHYT |
| | | | | QKSLSLSLGK |
| 161 | IGHA1 | Immunoglobulin | P01876 | ASPTSPKVFPLSLCSTQPDGNVVIACLVQGFFPQEPLSVT |
| | | heavy | | WSESGQGVTARNFPPSQDASGDLYTTSSQLTLPATQCLA |
| | | constant | | GKSVTCHVKHYTNPSQDVTVPCPVPSTPPTPSPSTPPTPS |
| | | alpha 1 | | PSCCHPRLSLHRPALEDLLLGSEANLTCTLTGLRDASGVTF |
| | | | | TWTPSSGKSAVQGPPERDLCGCYSVSSVLPGCAEPWNHG |
| | | | | KTFTCTAAYPESKTPLTATLSKSGNTFRPEVHLLPPPSEEL |
| | | | | ALNELVTLTCLARGFSPKDVLVRWLQGSQELPREKYLTW |
| | | | | ASRQEPSQGTTTFAVTSILRVAAEDWKKGDTFSCMVGHE |
| | | | | ALPLAFTQKTIDRLAGKPTHVNVSVVMAEVDGTCY |
| 162 | IGHA2 | Immunoglobulin | P01877 | ASPTSPKVFPLSLDSTPQDGNVVVACLVQGFFPQEPLSVT |
| | | heavy | | WSESGQNVTARNFPPSQDASGDLYTTSSQLTLPATQCPD |
| | | constant | | GKSVTCHVKHYTNSSQDVTVPCRVPPPPPCCHPRLSLHR |
| | | alpha 2 | | PALEDLLLGSEANLTCTLTGLRDASGATFTWTPSSGKSAV |
| | | | | QGPPERDLCGCYSVSSVLPGCAQPWNHGETFTCTAAHPE |
| | | | | LKTPLTANITKSGNTFRPEVHLLPPPSEELALNELVTLTCL |
| | | | | ARGFSPKDVLVRWLQGSQELPREKYLTWASRQEPSQGTT |
| | | | | TYAVTSILRVAAEDWKKGETFSCMVGHEALPLAFTQKTI |
| | | | | DRMAGKPTHINVSVVMAEADGTCY |
| 163 | SAMP | Serum | P02743 | MNKPLLWISVLTSLLEAFAHTDLSGKVFVFPRESVTDHVN |
| | | amyloid P- | | LITPLEKPLQNFTLCFRAYSDLSRAYSLFSYNTQGRDNELL |
| | | component | | VYKERVGEYSLYIGRHKVTSKVIEKFPAPVHICVSWESSS |
| | | | | GIAEFWINGTPLVKKGLRQGYFVEAQPKIVLGQEQDSYG |
| | | | | GKFDRSQSFVGEIGDLYMWDSVLPPENILSAYQGTPLPA |
| | | | | NILDWQALNYEIRGYVIIKPLVWV |
| 164 | CO9 | Complement | P02748 | MSACRSFAVAICILEISILTAQYTTSYDPELTESSGSASHIDC |
| | | component | | RMSPWSEWSQCDPCLRQMFRSRSIEVFGQFNGKRCTDA |
| | | C9 | | VGDRRQCVPTEPCEDAEDDCGNDFQCSTGRCIKMRLRC |
| | | | | NGDNDCGDFSDEDDCESEPRPPCRDRVVEESELARTAGY |
| | | | | GINILGMDPLSTPFDNEFYNGLCNRDRDGNTLTYYRRPW |
| | | | | NVASLIYETKGEKNFRTEHYEEQIEAFKSIIQEKTSNFNAAI |
| | | | | SLKFTPTETNKAEQCCEETASSISLHGKGSFRFSYSKNETY |
| | | | | QLFLSYSSKKEKMFLHVKGEIHLGRFVMRNRDVVLTTTF |
| | | | | VDDIKALPTTYEKGEYFAFLETYGTHYSSSGSLGGLYELIY |
| | | | | VLDKASMKRKGVELKDIKRCLGYHLDVSLAFSEISVGAEF |
| | | | | NKDDCVKRGEGRAVNITSENLIDDVVSLIRGGTRKYAFEL |
| | | | | KEKLLRGTVIDVTDFVNWASSINDAPVLISQKLSPIYNLVP |
| | | | | VKMKNAHLKKQNLERAIEDYINEFSVRKCHTCQNGGTVI |
| | | | | LMDGKCLCACPFKFEGIACEISKQKISEGLPALEFPNEK |
| 132 | TRFE | Serotransferrin | P02787 | MRLAVGALLVCAVLGLCLAVPDKTVRWCAVSEHEATKC |
| | | | | QSFRDHMKSVIPSDGPSVACVKKASYLDCIRAIAANEAD |
| | | | | AVTLDAGLVYDAYLAPNNLKPVVAEFYGSKEDPQTFYY |
| | | | | AVAVVKKDSGFQMNQLRGKKSCHTGLGRSAGWNIPIGL |
| | | | | LYCDLPEPRKPLEKAVANFFSGSCAPCADGTDFPQLCQLC |
| | | | | PGCGCSTLNQYFGYSGAFKCLKDGAGDVAFVKHSTIFEN |
| | | | | LANKADRDQYELLCLDNTRKPVDEYKDCHLAQVPSHTV |
| | | | | VARSMGGKEDLIWELLNQAQEHFGKDKSKEFQLFSSPHG |
| | | | | KDLLFKDSAHGFLKVPPRMDAKMYLGYEYVTAIRNLREG |
| | | | | TCPEAPTDECKPVKWCALSHHERLKCDEWSVNSVGKIEC |
| | | | | VSAETTEDCIAKIMNGEADAMSLDGGFVYIAGKCGLVPV |
| | | | | LAENYNKSDNCEDTPEAGYFAIAVVKKSASDLTWDNLKG |
| | | | | KKSCHTAVGRTAGWNIPMGLLYNKINHCRFDEFFSEGCA |
| | | | | PGSKKDSSLCKLCMGSGLNLCEPNNKEGYYGYTGAFRCL |
| | | | | VEKGDVAFVKHQTVPQNTGGKNPDPWAKNLNEKDYEL |
| | | | | LCLDGTRKPVEEYANCHLARAPNHAVVTRKDKEACVHK |
| | | | | ILRQQQHLFGSNVTDCSGNFCLFRSETKDLLFRDDTVCLA |
| | | | | KLHDRNTYEKYLGEEYVKAVGNLRKCSTSSLLEACTFRRP |
| 165 | APOB | Apolipoprotein | P04114 | MDPPRPALLALLALPALLLLLLAGARAEEEMLENVSLVCP |
| | | B-100 | | KDATRFKHLRKYTYNYEAESSSGVPGTADSRSATRINCKV |
| | | | | ELEVPQLCSFILKTSQCTLKEVYGFNPEGKALLKKTKNSEE |
| | | | | FAAAMSRYELKLAIPEGKQVFLYPEKDEPTYILNIKRGIIS |
| | | | | ALLVPPETEEAKQVLFLDTVYGNCSTHFTVKTRKGNVAT |
| | | | | EISTERDLGQCDRFKPIRTGISPLALIKGMTRPLSTLISSSQS |
| | | | | CQYTLDAKRKHVAEAICKEQHLFLPFSYKNKYGMVAQV |
| | | | | TQTLKLEDTPKINSRFFGEGTKKMGLAFESTKSTSPPKQAE |
| | | | | AVLKTLQELKKLTISEQNIQRANLFNKLVTELRGLSDEAV |
| | | | | TSLLPQLIEVSSPITLQALVQCGQPQCSTHILQWLKRVHA |
| | | | | NPLLIDVVTYLVALIPEPSAQQLREIFNMARDQRSRATLY |
| | | | | ALSHAVNNYHKTNPTGTQELLDIANYLMEQIQDDCTGD |
| | | | | EDYTYLILRVIGNMGQTMEQLTPELKSSILKCVQSTKPSL |
| | | | | MIQKAAIQALRKMEPKDKDQEVLLQTFLDDASPGDKRL |
| | | | | AAYLMLMRSPSQADINKIVQILPWEQNEQVKNFVASHIA |
| | | | | NILNSEELDIQDLKKLVKEALKESQLPTVMDFRKFSRNYQ |
| | | | | LYKSVSLPSLDPASAKIEGNLIFDPNNYLPKESMLKTTLTA |
| | | | | FGFASADLIEIGLEGKGFEPTLEALFGKQGFFPDSVNKALY |
| | | | | WVNGQVPDGVSKVLVDHFGYTKDDKHEQDMVNGIML |
| | | | | SVEKLIKDLKSKEVPEARAYLRILGEELGFASLHDLQLLGK |
| | | | | LLLMGARTLQGIPQMIGEVIRKGSKNDFFLHYIFMENAFE |
| | | | | LPTGAGLQLQISSSGVIAPGAKAGVKLEVANMQAELVAK |
| | | | | PSVSVEFVTNMGIIIPDFARSGVQMNTNFFHESGLEAHVA |
| | | | | LKAGKLKFIIPSPKRPVKLLSGGNTLHLVSTTKTEVIPPLIE |
| | | | | NRQSWSVCKQVFPGLNYCTSGAYSNASSTDSASYYPLTG |
| | | | | DTRLELELRPTGEIEQYSVSATYELQREDRALVDTLKFVT |
| | | | | QAEGAKQTEATMTFKYNRQSMTLSSEVQIPDFDVDLGTI |
| | | | | LRVNDESTEGKTSYRLTLDIQNKKITEVALMGHLSCDTKE |
| | | | | ERKIKGVISIPRLQAEARSEILAHWSPAKLLLQMDSSATAY |
| | | | | GSTVSKRVAWHYDEEKIEFEWNTGTNVDTKKMTSNFPV |
| | | | | DLSDYPKSLHMYANRLLDHRVPQTDMTFRHVGSKLIVA |
| | | | | MSSWLQKASGSLPYTQTLQDHLNSLKEFNLQNMGLPDF |
| | | | | HIPENLFLKSDGRVKYTLNKNSLKIEIPLPFGGKSSRDLKM |
| | | | | LETVRTPALHFKSVGFHLPSREFQVPTFTIPKLYQLQVPLL |
| | | | | GVLDLSTNVYSNLYNWSASYSGGNTSTDHFSLRARYHMK |
| | | | | ADSVVDLLSYNVQGSGETTYDHKNTFTLSYDGSLRHKFL |
| | | | | DSNIKFSHVEKLGNNPVSKGLLIFDASSSWGPQMSASVHL |
| | | | | DSKKKQHLFVKEVKIDGQFRVSSFYAKGTYGLSCQRDPN |
| | | | | TGRLNGESNLRFNSSYLQGTNQITGRYEDGTLSLTSTSDLQ |
| | | | | SGIIKNTASLKYENYELTLKSDTNGKYKNFATSNKMDMTF |
| | | | | SKQNALLRSEYQADYESLRFFSLLSGSLNSHGLELNADILG |
| | | | | TDKINSGAHKATLRIGQDGISTSATTNLKCSLLVLENELNA |
| | | | | ELGLSGASMKLTTNGRFREHNAKFSLDGKAALTELSLGSA |
| | | | | YQAMILGVDSKNIFNFKVSQEGLKLSNDMMGSYAEMKF |
| | | | | DHTNSLNIAGLSLDFSSKLDNIYSSDKFYKQTVNLQLQPYS |
| | | | | LVTTLNSDLKYNALDLTNNGKLRLEPLKLHVAGNLKGAY |
| | | | | QNNEIKHIYAISSAALSASYKADTVAKVQGVEFSHRLNTD |
| | | | | IAGLASAIDMSTNYNSDSLHFSNVFRSVMAPFTMTIDAHT |
| | | | | NGNGKLALWGEHTGQLYSKFLLKAEPLAFTFSHDYKGST |
| | | | | SHHLVSRKSISAALEHKVSALLTPAEQTGTWKLKTQENN |
| | | | | NEYSQDLDAYNTKDKIGVELTGRTLADLTLLDSPIKVPLL |
| | | | | LSEPINIIDALEMRDAVEKPQEFTIVAFVKYDKNQDVHSI |
| | | | | NLPFFETLQEYFERNRQTIIVVLENVQRNLKHINIDQFVR |
| | | | | KYRAALGKLPQQANDYLNSFNWERQVSHAKEKLTALTK |
| | | | | KYRITENDIQIALDDAKINFNEKLSQLQTYMIQFDQYIKDS |
| | | | | YDLHDLKIAIANIIDEIIEKLKSLDEHYHIRVNLVKTIHDLH |
| | | | | LFIENIDFNKSGSSTASWIQNVDTKYQIRIQIQEKLQQLKR |
| | | | | HIQNIDIQHLAGKLKQHIEAIDVRVLLDQLGTTISFERIND |
| | | | | ILEHVKHFVINLIGDFEVAEKINAFRAKVHELIERYEVDQ |
| | | | | QIQVLMDKLVELAHQYKLKETIQKLSNVLQQVKIKDYFE |
| | | | | KLVGFIDDAVKKLNELSFKTFIEDVNKFLDMLIKKLKSFDY |
| | | | | HQFVDETNDKIREVTQRLNGEIQALELPQKAEALKLFLEE |
| | | | | TKATVAVYLESLQDTKITLIINWLQEALSSASLAHMKAKF |
| | | | | RETLEDTRDRMYQMDIQQELQRYLSLVGQVYSTLVTYIS |
| | | | | DWWTLAAKNLTDFAEQYSIQDWAKRMKALVEQGFTVP |
| | | | | EIKTILGTMPAFEVSLQALQKATFQTPDFIVPLTDLRIPSV |
| | | | | QINFKDLKNIKIPSRFSTPEFTILNTFHIPSFTIDFVEMKVKII |
| | | | | RTIDQMLNSELQWPVPDIYLRDLKVEDIPLARITLPDFRL |
| | | | | PEIAIPEFIIPTLNLNDFQVPDLHIPEFQLPHISHTIEVPTFG |
| | | | | KLYSILKIQSPLFTLDANADIGNGTTSANEAGIAASITAKG |
| | | | | ESKLEVLNFDFQANAQLSNPKINPLALKESVKFSSKYLRTE |
| | | | | HGSEMLFFGNAIEGKSNTVASLHTEKNTLELSNGVIVKIN |
| | | | | NQLTLDSNTKYFHKLNIPKLDFSSQADLRNEIKTLLKAGHI |
| | | | | AWTSSGKGSWKWACPRFSDEGTHESQISFTIEGPLTSFGL |
| | | | | SNKINSKHLRVNQNLVYESGSLNFSKLEIQSQVDSQHVGH |
| | | | | SVLTAKGMALFGEGKAEFTGRHDAHLNGKVIGTLKNSLF |
| | | | | FSAQPFEITASTNNEGNLKVRFPLRLTGKIDFLNNYALFLS |
| | | | | PSAQQASWQVSARFNQYKYNQNFSAGNNENIMEAHVGI |
| | | | | NGEANLDFLNIPLTIPEMRLPYTIITTPPLKDFSLWEKTGL |
| | | | | KEFLKTTKQSFDLSVKAQYKKNKHRHSITNPLAVLCEFIS |
| | | | | QSIKSFDRHFEKNRNNALDFVTKSYNETKIKFDKYKAEKS |
| | | | | HDELPRTFQIPGYTVPVVNVEVSPFTIEMSAFGYVFPKAV |
| | | | | SMPSFSILGSDVRVPSYTLILPSLELPVLHVPRNLKLSLPDF |
| | | | | KELCTISHIFIPAMGNITYDFSFKSSVITLNTNAELFNQSDI |
| | | | | VAHLLSSSSSVIDALQYKLEGTTRLTRKRGLKLATALSLSN |
| | | | | KFVEGSHNSTVSLTTKNMEVSVATTTKAQIPILRMNFKQE |
| | | | | LNGNTKSKPTVSSSMEFKYDFNSSMLYSTAKGAVDHKLSL |
| | | | | ESLTSYFSIESSTKGDVKGSVLSREYSGTIASEANTYLNSKST |
| | | | | RSSVKLQGTSKIDDIWNLEVKENFAGEATLQRIYSLWEHS |
| | | | | TKNHLQLEGLFFTNGEHTSKATLELSPWQMSALVQVHAS |
| | | | | QPSSFHDFPDLGQEVALNANTKNQKIRWKNEVRIHSGSF |
| | | | | QSQVELSNDQEKAHLDIAGSLEGHLRFLKNIILPVYDKSL |
| | | | | WDFLKLDVTTSIGRRQHLRVSTAFVYTKNPNGYSFSIPVK |
| | | | | VLADKFIIPGLKLNDLNSVLVMPTFHVPFTDLQVPSCKLD |
| | | | | FREIQIYKKLRTSSFALNLPTLPEVKFPEVDVLTKYSQPEDS |
| | | | | LIPFFEITVPESQLTVSQFTLPKSVSDGIAALDLNAVANKIA |
| | | | | DFELPTIIVPEQTIEIPSIKFSVPAGIVIPSFQALTARFEVDSP |
| | | | | VYNATWSASLKNKADYVETVLDSTCSSTVQFLEYELNVL |
| | | | | GTHKIEDGTLASKTKGTFAHRDFSAEYEEDGKYEGLQEW |
| | | | | EGKAHLNIKSPAFTDLHLRYQKDKKGISTSAASPAVGTVG |
| | | | | MDMDEDDDFSKWNFYYSPQSSPDKKLTIFKTELRVRESD |
| | | | | EETQIKVNWEEEAASGLLTSLKDNVPKATGVLYDYVNKY |
| | | | | HWEHTGLTLREVSSKLRRNLQNNAEWVYQGAIRQIDDI |
| | | | | DVRFQKAASGTTGTYQEWKDKAQNLYQELLTQEGQASF |
| | | | | QGLKDNVFDGLVRVTQEFHMKVKHLIDSLIDFLNFPRFQ |
| | | | | FPGKPGIYTREELCTMFIREVGTVLSQVYSKVHNGSEILFS |
| | | | | YFQDLVITLPFELRKHKLIDVISMYRELLKDLSKEAQEVFK |
| | | | | AIQSLKTTEVLRNLQDLLQFIFQLIEDNIKQLKEMKFTYLI |
| | | | | NYIQDEINTIFSDYIPYVFKLLKENLCLNLHKFNEFIQNELQ |
| | | | | EASQELQQIHQYIMALREEYFDPSIVGWTVKYYELEEKIV |
| | | | | SLIKNLLVALKDFHSEYIVSASNFTSQLSSQVEQFLHRNIQE |
| | | | | YLSILTDPDGKGKEKIAELSATAQEIIKSQALATKKIISDYH |
| | | | | QQFRYKLQDFSDQLSDYYEKFIAESKRLIDLSIQNYHTFLI |
| | | | | YITELLKKLQSTTVMNPYMKLAPGELTIIL |
| 166 | CO4A | Complement | POCOL4 | MRLLWGLIWASSFFTLSLQKPRLLLFSPSVVHLGVPLSVG |
| | | C4-A | | VQLQDVPRGQVVKGSVFLRNPSRNNVPCSPKVDFTLSSE |
| | | | | RDFALLSLQVPLKDAKSCGLHQLLRGPEVQLVAHSPWLK |
| | | | | DSLSRTTNIQGINLLFSSRRGHLFLQTDQPIYNPGQRVRYR |
| | | | | VFALDQKMRPSTDTITVMVENSHGLRVRKKEVYMPSSIF |
| | | | | QDDFVIPDISEPGTWKISARFSDGLESNSSTQFEVKKYVLP |
| | | | | NFEVKITPGKPYILTVPGHLDEMQLDIQARYIYGKPVQG |
| | | | | VAYVRFGLLDEDGKKTFFRGLESQTKLVNGQSHISLSKAE |
| | | | | FQDALEKLNMGITDLQGLRLYVAAAIIESPGGEMEEAELT |
| | | | | SWYFVSSPFSLDLSKTKRHLVPGAPFLLQALVREMSGSPA |
| | | | | SGIPVKVSATVSSPGSVPEVQDIQQNTDGSGQVSIPIIIPQT |
| | | | | ISELQLSVSAGSPHPAIARLTVAAPPSGGPGFLSIERPDSRP |
| | | | | PRVGDTLNLNLRAVGSGATFSHYYYMILSRGQIVFMNRE |
| | | | | PKRTLTSVSVFVDHHLAPSFYFVAFYYHGDHPVANSLRV |
| | | | | DVQAGACEGKLELSVDGAKQYRNGESVKLHLETDSLALV |
| | | | | ALGALDTALYAAGSKSHKPLNMGKVFEAMNSYDLGCGP |
| | | | | GGGDSALQVFQAAGLAFSDGDQWTLSRKRLSCPKEKTTR |
| | | | | KKRNVNFQKAINEKLGQYASPTAKRCCQDGVTRLPMMR |
| | | | | SCEQRAARVQQPDCREPFLSCCQFAESLRKKSRDKGQAG |
| | | | | LQRALEILQEEDLIDEDDIPVRSFFPENWLWRVETVDRFQ |
| | | | | ILTLWLPDSLTTWEIHGLSLSKTKGLCVATPVQLRVFREF |
| | | | | HLHLRLPMSVRRFEQLELRPVLYNYLDKNLTVSVHVSPV |
| | | | | EGLCLAGGGGLAQQVLVPAGSARPVAFSVVPTAAAAVSL |
| | | | | KVVARGSFEFPVGDAVSKVLQIEKEGAIHREELVYELNPL |
| | | | | DHRGRTLEIPGNSDPNMIPDGDFNSYVRVTASDPLDTLGS |
| | | | | EGALSPGGVASLLRLPRGCGEQTMIYLAPTLAASRYLDKT |
| | | | | EQWSTLPPETKDHAVDLIQKGYMRIQQFRKADGSYAAW |
| | | | | LSRDSSTWLTAFVLKVLSLAQEQVGGSPEKLQETSNWLLS |
| | | | | QQQADGSFQDPCPVLDRSMQGGLVGNDETVALTAFVTI |
| | | | | ALHHGLAVFQDEGAEPLKQRVEASISKANSFLGEKASAGL |
| | | | | LGAHAAAITAYALTLTKAPVDLLGVAHNNLMAMAQET |
| | | | | GDNLYWGSVTGSQSNAVSPTPAPRNPSDPMPQAPALWI |
| | | | | ETTAYALLHLLLHEGKAEMADQASAWLTRQGSFQGGFR |
| | | | | STQDTVIALDALSAYWIASHTTEERGLNVTLSSTGRNGFK |
| | | | | SHALQLNNRQIRGLEEELQFSLGSKINVKVGGNSKGTLKV |
| | | | | LRTYNVLDMKNTTCQDLQIEVTVKGHVEYTMEANEDYE |
| | | | | DYEYDELPAKDDPDAPLQPVTPLQLFEGRRNRRRREAPK |
| | | | | VVEEQESRVHYTVCIWRNGKVGLSGMAIADVTLLSGFH |
| | | | | ALRADLEKLTSLSDRYVSHFETEGPHVLLYFDSVPTSRECV |
| | | | | GFEAVQEVPVGLVQPASATLYDYYNPERRCSVFYGAPSK |
| | | | | SRLLATLCSAEVCQCAEGKCPRQRRALERGLQDEDGYRM |
| | | | | KFACYYPRVEYGFQVKVLREDSRAAFRLFETKITQVLHFT |
| | | | | KDVKAAANQMRNFLVRASCRLRLEPGKEYLIMGLDGAT |
| | | | | YDLEGHPQYLLDSNSWIEEMPSERLCRSTRQRAACAQLN |
| | | | | DFLQEYGTQGCQV |
| 167 | CLUS | Clusterin | P10909 | MMKTLLLFVGLLLTWESGQVLGDQTVSDNELQEMSNQG |
| | | | | SKYVNKEIQNAVNGVKQIKTLIEKTNEERKTLLSNLEEAK |
| | | | | KKKEDALNETRESETKLKELPGVCNETMMALWEECKPCL |
| | | | | KQTCMKFYARVCRSGSGLVGRQLEEFLNQSSPFYFWMN |
| | | | | GDRIDSLLENDRQQTHMLDVMQDHFSRASSIIDELFQDR |
| | | | | FFTREPQDTYHYLPFSLPHRRPHFFFPKSRIVRSLMPFSPY |
| | | | | EPLNFHAMFQPFLEMIHEAQQAMDIHFHSPAFQHPPTEF |
| | | | | IREGDDDRTVCREIRHNSTGCLRMKDQCDKCREILSVDCS |
| | | | | TNNPSQAKLRRELDESLQVAERLTRKYNELLKSYQWKML |
| | | | | NTSSLLEQLNEQFNWVSRLANLTQGEDQYYLRVTTVASH |
| | | | | TSDSDVPSGVTEVVVKLFDSDPITVTVPVEVSRKNPKFME |
| | | | | TVAEKALQEYRKKHREE |
| 122 | CO6 | Complement | P13671 | MARRSVLYFILLNALINKGQACFCDHYAWTQWTSCSKT |
| | | component | | CNSGTQSRHRQIVVDKYYQENFCEQICSKQETRECNWQ |
| | | C6 | | RCPINCLLGDFGPWSDCDPCIEKQSKVRSVLRPSQFGGQP |
| | | | | CTAPLVAFQPCIPSKLCKIEEADCKNKFRCDSGRCIARKLE |
| | | | | CNGENDCGDNSDERDCGRTKAVCTRKYNPIPSVQLMGN |
| | | | | GFHFLAGEPRGEVLDNSFTGGICKTVKSSRTSNPYRVPAN |
| | | | | LENVGFEVQTAEDDLKTDFYKDLTSLGHNENQQGSFSSQ |
| | | | | GGSSFSVPIFYSSKRSENINHNSAFKQAIQASHKKDSSFIRI |
| | | | | HKVMKVLNFTTKAKDLHLSDVFLKALNHLPLEYNSALYS |
| | | | | RIFDDFGTHYFTSGSLGGVYDLLYQFSSEELKNSGLTEEEA |
| | | | | KHCVRIETKKRVLFAKKTKVEHRCTTNKLSEKHEGSFIQG |
| | | | | AEKSISLIRGGRSEYGAALAWEKGSSGLEEKTFSEWLESVK |
| | | | | ENPAVIDFELAPIVDLVRNIPCAVTKRNNLRKALQEYAAK |
| | | | | FDPCQCAPCPNNGRPTLSGTECLCVCQSGTYGENCEKQS |
| | | | | PDYKSNAVDGQWGCWSSWSTCDATYKRSRTRECNNPA |
| | | | | PQRGGKRCEGEKRQEEDCTFSIMENNGQPCINDDEEMKE |
| | | | | VDLPEIEADSGCPQPVPPENGFIRNEKQLYLVGEDVEISCL |
| | | | | TGFETVGYQYFRCLPDGTWRQGDVECQRTECIKPVVQE |
| | | | | VLTITPFQRLYRIGESIELTCPKGFVVAGPSRYTCQGNSWT |
| | | | | PPISNSLTCEKDTLTKLKGHCQLGQKQSGSECICMSPEEDC |
| | | | | SHHSEDLCVFDTDSNDYFTSPACKFLAEKCLNNQQLHFL |
| | | | | HIGSCQDGRQLEWGLERTRLSSNSTKKESCGYDTCYDWE |
| | | | | KCSASTSKCVCLLPPQCFKGGNQLYCVKMGSSTSEKTLNI |
| | | | | CEVGTIRCANRKMEILHPGKCLA |
| 18 | ITIH4 | Inter-alpha- | Q14624 | MKPPRPVRTCSKVLVLLSLLAIHQTTTAEKNGIDIYSLTVD |
| | | trypsin | | SRVSSRFAHTVVTSRVVNRANTVQEATFQMELPKKAFIT |
| | | inhibitor | | NFSMIIDGMTYPGIIKEKAEAQAQYSAAVAKGKSAGLVK |
| | | heavy chain | | ATGRNMEQFQVSVSVAPNAKITFELVYEELLKRRLGVYE |
| | | H4 | | LLLKVRPQQLVKHLQMDIHIFEPQGISFLETESTFMTNQL |
| | | | | VDALTTWQNKTKAHIRFKPTLSQQQKSPEQQETVLDGN |
| | | | | LIIRYDVDRAISGGSIQIENGYFVHYFAPEGLTTMPKNVVF |
| | | | | VIDKSGSMSGRKIQQTREALIKILDDLSPRDQFNLIVFSTEA |
| | | | | TQWRPSLVPASAENVNKARSFAAGIQALGGTNINDAML |
| | | | | MAVQLLDSSNQEERLPEGSVSLIILLTDGDPTVGETNPRSI |
| | | | | QNNVREAVSGRYSLFCLGFGFDVSYAFLEKLALDNGGLA |
| | | | | RRIHEDSDSALQLQDFYQEVANPLLTAVTFEYPSNAVEEV |
| | | | | TQNNFRLLFKGSEMVVAGKLQDRGPDVLTATVSGKLPTQ |
| | | | | NITFQTESSVAEQEAEFQSPKYIFHNFMERLWAYLTIQQL |
| | | | | LEQTVSASDADQQALRNQALNLSLAYSFVTPLTSMVVTK |
| | | | | PDDQEQSQVAEKPMEGESRNRNVHSGSTFFKYYLQGAKI |
| | | | | PKPEASFSPRRGWNRQAGAAGSRMNFRPGVLSSRQLGLP |
| | | | | GPPDVPDHAAYHPFRRLAILPASAPPATSNPDPAVSRVM |
| | | | | NMKIEETTMTTQTPAPIQAPSAILPLPGQSVERLCVDPRH |
| | | | | RQGPVNLLSDPEQGVEVTGQYEREKAGFSWIEVTFKNPL |
| | | | | VWVHASPEHVVVTRNRRSSAYKWKETLFSVMPGLKMT |
| | | | | MDKTGLLLLSDPDKVTIGLLFWDGRGEGLRLLLRDTDRFS |
| | | | | SHVGGTLGQFYQEVLWGSPAASDDGRRTLRVQGNDHSA |
| | | | | TRERRLDYQEGPPGVEISCWSVEL |

TABLE 13A
Details of glycopeptides with different abundances in healthy
control and colorectal cancer (CRC) samples

| | | | Linking | Linking | | |
|---|---|---|---|---|---|---|
| SEQ | | | Site Pos. | Site Pos. | Glycan | |
| ID | Peptide Structure (PS) | | in Protein | in Peptide | Structure | Glycopeptide |
| NO | NAME | Peptide Sequence | Sequence | Sequence | GL NO | Abundance |
| 168 | HPT (241) - 4310 | VVLHPNYSQVDIGLIK | 241 | 6 | 4310 | Mid |
| 169 | HPT (241) - 4501 | VVLHPNYSQVDIGLIK | 241 | 6 | 4501 | Mid |
| 170 | HPT (241) - 5301 | VVLHPNYSQVDIGLIK | 241 | 6 | 5301 | Mid |
| 171 | HPT (241) - 5401 | VVLHPNYSQVDIGLIKL | 241 | 6 | 5401 | Mid |
| | | KQKVSVNER | | | | |
| 172 | HPT (184) - 5501 | MVSHHNLTTGATLINE | 184 | 6 | 5501 | Mid |
| | | QWLLTTAK | | | | |
| 173 | A1AT (70) - 4401 | QLAHQSNSTNIFFSPVSI | 70 | 7 | 4401 | Mid |
| | | ATAFAMLSLGTK | | | | |
| 174 | A1AT (107) - 4311 | ADTHDEILEGLNFNLTE | 107 | 14 | 4311 | Low |
| | | IPEAQIHEGFQELLR | | | | |
| 175 | A1AT (107) - 5403 | ADTHDEILEGLNFNLTE | 107 | 14 | 5403 | Low |
| | | IPEAQIHEGFQELLR | | | | |
| 176 | A1AT (107) - 7624 | ADTHDEILEGLNFNLTE | 107 | 14 | 7624 | Mid |
| | | IPEAQIHEGFQELLR | | | | |
| 177 | A1AT (271) - 5502 | YLGNATAIFFLPDEGK | 271 | 4 | 5502 | Mid |
| 178 | A2MG (1424) - 6502 | TEVSSNHVLIYLDKVSN | 1424 | 17 | 6502 | Mid |
| | | QTLSLFFTVLQDVPVR | | | | |
| 179 | A2MG (1424) - 6503 | TEVSSNHVLIYLDKVSN | 1424 | 17 | 6503 | Mid |
| | | QTLSLFFTVLQDVPVR | | | | |
| 180 | A2MG (1424) - 6511 | TEVSSNHVLIYLDKVSN | 1424 | 17 | 6511 | Mid |
| | | QTLSLFFTVLQDVPVR | | | | |
| 181 | CO5 (911) - 5401 | VEGSSSHLVTFTVLPLEI | 911 | 23 | 5401 | Mid |
| | | GLHNINFSLETWFGK | | | | |
| 182 | PIGR (421) - 5402 | LSLLEEPGNGTFTVILN | 421 | 9 | 5402 | Mid |
| | | QLTSR | | | | |
| 183 | IGHG1 (180) - 3410 | EEQYNSTYRVVSVLTVL | 180 | 5 | 3410 | Mid |
| | | HQDWLNGKEYKCK | | | | |
| 184 | IGHG1 (180) - 3510 | TKPREEQYNSTYRVVS | 180 | 5 | 3510 | Mid |
| | | VLTVLHQDWLNGK | | | | |
| 185 | IGHG2 (176) - 3310 | EEQFNSTFRVVSVLTVV | 176 | 5 | 3310 | Low |
| | | HQDWLNGKEYK | | | | |
| 186 | IGHG2 (176) - 3510 | EEQFNSTFRVVSVLTVV | 176 | 5 | 3510 | Mid |
| | | HQDWLNGKEYK | | | | |
| 187 | IGHG4 (177) - 3300 | EEQFNSTYRVVSVLTVL | 177 | 5 | 3300 | Mid |
| | | HQDWLNGK | | | | |
| 188 | IGHA1 (340) - 3200 | LAGKPTHVNVSVVMA | 340 | 9 | 3200 | Low |
| | | EVDGTCY | | | | |
| 189 | IGHA2 (327) - 6200 | MAGKPTHINVSVVMA | 327 | 9 | 6200 | Low |
| | | EADGTCY | | | | |
| 190 | SAMP (51) - 5402 | ESVTDHVNLITPLEKPL | 51 | 19 | 5402 | Low |
| | | QNFTLCFRAYSDLSR | | | | |
| 191 | CO9 (415) - 6513 | AVNITSENLIDDVVSLIR | 415 | 3 | 6513 | Low |
| 192 | TRFE (432) - 5402 | IMNGEADAMSLDGGFV | 432 | 33 | 5402 | Mid |
| | | YIAGKCGLVPVLAENY | | | | |
| | | NK | | | | |
| 193 | APOB (3465) - 5401 | SKPTVSSSMEFKYDFNS | 3465 | 16 | 5401 | Low |
| | | SMLYSTAKGAVDHK | | | | |
| 194 | CO4A (226) - 9200 | FSDGLESNSSTQFEVKK | 226 | 8 | 9200 | Mid |
| | | YVLPNFEVK | | | | |
| 195 | CO4A (1328) - 5402 | STQDTVIALDALSAYWI | 1328 | 28 | 5402 | Low |
| | | ASHTTEERGLNVTLSST | | | | |
| | | GR | | | | |
| 196 | CLUS (354) - 6512 | MLNTSSLLEQLNEQFN | 354 | 3 | 6512 | Low |
| | | WVSR | | | | |
| 197 | CO6 (855) - 5401 | LSSNSTK | 855 | 4 | 5401 | Mid |
| 198 | ITIH4 (81) - 5412 | AFITNFSMIIDGMTYPG | 81 | 5 | 5412 | Low |
| | | IIK | | | | |

TABLE 13B
Details of subset of glycopeptides with different abundances in
healthy control and colorectal cancer (CRC) samples

| | | | Linking | Linking | | |
|---|---|---|---|---|---|---|
| SEQ | | | Site Pos. | Site Pos. | Glycan | |
| ID | Peptide Structure (PS) | | in Protein | in Peptide | Structure | Glycopeptide |
| NO | NAME | Peptide Sequence | Sequence | Sequence | GL NO | Abundance |
| 168 | HPT (241) - 4310 | VVLHPNYSQVDIGLIK | 241 | 6 | 4310 | Mid |
| 171 | HPT (241) - 5401 | VVLHPNYSQVDIGLIKL | 241 | 6 | 5401 | Mid |
| | | KQKVSVNER | | | | |
| 172 | HPT (184) - 5501 | MVSHHNLTTGATLINE | 184 | 6 | 5501 | Mid |
| | | QWLLTTAK | | | | |
| 176 | A1AT (107) - 7624 | ADTHDEILEGLNFNLTE | 107 | 14 | 7624 | Mid |
| | | IPEAQIHEGFQELLR | | | | |
| 181 | CO5 (911) - 5401 | VEGSSSHLVTFTVLPLEI | 911 | 23 | 5401 | Mid |
| | | GLHNINFSLETWFGK | | | | |
| 184 | IGHG1 (180) - 3510 | TKPREEQYNSTYRVVS | 180 | 5 | 3510 | Mid |
| | | VLTVLHQDWLNGK | | | | |
| 187 | IGHG4 (177) - 3300 | EEQFNSTYRVVSVLTVL | 177 | 5 | 3300 | Mid |
| | | HQDWLNGK | | | | |
| 192 | TRFE (432) - 5402 | IMNGEADAMSLDGGFV | 432 | 33 | 5402 | Mid |
| | | YIAGKCGLVPVLAENY | | | | |
| | | NK | | | | |
| 194 | CO4A (226) - 9200 | FSDGLESNSSTQFEVKK | 226 | 8 | 9200 | Mid |
| | | YVLPNFEVK | | | | |

Table 15 illustrate the symbol structure and composition of detected glycan moieties that correspond to glycopeptides of Table 13A and Table 13B based on the Glycan GL NO. The term Symbol Structure illustrates a geometric linking structure of the carbohydrates where the bottommost carbohydrate such as N-acetylglucosamine is bound to the designated amino acid for an N-linked glycan. For reference, N-linked glycans have a glycan attached to the amino acid asparagine.

The identity of the various monosaccharides is illustrated by the Legend section located at the end of Table 15. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

TABLE 15
Glycan structure GL NO, symbol structure, and composition

| Glycan Structure | Symbol Structure | Composition |
|---|---|---|
| 3200 | 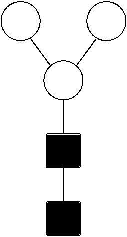 | Hex(3)HexNAc(2)Fuc(0)NeuAc(0) |
| 3300 | 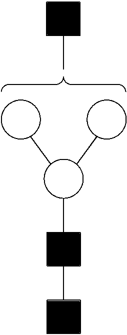 | Hex(3)HexNAc(3)Fuc(0)NeuAc(0) |
| 3310 | 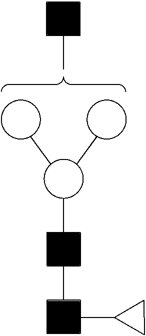 | Hex(3)HexNAc(3)Fuc(1)NeuAc(0) |
| 3410 | 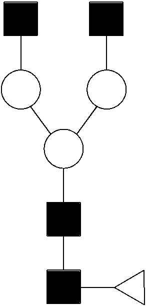 | Hex(3)HexNAc(4)Fuc(1)NeuAc(0) |
| 3510 | 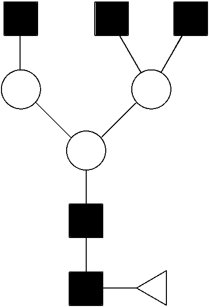 | Hex(3)HexNAc(5)Fuc(1)NeuAc(0) |
| 4310 | 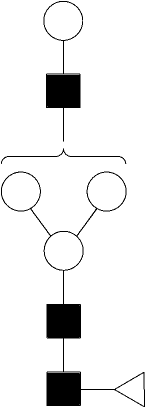 | Hex(4)HexNAc(3)Fuc(1)NeuAc(0) |
| 4311 | 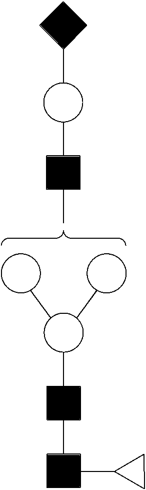 | Hex(4)HexNAc(3)Fuc(1)NeuAc(1) |
| 4401 | 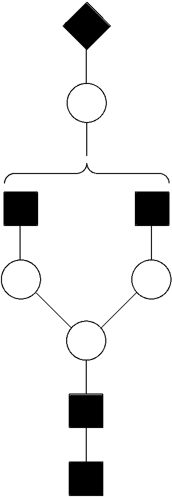 | Hex(4)HexNAc(4)Fuc(0)NeuAc(1) |
| 4501 | 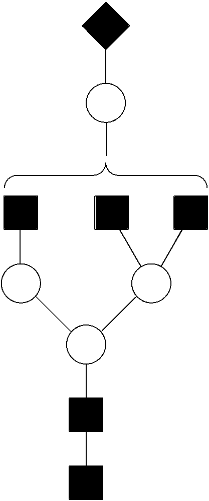 | Hex(4)HexNAc(5)Fuc(0)NeuAc(1) |
| 5301 | 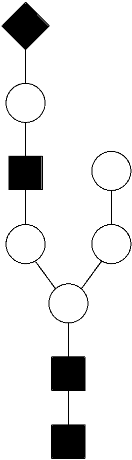 | Hex(5)HexNAc(3)Fuc(0)NeuAc(1) |
| 5401 | 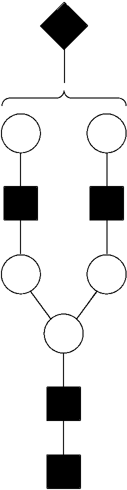 | Hex(5)HexNAc(4)Fuc(0)NeuAc(1) |
| 5402 | 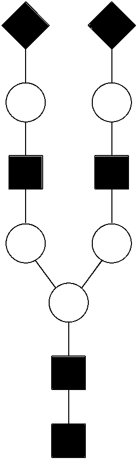 | Hex(5)HexNAc(4)Fuc(0)NeuAc(2) |
| 5412 | 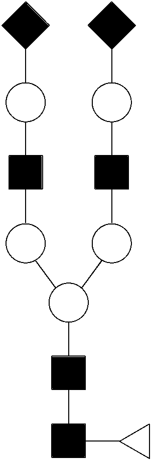 | Hex(5)HexNAc(4)Fuc(1)NeuAc(2) |
| 5501 | 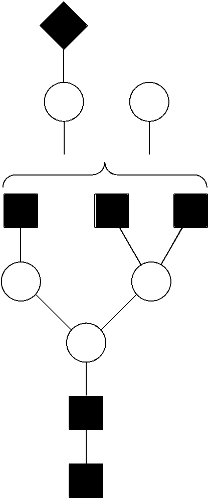 | Hex(5)HexNAc(5)Fuc(0)NeuAc(1) |
| 5502 | 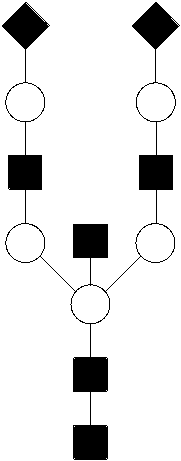 | Hex(5)HexNAc(5)Fuc(0)NeuAc(2) |
| 6200 | 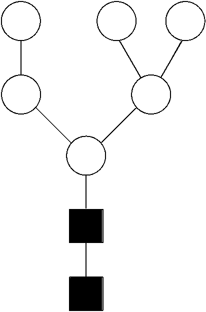 | Hex(6)HexNAc(2)Fuc(0)NeuAc(0) |
| 6502 | 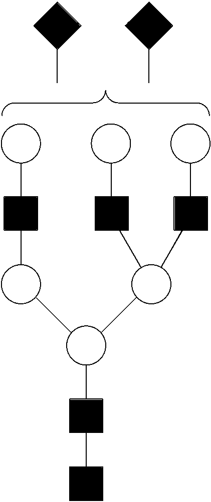 | Hex(6)HexNAc(5)Fuc(0)NeuAc(2) |
| 6503 | 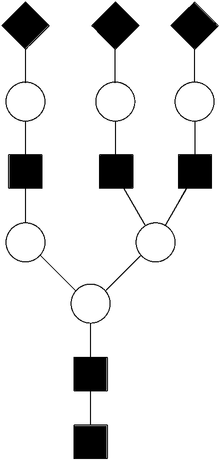 | Hex(6)HexNAc(5)Fuc(0)NeuAc(3) |
| 6511 | 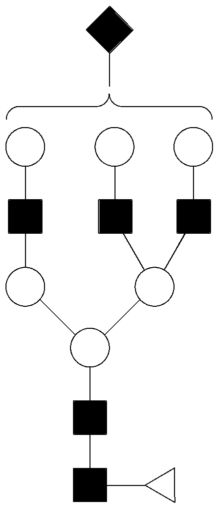 | Hex(6)HexNAc(5)Fuc(1)NeuAc(1) |
| 6512 | 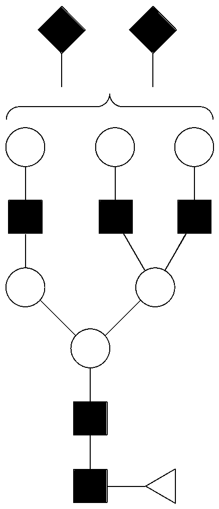 | Hex(6)HexNAc(5)Fuc(1)NeuAc(2) |
| 6513 | 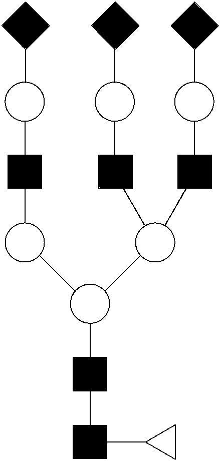 | Hex(6)HexNAc(5)Fuc(1)NeuAc(3) |
| 7624 | 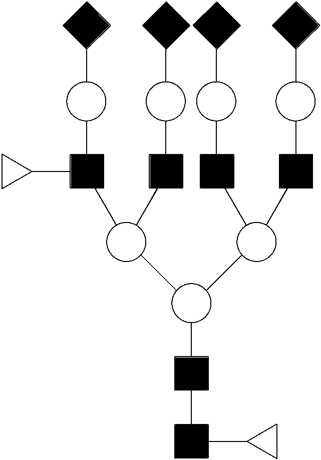 | Hex(7)HexNAc(6)Fuc(2)NeuAc(4) |
| 9200 | 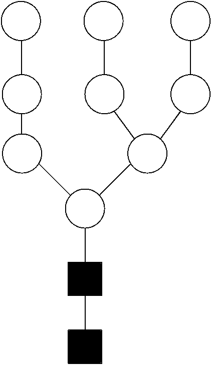 | Hex(9)HexNAc(2)Fuc(0)NeuAc(0) |

Legend for Table 15

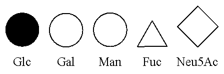

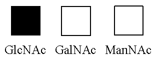

### XI. Additional Exemplary Embodiments

### Table 1

A1. A method for diagnosing a subject with respect to adenoma or colorectal cancer (CRC) disease state, the method comprising:
receiving peptide structure data corresponding to a biological sample obtained from the subject;
analyzing the peptide structure data using at least one supervised machine learning model to generate a disease indicator that indicates whether the biological sample evidences an adenoma or CRC disease state based on at least one peptide structure selected from a group of peptide structures identified in Table 1;
wherein the group of peptide structures in Table 1 is associated with the adenoma or CRC disease state; and
wherein the group of peptide structures is listed in Table 1 with respect to relative significance to the disease indicator; and
generating a diagnosis output based on the disease indicator.

A2. The method of claim A1, wherein the disease indicator comprises a score.

A3. The method of claim A2, wherein generating the diagnosis output comprises:
determining that the score falls above a selected threshold; and
generating the diagnosis output based on the score falling above the selected threshold, wherein the diagnosis output includes a positive diagnosis for the adenoma or CRC disease state.

A4. The method of claim A2, wherein generating the diagnosis output comprises:
determining that the score falls below a selected threshold; and
generating the diagnosis output based on the score falling below the selected threshold, wherein the diagnosis output includes a negative diagnosis for the adenoma or CRC disease state.

A5. The method of claim A3 or claim A4, wherein the score comprises a probability score and the selected threshold is 0.3267.

A6. The method of claim A3 or claim A4, wherein the selected threshold falls within a range between 0 and 1.

A7. The method of any one of claims A1-A6, wherein analyzing the peptide structure data comprises: analyzing the peptide structure data using a binary classification model.

A8. The method of any one of claims A1-A7, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 7-12 as defined in Table 1.

A9. The method of any one of claims A1-A8, further comprising:
training the at least one supervised machine learning model using training data,
wherein the training data comprises a plurality of peptide structure profiles for a plurality of subjects and a plurality of subject diagnoses for the plurality of subjects.

A10. The method of claim A9, wherein the plurality of subject diagnoses includes a positive diagnosis for any subject of the plurality of subjects determined to have the adenoma or CRC disease state and a negative diagnosis for any subject of the plurality of subjects determined not to have the adenoma or CRC disease state, wherein the adenoma or CRC disease state comprises at least one of CRC generally, early stage CRC, late stage CRC, stage 1 CRC, stage 2 CRC, stage 3 CRC, stage 4 CRC, or adenoma.

A11. The method of any one of claims A9-A10, further comprising:
performing a differential expression analysis using initial training data to compare a first portion of the plurality of subjects diagnosed with the positive diagnosis for the CRC or adenoma disease state versus a second portion of the plurality of subjects having the negative diagnosis for the adenoma or CRC disease state; and
identifying a training group of peptide structures based on the differential expression analysis for use as prognostic markers for the adenoma or CRC disease state; and
forming the training data based on the training group of peptide structures identified.

A12. The method of any one of claims A1-A11, wherein the peptide structure data comprises at least one of a raw abundance, an adjusted raw abundance, a peptide concentration, a glycopeptide concentration, or a normalized concentration.

A13. The method of any one of claims A1-A12, wherein the peptide structure data comprises normalized concentration data, wherein the normalized concentration data is a function of at least one of peptide abundance data, corresponding internal standard abundance data, a spike-in concentration value, and a dilution factor.

A14. The method of any one of claims A1-A13, wherein the at least one supervised machine learning model comprises a logistic regression model, and wherein the at least one supervised learning model compares the negative diagnosis versus the positive diagnosis, wherein the comparison can be at least one non-adenoma or non-CRC state vs at least one adenoma or CRC state.

A15. The method of any one of claims A1-A14, wherein the at least one supervised machine learning model comprises a logistic regression model, and wherein the at least one supervised learning model compares negative diagnoses versus positive diagnoses, wherein the comparison can be at least one healthy state versus adenoma or CRC generally, healthy state versus adenoma or early stage CRC, healthy state vs adenoma or stage 1 CRC, healthy state versus adenoma or stage 2 CRC, healthy state versus adenoma or stage 3 CRC, or healthy state versus adenoma or stage 4 CRC.

A16. The method of any one of claims A1-A15, wherein the peptide structure data is generated using multiple reaction monitoring mass spectrometry (MRM-MS).

A17. The method of any one of claims A1-A16, further comprising:
creating a sample from the biological sample; and
preparing the sample using reduction, alkylation, and enzymatic digestion to form a prepared sample that includes a set of peptide structures.

A18. The method of claim A17, further comprising: generating the peptide structure data from the prepared sample using multiple reaction monitoring mass spectrometry (MRM-MS).

A19. The method of any one of claims A1-A18, wherein generating the diagnosis output comprises: generating a report identifying that the biological sample evidences the adenoma or CRC disease state.

A20. The method of any one of claims A1-A19, further comprising: generating a treatment output based on at least one of the diagnosis output or the disease indicator.

A21. The method of claim A20, wherein the treatment output comprises at least one of an identification of a treatment to treat the subject or a treatment plan.

A22. The method of claim A21, wherein the treatment comprises at least one of radiation therapy, chemoradiotherapy, surgery, hormone therapy, or a targeted drug therapy.

A23. A method of training a model to diagnose a subject with respect to an adenoma or CRC disease state, the method comprising:
receiving peptide structure data for a panel of peptide structures for a plurality of subjects,
wherein the plurality of subjects includes a first portion having a negative diagnosis of an adenoma or CRC disease state and a second portion having a positive diagnosis of the adenoma or CRC disease state;
wherein the peptide structure data comprises a plurality of peptide structure profiles for the plurality of subjects; and
training at least one machine learning model using the peptide structure data to diagnose a biological sample with respect to the adenoma or CRC disease state using a group of peptide structures associated with the adenoma or CRC disease state,
wherein the group of peptide structures is identified in Table 1; and
wherein the group of peptide structures is listed in Table 1 with respect to relative significance to diagnosing the biological sample.

A24. The method of claim A23, wherein the at least one machine learning model comprises a logistic regression model, and wherein the at least one machine learning model compares the negative diagnosis versus the positive diagnosis, wherein the comparison can be at least one non-adenoma or non-CRC state vs at least one adenoma or CRC state.

A25. The method of claim A23, wherein the at least one supervised machine learning model comprises a logistic regression model, and wherein the at least one supervised learning model compares negative diagnoses versus positive diagnoses, wherein the comparison can be at least one healthy state versus adenoma or CRC generally, healthy state versus adenoma or early stage CRC, healthy state vs adenoma or stage 1 CRC, healthy state versus adenoma or stage 2 CRC, healthy state versus adenoma or stage 3 CRC, or healthy state versus adenoma or stage 4 CRC.

A26. The method of any one of claims A23-A25, wherein training the at least one machine learning model comprises:
training the at least one machine learning model using a portion of the peptide structure data corresponding to a training group of peptide structures included in the plurality of peptide structures.

A27. The method of claim A26, further comprising:
performing a differential expression analysis using the peptide structure data for the plurality of subjects.

A28. The method of claim A27, further comprising:
identifying the training group of peptide structures based on the differential expression analysis, wherein the training group of peptide structures is a subset of the plurality of peptide structures that has been determined to be relevant to diagnosing the adenoma or CRC disease state.

A29. The method of any one of claims A25-A28, wherein the peptide structure data comprises at least one of a raw abundance, an adjusted raw abundance, a peptide concentration, a glycopeptide concentration, or a normalized concentration.

A30. The method of any one of claims A25-A29, wherein the peptide structure data comprises normalized concentration data, wherein the normalized concentration data is a function of at least one of peptide abundance data, corresponding internal standard abundance data, a spike-in concentration value, and a dilution factor.

A31. A method of monitoring a subject for an adenoma or CRC disease state, the method comprising:
receiving first peptide structure data for a first biological sample obtained from a subject at a first timepoint;
analyzing the first peptide structure data using at least one supervised machine learning model to generate a first disease indicator based on at least one peptide structure selected from a group of peptide structures identified in Table 1, wherein the group of peptide structures in Table 1 comprises a group of peptide structures associated with an adenoma or CRC disease state;
receiving second peptide structure data of a second biological sample obtained from the subject at a second timepoint;
analyzing the second peptide structure data using the at least one supervised machine learning model to generate a second disease indicator based on the at least one peptide structure selected from the group of peptide structures identified in Table 1; and
generating a diagnosis output based on the first disease indicator and the second disease indicator.

A32. The method of claim A31, wherein generating the diagnosis output comprises: comparing the second disease indicator to the first disease indicator.

A33. The method of claim A31 or A32, wherein the first disease indicator indicates that the first biological sample evidences a negative diagnosis for the adenoma or CRC disease state and the second biological sample evidences a positive diagnosis for the adenoma or CRC disease state.

A34. The method of any one of claims A31-A33, wherein the plurality of subject diagnoses includes a positive diagnosis for any subject of the plurality of subjects determined to have the adenoma or CRC disease state and a negative diagnosis for any subject of the plurality of subjects determined not to have the adenoma or CRC disease state, wherein the adenoma or CRC disease state comprises at least one of adenoma or CRC cancer generally, adenoma or early stage CRC, adenoma or late stage CRC, adenoma or stage 1 CRC, adenoma or stage 2 CRC, adenoma or stage 3 CRC, or adenoma or stage 4 CRC.

A35. The method of any one of claims A31-A34, wherein the at least one supervised machine learning model comprises a logistic regression model, and wherein the at least one supervised learning model compares the negative diagnosis versus the positive diagnosis, wherein the comparison can be at least one non-adenoma or non-CRC state vs at least one adenoma or CRC state.

A36. The method of any one of claims A31-A35, wherein the at least one supervised machine learning model comprises a logistic regression model, and wherein the at least one supervised learning model compares negative diagnoses versus positive diagnoses, wherein the comparison can be at least one healthy state versus adenoma or CRC generally, healthy state versus adenoma or early stage CRC, healthy state vs adenoma or stage 1 CRC cancer, healthy state versus adenoma or stage 2 CRC, healthy state versus adenoma or stage 3 CRC, or healthy state versus adenoma or stage 4 CRC.

A37. A composition comprising at least one of peptide structures PS-1, PS-2, PS-3, PS-4, PS-5, or PS-6 identified in Table 1.

A38. A composition comprising a peptide structure or a product ion, wherein:
the peptide structure or the product ion comprises an amino acid sequence having at least 90% sequence identity to any one of SEQ ID NOS: 7-12, corresponding to peptide structures PS-1, PS-2, PS-3, PS-4, PS-5, or PS-6 in Table 1; and
the product ion is selected as one from a group consisting of product ions identified in Table 2 including product ions falling within an identified m/z range.

A39. A composition comprising a glycopeptide structure selected as one peptide structure from a group consisting of PS-1, PS-2, PS-3, PS-4, PS-5, or PS-6 identified in Table 1, wherein:
the glycopeptide structure comprises:
   an amino acid peptide sequence identified in Table 3A as corresponding to the glycopeptide structure; and
   a glycan structure identified in Table 5 as corresponding to the glycopeptide structure in which the glycan structure is linked to a residue of the amino acid peptide sequence at a corresponding position identified in Table 1; and
wherein the glycan structure has a glycan composition.

A40. The composition of claim A39, wherein the glycan composition is identified in Table 5.

A41. The composition of claim A39 or claim A40, wherein: the glycopeptide structure has a precursor ion having a charge identified in Table 3A as corresponding to the glycopeptide structure.

A42. The composition of any one of claims A39-A41, wherein: the glycopeptide structure has a precursor ion with an m/z ratio within ±1.5 of the m/z ratio listed for the precursor ion in Table 2 as corresponding to the glycopeptide structure.

A43. The composition of any one of claims A39-A42, wherein: the glycopeptide structure has a precursor ion with an m/z ratio within ±1.0 of the m/z ratio listed for the precursor ion in Table 2 as corresponding to the glycopeptide structure.

A44. The composition of any one of claims A39-A43, wherein: the glycopeptide structure has a precursor ion with an m/z ratio within ±0.5 of the m/z ratio listed for the precursor ion in Table 2 as corresponding to the glycopeptide structure.

A45. The composition of any one of claims A39-A44, wherein: the glycopeptide structure has a product ion with an m/z ratio within ±1.0 of the m/z ratio listed for the product ion in Table 2 as corresponding to the glycopeptide structure.

A46. The composition of any one of claims A39-A45, wherein: the glycopeptide structure has a product ion with an m/z ratio within ±0.8 of the m/z ratio listed for the product ion in Table 3A as corresponding to the glycopeptide structure.

A47. The composition of any one of claims A39-A46, wherein: the glycopeptide structure has a product ion with an m/z ratio within ±0.5 of the m/z ratio listed for the product ion in Table 2 as corresponding to the glycopeptide structure.

A48. The composition of any one of claims A39-A47, wherein the glycopeptide structure has a monoisotopic mass identified in Table 1 as corresponding to the glycopeptide structure.

A49. A composition comprising a peptide structure selected as one from a plurality of peptide structures identified in Table 1, wherein:
the peptide structure has a monoisotopic mass identified as corresponding to the peptide structure in Table 1; and
the peptide structure comprises the amino acid sequence of SEQ ID NOS: 7-12 identified in Table 1 as corresponding to the peptide structure.

A50. The composition of claim A49, wherein: the peptide structure has a precursor ion having a charge identified in Table 3 as corresponding to the peptide structure.

A51. The composition of claim A49 or claim A50, wherein: the peptide structure has a precursor ion with an m/z ratio within ±1.5 of the m/z ratio listed for the precursor ion in Table 2 as corresponding to the peptide structure.

A52. The composition of claim A49 or claim A50, wherein: the peptide structure has a precursor ion with an m/z ratio within ±1.0 of the m/z ratio listed for the precursor ion in Table 2 as corresponding to the peptide structure.

A53. The composition of claim A49 or claim A50, wherein: the peptide structure has a precursor ion with an m/z ratio within ±0.5 of the m/z ratio listed for the precursor ion in Table 2 as corresponding to the peptide structure.

A54. The composition of any one of claims A49-A53, wherein: the peptide structure has a product ion with an m/z ratio within ±1.0 of the m/z ratio listed for the product ion in Table 2 as corresponding to the peptide structure.

A55. The composition of any one of claims A49-A53, wherein: the peptide structure has a product ion with an m/z ratio within ±0.8 of the m/z ratio listed for the product ion in Table 2 as corresponding to the peptide structure.

A56. The composition of any one of claims A49-A53, wherein: the peptide structure has a product ion with an m/z ratio within ±0.5 of the m/z ratio listed for the product ion in Table 2 as corresponding to the peptide structure.

A57. A kit comprising at least one agent for quantifying at least one peptide structure identified in Table 1 to carry out part or all of the method of any one of claims A1-A36.

A58. A kit comprising at least one of a glycopeptide standard, a buffer, or a set of peptide sequences to carry out part or all of the method of any one of claims A1-A36, a peptide sequence of the set of peptide sequences identified by a corresponding one of SEQ ID NOS: 7-12, defined in Table 1.

A59. A system comprising:
one or more data processors; and
a non-transitory computer readable storage medium containing instructions which, when executed on the one or more data processors, cause the one or more data processors to perform part or all of the method of any one of claims A1-A36.

A60. A computer-program product tangibly embodied in a non-transitory machine-readable storage medium, including instructions configured to cause one or more data processors to perform part or all of the method of any one of claims A1-A36.

A61. A method of treating adenoma or CRC in a subject, the method comprising:
receiving a biological sample from the subject;
determining a quantity of at least 1 peptide structure identified in Table 1 in the biological sample using a multiple reaction monitoring mass spectrometry (MRM-MS) system;
analyzing the quantity of each peptide structure using at least one machine learning model to generate a disease indicator;
generating a diagnosis output based on the disease indicator that classifies the biological sample as evidencing that the subject has adenoma or CRC; and
administering a therapeutically effective amount of the treatment for adenoma or CRC, respectively.

A62. The method of claim A61, wherein the treatment comprises at least one of radiation therapy, chemotherapy, chemoradiotherapy, surgery, hormone therapy, or a targeted drug therapy.

A63. The method of claim A61 or A62, further comprising: preparing the biological sample to form a prepared sample comprising a set of peptide structures; and inputting the prepared sample into the MRM-MS system using a liquid chromatography system.

A64. The method of any one of claims A61-A63, further defined as determining a quantity of at least 1 peptide structure identified in Table 1 in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has adenoma or CRC; and administering a therapeutically effective amount of the treatment for adenoma or CRC, respectively.

A65. A method of identifying a need for one or more medical tests for a subject suspected of being at risk for or having an adenoma or CRC state, the method comprising: subjecting the subject to the one or more medical tests in response to measuring that a biological sample obtained from the subject evidences the state using part or all of the method of any one of claims A1-A36.

A66. The method of claim A65, wherein the one or more medical tests comprises colonoscopy, physical exam, CT scan, MRI scan, PET scan, or a combination thereof.

A67. A method of designing a treatment for a subject having an adenoma or CRC state, the method comprising: designing a therapeutic regimen for treating the subject in response to measuring that a biological sample obtained from the subject evidences the state using part or all of the method of any one of claims A1-A36.

A68. The method of claim A67, wherein the treatment comprises at least one of radiation therapy, chemotherapy, chemoradiotherapy, surgery, hormone therapy, or a targeted drug therapy.

A69. A method of treating a subject diagnosed with an adenoma or CRC state, the method comprising: administering to the subject a therapeutic to treat the subject based on measuring that a biological sample obtained from the subject evidences the state using part or all of the method of any one of claims A1-A36.

A70. The method of claim A69, wherein the treatment comprises at least one of radiation therapy, chemotherapy, chemoradiotherapy, surgery, hormone therapy, or a targeted drug therapy.

A71. A method of treating a subject having an adenoma or CRC state, the method comprising: selecting a therapeutic to treat the subject based on determining that the subject is responsive to the therapeutic using the method of any of claims A1-A36.

A72. The method of claim A71, wherein the treatment comprises at least one of radiation therapy, chemotherapy, chemoradiotherapy, surgery, hormone therapy, or a targeted drug therapy.

A73. A method of classifying a sample from an individual suspected of having, known to have, or at risk for an adenoma or CRC, comprising the step of measuring from the sample for one or more glycopeptides and/or non-glycosylated peptides in Table 1.

A74. The method of claim A73, wherein the measuring identifies the individual as not having adenoma or CRC.

A75. The method of claim A73, wherein the measuring identifies the individual as having adenoma or CRC.

A76. The method of claim A73, wherein the measuring identifies the individual as having early stage CRC or late stage CRC.

A77. The method of claim A73, wherein the measuring comprises successive or concomitant steps of identifying that the individual has CRC and that the individual has early stage CRC.

A78. The method of any one of claims A73-A77, wherein the sample comprises stool, peripheral blood, plasma, or serum.

A79. The method of claim A73, wherein the individual at risk for adenoma or CRC.

A80. The method of any one of claims A73-A79, wherein when the measuring identifies the individual as having adenoma or CRC, the individual is administered an effective amount of at least one of radiation therapy, chemotherapy, chemoradiotherapy, surgery, hormone therapy, or a targeted drug therapy.

A81. The method of any one of claims A73-A80, wherein the sample is measured for 2, 3, 4, 5, or all of the glycopeptides and/or non-glycosylated peptides of Table 1.

A82. A method of predicting a risk for adenoma or CRC in a subject, the method comprising:
receiving a biological sample from the subject;
determining a quantity of at least 1 peptide structure identified in Table 1 in the biological sample using a multiple reaction monitoring mass spectrometry (MRM-MS) system;
analyzing the quantity of each peptide structure using at least one machine learning model to generate a disease indicator; and
generating a diagnosis output based on the disease indicator that classifies the biological sample as evidencing that the subject has a risk for adenoma or CRC.

A83. A method of diagnosing adenoma or CRC or predicting a risk for adenoma or CRC in an individual, comprising the step of identifying one or more peptide structures identified in Table 1 from a sample from the individual.

A84. A method of identifying and managing an at-risk subject for adenoma or CRC, the method comprising measuring whether a biological sample obtained from the subject evidences an adenoma or CRC state using part or all of the method of any one of claims 1-36 and subjecting the subject to one or more medical tests or procedures in response to the identification of the CRC state.

A85. The method of claim A84, wherein the subject is subjected to a colonoscopy.

A86. The method of claim A84 or A85, wherein the subject is asymptomatic.

A87. The method of claim A84 or A85, wherein the subject has one or more symptoms of adenoma or CRC.

A88. A method of identifying a subject suitable for or in need of colonoscopy, the method comprising a step of detecting in a biological sample taken from the subject for the presence of one or a combination of peptide structures identified in Table 1, wherein their detection indicates that the subject should undergo a colonoscopy.

A89. The method of claim A88, wherein the subject is subjected to a colonoscopy.

A90. The method of claim A88 or A89, wherein the subject is asymptomatic.

A91. The method of claim A88 or A89, wherein the subject has one or more symptoms of adenoma or CRC.

A92. A method of identifying and managing a subject at risk of an adenoma or CRC disease state, the method comprising:
receiving a biological sample from the subject;
determining a quantity of at least 1 peptide structure identified in Table 1 in the biological sample;
analyzing the quantity of each peptide structure using at least one machine learning model to generate a disease indicator;
generating a diagnosis output based on the disease indicator that classifies the biological sample as evidencing that the subject has a risk for adenoma or CRC, and
identifying a need for a colonoscopy of the subject based on the classified risk of adenoma or CRC.

A93. The method of claim A92, wherein the disease indicator comprises a disease score.

A94. The method of claim A93, wherein generating the diagnosis output comprises:
determining that the disease score falls above a selected threshold; and
generating the diagnosis output based on the disease score falling above the selected threshold, wherein the diagnosis output includes a positive diagnosis for the adenoma or CRC disease state.

A95. The method of claim A93, wherein generating the diagnosis output comprises:
determining that the disease score falls below a selected threshold; and
generating the diagnosis output based on the disease score falling below the selected threshold, wherein the diagnosis output includes a negative diagnosis for the adenoma or CRC disease state.

A96. The method of claim 92, further comprising: identifying a need for a colonoscopy of the subject based on the classified risk of adenoma or CRC when the disease indicator falls above a risk threshold.

A97. The method of claim A92, further comprising: identifying a need for a colonoscopy of the subject based on the classified risk of adenoma or CRC when the disease indicator falls above the selected threshold.

A98. The method of claim A92, wherein the disease indicator comprises a risk score, the method further comprising: identifying a need for a colonoscopy of the subject based on the classified risk of adenoma or CRC when the risk score falls above a risk threshold.

A99. The method of claim A92, further comprising:
receiving medical information for the subject, the information including at least one of: personal and family medical history for the subject, and presence of hereditary medical conditions for the subject, and
analyzing (1) the quantity of each peptide structure using at least one machine learning model, and (2) the received medical information, to generate a disease indicator.

A100. The method of claim A99, wherein the medical information for the subject includes one or more of: demographic information for the subject, coded list of medical problems for the subject, previous colonoscopy findings, and answers provided by the subject to a questionnaire.

A101. The method of claims A99 and A100, wherein the personal and family medical history for the subject includes information that identifies whether the subject or a member of the subject's family has a history of adenomatous polyps or colorectal cancer.

A102. The method of any one of claims A99 to A101, wherein the presence of hereditary medical conditions for the subject includes information that identifies whether the subject has colorectal cancer syndrome or inflammatory bowel disease.

### Table 1B

B1. A method for diagnosing a subject with respect to advanced precancerous lesion (APL) or colorectal cancer (CRC) disease state, the method comprising:
receiving peptide structure data corresponding to a biological sample obtained from the subject;
analyzing the peptide structure data using at least one supervised machine learning model to generate a disease indicator that indicates whether the biological sample evidences an APL or CRC disease state based on at least one peptide structure selected from a group of peptide structures identified in Table 1B;
wherein the group of peptide structures in Table 1B is associated with the APL or CRC disease state; and
generating a diagnosis output based on the disease indicator.

B2. The method of claim B1, wherein the disease indicator comprises a score.

B3. The method of claim B2, wherein generating the diagnosis output comprises:
determining that the score falls above a selected threshold; and
generating the diagnosis output based on the score falling above the selected threshold, wherein the diagnosis output includes a positive diagnosis for the APL or CRC disease state.

B4. The method of claim B2, wherein generating the diagnosis output comprises:
determining that the score falls below a selected threshold; and
generating the diagnosis output based on the score falling below the selected threshold, wherein the diagnosis output includes a negative diagnosis for the APL or CRC disease state.

B5. The method of claim B3 or claim B4, wherein the score comprises a support vector machine score and the selected threshold is 0.

B6. The method of claim B3 or claim B4, wherein the selected threshold falls within a range between −0.1 and +0.1.

B7. The method of any one of claims B1-B6, wherein analyzing the peptide structure data comprises: analyzing the peptide structure data using a binary classification model.

B8. The method of any one of claims B1-B7, wherein the at least one peptide structure comprises a glycopeptide structure defined by a peptide sequence and a glycan structure linked to the peptide sequence at a linking site of the peptide sequence, as identified in Table 1B, with the peptide sequence being one of SEQ ID NOS: 27-41 as defined in Table 1B.

B9. The method of any one of claims B1-B8, further comprising:
training the at least one supervised machine learning model using training data,
wherein the training data comprises a plurality of peptide structure profiles for a plurality of subjects and a plurality of subject diagnoses for the plurality of subjects.

B10. The method of claim B9, wherein the plurality of subject diagnoses includes a positive diagnosis for any subject of the plurality of subjects determined to have the APL or CRC disease state and a negative diagnosis for any subject of the plurality of subjects determined not to have the APL or CRC disease state, wherein the APL or CRC disease state comprises at least one of CRC generally, early stage CRC, late stage CRC, stage 1 CRC, stage 2 CRC, stage 3 CRC, stage 4 CRC, or APL.

B11. The method of any one of claims B9-B10, further comprising:
performing a differential expression analysis using initial training data to compare a first portion of the plurality of subjects diagnosed with the positive diagnosis for the CRC or APL disease state versus a second portion of the plurality of subjects having the negative diagnosis for the APL or CRC disease state; and
identifying a training group of peptide structures based on the differential expression analysis for use as prognostic markers for the APL or CRC disease state; and
forming the training data based on the training group of peptide structures identified.

B12. The method of any one of claims B1-B11, wherein the peptide structure data comprises at least one of a raw abundance, an adjusted raw abundance, a peptide concentration, a glycopeptide concentration, or a normalized concentration.

B13. The method of any one of claims B1-B12, wherein the peptide structure data comprises normalized concentration data, wherein the normalized concentration data is a function of at least one of peptide abundance data, corresponding internal standard abundance data, a spike-in concentration value, and a dilution factor.

B14. The method of any one of claims B1-B13, wherein the at least one supervised machine learning model comprises a logistic regression model, and wherein the at least one supervised learning model compares the negative diagnosis versus the positive diagnosis, wherein the comparison can be at least one non-APL or non-CRC state vs at least one APL or CRC state.

B15. The method of any one of claims B1-B14, wherein the at least one supervised machine learning model comprises a logistic regression model, and wherein the at least one supervised learning model compares negative diagnoses versus positive diagnoses, wherein the comparison can be at least one healthy state versus APL or CRC generally, healthy state versus APL or early stage CRC, healthy state vs APL or stage 1 CRC, healthy state versus APL or stage 2 CRC, healthy state versus APL or stage 3 CRC, or healthy state versus APL or stage 4 CRC.

B16. The method of any one of claims B1-B15, wherein the peptide structure data is generated using multiple reaction monitoring mass spectrometry (MRM-MS).

B17. The method of any one of claims B1-B16, further comprising:
creating a sample from the biological sample; and
preparing the sample using reduction, alkylation, and enzymatic digestion to form a prepared sample that includes a set of peptide structures.

B18. The method of claim B17, further comprising: generating the peptide structure data from the prepared sample using multiple reaction monitoring mass spectrometry (MRM-MS).

B19. The method of any one of claims B1-B18, wherein generating the diagnosis output comprises: generating a report identifying that the biological sample evidences the APL or CRC disease state.

B20. The method of any one of claims B1-B19, further comprising: generating a treatment output based on at least one of the diagnosis output or the disease indicator.

B21. The method of claim B20, wherein the treatment output comprises at least one of an identification of a treatment to treat the subject or a treatment plan.

B22. The method of claim B21, wherein the treatment comprises at least one of radiation therapy, chemoradiotherapy, surgery, hormone therapy, or a targeted drug therapy.

B23. A method of training a model to diagnose a subject with respect to an APL or CRC disease state, the method comprising: receiving peptide structure data for a panel of peptide structures for a plurality of subjects,
wherein the plurality of subjects includes a first portion having a negative diagnosis of an APL or CRC disease state and a second portion having a positive diagnosis of the APL or CRC disease state;
wherein the peptide structure data comprises a plurality of peptide structure profiles for the plurality of subjects; and
training at least one machine learning model using the peptide structure data to diagnose a biological sample with respect to the APL or CRC disease state using a group of peptide structures associated with the APL or CRC disease state, and
wherein the group of peptide structures is identified in Table 1B.

B24. The method of claim B23, wherein the at least one machine learning model comprises a logistic regression model, and wherein the at least one machine learning model compares the negative diagnosis versus the positive diagnosis, wherein the comparison can be at least one non-APL or non-CRC state vs at least one APL or CRC state.

B25. The method of claim B23, wherein the at least one supervised machine learning model comprises a logistic regression model, and wherein the at least one supervised learning model compares negative diagnoses versus positive diagnoses, wherein the comparison can be at least one healthy state versus APL or CRC generally, healthy state versus APL or early stage CRC, healthy state vs APL or stage 1 CRC, healthy state versus APL or stage 2 CRC, healthy state versus APL or stage 3 CRC, or healthy state versus APL or stage 4 CRC.

B26. The method of any one of claims B23-B25, wherein training the at least one machine learning model comprises: training the at least one machine learning model using a portion of the peptide structure data corresponding to a training group of peptide structures included in the plurality of peptide structures.

B27. The method of claim B26, further comprising: performing a differential expression analysis using the peptide structure data for the plurality of subjects.

B28. The method of claim B27, further comprising: identifying the training group of peptide structures based on the differential expression analysis, wherein the training group of peptide structures is a subset of the plurality of peptide structures that has been determined to be relevant to diagnosing the APL or CRC disease state.

B29. The method of any one of claims B25-B28, wherein the peptide structure data comprises at least one of a raw abundance, an adjusted raw abundance, a peptide concentration, a glycopeptide concentration, or a normalized concentration.

B30. The method of any one of claims B25-B29, wherein the peptide structure data comprises normalized concentration data, wherein the normalized concentration data is a function of at least one of peptide abundance data, corresponding internal standard abundance data, a spike-in concentration value, and a dilution factor.

B31. A method of monitoring a subject for an APL or CRC disease state, the method comprising:
receiving first peptide structure data for a first biological sample obtained from a subject at a first timepoint;
analyzing the first peptide structure data using at least one supervised machine learning model to generate a first disease indicator based on at least one peptide structure selected from a group of peptide structures identified in Table 1B, wherein the group of peptide structures in Table 1B comprises a group of peptide structures associated with an APL or CRC disease state;
receiving second peptide structure data of a second biological sample obtained from the subject at a second timepoint;
analyzing the second peptide structure data using the at least one supervised machine learning model to generate a second disease indicator based on the at least one peptide structure selected from the group of peptide structures identified in Table 1B; and
generating a diagnosis output based on the first disease indicator and the second disease indicator.

B32. The method of claim B31, wherein generating the diagnosis output comprises: comparing the second disease indicator to the first disease indicator.

B33. The method of claim B31 or B32, wherein the first disease indicator indicates that the first biological sample evidences a negative diagnosis for the APL or CRC disease state and the second biological sample evidences a positive diagnosis for the APL or CRC disease state.

B34. The method of any one of claims B31-B33, wherein the plurality of subject diagnoses includes a positive diagnosis for any subject of the plurality of subjects determined to have the APL or CRC disease state and a negative diagnosis for any subject of the plurality of subjects determined not to have the APL or CRC disease state, wherein the APL or CRC disease state comprises at least one of APL or CRC cancer generally, APL or early stage CRC, APL or late stage CRC, APL or stage 1 CRC, APL or stage 2 CRC, APL or stage 3 CRC, or APL or stage 4 CRC.

B35. The method of any one of claims B31-B34, wherein the at least one supervised machine learning model comprises a logistic regression model, and wherein the at least one supervised learning model compares the negative diagnosis versus the positive diagnosis, wherein the comparison can be at least one non-APL or non-CRC state vs at least one APL or CRC state.

B36. The method of any one of claims B31-B35, wherein the at least one supervised machine learning model comprises a logistic regression model, and wherein the at least one supervised learning model compares negative diagnoses versus positive diagnoses, wherein the comparison can be at least one healthy state versus APL or CRC generally, healthy state versus APL or early stage CRC, healthy state vs APL or stage 1 CRC cancer, healthy state versus APL or stage 2 CRC, healthy state versus APL or stage 3 CRC, or healthy state versus APL or stage 4 CRC.

B37. A composition comprising at least one of peptide structures PS-7, PS-8, PS-9, PS-10, PS-11, PS-12, PS-13, PS-14, PS-15, PS-16, PS-17, PS-18, PS-19, PS-20, or PS-2 lidentified in Table 1B.

B38. A composition comprising a peptide structure or a product ion, wherein:
the peptide structure or the product ion comprises an amino acid sequence having at least 90% sequence identity to any one of SEQ ID NOS: 27-41, corresponding to peptide structures PS-7, PS-8, PS-9, PS-10, PS-11, PS-12, PS-13, PS-14, PS-15, PS-16, PS-17, PS-18, PS-19, PS-20, or PS-21 in Table 1B; and
the product ion is selected as one from a group consisting of product ions identified in Table 2B including product ions falling within an identified m/z range.

B39. A composition comprising a glycopeptide structure selected as one peptide structure from a group consisting of PS-7, PS-8, PS-9, PS-10, PS-11, PS-12, PS-13, PS-14, PS-15, PS-16, PS-17, PS-18, PS-19, PS-20, or PS-21 identified in Table 1B, wherein:
the glycopeptide structure comprises:
an amino acid peptide sequence identified in Table 3C as corresponding to the glycopeptide structure; and
a glycan structure identified in Tables 5B and/or 5C as corresponding to the glycopeptide structure in which the glycan structure is linked to a residue of the amino acid peptide sequence at a corresponding position identified in Table 1B; and
wherein the glycan structure has a glycan composition.

B40. The composition of claim B39, wherein the glycan composition is identified in Tables 5B and/or 5C.

B41. The composition of claim B39 or claim 40, wherein: the glycopeptide structure has a precursor ion having a charge identified in Table 2B as corresponding to the glycopeptide structure.

B42. The composition of any one of claims B39-B41, wherein: the glycopeptide structure has a precursor ion with an m/z ratio within ±1.5 of the m/z ratio listed for the precursor ion in Table 2B as corresponding to the glycopeptide structure.

B43. The composition of any one of claims B39-B42, wherein: the glycopeptide structure has a precursor ion with an m/z ratio within ±1.0 of the m/z ratio listed for the precursor ion in Table 2B as corresponding to the glycopeptide structure.

B44. The composition of any one of claims B39-B43, wherein: the glycopeptide structure has a precursor ion with an m/z ratio within ±0.5 of the m/z ratio listed for the precursor ion in Table 2B as corresponding to the glycopeptide structure.

B45. The composition of any one of claims B39-B44, wherein: the glycopeptide structure has a product ion with an m/z ratio within ±1.0 of the m/z ratio listed for the product ion in Table 2B as corresponding to the glycopeptide structure.

B46. The composition of any one of claims B39-B45, wherein: the glycopeptide structure has a product ion with an m/z ratio within ±0.8 of the m/z ratio listed for the product ion in Table 2B as corresponding to the glycopeptide structure.

B47. The composition of any one of claims B39-B46, wherein: the glycopeptide structure has a product ion with an m/z ratio within ±0.5 of the m/z ratio listed for the product ion in Table 2B as corresponding to the glycopeptide structure.

B48. The composition of any one of claims B39-B47, wherein the glycopeptide structure has a monoisotopic mass identified in Table 1B as corresponding to the glycopeptide structure.

B49. A composition comprising a peptide structure selected as one from a plurality of peptide structures identified in Table 1B, wherein: the peptide structure has a monoisotopic mass identified as corresponding to the peptide structure in Table 1B; and the peptide structure comprises the amino acid sequence of SEQ ID NOS: 27-41 identified in Table 1B as corresponding to the peptide structure.

B50. The composition of claim B49, wherein: the peptide structure has a precursor ion having a charge identified in Table 2B as corresponding to the peptide structure.

B51. The composition of claim B49 or claim B50, wherein: the peptide structure has a precursor ion with an m/z ratio within ±1.5 of the m/z ratio listed for the precursor ion in Table 2B as corresponding to the peptide structure.

B52. The composition of claim B49 or claim B50, wherein: the peptide structure has a precursor ion with an m/z ratio within ±1.0 of the m/z ratio listed for the precursor ion in Table 2B as corresponding to the peptide structure.

B53. The composition of claim B49 or claim B50, wherein: the peptide structure has a precursor ion with an m/z ratio within ±0.5 of the m/z ratio listed for the precursor ion in Table 2B as corresponding to the peptide structure.

B54. The composition of any one of claims B49-B53, wherein: the peptide structure has a product ion with an m/z ratio within ±1.0 of the m/z ratio listed for the product ion in Table 2B as corresponding to the peptide structure.

B55. The composition of any one of claims B49-B53, wherein: the peptide structure has a product ion with an m/z ratio within ±0.8 of the m/z ratio listed for the product ion in Table 2B as corresponding to the peptide structure.

B56. The composition of any one of claims B49-B53, wherein: the peptide structure has a product ion with an m/z ratio within ±0.5 of the m/z ratio listed for the product ion in Table 2B as corresponding to the peptide structure.

B57. A kit comprising at least one agent for quantifying at least one peptide structure identified in Table 1B to carry out part or all of the method of any one of claims B1-B36.

B58. A kit comprising at least one of a glycopeptide standard, a buffer, or a set of peptide sequences to carry out part or all of the method of any one of claims 1-36, a peptide sequence of the set of peptide sequences identified by a corresponding one of SEQ ID NOS: 27-41, defined in Table 1B.

B59. A system comprising:
one or more data processors; and
a non-transitory computer readable storage medium containing instructions which, when executed on the one or more data processors, cause the one or more data processors to perform part or all of the method of any one of claims B1-B36.

B60. A computer-program product tangibly embodied in a non-transitory machine-readable storage medium, including instructions configured to cause one or more data processors to perform part or all of the method of any one of claims B1-B36.

B61. A method of treating APL or CRC in a subject, the method comprising:
receiving a biological sample from the subject;
determining a quantity of at least 1 peptide structure identified in Table 1B in the biological sample using a multiple reaction monitoring mass spectrometry (MRM-MS) system;
analyzing the quantity of each peptide structure using at least one machine learning model to generate a disease indicator;
generating a diagnosis output based on the disease indicator that classifies the biological sample as evidencing that the subject has APL or CRC; and
administering a therapeutically effective amount of the treatment for APL or CRC, respectively.

B62. The method of claim B61, wherein the treatment comprises at least one of radiation therapy, chemotherapy, chemoradiotherapy, surgery, hormone therapy, or a targeted drug therapy.

B63. The method of claim B61 or B62, further comprising: preparing the biological sample to form a prepared sample comprising a set of peptide structures; and inputting the prepared sample into the MRM-MS system using a liquid chromatography system.

B64. The method of any one of claims B61-B63, further defined as determining a quantity of at least 1 peptide structure identified in Table 1B in the biological sample using a multiple reaction monitoring mass spectrometry (MRM-MS) system; analyzing the quantity of each peptide structure using at least one machine learning model to generate a disease indicator; generating a diagnosis output based on the disease indicator that classifies the biological sample as evidencing that the subject has APL or CRC; and administering a therapeutically effective amount of the treatment for APL or CRC, respectively.

B65. A method of identifying a need for one or more medical tests for a subject suspected of being at risk for or having an APL or CRC state, the method comprising: subjecting the subject to the one or more medical tests in response to measuring that a biological sample obtained from the subject evidences the state using part or all of the method of any one of claims 1-36.

B66. The method of claim B65, wherein the one or more medical tests comprises colonoscopy, physical exam, CT scan, MRI scan, PET scan, or a combination thereof.

B67. A method of designing a treatment for a subject having an APL or CRC state, the method comprising: designing a therapeutic regimen for treating the subject in response to measuring that a biological sample obtained from the subject evidences the state using part or all of the method of any one of claims B1-B36.

B68. The method of claim B67, wherein the treatment comprises at least one of radiation therapy, chemotherapy, chemoradiotherapy, surgery, hormone therapy, or a targeted drug therapy.

B69. A method of treating a subject diagnosed with an APL or CRC state, the method comprising: administering to the subject a therapeutic to treat the subject based on measuring that a biological sample obtained from the subject evidences the state using part or all of the method of any one of claims B1-B36.

B70. The method of claim B69, wherein the treatment comprises at least one of radiation therapy, chemotherapy, chemoradiotherapy, surgery, hormone therapy, or a targeted drug therapy.

B71. A method of treating a subject having an APL or CRC state, the method comprising: selecting a therapeutic to treat the subject based on determining that the subject is responsive to the therapeutic using the method of any of claims B1-B36.

B72. The method of claim B71, wherein the treatment comprises at least one of radiation therapy, chemotherapy, chemoradiotherapy, surgery, hormone therapy, or a targeted drug therapy.

B73. A method of classifying a sample from an individual suspected of having, known to have, or at risk for an APL or CRC, comprising the step of measuring from the sample for one or more glycopeptides and/or non-glycosylated peptides in Table 1B.

B74. The method of claim B73, wherein the measuring identifies the individual as not having APL or CRC.

B75. The method of claim B73, wherein the measuring identifies the individual as having APL or CRC.

B76. The method of claim B73, wherein the measuring identifies the individual as having early stage CRC or late stage CRC.

B77. The method of claim B73, wherein the measuring comprises successive or concomitant steps of identifying that the individual has CRC and that the individual has early stage CRC.

B78. The method of any one of claims B73-B77, wherein the sample comprises stool, peripheral blood, plasma, or serum.

B79. The method of claim B73, wherein the individual at risk for APL or CRC.

B80. The method of any one of claims B73-B79, wherein when the measuring identifies the individual as having APL or CRC, the individual is administered an effective amount of at least one of radiation therapy, chemotherapy, chemoradiotherapy, surgery, hormone therapy, or a targeted drug therapy.

B81. The method of any one of claims B73-B80, wherein the sample is measured for 2, 3, 4, 5, or all of the glycopeptides and/or non-glycosylated peptides of Table 1B.

B82. A method of predicting a risk for APL or CRC in a subject, the method comprising:
receiving a biological sample from the subject;
determining a quantity of at least 1 peptide structure identified in Table 1B in the biological sample using a multiple reaction monitoring mass spectrometry (MRM-MS) system;
analyzing the quantity of each peptide structure using at least one machine learning model to generate a disease indicator; and
generating a diagnosis output based on the disease indicator that classifies the biological sample as evidencing that the subject has a risk for APL or CRC.

B83. A method of diagnosing APL or CRC or predicting a risk for APL or CRC in an individual, comprising the step of identifying one or more peptide structures identified in Table 1B from a sample from the individual.

B84. A method of identifying and managing an at-risk subject for APL or CRC, the method comprising measuring whether a biological sample obtained from the subject evidences an APL or CRC state using part or all of the method of any one of claims B1-B36 and subjecting the subject to one or more medical tests or procedures in response to the identification of the CRC state.

B85. The method of claim B84, wherein the subject is subjected to a colonoscopy.

B86. The method of claim B84 or B85, wherein the subject is asymptomatic.

B87. The method of claim B84 or B85, wherein the subject has one or more symptoms of APL or CRC.

B88. A method of screening a subject, the method comprising
analyzing a peptide structure data using at least one supervised machine learning model to generate a disease indicator that indicates whether the biological sample evidences an APL or CRC disease state based on at least one peptide structure selected from a group of peptide structures identified in Table 1B, wherein peptide structure data corresponds to a biological sample obtained from the subject; and
outputting either a recommendation to perform a colonoscopy or to not perform the colonoscopy based on the disease indicator.

B89. The method of claim B88, wherein the group of peptide structures in Table 1B is associated with the APL or CRC disease state.

B90. The method of claims B88-B89, wherein the group of peptide structures is listed in Table 1B with respect to relative significance to the disease indicator.

B91. The method of claims B88-B90, wherein the subject is subjected to a colonoscopy when the recommendation to perform the colonoscopy is outputted.

B92. The method of claims B88-B91, wherein the subject does not have any symptoms of APL and CRC.

B93. The method of claims B88-B92 further comprising: receiving peptide structure data corresponding to the biological sample obtained from the subject.

B94. The method of claims B88-B93, wherein the disease indicator comprises a score, wherein generating the diagnosis output comprises:
determining that the score falls above a selected threshold; and
generating the diagnosis output based on the score falling above the selected threshold, wherein the diagnosis output includes a positive diagnosis for the APL or CRC disease state.

B95. The method of any one of claims B88-B94, wherein analyzing the peptide structure data comprises: analyzing the peptide structure data using a binary classification model.

B96. The method of any one of claims B88-B95, wherein the at least one peptide structure comprises a glycopeptide structure defined by a peptide sequence and a glycan structure linked to the peptide sequence at a linking site of the peptide sequence, as identified in Table 1B, with the peptide sequence being one of SEQ ID NOS: 27-41 as defined in Table 1B and/or Table 3C.

B97. The method of any one of claims B88-B96, wherein the peptide structure data comprises at least one of a raw abundance, an adjusted raw abundance, a peptide concentration, a glycopeptide concentration, or a normalized concentration.

B98. The method of any one of claims B88-B97, wherein the peptide structure data comprises normalized concentration data, wherein the normalized concentration data is a function of at least one of peptide abundance data, corresponding internal standard abundance data, a spike-in concentration value, and a dilution factor.

B99. The method of any one of claims B88-B98, wherein the peptide structure data is generated using multiple reaction monitoring mass spectrometry (MRM-MS).

B100. The method of any one of claims B88-B99, further comprising:
creating a sample from the biological sample; and
preparing the sample using reduction, alkylation, and enzymatic digestion to form a prepared sample that includes a set of peptide structures.

B101. The method of claim B100, further comprising:
generating the peptide structure data from the prepared sample using multiple reaction monitoring mass spectrometry (MRM-MS).

B102. The method of any one of claims B88-B101, wherein the recommendation is a report identifying that the biological sample evidences the APL or CRC disease state.

B103. A method of identifying and managing a subject at risk of an APL or CRC disease state, the method comprising:
receiving a biological sample from the subject;
determining a quantity of at least 1 peptide structure identified in Table 1B in the biological sample;
analyzing the quantity of each peptide structure using at least one machine learning model to generate a disease indicator;
generating a diagnosis output based on the disease indicator that classifies the biological sample as evidencing that the subject has a risk for APL or CRC, and
identifying a need for a colonoscopy of the subject based on the classified risk of APL or CRC.

B104. The method of claim B103, wherein the disease indicator comprises a disease score.

B105. The method of claim B104, wherein generating the diagnosis output comprises:
determining that the disease score falls above a selected threshold; and
generating the diagnosis output based on the disease score falling above the selected threshold, wherein the diagnosis output includes a positive diagnosis for the APL or CRC disease state.

B106. The method of claim B104, wherein generating the diagnosis output comprises:
determining that the disease score falls below a selected threshold; and
generating the diagnosis output based on the disease score falling below the selected threshold, wherein the diagnosis output includes a negative diagnosis for the APL or CRC disease state.

B107. The method of claim B103, further comprising: identifying a need for a colonoscopy of the subject based on the classified risk of APL or CRC when the disease indicator falls above a risk threshold.

B108. The method of claim B105, further comprising: identifying a need for a colonoscopy of the subject based on the classified risk of APL or CRC when the disease indicator falls above the selected threshold.

B109. The method of claim B103, wherein the disease indicator comprises a risk score, the method further comprising: identifying a need for a colonoscopy of the subject based on the classified risk of APL or CRC when the risk score falls above a risk threshold.

B110. The method of claim B103, further comprising:
receiving medical information for the subject, the information including at least one of: personal and family medical history for the subject, and presence of hereditary medical conditions for the subject, and
analyzing (1) the quantity of each peptide structure using at least one machine learning model, and (2) the received medical information, to generate a disease indicator.

B111. The method of claim B110, wherein the medical information for the subject includes one or more of: demographic information for the subject, coded list of medical problems for the subject, previous colonoscopy findings, and answers provided by the subject to a questionnaire.

B112. The method of claims B110 and B112, wherein the personal and family medical history for the subject includes information that identifies whether the subject or a member of the subject's family has a history of adenomatous polyps or colorectal cancer.

B113. The method of any one of claims B110 to B112, wherein the presence of hereditary medical conditions for the subject includes information that identifies whether the subject has colorectal cancer syndrome or inflammatory bowel disease.

B114. The method of claims B7 or B95, in which the binary classification model includes a first classification where the subject is healthy and a second classification where the subject has APL or CRC.

B115. The method of any one of embodiments described herein, wherein the biological sample is in a tube, wherein the tube comprises an anticoagulant and a preserving agent, the method further comprising:
isolating a plasma fraction from the tube to create a sample from the biological sample;
preparing the sample using reduction, alkylation, and enzymatic digestion to form a prepared sample that includes a set of peptide structures.

B116. The method of claim B115, wherein the anticoagulant includes EDTA salt and the preserving agent includes imidazolidinyl urea

B117. The method of claims B116, wherein before the isolating the plasma fraction, the biological sample had contacted the preserving agent for a period of time ranging from 24 hours to 7 days.

B118. The method of claim B116, wherein the tube further comprises glycine.

B119. The method of any one of embodiments described herein, wherein the biological sample is in a tube, wherein the tube comprises silica particles, the method further comprising:
isolating a serum fraction from the tube to create a sample from the biological sample;
preparing the sample using reduction, alkylation, and enzymatic digestion to form a prepared sample that includes a set of peptide structures.

B120. The method of claims B119, wherein the silica particles were spray-coated onto an inner surface of the tube.

B121. The method of claim B120, wherein the tube further comprises a polyester gel configured to form a barrier between a serum fraction and blood cells during a centrifugation process.

B122. The method of claims B121, wherein the biological sample formed a clot in the tube before the isolating the serum fraction from the tube.

### Tables 13A and 13B

E1. A method of diagnosing an individual with colorectal cancer comprising
detecting the presence or amount of at least one peptide structure from Table 13A or Table 13B in a biological sample obtained from the individual and thereby diagnosing the individual as having colorectal cancer or not having colorectal cancer based upon the presence or amount of the at least one peptide structure from Table 13A or Table 13B.

E2. A method for determining a risk of an individual for developing colorectal cancer in an individual comprising
detecting the presence or amount of at least one peptide structure from Table 13A or Table 13B in a biological sample obtained from the individual and thereby determining the risk of the individual for developing colorectal cancer based upon the presence or amount of the at least one peptide structure from Table 13A or Table 13B.

E3. The method of claim E2, further comprising providing a recommendation to the individual to undergo an endoscopy or structural examination based upon the determined risk.

E4. The method of claim E2 or claim E3, further comprising performing an endoscopy on the individual to diagnose colorectal cancer.

E5. The method of claim E4, wherein the endoscopy is a sigmoidoscopy or a colonoscopy.

E6. The method of any one of claims E1-E5, further comprising providing a treatment to the individual for colorectal cancer if the individual is determined to have colorectal cancer.

E7. A method of treating colorectal cancer in an individual comprising
diagnosing the individual as having colorectal cancer based upon the presence or amount of at least one peptide structure from Table 13A or Table 13B in a biological sample obtained from the individual; and
providing a recommendation for a treatment for colorectal cancer based upon the diagnosis.

E8. The method of claim E7, further comprising administering the treatment.

E9. A method of treating colorectal cancer in an individual comprising detecting the presence or amount of at least one peptide structure from Table 13A or Table 13B in a biological sample obtained from the individual and administering a treatment for colorectal cancer.

E10. The method of any one of claims E1-E9, wherein the amount of at least one peptide structure is none, or below a detection limit.

E11. The method of any one of claims E1-E10, wherein the biological sample is serum or plasma.

E12. The method of any one of claims E1-E11, wherein the at least one peptide structure is a glycopeptide.

E13. The method of any one of claims E1-1E2, wherein the at least one peptide structure comprises three or more, five or more, 10 or more, or 20 or more peptide structures from Table 13A.

E14. The method of any one of claims E1-E13, wherein the at least one peptide structure comprises a sequence set forth in SEQ ID NOs: 168-198.

E15. The method of any one of claims E1-E14 wherein the at least one peptide structure comprises three or more, five or more, 10 or more, or 20 or more different peptide structures comprising a sequence set forth in SEQ ID NOs: 168-198.

E16. The method of any one of claims E1-E15, wherein the at least one peptide structure comprises two or more, three or more, four or more, five or more, six or more, seven or more, eight or more or nine peptide structures from Table 13B.

E17. The method of any one of claims E1-E16, wherein the at least one peptide structure comprises a sequence set forth in SEQ ID NOs: 168, 171, 172, 176, 181, 184, 187, 192, and 194.

E18. The method of any one of claims E1-E17 wherein the at least one peptide structure comprises two or more, three or more, four or more, five or more, six or more, seven or more, eight or more or nine peptide structures comprising a sequence set forth in SEQ ID NOs: 168, 171, 172, 176, 181, 184, 187, 192, and 194.

E19. The method of any one of claims E1-E18, wherein the at least one peptide structure comprises a peptide sequence and a glycan structure, wherein the glycan structure is attached to a linking site position in the peptide sequence in accordance with Table 13A or 13B.

E20. The method of claim E19, wherein the glycan structure of the peptide sequence comprises a glycan structure GL number in accordance with Table 13A or Table 13B, wherein the glycan structure comprises a composition in accordance with the glycan structure GL number and Table 15.

E21. The method of claim E20, wherein the glycan structure of the peptide sequence comprises a glycan structure GL number in accordance with Table 13A or Table 13B, wherein the glycan structure comprises a symbol structure in accordance with the glycan structure GL number and Table 15.

E22. The method of any one of claims E1-E21, wherein the presence or amount of the at least one peptide structure is detected using western blot, mass spectrometry or ELISA. E23. The method of claim E22, wherein the presence or amount of the at least one peptide structure is detected using MS/MS or MRM mass spectrometry.

E24. The method of any one of claims E1-E23, further comprising comparing the presence or amount of the one or more peptide structure from Table 13A or Table 13B between the biological sample from the individual and a control sample, wherein the control sample is a sample from one or more individuals who do not have colorectal cancer.

E25. The method of any one of claims E1-E24, wherein the individual is human.

E26. The method of any one of claims E6-E25, wherein the treatment comprises, chemotherapy, immunotherapy, radiation, or surgery.

E27. The method of any one of claims E1-E26, wherein the individual is age 40 and over, age 50 and over, or age 60 and over.

E28. The method of any one of claims E1-E27, further comprising assessing one or more risk factors or clinical indicators for colorectal cancer.

E29. The method of any one of claims E1-28, wherein the individual has one or more clinical indicators associated with colorectal cancer.

E30. The method of claim E29, wherein the one or more clinical indicators is selected from the group consisting of changes in bowel habits, bloody stool, diarrhea, constipation, persistent abdominal pain, persistent abdominal cramps, and unexplained weight loss.

E31. The method of any one of claims E1-E30, wherein the individual has one or more risk factors associated with colorectal cancer.

E32. The method of claim E31, wherein the risk factor is selected from the group consisting of, age, irritable bowel syndrome, type 2 diabetes, a family history of CRC, Lynch syndrome, obesity, smoking, and alcohol consumption.

E33. The method of any one of claims E1-E32, wherein the individual is determined to have a healthy state, wherein the healthy state comprises an absence of colorectal cancer and/or a low risk for colorectal cancer.

E34. The method of any one of claims E1-E33,
wherein a bottommost N-acetylglucosamine of the glycan structure in Table 15 is attached to a linking site position in the peptide sequence in accordance with Table 13A or Table 13B.

E35. The method of any one of claims E1-E34, wherein the method further comprises
digesting the biological sample with a protease, and
performing liquid chromatography mass spectrometry (LC/MS) on the biological sample to detect the presence or amount of the at least one peptide structure from Table 13A or Table 13B.

E36. The method of any one of claims E24-E35 wherein the method further comprises
digesting the control sample with a protease, and
performing liquid chromatography mass spectrometry (LC/MS) on the control sample to detect the presence or amount of the at least one peptide structure from Table 13A or Table 13B

E37. The method of claim E35 or claim E36, wherein the method further comprises denaturing the biological sample and/or the control sample prior to digesting the biological sample and/or the control sample.

E38. The method of claim E37, wherein the denaturing the biological sample and/or the control sample comprises heating the biological sample and/or the control sample to at least 100° C.

E39. The method of any one of claims E35-E38, further comprising reducing the biological sample and/or the control sample after denaturing the biological sample and the control sample prior to digesting the biological sample and the control sample.

E40. The method of claim E39, wherein the reducing the biological sample and/or the control sample comprises incubating the biological sample and/or the control sample with a reducing agent.

E41. The method of claim E39 or claim E40, wherein the reducing agent is dithiothreitol (DTT).

E42. The method of claim E40 or claim E41, further comprising incubating the biological sample and/or the control sample with an alkylating agent following reducing the biological sample and/or the control sample, and then, quenching a remaining portion of the alkylating agent with DTT for both the biological sample and/or the control sample prior to digesting the biological sample and/or the control sample.

E43. The method of any one of claims E35-E42, wherein the enriching for glycopeptides comprises loading the proteolytic digest onto a HILIC (hydrophilic interaction liquid chromatography) column, washing the HILIC column with a wash liquid, and eluting an enriched glycopeptide eluate from the HILIC column with an eluting liquid.

E44. The method of any one of claims E35-E43, wherein the enriching the biological sample and/or the control sample for the at least one glycopeptide is performed after the digesting the biological sample and/or the control sample with the protease.

E45. A composition comprising one or more, two or more, three or more, five or more, 10 or more, or 20 or more different peptide structures set forth in Table 13A.

E46. A composition comprising one or more, two or more, three or more, four or more, five or more, six or more, seven or more, eight or more, or nine peptide structure set forth in Table 13B.

E47. The composition of claim E45 or claim E46, wherein the one or more peptide structure comprises a peptide sequence and a glycan structure, wherein the glycan structure is attached to a linking site position in the peptide sequence, wherein the peptide sequence and the linking site position in the peptide sequence are in accordance with Table 13A or Table 13B.

E48. The composition of claim E47, wherein the glycan structure of the peptide sequence comprises a glycan structure GL number in accordance with Table 13A or Table 13B, wherein the glycan structure comprises a composition in accordance with the glycan structure GL number and Table 15.

E49. The composition of claim E48, wherein the glycan structure of the peptide sequence comprises a glycan structure GL number in accordance with Table 13A or Table 13B, wherein the glycan structure comprises a symbol structure in accordance with the glycan structure GL number and Table 15.

E50. A method of identifying one or more glycopeptide biomarker associated with colorectal cancer comprising obtaining a first set of biological samples from one or more individuals with colorectal cancer and a second set of control biological samples from one or more individuals who do not have colorectal cancer,
digesting the first set of biological samples and the second set of control biological samples with a protease,
enriching the first set of biological samples and the second set of control biological samples for at least one glycopeptide,
performing liquid chromatography mass spectrometry (LC/MS) on the first set of biological samples and the second set of control biological samples to identify glycopeptides present in the first set of biological samples and second set of control samples,
determining which glycopeptides are present in the first set of biological samples and are not present in the second set of control samples, and thereby identifying one or more glycopeptide biomarker associated with colorectal cancer.

E51. The method of claim E50, wherein the first set of biological samples and second set of control biological samples each comprise biological samples from at least three individuals.

E52. The method of claim E51, wherein the one or more glycopeptide biomarkers associated with colorectal cancer are present in biological samples from at least three individuals with colorectal cancer.

E53. The method of claim E51 or claim E52, wherein the one or more glycopeptide biomarkers associated with colorectal cancer are present in at least 30%, at least 40%, at least 50%, at least 60%, at least 70%, at least 80%, or at least 90% of the first set of biological samples from the individuals with colorectal cancer.

E54. The method of any one of claims E50-E53, further comprising denaturing the first set of biological samples and the second set of control biological samples prior to digesting first set of biological samples and the second set of control biological samples.

E55. The method of claim E54, wherein the denaturing the first set of biological samples and the second set of control biological samples comprises heating the first set of biological samples and the second set of control biological samples to at least 100° C.

E56. The method of claim E54 or claim E55, further comprising reducing the first set of biological samples and the second set of control biological samples after denaturing the first set of biological samples and the second set of control biological samples prior to digesting the first set of biological samples and the second set of control biological samples.

E57. The method of claim E56, wherein the reducing the first set of biological samples and the second set of control biological samples comprises incubating the first set of biological samples and the second set of control biological samples with a reducing agent.

E58. The method of claim E57, wherein the reducing agent is dithiothreitol (DTT).

E59. The method of claim E57 or claim E58, further comprising incubating the first set of biological samples and the second set of control biological samples with an alkylating agent following reducing the first set of biological samples and the second set of control biological samples, and then, quenching a remaining portion of the alkylating agent with DTT for both the first set of biological samples and the second set of control biological samples prior to digesting the first set of biological samples and the second set of control biological samples.

E60. The method of any one of claims E50-E59, wherein the enriching for glycopeptides comprises loading the proteolytic digest onto a HILIC (hydrophilic interaction liquid chromatography) column, washing the HILIC column with a wash liquid, and eluting an enriched glycopeptide eluate from the HILIC column with an eluting liquid.

E61. The method of any one of claims E50-E60, wherein the enriching the first set of biological samples and the second set of control biological samples for the at least one glycopeptide is performed after the digesting the first set of biological samples and the second set of control biological samples with the protease.

E62. The method of any one of claims E35-E61, wherein the performing liquid chromatography mass spectrometry (LC/MS) uses an ion trap mass analyzer, the ion trap mass analyzer comprising an outer barrel-like electrode and a coaxial inner spindle-like electrode, the ion trap mass analyzer configured to trap ions in an orbital motion around the spindle.

What is claimed:

1. A method for diagnosing a subject with respect to high-grade advanced pre-malignant lesions or colorectal cancer (CRC) disease state, the method comprising:
receiving peptide structure data corresponding to a biological sample obtained from the subject;
analyzing the peptide structure data using at least one supervised machine learning model to generate a disease indicator that indicates whether the biological sample evidences an high-grade advanced pre-malignant lesions or CRC disease state based on at least one peptide structure selected from a group of peptide structures identified in Table 1C;
wherein the group of peptide structures in Table 1C is associated with the high-grade advanced pre-malignant lesions or CRC disease state; and
generating a diagnosis output based on the disease indicator.

2. The method of claim 1, wherein the disease indicator comprises a score.

3. The method of claim 2, wherein generating the diagnosis output comprises:
determining that the score falls above a selected threshold; and
generating the diagnosis output based on the score falling above the selected threshold, wherein the diagnosis output includes a positive diagnosis for the high-grade advanced pre-malignant lesions or CRC disease state.

4. The method of claim 2, wherein generating the diagnosis output comprises:
determining that the score falls below a selected threshold; and
generating the diagnosis output based on the score falling below the selected threshold, wherein the diagnosis output includes a negative diagnosis for the high-grade advanced pre-malignant lesions or CRC disease state.

5. The method of claim 3 or claim 4, wherein the score comprises a support vector machine score and the selected threshold is 0.

6. The method of claim 3 or claim 4, wherein the selected threshold falls within a range between −0.1 and +0.1.

7. The method of any one of claims 1-6, wherein analyzing the peptide structure data comprises:
analyzing the peptide structure data using a binary classification model.

8. The method of any one of claims 1-7, wherein the at least one peptide structure comprises a glycopeptide structure defined by a peptide sequence and a glycan structure linked to the peptide sequence at a linking site of the peptide sequence, as identified in Table 1C, with the peptide sequence being one of SEQ ID NOS: 42-111 as defined in Table 3E.

9. The method of any one of claims 1-8, further comprising:
training the at least one supervised machine learning model using training data,
wherein the training data comprises a plurality of peptide structure profiles for a plurality of subjects and a plurality of subject diagnoses for the plurality of subjects.

10. The method of claim 9, further comprising:
performing a differential expression analysis using initial training data to compare a first portion of the plurality of subjects diagnosed with the positive diagnosis for the CRC or high-grade advanced pre-malignant lesions disease state versus a second portion of the plurality of subjects having the negative diagnosis for the high-grade advanced pre-malignant lesions or CRC disease state; and
identifying a training group of peptide structures based on the differential expression analysis for use as prognostic markers for the high-grade advanced pre-malignant lesions or CRC disease state; and
forming the training data based on the training group of peptide structures identified.

11. The method of any one of claims 1-10, wherein the peptide structure data comprises at least one of a raw abundance, an adjusted raw abundance, a peptide concentration, a glycopeptide concentration, or a normalized concentration.

12. The method of any one of claims 1-11, wherein the peptide structure data comprises normalized concentration data, wherein the normalized concentration data is a function of at least one of peptide abundance data, corresponding internal standard abundance data, a spike-in concentration value, and a dilution factor.

13. The method of any one of claims 1-12, wherein the peptide structure data is generated using multiple reaction monitoring mass spectrometry (MRM-MS).

14. The method of any one of claims 1-13, further comprising:
creating a sample from the biological sample; and
preparing the sample using reduction, alkylation, and enzymatic digestion to form a prepared sample that includes a set of peptide structures.

15. The method of claim 14, further comprising:
generating the peptide structure data from the prepared sample using multiple reaction monitoring mass spectrometry (MRM-MS).

16. The method of any one of claims 1-15, wherein generating the diagnosis output comprises:
generating a report identifying that the biological sample evidences the high-grade advanced pre-malignant lesions or CRC disease state.

17. The method of any one of claims 1-16, further comprising:
generating a treatment output based on at least one of the diagnosis output or the disease indicator.

18. The method of claim 17, wherein the treatment output comprises at least one of an identification of a treatment to treat the subject or a treatment plan.

19. The method of claim 18, wherein the treatment comprises at least one of radiation therapy, chemoradiotherapy, surgery, hormone therapy, or a targeted drug therapy.

20. A composition comprising at least one of peptide structures of PS-ID No's. 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91 identified in Table 1C.

20. A composition comprising a peptide structure or a product ion, wherein:
the peptide structure or the product ion comprises an amino acid sequence having at least 90% sequence identity to any one of SEQ ID NOS: 42-111, corresponding to peptide structures PS-ID No's. 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91 in Table 1C; and
the product ion is selected as one from a group consisting of product ions identified in Table 2C including product ions falling within an identified m/z range.

21. A composition comprising a glycopeptide structure selected as one peptide structure from a group consisting of PS-ID No's. 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91 identified in Table 1C, wherein:
the glycopeptide structure comprises:
an amino acid peptide sequence identified in Table 3E as corresponding to the glycopeptide structure; and
a glycan structure identified in Tables 5D and 5E as corresponding to the glycopeptide structure in which the glycan structure is linked to a residue of the amino acid peptide sequence at a corresponding position identified in Table 1C; and
wherein the glycan structure has a glycan composition.

22. The composition of claim 21, wherein the glycan composition is identified in Tables 5D and 5E.

23. The composition of claim 21, wherein:
the glycopeptide structure has a precursor ion having a charge identified in Table 2C as corresponding to the glycopeptide structure.

24. The composition of claim 21, wherein:
the glycopeptide structure has a precursor ion with an m/z ratio within ±1.5 of the m/z ratio listed for the precursor ion in Table 2C as corresponding to the glycopeptide structure.

25. The composition of claim 21, wherein:
the glycopeptide structure has a precursor ion with an m/z ratio within ±1.0 of the m/z ratio listed for the precursor ion in Table 2C as corresponding to the glycopeptide structure.

26. The composition of claim 21, wherein:
the glycopeptide structure has a precursor ion with an m/z ratio within ±0.5 of the m/z ratio listed for the precursor ion in Table 2C as corresponding to the glycopeptide structure.

27. The composition of claim 21, wherein:
the glycopeptide structure has a product ion with an m/z ratio within ±1.0 of the m/z ratio listed for the product ion in Table 2C as corresponding to the glycopeptide structure.

28. The composition of claim 21, wherein:
the glycopeptide structure has a product ion with an m/z ratio within ±0.8 of the m/z ratio listed for the product ion in Table 2C as corresponding to the glycopeptide structure.

29. The composition of claim 21, wherein:
the glycopeptide structure has a product ion with an m/z ratio within ±0.5 of the m/z ratio listed for the product ion in Table 2C as corresponding to the glycopeptide structure.

30. The composition of any one of claims 21-29, wherein the glycopeptide structure has a monoisotopic mass identified in Table 1C as corresponding to the glycopeptide structure.

31. A method of screening a subject, the method comprising
analyzing a peptide structure data using at least one supervised machine learning model to generate a disease indicator that indicates whether the biological sample evidences an high-grade advanced pre-malignant lesions or CRC disease state based on at least one peptide structure selected from a group of peptide structures identified in Table 1C, wherein peptide structure data corresponds to a biological sample obtained from the subject; and
outputting either a recommendation to perform a colonoscopy or to not perform the colonoscopy based on the disease indicator.

32. The method of claim 31, wherein the group of peptide structures in Table 1C is associated with the high-grade advanced pre-malignant lesions or CRC disease state.

33. The method of claims 31-32, wherein the group of peptide structures is listed in Table 1C with respect to relative significance to the disease indicator.

34. The method of claims 31-33, wherein the subject is subjected to a colonoscopy when the recommendation to perform the colonoscopy is outputted.

35. The method of claims 31-34, wherein the subject does not have any symptoms of high-grade advanced pre-malignant lesions and CRC.

36. The method of claims 31-35 further comprising: receiving peptide structure data corresponding to the biological sample obtained from the subject.

37. The method of claims 31-36, wherein the disease indicator comprises a score, wherein generating the diagnosis output comprises:
determining that the score falls above a selected threshold; and
generating the diagnosis output based on the score falling above the selected threshold, wherein the diagnosis output includes a positive diagnosis for the high-grade advanced pre-malignant lesions or CRC disease state.

38. The method of any one of claims 31-37, wherein analyzing the peptide structure data comprises:
analyzing the peptide structure data using a binary classification model.

39. The method of any one of claims 31-38, wherein the at least one peptide structure comprises a glycopeptide structure defined by a peptide sequence and a glycan structure linked to the peptide sequence at a linking site of the peptide sequence, as identified in Table 1C, with the peptide sequence being one of SEQ ID NOS: 42-111 as defined in Table 1C and Table 3E.

40. The method of any one of claims 31-39, wherein the peptide structure data comprises at least one of a raw abundance, an adjusted raw abundance, a peptide concentration, a glycopeptide concentration, or a normalized concentration.

41. The method of any one of claims 31-40, wherein the peptide structure data comprises normalized concentration data, wherein the normalized concentration data is a function of at least one of peptide abundance data, corresponding internal standard abundance data, a spike-in concentration value, and a dilution factor.

42. The method of any one of claims 31-41, wherein the peptide structure data is generated using multiple reaction monitoring mass spectrometry (MRM-MS).

43. The method of any one of claims 31-42, further comprising:
creating a sample from the biological sample; and
preparing the sample using reduction, alkylation, and enzymatic digestion to form a prepared sample that includes a set of peptide structures.

44. The method of claim 43, further comprising:
generating the peptide structure data from the prepared sample using multiple reaction monitoring mass spectrometry (MRM-MS).

45. The method of any one of claims 31-44, wherein the recommendation is a report identifying that the biological sample evidences the high-grade advanced pre-malignant lesions or CRC disease state.

46. A method for diagnosing a subject with respect to colorectal cancer (CRC) disease state that optionally includes one of adenoma, APL, and high-grade advanced pre-malignant lesion disease state, the method comprising:
receiving peptide structure data corresponding to a biological sample obtained from the subject;
analyzing the peptide structure data using at least one supervised machine learning model to generate a disease indicator that indicates whether the biological sample evidences the colorectal cancer (CRC) disease state that optionally includes one of adenoma, APL, and high-grade advanced pre-malignant lesion disease state based on at least one peptide structure selected from a group of peptide structures identified in Tables 1, 1B, 1C, 1D, and 13A;
wherein the group of peptide structures in Tables 1, 1B, 1C, 1D, and 13A is associated with colorectal cancer (CRC) disease state that optionally includes one of adenoma, APL, and high-grade advanced pre-malignant lesion disease state; and
generating a diagnosis output based on the disease indicator.
